(19) United States
(12) Patent Application Publication
(10) Pub. No.: US 2022/0144848 A1
(43) Pub. Date: May 12, 2022

(54) SUBSTITUTED OXOPYRIDINE DERIVATIVES

(71) Applicants: Bayer Aktiengesellschaft, Leverkusen (DE); Bayer Pharma Aktiengesellschaft, Berlin (DE)

(72) Inventors: Susanne ROEHRIG, Hilden (DE); Sebastian ESSIG, Erkrath (DE); Pascal ELLERBROCK, Duesseldorf (DE); Sonja ANLAUF, Wermelskirchen (DE); Thomas NEUBAUER, Wuppertal (DE); Alexander HILLISCH, Solingen (DE); Katharina MEIER, Mettmann (DE); Stefan HEITMEIER, Wuelfrath (DE); Adrian TERSTEEGEN, Wuppertal (DE); Martina SCHAFER, Berlin (DE); Jan STAMPFUSS, Duesseldorf (DE); Dieter LANG, Velbert (DE); Eloisa JIMENEZ NUNEZ, Freiburg im Breisgau (DE); Jens ACKERSTAFF, Duesseldorf (DE); Henrik TELLER, Hohenfelde (DE); Zengqiang ZOU, Beijing (CN); Ping LIU, Beijing (CN); Xianghai MENG, Beijing (CN); Frank LESSMANN, Wolfen (DE)

(21) Appl. No.: 17/416,274

(22) PCT Filed: Dec. 18, 2019
(86) PCT No.: PCT/EP2019/085989

§ 371 Date: Jun. 18, 2021

(30) Foreign Application Priority Data
Dec. 21, 2018 (CN) PCT/CN2018/122826

(51) Int. Cl. C07D 491/044

(52) U.S. Cl. CPC C07D 491/044

(57) ABSTRACT

The invention relates to substituted oxopyridine derivatives and to processes for their preparation, and also to their use for preparing medicaments for the treatment and/or prophylaxis of diseases, in particular vascular disorders, preferably thrombotic or thromboembolic disorders and/or thrombotic or thromboembolic complications.

The invention relates to substituted oxopyridine derivatives and to processes for their preparation, and also to their use for preparing medicaments for the treatment and/or prophylaxis of diseases, in particular vascular disorders, preferably thrombotic or thromboembolic disorders and/or thrombotic or thromboembolic complications.

Haemostasis is a protective mechanism of the organism, which helps to “seal” leaking damages in the blood vessel wall quickly and reliably. Thus, excessive loss of blood can often be avoided or kept to a minimum. After injury of a blood vessel, hemostasis is conducted mainly by activation and aggregation of platelets and activation the coagulation system, which consists of an enzymatic “waterfall” cascade leading one after another to the activation of the next coagulation factor until thrombin is formed, which leads to the generation of insoluble fibrin, which is an important part of the clot.

In the more recent past, the traditional theory of two separate starting points of the coagulation cascade (extrinsic and intrinsic path) has been modified owing to new findings: In these models, coagulation is initiated by binding of activated factor VIIa to tissue factor (TF). The resulting complex activates factor X, which in turn leads to generation of thrombin with subsequent production of fibrin and platelet activation (via PAR-1) as injury-sealing end products of haemostasis. Compared to the subsequent amplification/propagation phase, the thrombin production rate in this first phase is low and as a result of the occurrence of TFPI as inhibitor of the TF-FVIIa-FX complex is limited in time. A central component of the transition from initiation to amplification of coagulation and thereby thrombus propagation is factor XIa: in positive feedback loops, thrombin activates not only factor V and factor VIII, but also factor XI to factor XIa, which in turn converts factor IX into factor IXa, which in turn in a factor IXa/factor VIIIa complex generates factor Xa and finally to large amounts of thrombin, resulting in strong thrombus growth and stabilization of the thrombus. This is supported by TAFIa and FXIIIa, which are activated by thrombin as well and lead to inhibition of clot lysis and further clot stabilisation.

In addition to the stimulation via tissue factor, the coagulation system can be activated particularly on negatively charged surfaces, which include not only surface structures of foreign cells (e.g. bacteria) but also artificial surfaces such as vascular prostheses, stents and extracoporeal circulation. On these surfaces, factor XII (FXII) is activated to factor XIIa, which subsequently activates factor XI to factor XIa. This leads to further activation of the coagulation cascade as described above. In addition, factor XIIa also activates bound plasma prokallikrein to plasma kallikrein (PK) which, in a potentiation loop, firstly leads to further factor XII activation, overall resulting in amplification of the initiation of this intrinsic part of the coagulation cascade.

Uncontrolled activation of the coagulation system or defective inhibition of the activation processes may lead to the formation of local thrombi or emboli in vessels (e.g. arteries, veins, lymph vessels) or in organ cavities (e.g. cardiac atrium). In addition, systemic hypercoagulability may lead to system-wide formation of microthrombi and finally to a consumption coagulopathy in the context of a disseminated intravasal coagulation. Thromboembolic complications may also occur in extracorporeal circulatory systems, such as haemodialysis, and also in vascular prostheses or prosthetic heart valves and stents.

In the course of many cardiovascular and metabolic disorders, increased tendency for coagulation and platelet activation occur owing to either systemic factors such as hyperlipidaemia, diabetes, inflammation, infection or smoking, or to changes in blood flow with stasis, for example in in diseased leg veins or in atrial fibrillation, or owing to pathological changes in vessel walls, for example endothelial dysfunctions or atherosclerosis. This unwanted and excessive activation of coagulation may, by formation of fibrin- and platelet-rich thrombi, lead to thromboembolic disorders and thrombotic complications with often life-threatening events. Inflammation processes may also be involved by triggering the coagulation system. On the other hand, thrombin is known to activate inflammatory pathways, as well.

Accordingly, thromboembolic disorders are still the most frequent cause of morbidity and mortality in most industrialized countries.

The anticoagulants known from the prior art, that is to say substances for inhibiting or preventing blood coagulation, have various disadvantages. Accordingly, in practice, efficient treatment methods or the prophylaxis of thrombotic/thromboembolic disorders is found to be difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described herein below encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicomentosa or osteoporosis. Low-molecular-weight heparins do have a lower probability of leading to the development of heparin-induced thrombocytopaenia; however, they can also only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of vitamin K-dependent coagulation factors in the liver. Owing to the mechanism of action, the onset of action is only very slow (latency to the onset of action 36 to 48 hours). The compounds can be administered orally; however, owing to the high risk of bleeding and the narrow therapeutic index complicated individual adjustment and monitoring of the patient are required. In addition, other side-effects such as gastrointestinal problems, hair loss and skin necroses have been described.

Today, approaches for Non-vitamin K dependent oral anticoagulantion (NOACs) are in clinical use, and have demonstrated their effectiveness in various studies. However, taking these medicaments can also lead to bleeding complications, particularly in predisposed patients.

Thus, for antithrombotic medicaments, the therapeutic window is of central importance: The interval between the therapeutically active dose for coagulation inhibition and the dose where bleeding may occur should be as large as possible so that maximum therapeutic activity is achieved at a minimum risk profile.

In various in vitro and in vivo models with, for example, antibodies as factor XIa inhibitors, but also in factor XIa knock-out animal models, the antithrombotic effect with small/no prolongation of bleeding time or extension of blood volume was confirmed. In clinical studies, elevated factor XIa concentrations were associated with an increased thrombotic event rate. In contrast, factor XI deficiency (haemophilia C) did not lead to spontaneous bleeding and was apparent only in the course of surgical operations and traumata, but did show protection with respect to certain thromboembolic events.

Furthermore, for many disorders the combination of antithrombotic and antiinflammtory principles may also be particularly attractive to prevent the mutual enhancement of coagulation and inflammation.

It is therefore an object of the present invention to provide novel compounds for the treatment of cardiovascular disorders, in particular of thrombotic or thromboembolic disorders and/or thrombotic or thromboembolic complications, in humans and animals, which compounds have a wide therapeutic window.

WO 2006/030032 describes inter alia substituted pyridinones as allosteric modulators of the mGluR2 receptor, and WO 2008/079787 describes substituted pyridin-2-ones and their use as glucokinase activators. WO 2014/154794, WO 2014/160592, WO 2015/011087, WO 2015/063093, WO 2016/046158, WO 2016/046157, WO 2016/046159, WO 2016/046164, WO 2016/046166, WO 2016/046156, WO 2017/005725 and WO 2017/037051 describe substituted pyridin-2-ones and their use as factor XIa inhibitors.

The invention provides compounds of the formula

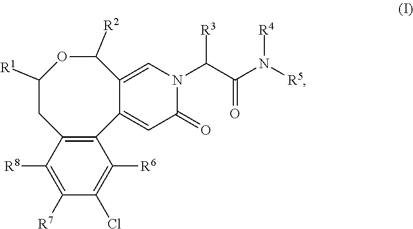

in which
R¹ represents methyl, ethyl, iso-propyl, hydroxymethyl, difluoromethyl or trifluoromethyl,
R² represents hydrogen or methyl,
R³ represents methyl, ethyl or n-propyl,
   where methyl may be substituted with one substituent selected from the group consisting of cyclopropyl, cyclobutyl, oxetan-2-yl, oxetan-3-yl, tetrahydrofuran-2-yl, tetrahydro-2H-pyran-2-yl, tetrahydro-2H-pyran-4-yl and 1,4-dioxan-2-yl,
      where oxetan-2-yl, tetrahydrofuran-2-yl, tetrahydro-2H-pyran-2-yl and 1,4-dioxan-2-yl may be substituted by 1 to 2 substituents independently of one another selected from the group consisting of fluorine and methyl,
   or
   where methyl may be substituted with one substituent of the group of the formula

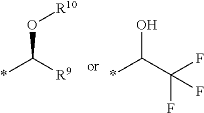

where
* is the attachment site to the methyl group,
R⁹ represents methyl, ethyl, iso-propyl, cyclopropyl, difluoromethyl or trifluoromethyl,
R¹⁰ represents methyl or difluoromethyl,
and
where ethyl may be substituted with one substituent selected from the group consisting of methoxy, ethoxy, iso-propoxy, tert-butoxy, difluoromethoxy, trifluoromethoxy, 2,2-difluoroethoxy, 2,2,2-trifluoroethoxy, cyclopropyloxy and cyclobutyloxy,
   where cyclopropyloxy and cyclobutyloxy may be substituted with one substituent selected from the group consisting of fluorine and methyl,
R⁴ represents hydrogen,
R⁵ represents a group of the formula

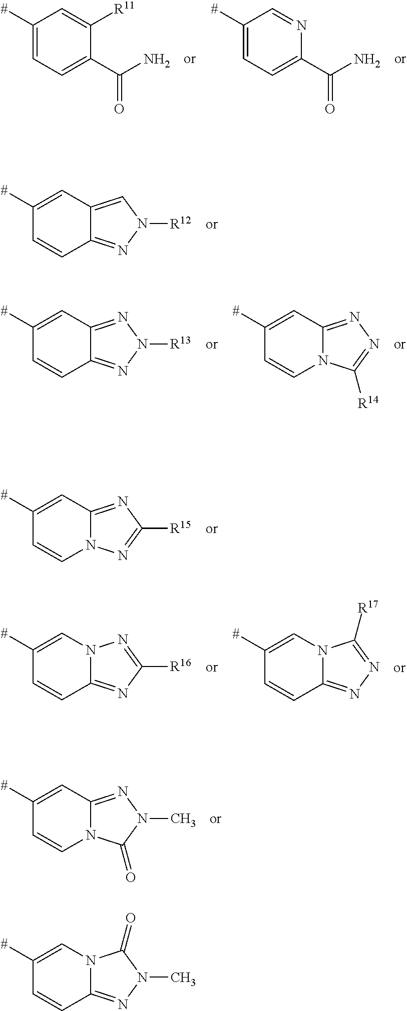

where
# is the attachment site to the nitrogen atom,
R¹¹ represents hydrogen or fluorine,
R¹² represents methyl, difluoromethyl or trifluoromethyl,
R¹³ represents methyl, difluoromethyl or trifluoromethyl,
R¹⁴ represents hydrogen or methyl,
R¹⁵ represents hydrogen or methyl,
R¹⁶ represents hydrogen or methyl,
R¹⁷ represents hydrogen or methyl.
R⁶, R⁷ and R⁸ represent the following:
R⁶ represents hydrogen, fluorine or chlorine,
R⁷ represents hydrogen,
R⁸ represents hydrogen,
or
R⁶ represents hydrogen,
R⁷ represents fluorine or chlorine,
R⁸ represents hydrogen,
or
R⁶ represents hydrogen,
R⁷ represents hydrogen,
R⁸ represents fluorine,
and the salts thereof, the solvates thereof and the solvates of the salts thereof.

Compounds according to the invention are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The inventive compounds may, depending on their structure, exist in different stereoisomeric forms, i.e. in the form of configurational isomers or else, if appropriate, of conformational isomers (enantiomers and/or diastereomers, including those in the case of rotamers and atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the compounds according to the invention can occur in tautomeric forms, the present invention encompasses all the tautomeric forms.

In the context of the present invention, the term “enantiomerically pure” is understood to mean that the compound in question with respect to the absolute configuration of the chiral centre is present in an enantiomeric excess of more than 95%, preferably more than 97%. The enantiomeric excess (ee value) is calculated in this case by evaluation of the corresponding HPLC chromatogram on a chiral phase with the aid of the formula below:

ee=[E^{A}(area %)−E^{B}(area %)]×100%/[E^{A}(area %)+E^{B}(area %)]

(E^{A}: enantiomer in excess, E^{B}: enantiomer in deficiency)

The present invention also encompasses all suitable isotopic variants of the compounds according to the invention. An isotopic variant of an inventive compound is understood here as meaning a compound in which at least one atom within the inventive compound has been exchanged for another atom of the same atomic number, but with a different atomic mass than the atomic mass which usually or predominantly occurs in nature. Examples of isotopes which can be incorporated into a compound according to the invention are those of hydrogen, carbon, nitrogen, oxygen, phosphorus, sulfur, fluorine, chlorine, bromine and iodine, such as ²H (deuterium), ³H (tritium), ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I. Particular isotopic variants of a compound according to the invention, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, may lead to particular therapeutic benefits as a consequence of greater metabolic stability of the compound, for example an extension of the half-life in the body or a reduction in the active dose required; such modifications of the inventive compounds may therefore in some cases also constitute a preferred embodiment of the present invention. Isotopic variants of the compounds according to the invention can be prepared by the processes known to those skilled in the art, for example by the methods described further below and the procedures described in the working examples, by using corresponding isotopic modifications of the respective reagents and/or starting compounds.

Preferred sails in the context of the present invention are physiologically acceptable salts of the compounds according to the invention. However, the invention also encompasses salts which themselves are unsuitable for pharmaceutical applications but which can be used, for example, for the isolation or purification of the compounds according to the invention.

Physiologically acceptable salts of the compounds according to the invention include acid addition salts of mineral acids, carboxylic acids and sulfonic acids, for example salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, trifluoroacetic acid, propionic acid, lactic acid, tartaric acid, malic acid, citric acid, fumaric acid, maleic acid and benzoic acid.

Physiologically acceptable salts of the compounds according to the invention also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

Solvatcs in the context of the invention are described as those forms of the inventive compounds which form a complex in the solid or liquid state by coordination with solvent molecules. Hydrates are a specific form of the solvates in which the coordination is with water.

The present invention additionally also encompasses prodrugs of the inventive compounds. The term “prodrugs” encompasses compounds which for their part may be biologically active or inactive but are converted during their residence time in the body into compounds according to the invention (for example by metabolism or hydrolysis).

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” and “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the formulae of the group which may represent a substituent to the methyl group in R³, the end point of the line marked by * in each case does not represent a carbon atom or a CH₂ group, but is part of the bond to the atom to which the group is attached.

In the formulae of the group which may represent R⁵, the end point of the line marked by # in each case does not represent a carbon atom or a CH₂ group, but is part of the bond to the atom to which R⁵ is attached.

Preference is given to compounds of the formula (I) in which
R¹ represents methyl, ethyl, iso-propyl, hydroxymethyl, difluoromethyl or trifluoromethyl,
R² represents hydrogen or methyl,
R³ represents methyl or ethyl,
   where methyl may be substituted with one substituent selected from the group consisting of tetrahydro-2H-pyran-2-yl and 1,4-dioxan-2-yl,
   or
   where methyl may be substituted with one substituent of the group of the formula

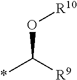

where
* is the attachment site to the methyl group,
R⁹ represents methyl, cyclopropyl or difluoromethyl,
R¹⁰ represents methyl or difluoromethyl,
and
where ethyl may be substituted with one substituent selected from the group consisting of methoxy, iso-propoxy, tert-butoxy, difluoromethoxy and cyclopropyloxy,
R⁴ represents hydrogen,
R⁵ represents a group of the formula

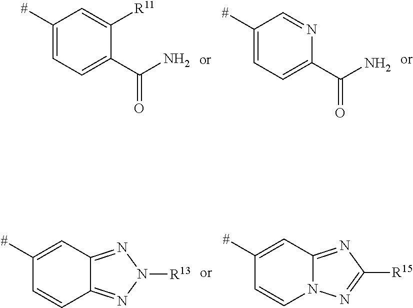

where
# is the attachment site to the nitrogen atom,
R¹¹ represents hydrogen or fluorine,
R¹³ represents methyl,
R¹⁵ represents hydrogen,
R⁶, R⁷ and R⁸ represent the following:
R⁶ represents hydrogen, fluorine or chlorine,
R⁷ represents hydrogen,
R⁸ represents hydrogen,
and the salts thereof, the solvates thereof and the solvates of the salts thereof.

Preference is also given to compounds of the formula (I) in which
R¹ represents methyl, difluoromethyl or trifluoromethyl,
R² represents hydrogen or methyl,
R³ represents methyl or ethyl,
   where methyl is substituted with one substituent selected from the group consisting of tetrahydro-2H-pyran-2-yl and 1,4-dioxan-2-yl,
   or
   where methyl is substituted with one substituent of the group of the formula

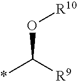

where
* is the attachment site to the methyl group,
R⁹ represents methyl,
R¹⁰ represents methyl or difluoromethyl,
and
where ethyl is substituted with one substituent selected from the group consisting of methoxy, iso-propoxy, tert-butoxy, difluoromethoxy and cyclopropyloxy,
R⁴ represents hydrogen,
R⁵ represents a group of the formula

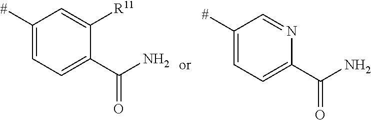

where
# is the attachment site to the nitrogen atom,
R¹¹ represents hydrogen or fluorine,
R⁶, R⁷ and R⁸ represent the following:
R⁶ represents hydrogen or fluorine,
R⁷ represents hydrogen,
R⁸ represents hydrogen,
   and the salts thereof, the solvates thereof and the solvates of the salts thereof.

Preference is also given to compounds of the formula (I) in which
R¹ represents methyl, difluoromethyl or trifluoromethyl,
R² represents hydrogen or methyl,
R³ represents methyl,
   where methyl is substituted with one substituent selected from the group consisting of tetrahydro-2H-pyran-2-yl and 1,4-dioxan-2-yl,
   or
   where methyl is substituted with one substituent of the group of the formula

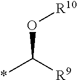

where
* is the attachment site to the methyl group,
R⁹ represents methyl,
R¹⁰ represents methyl,
R⁴ represents hydrogen,
R⁵ represents a group of the formula

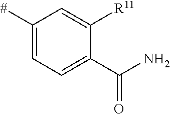

where
# is the attachment site to the nitrogen atom,
R¹¹ represents hydrogen,
R⁶, R⁷ and R⁸ represent the following:
R⁶ represents hydrogen or fluorine,
R⁷ represents hydrogen,
R⁸ represents hydrogen,
   and the salts thereof, the solvates thereof and the solvates of the salts thereof.

Preference is also given to compounds of the formula (I) in which
R⁶, R⁷ and R⁸ represent the following:
R⁶ represents hydrogen or fluorine,
R⁷ represents hydrogen,
R⁸ represents hydrogen.

Preference is also given to compounds of the formula (I) in which
R⁶, R⁷ and R⁸ represent the following:
R⁶ represents hydrogen,
R⁷ represents hydrogen,
R⁸ represents hydrogen.

Also preferred are compounds having the formula (Ia)

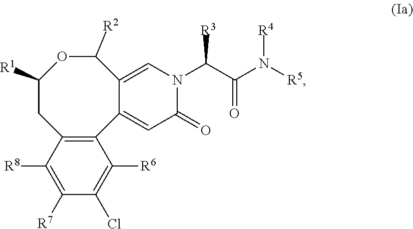

in which R¹, R², R³, R⁴, R⁵, R⁶, R⁷ and R⁸ are as defined above.

Preference is also given to the compound 4-({(2S)-2-[(7R)-11-Chloro-7-methyl-2-oxo-7,8-dihydro-2H-[3]benzoxocino[5,6-c]pyridin-3(5H)-yl]-3-[(2S)-tetrahydro-2H-pyran-2-yl]propanoyl}-amino)benzamide (single stereoisomer) of the formula below

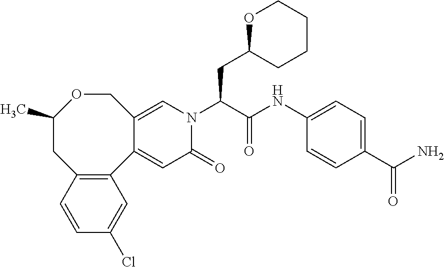 [SMILES: C[C@@H]1Cc2ccc(Cl)cc2-c2cc(=O)n([C@@H](C[C@@H]3CCCCO3)C(=O)Nc3ccc(C(N)=O)cc3)cc2CO1]

or one of the salts thereof, solvates thereof or solvates of the salts thereof.

Preference is also given to the compound 4-({(2S)-2-[(7S)-11-Chloro-2-oxo-7-(trifluoro-methyl)-7,8-dihydro-2H-[3]benzoxocino[5,6-c]pyridin-3(5H)-yl]-3-[(2S)-tetrahydro-2H-pyran-2-yl]propanoyl}amino)benzamide (single stereoisomer) of the formula below

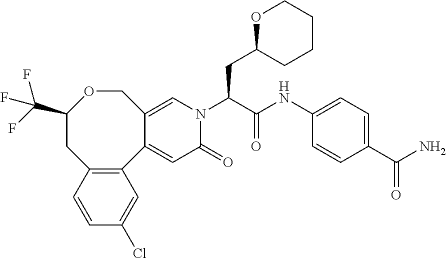 [SMILES: NC(=O)c1ccc(NC(=O)[C@H](C[C@@H]2CCCCO2)n2cc3c(cc2=O)-c2cc(Cl)ccc2C[C@@H](C(F)(F)F)OC3)cc1]

or one of the salts thereof, solvates thereof or solvates of the salts thereof.

Preference is also given to the compound 4-({(2S)-2-[(7S)-11-Chloro-2-oxo-7-(trifluoroethyl)-7,8-dihydro-2H-[3]benzoxocino[5,6-c]pyridin-3(5H)-yl]-3-[(2R)-1,4-dioxan-2-yl]propanoyl}-amino)benzamide (single stereoisomer) of the formula below

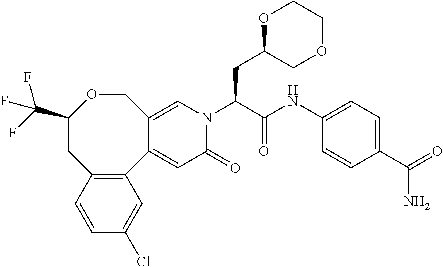 [SMILES: NC(=O)c1ccc(NC(=O)[C@H](C[C@@H]2COCCO2)n2cc3c(cc2=O)-c2cc(Cl)ccc2C[C@@H](C(F)(F)F)OC3)cc1]

or one of the salts thereof, solvates thereof or solvates of the salts thereof.

Preference is also given to the compound 4-({(2S)-2-[(5S,7R)-11-Chloro-5,7-dimethyl-2-oxo-7,8-dihydro-2H-[3]benzoxocino[5,6-c]pyridin-3(5H)-yl]-3-[(2S)-tetrahydro-2H-pyran-2-yl]propanoyl}-amino)benzamide (single stereoisomer) of the formula below

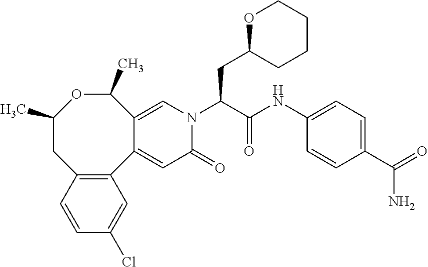 [SMILES: C[C@@H]1Cc2ccc(Cl)cc2-c2cc(=O)n([C@@H](C[C@@H]3CCCCO3)C(=O)Nc3ccc(C(N)=O)cc3)cc2[C@H](C)O1]

or one of the salts thereof, solvates thereof or solvates of the salts thereof.

Preference is also given to the compound 4-({(2S,4S)-2-[(5S,7R)-11-Chloro-5,7-dimethyl-2-oxo-7,8-dihydro-2H-[3]benzoxocino[5,6-c]pyridin-3(5H)-yl]-4-methoxypentanoyl}-amino)benzamide (single stereoisomer) of the formula below

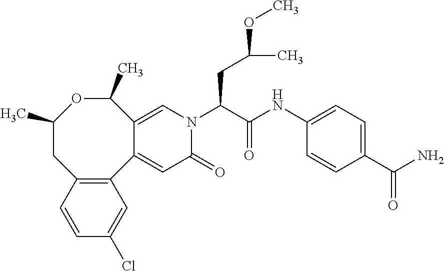 [SMILES: CO[C@@H](C)C[C@@H](C(=O)Nc1ccc(C(N)=O)cc1)n1cc2c(cc1=O)-c1cc(Cl)ccc1C[C@@H](C)O[C@H]2C]

or one of the salts thereof, solvates thereof or solvates of the salts thereof.

The invention further provides a method for preparing compounds of the formula (I), or salts thereof, solvates thereof or solvates of the salts thereof, wherein

[A] the compounds of the formula

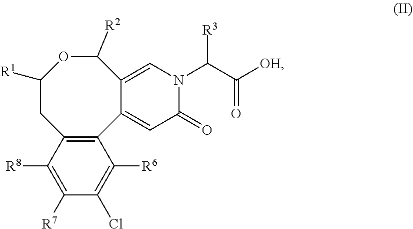

in which
R¹, R², R³, R⁶, R⁷ and R⁸ are as defined above,
are reacted with compounds of the formula

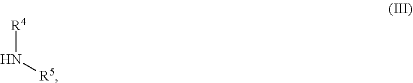

in which
R⁴ and R⁵ are as defined above,
in the presence of a dehydrating agent to give compounds of the formula (I)
or
[B] the compounds of the formula (II) are converted in a one-pot reaction to the acid chloride of compounds of the formula (II) and then the acid chlorides are reacted with compounds of the formula (III) to give compounds of the formula (I)
or
[C] the compounds of the formula

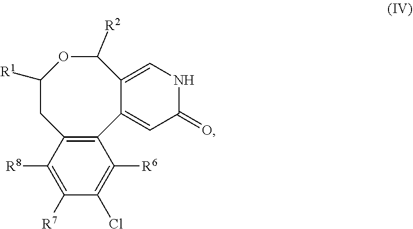

in which
R¹, R², R⁶, R⁷ and R⁸ are as defined above,
are reacted with compounds of the formula

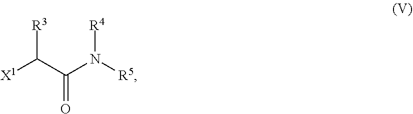

in which
X¹ represents bromine, iodine or trifluoromethane-sulfonyloxy,
in the presence of a base to give compounds of the formula (I).

The reaction according to process [A] is generally carried out in inert solvents, if appropriate in the presence of a base, preferably in a temperature range from −20° C. to 80° C. at atmospheric pressure.

Alternatively, the reaction can also be carried out without a solvent only in one base if the base is a liquid at RT.

Suitable dehydrating agents here are, for example, carbodiimides such as N,N′-diethyl-, N,N′-dipropyl-, N,N′-diisopropyl-, N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimide-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulfate or 2-tert-butyl-5-methyl-isoxazolium perchlorate, or acylamino compounds such as 2-ethoxy-1-ethoxycarbonyl-1,2-dihydroquinoline, or isobutyl chloroformate, or bis-(2-oxo-3-oxazolidinyl)phosphoryl chloride or benzotriazolyloxytri(dimethylamino)-phosphonium hexafluorophosphate, or O-(benzotriazol-1-yl)-N,N,N,N′-tetramethyluronium hexafluorophosphate (HBTU), 2-(2-oxo-1-(2H)-pyridyl)-1,1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluoro-phosphate (HATU), or 1-hydroxybenzotriazole (HOBt), or benzotriazol-1-yloxytris(dimethyl-amino)phosphonium hexafluorophosphate (BOP), or ethyl cyano(hydroxyimino)acetate (Oxyma), or (1-cyano-2-ethoxy-2-oxoethylidenaminooxy)dimethylamino-morpholino-carbenium hexafluorophosphate (COMU), or N-[(dimethylamino)(3H-[1,2,3]triazolo[4,5-b]pyridin-3-yloxy)methylidene]-N-methylmethanaminium hexafluorophosphate, or 2,4,6-tripropyl-1,3,5,2,4,6-trioxatriphosphinane-2,4,6-trioxide (T3P), or mixtures of these with bases, the condensation with HATU or with T3P being preferred.

Bases are, for example, organic bases such as trialkylamines, for example triethylamine, N-methylmorpholine, N-methylpiperidine, 4-dimethylaminopyridine or diisopropylethylamin, or pyridine, preference is given to condensation with diisopropylethylamine or pyridine.

Inert solvents are, for example, halogenated hydrocarbons such as dichloromethane or trichloromethane, hydrocarbons such as benzene or toluene, or other solvents such as 1,4-dioxane, diethyl ether, tetrahydrofuran, ethyl acetate, N,N-dimethylformamide, dimethyl sulfoxide or acetonitrile, or mixtures of the solvents, preference being given to N,N-dimethylformamide or tetrahydrofuran.

The reaction according to process [B] is generally carried out in inert solvents, in the presence of a chlorination agent, preferably in a temperature range from −20° C. to 80° C. at atmospheric pressure.

Inert solvents are, for example, halogenated hydrocarbons such as dichloromethane or trichloromethane, hydrocarbons such as benzene or toluene, or other solvents such as 1,4-dioxane, diethyl ether, tetrahydrofuran, ethyl acetate or N,N-dimethylformamide, or mixtures of the solvents, preference being given to dichloromethane.

Chlorination agents are, for example, 1-chloro-N,N,2-trimethylprop-1-en-1-amine, oxalyl chloride, sulfurous dichloride, preference being given to 1-chloro-N,N,2-trimethylprop-1-en-1-amine.

The reaction according to process [C] is generally carried out in inert solvents, preferably in a temperature range from room temperature to reflux of the solvents at atmospheric pressure.

Bases are, for example, alkali metal hydroxides such as sodium hydroxide, lithium hydroxide or potassium hydroxide, or alkali metal carbonates such as caesium carbonate, sodium carbonate or potassium carbonate, or potassium tert-butoxide or sodium tert-butoxide, sodium hydride or a mixture of these bases or a mixture of sodium hydride and lithium bromide, or organic bases such as 1,1,3,3-tetramethylguanidine or 2-tert-butylimino-2-diethylamino-1,3-dimethylperhydro-1,3,2-diazaphosphorine (BEMP), preference is given to potassium carbonate or sodium hydride or 1,1,3,3-tetramethylguanidine.

Inert solvents are, for example, halogenated hydrocarbons such as dichloromethane, trichloromethane, carbon tetrachloride or 1,2-dichloroethane, alcohols such as methanol, ethanol or 2-propanol, ethers such as diethyl ether, methyl tert-butyl ether, 1,2-dimethoxyethane, 1,4-dioxane or tetrahydrofuran, or other solvents such as N,N-dimethylformamide, N,N-dimethylacetamide, acetonitrile, pyridine or acetone, or mixtures of solvents, or mixtures of solvents with water, preference is given to N,N-dimethylformamide or to a mixture of acetone and 2-propanol.

The compounds of the formula (III) are known or can be synthesized from the corresponding starting compounds by known processes.

The compounds of the formula (V) are known, can be synthesized from the corresponding starting compounds by known processes or can be prepared analogously to the processes described in the Examples section.

The compounds of the formula (II) are known or can be prepared by reacting
[D] compounds of the formula

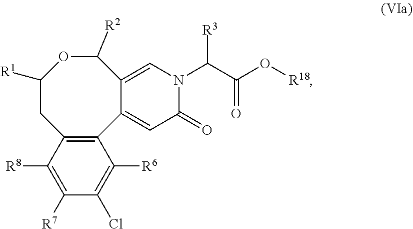

in which
R¹, R², R³, R⁶, R⁷ and R⁸ are each as defined above, and
R¹⁸ represents tert-butyl,
with an acid to give compounds of the formula (II)
or
[E] compounds of the formula

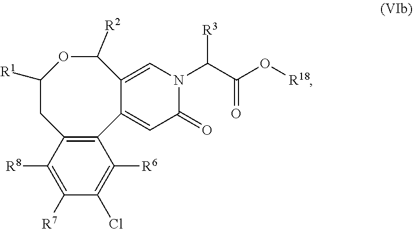

in which
R¹, R², R³, R⁶, R⁷ and R⁸ are each as defined above, and
R¹⁸ represents methyl, ethyl, tert-butyl or benzyl,
with a base to give compounds of the formula (II).

The reaction according to process [D] is generally carried out in inert solvents, preferably in a temperature range from 0° C. to 60° C. at atmospheric pressure.

Inert solvents are, for example, halogenated hydrocarbons such as dichloromethane, trichloromethane, carbon tetrachloride or 1,2-dichloroethane, or ethers such as tetrahydrofuran or 1,4-dioxane, preference being given to dichloromethane.

Acids are, for example, trifluoroacetic acid or hydrogen chloride in 1,4-dioxane, preference being given to trifluoroacetic acid.

The reaction according to process [E] is generally carried out in solvents, preferably in a temperature range from room temperature up to reflux of the solvents at atmospheric pressure.

Inert solvents are, for example, alcohols such as methanol or ethanol, ethers such as diethyl ether, methyl tert-butyl ether, 1,2-dimethoxyethane, 1,4-dioxane or tetrahydrofuran, or mixtures of solvents, or mixtures of solvents with water, preference being given to a mixture of tetrahydrofuran and water.

Bases are, for example, alkali metal hydroxides such as sodium hydroxide, lithium hydroxide or potassium hydroxide, or alkali metal carbonates such as caesium carbonate, sodium carbonate or potassium carbonate, preference being given to lithium hydroxide.

The compounds of the formulae (VIa) and (VIb) together form the group of the compounds of the formula (VI).

The compounds of the formula (VIa) are known or can be prepared by reacting compounds of the formula

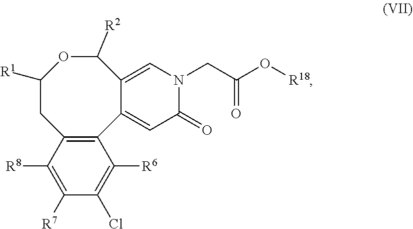

in which
R¹, R², R⁶, R⁷ and R⁸ are each as defined above, and
R¹⁸ represents tert-butyl,
with compounds of the formula

R³—X² (VIII),
in which
R³ is as defined above, and
X² represents chlorine, bromine, iodine or trifluoromethanesulfonyloxy.

The reaction is generally carried out in inert solvents, in the presence of a base, preferably in a temperature range from −78° C. to room temperature at atmospheric pressure.

Inert solvents are, for example, ethers such as diethyl ether, methyl tert-butyl ether, 1,2-dimethoxyethane, 1,4-dioxane or tetrahydrofuran, or mixtures of solvents, or mixtures of solvent with water, preference is given to tetrahydrofuran.

Bases are, for example, potassium tert-butoxide or sodium tert-butoxide, sodium hydride, n-butyllithium, lithium bis(trimethylsilyl)amide or sodium bis(trimethylsilyl)amide, preference is given to lithium bis(trimethylsilyl)amide or sodium bis(trimethylsilyl)amide.

The compounds of the formula (VIII) are known or can be synthesized from the corresponding starting compounds by known processes.

The compounds of the formula (VII) are known or can be prepared by reacting compounds of the formula

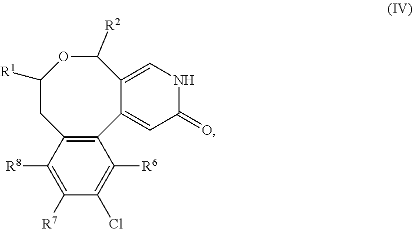

in which
R¹, R², R⁶, R⁷ and R⁸ are as defined above,
with compounds of the formula

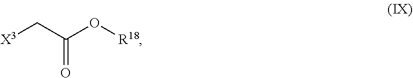

in which
X³ represents chlorine, bromine, iodine, methanesulfonyloxy or trifluoromethane-sulfonyloxy and
R¹⁸ represents tert-butyl.

The reaction is carried out as described for process [C].

The compounds of the formula (IX) are known or can be synthesized from the corresponding starting compounds by known processes.

In an alternative process, the compounds of the formula (VI) can be prepared by reacting compounds of the formula (IV) with compounds of the formula

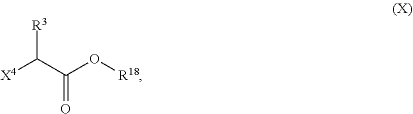

in which
R³ is as defined above,
X⁴ represents chlorine, bromine, iodine, methanesulfonyloxy or trifluoromethane-sulfonyloxy and
R¹⁸ represents methyl, ethyl, tert-butyl or benzyl.

The reaction is carried out as described for process [C].

The compounds of the formula (X) are known, can be synthesized from the corresponding starting compounds by known processes or can be prepared analogously to the processes described in the Examples section.

The compounds of the formula (IV) are known or can be prepared by reacting compounds of the formula

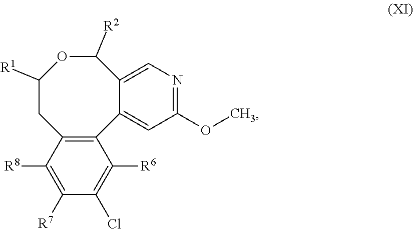

in which
R¹, R², R⁶, R⁷ and R⁸ are as defined above,
with pyridinium hydrochloride or pyridinium hydrobromide or sodium iodide and an acid or lithium iodide and an acid or with a thiol, such as ethanethiol, and a base.

The reaction is generally carried out in inert solvents or without solvents, preferably in a temperature range of from 80° C. to 120° C. at atmospheric pressure.

Inert solvents are, for example, hydrocarbons such as benzene, or alcohols such as methanol, ethanol or 1-butanol, or other solvents such as nitromethane, 1,4-dioxane, N,N-dimethylformamide, dimethyl sulfoxide or acetonitrile, or a mixture of the solvents, preference is given to N,N-dimethylformamide or 1-butanol.

Acids are, for example, 4-toluenesulfonic acid monohydrate, formic acid, acetic acid, trifluoroacetic acid, preference is given to acetic acid and 4-toluenesulfonic acid monohydrate.

Bases are, for example, potassium tert-butoxide or sodium tert-butoxide or sodium hydride, preference is given to sodium hydride.

The compounds of the formula (XI) are known or can be prepared by reacting compounds of the formula

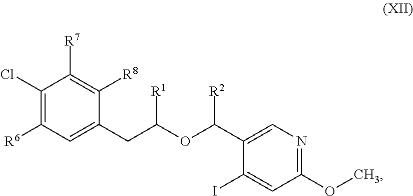

in which
R¹, R², R⁶, R⁷ and R⁸ are as defined above,
with a base in the presence of a catalyst.

The reaction is generally carried out in inert and degassed solvents, preferably within a temperature range from 80° C. to 150° C. at atmospheric pressure.

Bases are, for example, alkali metal carbonates such as sodium carbonate or potassium carbonate, organic bases such as sodium pivalate, potassium pivalate, caesium pivalate, sodium acetate, potassium acetate, caesium acetate, preference is given to potassium pivalate or potassium acetate.

Catalysts are, for example, palladium catalysts customary for CH-activation conditions, such as dichlorobis(triphenylphosphine)palladium, tetrakis(triphenylphosphine)palladium(0), palladium(II) acetate/triscyclohexylphosphine, bis(tri-tert-butylphosphine)palladium(0), tris(dibenzylidene-acetone)dipalladium, bis(diphenylphosphaneferrocenyl)palladium(II) chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, XPhos precatalyst [(2′-aminobiphenyl-2-yl)(chloro)palladium dicyclohexyl(2′,4′,6′-triisopropylbiphenyl-2-yl)phosphane (1:1)], DavePhos precatalyst [Methanesulfonato 2-dicyclohexylphosphino-2-(N,N-dimethylamino)biphenyl(2′-amino-1,1′-biphenyl-2-yl) palladium(II)], PEPPSI®-catalysts such as [1,3-bis(2,6-di-3-pentylphenyl)imidazol-2-ylidene](3-chloropyridyl)palladium(II) dichloride or [1,3-bis(2,6-diisopropylphenyl)imidazol-2-ylidene](3-chloropyridyl)palladium(II) dichloride, preference being given to tetrakis(triphenylphosphine)palladium(0) or [1,3-bis(2,6-di-3-pentylphenyl)imidazol-2-ylidene](3-chloropyridyl)palladium(II dichloride.

Inert solvents are, for example, ethers such as 1,4-dioxane, tetrahydrofuran or 1,2-dimethoxyethane, hydrocarbons such as benzene, xylene or toluene, or carboxamides such as N,N-dimethylformamide or N,N-dimethylacetamide, alkyl sulfoxides such as dimethyl sulfoxide, or mixtures of the solvents with water, preference is given to N,N-dimethylformamide or N,N-dimethylacetamide.

In an alternative process, the compounds of the formula (XI) can be prepared by reacting compounds of the formula

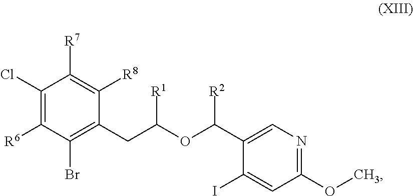

in which
R¹, R², R⁶, R⁷ and R⁸ are as defined above,
with bis(pinacolato)diboran in the presence of a base and a catalyst.

The reaction is generally carried out in inert solvents, preferably within a temperature range from room temperature to 150° C. at atmospheric pressure.

Catalysts are, for example, palladium catalysts customary for Suzuki reaction conditions, preference being given to catalysts such as dichlorobis(triphenylphosphine)palladium, tetrakistriphenylphosphinepalladium(0), bis(tri-tert-butylphosphine)palladium(0), palladium(II) acetate, palladium(II) acetate/triscyclohexylphosphine, tris(dibenzylideneacetone)dipalladium, bis(diphenylphosphaneferrocenyl)palladium(II) chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)-ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)], preference being given to palladium(II) acetate or palladium(II) acetate/triscyclohexylphosphine or tetrakistriphenylphosphinepalladium(0).

Bases are, for example, alkali metal carbonates such as caesium carbonate, potassium carbonate or sodium carbonate, alkali metal bicarbonates such as, sodium bicarbonate, alkali or alkaline earth metal hydroxides such as sodium hydroxide, potassium hydroxide, barium hydroxide or potassium acetate, potassium tert-butoxide or potassium phosphate, where these may be present in aqueous solution, preference being given to an aqueous sodium bicarbonate solution or potassium acetate.

Inert solvents are, for example, ethers such as 1,4-dioxane, tetrahydrofuran or 1,2-dimethoxyethane, hydrocarbons such as benzene, xylene or toluene, or carboxamides such as N,N-dimethylformamide or N,N-dimethylacetamide, alkyl sulfoxides such as dimethyl sulfoxide, or N-methylpyrrolidone or acetonitrile, or mixtures of the solvents with alcohols such as methanol or ethanol and/or water, preference is given to N,N-dimethylformamide.

The compounds of the formula (XII) are known or can be prepared by reacting compounds of the formula

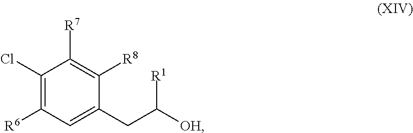

in which
R¹, R⁶, R⁷ and R⁸ are as defined above,
with compounds of the formula

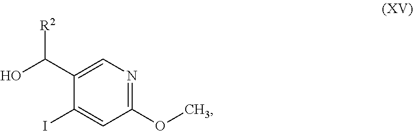

in which
R² is as defined above.

The reaction is carried out in the presence of a Lewis acid, in inert solvents, optionally in a microwave, preferably within a temperature range from 80° C. to 160° C. at atmospheric pressure to 3 bar or higher than 3 bar using a microwave.

Lewis acids are, for example, scandium(III) triflate, indium(III) chloride or tris(pentafluorophenyl)borane, preference is given to scandium(III) triflate.

Inert solvents are, for example, benzene, xylene, toluene, acetonitrile, tetrahydrofurane, nitromethane or N,N-dimethylacetamide, preference is given to nitromethane.

The compounds of the formulae (XIV) and (XV) are known, can be synthesized from the corresponding starting compounds by known processes or can be prepared analogously to the processes described in the Examples section.

The compounds of the formula (XIII) are known or can be prepared by reacting compounds of the formula

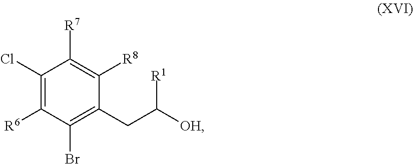

in which

R¹, R⁶, R⁷ and R⁸ are as defined above, with a compound of the formula (XV).

The reaction is carried out as described for the reaction of compounds of the formula (XIV) with compounds of the formula (XV).

The compounds of the formula (XVI) are known, can be synthesized from the corresponding starting compounds by known processes or can be prepared analogously to the processes described in the Examples section.

In an alternative process, the compounds of the formula (XII) can be prepared by reacting compounds of the formula (XIV) with compounds of the formula

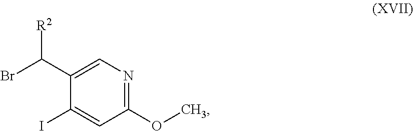

in which
R² is as defined above.

The reaction is generally carried out in inert solvents, in the presence of a base, preferably within a temperature range from room 0° C. to 80° C. at atmospheric pressure.

Inert solvents are, for example, ethers such as diethyl ether, methyl tert-butyl ether, 1,2-dimethoxyethane, 1,4-dioxane, tetrahydrofuran, N,N-dimethylformamide or N,N-dimethylacetamide, or mixtures of solvents, preference is given to tetrahydrofuran or N,N-dimethylformamide.

Bases are, for example, potassium tert-butoxide or sodium tert-butoxide, sodium hydride, n-butyllithium, lithium bis(trimethylsilyl)amide or sodium bis(trimethylsilyl)amide, preference is given to sodium hydride.

The compounds of the formula (XVI) are known, can be synthesized from the corresponding starting compounds by known processes or can be prepared analogously to the processes described in the Examples section.

In an alternative process, the compounds of the formula (XII) can be prepared by reacting compounds of the formula (XVI) with compounds of the formula (XVI).

The reaction is carried out as described for the reaction of compounds of the formula (XIV) with compounds of the formula (XVII).

In an alternative process, the compounds of the formula (IV), in which R² represents hydrogen, can be prepared by reacting compounds of the formula

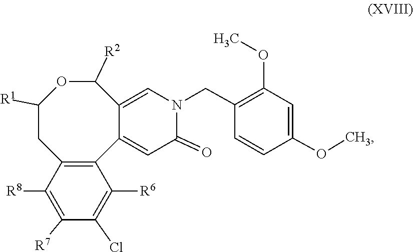

in which
R¹, R⁶, R⁷ and R⁸ are each as defined above, and
R² represents hydrogen,
with an acid.

The reaction is generally carried out in inert solvents, preferably in a temperature range from 0° C. to 80° C. at atmospheric pressure.

Inert solvents are, for example, halogenated hydrocarbons such as dichloromethane, trichloromethane, carbon tetrachloride or 1,2-dichloroethane, or ethers such as tetrahydrofuran or dioxane, preference being given to dichloromethane.

Acids are, for example, trifluoroacetic acid or hydrogen chloride in dioxane, preference being given to trifluoroacetic acid.

The compounds of the formula (XVIII) are known or can be prepared by reacting compounds of the formula

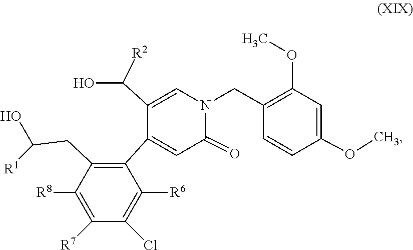

in which
R¹, R⁶, R⁷ and R⁸ are each as defined above, and
R² represents hydrogen,
with a base and a sulfonyl chloride.

The reaction is generally carried out in inert solvents, preferably in a temperature range of −20° C. to 50° C. at standard pressure, particularly preferably at room temperature and at standard pressure.

Inert solvents are, for example, halohydrocarbons such as dichloromethane, trichloromethane, tetrachloromethane or 1,2-dichloroethane, or ethers such as tetrahydrofuran or dioxane, preference being given to dichloromethane.

Bases are, for example, organic bases such as trialkylamines, e.g. triethylamine, N-methylmorpholine, N-methylpiperidine, 4-dimethylaminopyridine or diisopropylethylamine, preference being given to triethylamine.

Sulfonyl chlorides are, for example, methanesulfonyl chloride, para-toluenesulfonyl chloride or trifluoromethanesulfonyl chloride, preference being given to methanesulfonyl chloride.

The compounds of the formula (XIX) are known or can be prepared by reacting compounds of the formula

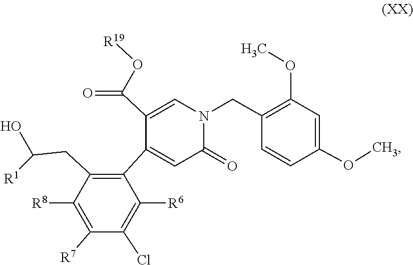

in which
R¹, R⁶, R⁷ and R⁸ are each as defined above, and
R¹⁹ represents methyl or ethyl,
with a reducing reagent.

The reaction is generally effected in inert solvents, preferably within a temperature range from −20° C. to 40° C. at standard pressure, preferably at room temperature and at standard pressure.

Inert solvents are, for example, dichloromethane, toluene or alcohols such as methanol, ethanol, n-propanol or isopropanol, preference being given to ethanol.

Reducing reagents are, for example, complex borohydrides or aluminium hydrides such as sodium borohydride, lithium borohydride, lithium aluminium hydride, sodium bis(2-methoxyethoxy)aluminium hydride, diisobutylaluminium hydride or borane tetrahydrofuran, preference being given to sodium borohydride.

The compounds of the formula (XX) are known or can be prepared by reacting compounds of the formula

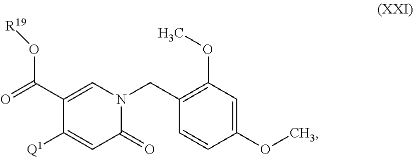

in which
R¹⁹ represents methyl or ethyl, and
Q¹ represents —B(OH)₂, a boronic acid ester, preferably boronic acid pinacol ester, or —BF₃⁻K⁺, with compounds of the formula

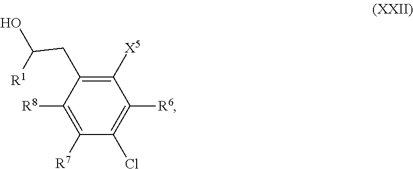

in which
R¹, R⁶, R⁷ and R⁸ are each as defined above, and
X⁵ represents chlorine, bromine, iodine, or trifluoromethanesulfonyloxy, under Suzuki coupling conditions to give compounds of the formula (XX).

The reaction is generally effected in inert solvents, in the presence of a catalyst, optionally in the presence of an additional reagent, optionally in a microwave, preferably within a temperature range from room temperature to 150° C. at standard pressure to 3 bar.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetateltriscyclohexylphosphine, tris(dibenzylideneacetone)dipalladium, bis(diphenylphosphaneferrocenyl)palladium(II) chloride, 1,3-bis(2,6-diisopropylphenyl)imidazol-2-ylidene(1,4-naphthoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)-ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)], preference being given to tetrakistriphenylphosphinepalladium(0), [1,1-bis-(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphane (1:1)] or [1,1-bis(diphenylphosphino)ferrocene]dichloropalladium dichloromethane complex, preference is given to [1,1-bis(diphenylphosphino)ferrocene]dichloropalladium dichloromethane complex.

Additional reagents are, for example, potassium acetate, caesium carbonate, potassium carbonate or sodium carbonate, potassium tert-butoxide, caesium fluoride or potassium phosphate, where these may be present in aqueous solution, preferred are additional reagents such as aqueous sodium carbonate solution or potassium acetate.

Inert solvents are, for example, ethers such as 1,4-dioxane, tetrahydrofuran or 1,2-dimethoxyethane, hydrocarbons such as benzene, xylene or toluene, or carboxamides such as N,N-dimethylformamide or N,N-dimethylacetamide, alkyl sulfoxides such as dimethyl sulfoxide, or N-methylpyrrolidone or acetonitrile, or mixtures of the solvents with alcohols such as methanol or ethanol and/or water, preference is given to 1,4-dioxane or N,N-dimethylformamide.

The compounds of the formula (XXI) are known, can be synthesized by known processes from the appropriate starting materials or can be prepared analogously to the processes described in the Examples section.

The preparation of the starting compounds and of the compounds of the formula (I) can be illustrated by the synthesis scheme which follows.

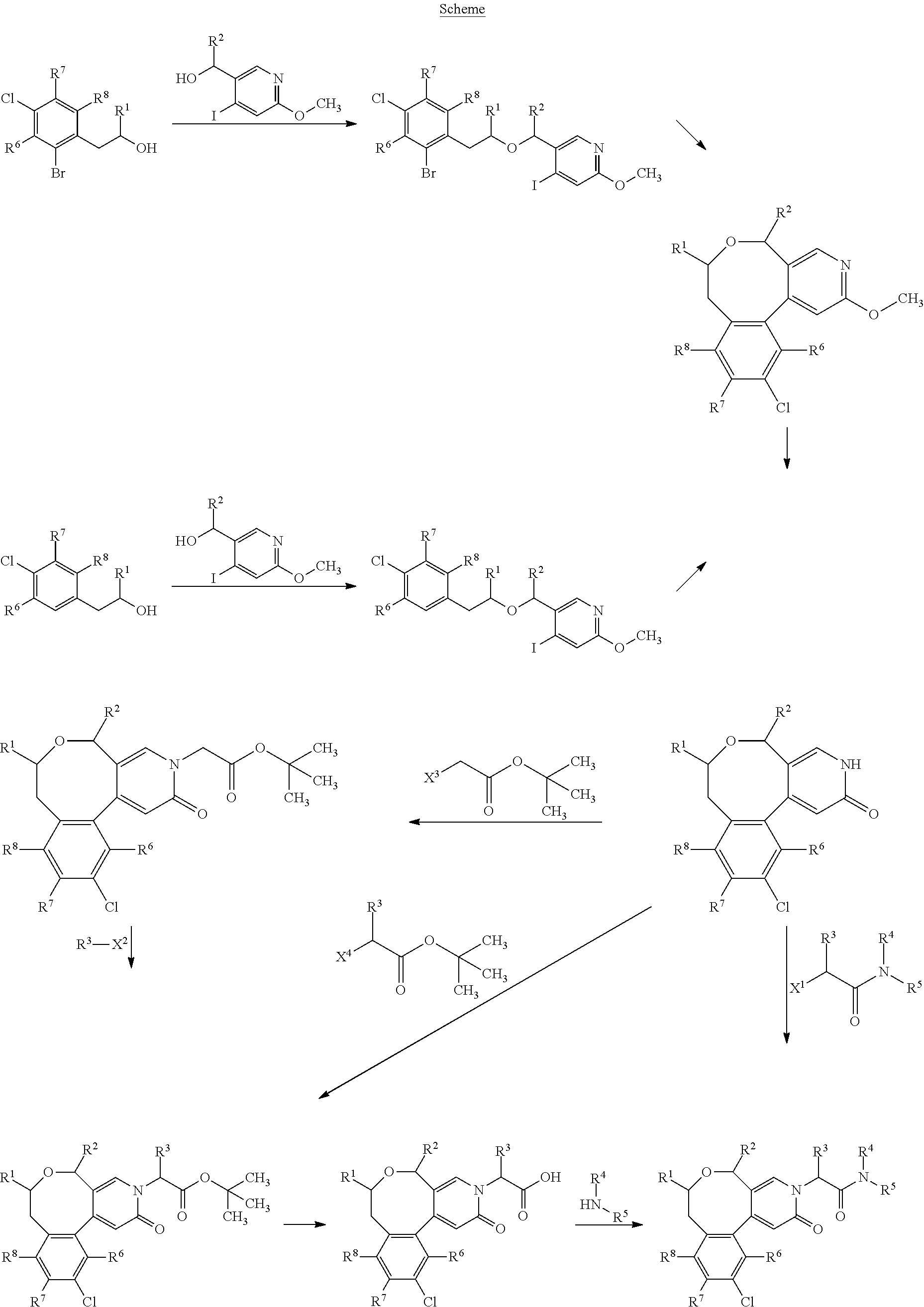

The compounds according to the invention have an unforeseeable useful pharmacological activity spectrum and good pharmacokinetic properties. They are compounds that influence the proteolytic activity of the serine protease factor XIa (FXIa). The compounds according to the invention inhibit the enzymatic cleavage of FXIa-substrates, such as factor IX (FIX), which have essential roles in the activation of blood coagulation, in the aggregation of blood platelets via PAR*1 activation of the platelets, and in inflammatory processes, which particularly involve an increase in vascular permeability.

They are therefore suitable for use as medicaments for the treatment and/or prophylaxis of diseases in humans and animals.

The present invention further provides for the use of the compounds according to the invention for the treatment and/or prophylaxis of disorders, in particular vascular disorders, preferably thrombotic or thromboembolic disorders and/or thrombotic or thromboembolic complications.

Factor XIa (FXIa) is an important enzyme in the context of coagulation, which can be activated by both thrombin and factor XIIa (FXIIa), and is therefore involved in two essential processes of coagulation. It is a central component of the transition from initiation to amplification of the coagulation and propagation of the clot: in positive feedback loops, thrombin activates, in addition to factor V and factor VIII, also factor XI to factor XIa, whereby factor IX is converted into factor IXa, and, via the factor IXa/factor VIIIa complex generated in this manner, factor Xa and subsequently thrombin are formed, leading to strong thrombus growth and stabilization of the thrombus.

Moreover, factor XIa is an important component for the intrinsic initiation of coagulation: In addition to the stimulation via tissue factor (TF) in the extrinsic pathway, the coagulation system can be activated also particularly on negatively charged surfaces, which include not only surface structures of foreign cells (e.g. bacteria) but also artificial surfaces such as vascular prostheses, stents and parts of extracorporeal circulation systems. On these surfaces, factor XII (FXII) is activated to factor XIIa (FXIIa) which subsequently activates FXI to FXIa. This leads to further activation of the coagulation cascade as described above.

In contrast, thrombin generation triggered by TF/factor VIIa via factor X activation and finally thrombin formation, which represents the early physiological reaction to vascular wall injuries, remains uninfluenced. This could explain why no prolongations of bleeding times were found in FXIa knockout mice, as in rabbits and other species, with administration of FXIa inhibitor. This low bleeding tendency caused by the substance is of great advantage for use in humans, particularly in patients with increased risk of bleeding.

Accordingly, the compounds according to the invention are suitable for the treatment and/or prophylaxis of disorders or complications which may arise from the formation of clots.

For the purpose of the present invention, the “thrombotic or thromboembolic disorders and/or thrombotic or thromboembolic complications” include disorders and complications, which occur in the arterial, the venous vascular system and the lymphatic system, which can be treated with the compounds according to the invention. This includes in particular disorders in the coronary arteries of the heart, such as acute coronary syndrome (ACS), myocardial infarction with ST segment elevation (STEMI) and without ST segment elevation (non-STEMI), stable angina pectoris, unstable angina pectoris, stent thrombosis, reocclusions and restenoses after coronary interventions such as angioplasty, stent implantation or aortocoronary bypass, disorders in the cerebrovascular arteries, such as transitory ischaemic attacks (TIA), ischemic strokes including cardioembolic strokes, such as strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and disorders of peripheral arteries, leading to peripheral artery disease, including peripheral artery occlusion, acute limb ischemia, amputation, reocclusions and restenoses after interventions such as angioplasty, stent implantation or surgery and bypass.

In addition, this includes thrombotic or thromboembolic disorders in particular in veins of the extremities, kidneys, mesenterium, liver, brain and eye, leading to pulmonary embolisms, venous thromboembolisms and/or venous thrombosis.

Stimulation of the coagulation system may occur by various causes or associated disorders. In the context of surgical interventions, immobility, confinement to bed, infections, inflammation or cancer or cancer therapy, inter alia, the coagulation system can be highly activated, and there may be thrombotic complications, in particular venous thromboses. The compounds according to the invention are therefore suitable for the prophylaxis of thrombosis in the context of surgical interventions in patients suffering from cancer. The compounds according to the invention are therefore also suitable for the prophylaxis of thrombosis in patients having an activated coagulation system, for example in the situations described above.

The inventive compounds are therefore also suitable for the prevention and treatment of cardiogenic thromboembolisms, for example brain ischaemias, stroke and systemic thromboembolisms and ischaemias, in patients with acute, intermittent or persistent cardiac arrhythmias, for example atrial fibrillation, and in patients undergoing cardioversion, and also in patients with heart valve disorders or with artificial heart valves.

In addition, the inventive compounds are suitable for the treatment and prevention of disseminated intravascular coagulation (DIC) which may occur in connection with sepsis inter alia, but also owing to surgical interventions, neoplastic disorders, burns or other injuries and may lead to severe organ damage through microthrombosis.

Thromboembolic complications furthermore occur in microangiopathic haemolytical anaemias and by blood coming into contact with artificial surfaces in the context of extracorporeal circulation such as, for example, haemodialysis and ECMO (“extracorporeal membrane oxygenation”), LVAD (“left ventricular assist device”) and similar devices, AV fistulas, vascular and heart valve prostheses.

Moreover, the compounds according to the invention are suitable for the treatment and/or prophylaxis of disorders involving microclot formation or fibrin deposits in cerebral blood vessels or asymptomatic, covert strokes, which may lead to dementia disorders such as vascular dementia or Alzheimer's disease. Here, the clot may contribute to the disorder both via occlusions and by binding disease-relevant factors.

Moreover, the compounds according to the invention are suitable for the treatment and/or prophylaxis of disorders where, in addition to the pro-coagulant component, the pro-inflammatory component also plays an essential role. Mutual enhancement of coagulation and inflammation in particular can be prevented by the compounds according to the invention, thus decisively lowering the probability of thrombotic complications. Therefore, the compounds according to the invention are suitable for the treatment and/or prophylaxis in the context of atherosclerotic vascular disorders, inflammatory diseases, such as rheumatic disorders of the locomotor system, inflammatory disorders of the lung, such as pulmonary fibroses, inflammatory disorders of the kidney, such as glomerulonephritides, inflammatory disorders of the intestine, such as Crohn's disease or ulcerative colitis, or disorders, which may be present in the context of an underlying diabetic disease, such as diabetic retinopathy or nephropathy.

Moreover, the compounds according to the invention can be used for inhibiting tumor growth and the formation of metastases, and also for the prophylaxis and/or treatment of thromboembolic complications, such as, for example, venous thromboembolisms, for cancer patients, in particular those undergoing major surgical interventions or chemo- or radiotherapy.

In addition, the inventive compounds are also suitable for the prophylaxis and/or treatment of pulmonary hypertension.

In the context of the present invention, the term “pulmonary hypertension” includes pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for the treatment and/or prophylaxis of pulmonary hypertension associated with sarcoidosis, histiocytosis X and lymphangiomatosis.

In addition, the compounds according to the invention may also be useful for the treatment of lung, liver and kidney fibrosis.

The compounds according to the invention are also suitable for the primary prophylaxis of thrombotic or thromboembolic disorders and/or thrombo-inflammatory disorders and/or disorders with increased vascular permeability in patients, in which gene mutations lead to enhanced activity of the enzymes or increased levels of the zymogens—and these are established by relevant tests/measurements of the enzyme activity or zymogen concentrations.

The present invention further provides for the use of the compounds according to the invention for the treatment and/or prophylaxis of disorders, especially the disorders mentioned above.

The present invention further provides for the use of the compounds according to the invention for production of a medicament for the treatment and/or prophylaxis of disorders, especially the disorders mentioned above.

The present invention further provides a method for the treatment and/or prophylaxis of disorders, especially the disorders mentioned above, using a therapeutically effective amount of a compound according to the invention.

The present invention further provides the compounds according to the invention for use in a method for the treatment and/or prophylaxis of disorders, especially the disorders mentioned above, using a therapeutically effective amount of a compound according to the invention.

The present invention further provides medicaments comprising a compound according to the invention and one or more further active compounds.

In addition, the compounds according to the invention can also be used for preventing coagulation ex vivo, for example for the protection of organ transplants against damage caused by formation of clots and for protecting the organ recipient against thromboemboli from the transplanted organ, for preserving blood and plasma products, for cleaning/pretreating catheters and other medical auxiliaries and instruments, for coating synthetic surfaces of medical auxiliaries and instruments used in vivo or ex vivo or for biological samples which may contain factor XIa.

The present invention furthermore provides a method for preventing the coagulation of blood in vitro, in particular in banked blood or biological samples which may comprise factor XIa, which method is characterized in that an anticoagulatory effective amount of the compound according to the invention is added.

The present invention further provides medicaments comprising a compound according to the invention and one or more further active compounds, in particular for the treatment and/or prophylaxis of the disorders mentioned above. Preferred examples of active compounds suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or β-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;
plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors) such as, for example, tissue plasminogen activator (t-PA, for example Actilyse®), streptokinase, reteplase and urokinase or plasminogen-modulating substances causing increased formation of plasmin;
anticoagulatory substances (anticoagulants), for example heparin (UFH), low-molecular-weight heparins (LMW), for example tinzaparin, certoparin, pamaparin, nadroparin, ardeparin, enoxaparin, reviparin, dalteparin, danaparoid, semuloparin (AVE 5026), adomiparin (Ml18) and EP-42675/ORG42675;
direct thrombin inhibitors (DTI) such as, for example, Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors, for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux,
substances which inhibit the aggregation of platelets (platelet aggregation inhibitors, thrombocyte aggregation inhibitors), such as, for example, acetylsalicylic acid (such as, for example, aspirin), P2Y12 antagonists such as, for example, ticlopidine (Ticlid), clopidogrel (Plavix), prasugrel, ticagrelor, cangrelor, elinogrel, PAR-1 antagonists such as, for example, vorapaxar, PAR-4 antagonists, EP3 antagonists such as, for example, DG041;
platelet adhesion inhibitors such as GPVI and/or GPIb antagonists such as, for example, Revacept or caplacizumab;
fibrinogen receptor antagonists (glycoprotein-Ib/IIIa antagonists), for example abciximab, eptifibatide, tirofiban, lamifiban, lefradafiban and fradafiban;
recombinant human activated protein C such as, for example, Xigris or recombinant thrombomudulin;
and also antiarrhythmics.

“Combinations” for the purpose of the invention mean not only dosage forms which contain all the components (so-called fixed combinations) and combination packs which contain the components separate from one another, but also components which are administered simultaneously or sequentially, provided that they are used for prophylaxis and/or treatment of the same disease. It is likewise possible to combine two or more active ingredients with one another, meaning that they are thus each in two-component or multicomponent combinations.

The inventive compounds can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The inventive compounds can be administered in administration forms suitable for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay, which control the release of the compound according to the invention), tablets which disintegrate rapidly in the mouth, or films/wafers, films/lyophilisates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

Preference is given to oral administration.

Suitable administration forms for the other administration routes are, for example, pharmaceutical forms for inhalation (including powder inhalers, nebulizers), nasal drops, solutions or sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be accomplished in a manner known per se by mixing with inert, nontoxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulfate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

In spite of this, it may be necessary, if appropriate, to deviate from the amounts specified, specifically depending on body weight, administration route, individual behaviour towards the active ingredient, type of formulation, and time or interval of administration.

Unless stated otherwise, the percentages in the tests and examples which follow are percentages by weight; parts are parts by weight. Solvent ratios, dilution ratios and concentration data for the liquid/liquid solutions are based in each case on volume. “w/v” means “weight/volume”. For example, “10% w/v” means: 100 ml of solution or suspension comprise 10 g of substance.

### A) EXAMPLES

### Abbreviations

Boc tert-Butyloxycarbonyl
br s broad singlet (in NMR)
d day(s), doublet (in NMR)
TLC thin-layer chromatography
DCI direct chemical ionization (in MS)
dd doublet of doublets (in NMR)
DMSO dimethyl sulfoxide
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure, high-performance liquid chromatography
LC/MS liquid chromatography-coupled mass spectroscopy
m multiplet (in NMR)
min minute(s)
MS mass spectroscopy
NMR nuclear magnetic resonance spectroscopy
q quartet or quadruplet (in NMR)
quin quintet (in NMR)
RP reverse phase (in HPLC)
RT room temperature
Rₜ retention time (in HPLC)
s singlet (in NMR)
sxt sextet (in NMR)
SFC supercritical fluid chromatography (with supercritical carbon dioxide as a eluent)
t triplet (in NMR)
TFA trifluoroacetic acid
T3P 2,4,6-tripropyl-1,3,5,2,4,6-trioxatriphosphinane 2,4,6-trioxide

HPLC, LC-MS and GC Methods:

Method 1: Instrument: Waters ACQUITY SQD UPLC system; column: Waters Acquity UPLC HSS T3 C18 1.8 μm, 50 mm×1.0 mm; eluent A: water+0.025% formic acid, eluent B: acetonitrile+0.025% formic acid; gradient: 0.0 min 10% B→1.2 min 95% B→2.0 min 95% B; oven: 50° C.; flow rate: 0.40 ml/min; UV detection: 210-400 nm.

Method 2: Instrument: Thermo Scientific DSQII; GC: Thermo Scientific Trace GC Ultra; column: Restek RTX-35MS, 15 m×200 μm×0.33 μm; constant helium flow rate: 1.20 ml/min; oven: 60° C.; inlet: 220° C.; gradient: 60° C., 30° C./min→300° C. (maintained for 3.33 min).

Method 3: Instrument: Waters ACQUITY SQD UPLC system; column: Waters Acquity UPLC HSS T3 C18 1.8 μm, 50 mm×1.0 mm; eluent A: water+0.025% formic acid, eluent B: acetonitrile+0.025% formic acid; gradient: 0.0 min 5% B→6.0 min 95% B→7.5 min 95% B; oven: 50° C.; flow rate: 0.35 ml/min; UV detection: 210-400 nm.

Method 4: Instrument: Thermo Scientific FT-MS; UHPLC: Thermo Scientific UltiMate 3000; column: Waters HSS T3 C18 1.8 μm, 75 mm×2.1 mm; eluent A: water+0.01% formic acid; eluent B: acetonitrile+0.01% formic acid; gradient: 0.0 min 10% B→2.5 min 95% B→3.5 min 95% B; oven: 50° C.; flow rate: 0.90 ml/min; UV detection: 210-400 nm.

Method 5: Instrument: Agilent MS Quad 6150; HPLC: Agilent 1290; column: Waters Acquity UPLC HSS T3 1.8 μm, 50 mm×2.1 mm; eluent A: water+0.025% formic acid, eluent B: acetonitrile+0.025% formic acid; gradient: 0.0 min 10% B→0.3 min 10% B→1.7 min 95% B→3.0 min 95% B; oven: 50° C.; flow rate: 1.20 ml/min; UV detection: 205-305 nm.

Method 6: Instrument: Waters Micromass QM; HPLC: Agilent 1100 series; column: Agilent ZORBAX Extend C18 3.5 μm, 50 mm×3.0 mm; eluent A: water+0.01 mol ammonium carbonate, eluent B: acetonitrile; gradient: 0.0 min 2% B→0.2 min 2% B→3.0 min 95% B→4.5 min 95% B; oven: 40° C.; flow rate: 1.75 ml/min; UV detection: 210-400 nm.

Method 7: Instrument: ThermoFisherScientific LTQ-Orbitrap-XL; HPLC: HPLC instrument: Agilent 1200SL; column: POROSHELL 120 SB—C18 2.7 μm, 150 mm×3.0 mm; eluent A: water+0.1% formic acid; eluent B: acetonitrile+0.1% formic acid; gradient: 0.0 min 2% B→0.3 min 2% B→5.0 min 95% B→10.0 min 95% B; oven: 40° C.; flow rate: 0.75 ml/min; UV detection: 210-400 nm.

Method 8: Instrument: Shimadzu LCMS-2020; column: Ascentis Express C18 2.7 μm, 50 mm×3.0 mm; eluent A: water+0.05% trifluoroacetic acid, eluent B: acetonitrile+0.05% trifluoroacetic; gradient: 0.0 min 5% B→1.2 min 95% B→2.0 min 95% B; oven: 40° C.; flow rate: 1.5 ml/min; UV detection: 210-400 nm.

Method 9: Instrument: Shimadzu LCMS-2020; column: Ascentis Express C18 2.7 μm, 50 mm×3.0 mm; eluent A: water+0.05% trifluoroacetic acid, eluent B: acetonitrile+0.05% trifluoroacetic; gradient: 0.0 min 30% B→4.0 min 100% B→5.0 min 100% B; oven: 40° C.; flow rate: 1.5 ml/min; UV detection: 210-400 nm.

Method 10: Instrument: Waters Single Quad MS; HPLC: Waters UPLC Acquity; column: Waters BEH C18 1.7 μm, 50 mm×2.1 mm; eluent A: water+0.025% ammonia, eluent B: acetonitrile; gradient: 0.0 min 8% B→0.1 min 8% B→1.8 min 95% B→3.5 min 95% B; oven: 50° C.; flow rate: 0.45 ml/min; UV detection: 210-400 nm.

Method 11: Instrument: Shimadzu LCMS-2020; column: Shim-pack XR-ODS 2.2 μm, 50 mm×3.0 mm; eluent A: water+0.05% trifluoroacetic acid, eluent B: acetonitrile+0.05% trifluoroacetic; gradient: 0.0 min 5% B→1.2 min 100% B→2.0 min 100% B; oven: 40° C.; flow rate: 1.2 ml/min; UV detection: 210-400 nm.

Method 12: Instrument: Shimadzu LCMS-2020; column: Ascentis Express C18 2.7 μm, 50 mm×2.1 mm; eluent A: water+0.05% trifluoroacetic acid, eluent B: acetonitrile+0.05% trifluoroacetic; gradient: 0.0 min 5% B→1.2 min 100% B→2.0 min 100% B; oven: 40° C.; flow rate: 1.5 ml/min; UV detection: 210-400 nm.

Method 13: Instrument: Shimadzu LCMS-2020; column: Kinetex EVO-C18 2.6 μm, 50 mm×3.0 mm; eluent A: water+0.1% trifluoroacetic acid, eluent B: acetonitrile+0.1% trifluoroacetic; gradient: 0.0 min 10% B→1.1 min 100% B→2.0 min 100% B; oven: 40° C.; flow rate: 1.5 ml/min; UV detection: 210-400 nm.

Method 14: Instrument: Shimadzu LCMS-2020; column: Ascentis Express C18 2.7 μm, 50 mm×3.0 mm; eluent A: water+0.05% trifluoroacetic acid, eluent B: acetonitrile+0.05% trifluoroacetic; gradient: 0.0 min 5% B→1.2 min 95% B→3.0 min 95% B; oven: 40° C.; flow rate: 1.5 ml/min; UV detection: 210-400 nm.

Method 15: Instrument: Shimadzu LCMS-2020; column: Ascentis Express C18 2.7 μm, 50 mm×2.1 mm; eluent A: water+0.05% trifluoroacetic acid, eluent B: acetonitrile+0.05% trifluoroacetic; gradient: 0.0 min 5% B→3.2 min 95% B→4.5 min 95% B; oven: 40° C.; flow rate: 1.5 ml/min; UV detection: 210-400 nm.

Method 16: Instrument: Shimadzu LCMS-2020; column: Ascentis Express C18 2.7 μm, 50 mm×3.0 mm; eluent A: water+0.05% trifluoroacetic acid, eluent B: acetonitrile+0.05% trifluoroacetic; gradient: 0.0 min 5% B→1.1 min 100% B→2.0 min 100% B; oven: 40° C.; flow rate: 1.5 ml/min; UV detection: 210-400 nm.

Method 17: Instrument: Waters MS SQ detector 2; GC: Agilent A7890; column: Restek RTX-35 MS, 15 m×200 μm×0.33 μm, gas: helium; oven: 60° C.; flow rate: 1.20 m/min; inlet: 240° C.; gradient: 30° C./min→300° C.

Microwave: The microwave reactor used was a “single-mode” instrument of the Emrys' Optimizer type.

When compounds according to the invention are purified by preparative HPLC by the above-described methods in which the eluents contain additives, for example trifluoroacetic acid, formic acid or ammonia, the compounds according to the invention may be obtained in salt form, for example as trifluoroacetate, formate or ammonium salt, if the compounds according to the invention contain a sufficiently basic or acidic functionality. Such a salt can be converted to the corresponding free base or acid by various methods known to the person skilled in the art.

In the case of the synthesis intermediates and working examples of the invention described hereinafter, any compound specified in the form of a salt of the corresponding base or acid is generally a salt of unknown exact stoichiometric composition, as obtained by the respective preparation and/or purification process. Unless specified in more detail, additions to names and structural formulae, such as “hydrochloride”, “trifluoroacetate”, “sodium salt” or “x HCl”, “x CF₃COOH”, “x Na*” should not therefore be understood in a stoichiometric sense in the case of such salts, but have merely descriptive character with regard to the salt-forming components present therein.

This applies correspondingly if synthesis intermediates or working examples or salts thereof were obtained in the form of solvates, for example hydrates, of unknown stoichiometric composition (if they are of a defined type) by the preparation and/or purification processes described.

In NMR spectra of mixtures of stereoisomers, numbers mentioned with “/” indicate that the stereoisomers show separate signals for the respective hydrogen atom, i.e. “ . . . / . . . (2s, 1H)” means that one hydrogen atom is represented by 2 singlets, each singlet from one or more different stereoisomer(s).

### General Synthesis Methods

### General Method 1: Etherification Using Sodium Hydride

The respective alcohol (1.0 eq.) and the respective bromide (1.0-1.25 eq.) were dissolved in tetrahydrofuran (0.2-0.4 M) and cooled to 0° C. Then sodium hydride (60% dispersion in mineral oil, 1.5-3.0 eq.) was added and stirring was continued for 1-3 h at 0° C. and overnight at RT. The reaction mixture was diluted with ethyl acetate and saturated aqueous ammonium chloride solution, subsequently washed with water and saturated aqueous sodium chloride solution, dried over anhydrous sodium sulfate and filtered. The filtrate was concentrated under reduced pressure and the crude product was either purified by column chromatography (silica gel, eluent: cyclohexane/ethyl acetate mixtures or dichloromethane/methanol mixtures) or preparative HPLC (reversed phase, eluent: water/acetonitrile gradients or water/methanol gradients).

### General Method 2: Mitsunobu Reaction Using (Tributylphosphoranylidene)Acetonitrile

(Tributylphosphoranylidene)acetonitrile (1.5-3.0 eq.) was added to a solution of the respective primary or secondary alcohol (1.0 eq.) and the respective phenol (1.0-1.5 eq.) in toluene (0.1-0.5 M) in a microwave vessel. The reaction vessel was sealed, placed into a microwave reactor, irradiated at 100 to 160° C. and stirred at this temperature for 1-8 h. The reaction mixture was cooled to RT and concentrated under reduced pressure. The crude product was either purified by column chromatography (silica gel, eluent: cyclohexane/ethyl acetate mixtures or dichloromethane/methanol mixtures) or preparative HPLC (reversed phase, eluent: water/acetonitrile gradients or water/methanol gradients).

### General Method 3a: Ring Closure Using Miyaura Borylation Reaction and Suzuki Reaction

The respective ether (1.0 eq.), bis(pinacolato)diboron (1.25-2.0 eq.), palladium(II) acetate (0.03-0.1 eq.), tricyclohexylphosphine (0.1-0.15 eq.) and potassium acetate (3.0-4.5 eq.) were dissolved in N,N-dimethylformamide (0.25-0.4 M) and argon was passed through the resulting suspension for 10 min. The reaction mixture was heated to 80-100° C. and stirred at this temperature overnight, followed by the addition of saturated aqueous sodium bicarbonate solution (5-7 ml/mmol of the respective ether) and tetrakis(triphenylphosphine)palladium(0) (0.03-0.05 eq.). Stirring was continued at 80-100° C. for additional 2-72 h and the reaction mixture was either filtered over silica gel and eluted with dichloromethane or extracted with ethyl acetate, dried over anhydrous sodium sulfate and filtered. The filtrate was concentrated under reduced pressure and the crude product was either purified by column chromatography (silica gel, eluent: cyclohexane/ethyl acetate mixtures or dichloromethane/methanol mixtures) or preparative HPLC (reversed phase, eluent: water/acetonitrile gradients or water/methanol gradients).

### General Method 3b: Ring Closure Using CH-Activation

The respective ether (1.0 eq.) and potassium pivalate or potassium acetate (2.0-6.0 eq.) were dissolved in N,N-dimethylacetamide (0.01-0.1 M) and argon was passed through the mixture for 10 min. Subsequently, tetrakis(triphenylphosphine)palladium(0) (0.05-0.2 eq.) was added and the mixture was stirred for 2-48 h at 100-150° C. The residue was diluted with water and extracted with diethyl ether. The combined organic phases were washed with brine, dried over anhydrous magnesium sulfate, filtered and concentrated under reduced pressure. The crude product was either purified by column chromatography (silica gel, eluent: cyclohexane/ethyl acetate mixtures or dichloromethane/methanol mixtures) or preparative HPLC (reversed phase, eluent: water/acetonitrile gradients or water/methanol gradients).

### General Method 4: Pyridinone Formation Using Acetic Add and Sodium Iodine

The respective 2-methoxypyridine derivative (1.0 eq.) was dissolved in acetic acid (0.05-0.2 M), sodium iodide (2.0-4.0 eq.) was added and the resulting mixture was stirred at 80-100° C. for 2-24 h. Then dichloromethane and saturated aqueous sodium bicarbonate solution were added and the aqueous phase was extracted with dichloromethane. The combined organic phases were washed with saturated aqueous sodium chloride solution, dried over anhydrous sodium sulfate, filtered and concentrated under reduced pressure. The crude product was purified either by column chromatography (silica gel, eluent: cyclohexane/ethyl acetate mixtures or dichloromethane/methanol mixtures) or preparative HPLC (reversed phase, eluent: water/acetonitrile gradients or water/methanol gradients) or used in the subsequent reaction without further purification.

### General Method 5: Pyridinone Formation Using 4-Toluenesulfonic Add Monohydrate and Lithium Iodine

The respective 2-methoxypyridine derivative (1.0 eq.) was dissolved in 1-butanol (0.1-0.25 M), 4-toluenesulfonic acid monohydrate (1.5-2.5 eq.) and lithium iodide (5.0-10.0 eq.) were added and the resulting mixture was stirred at 80-100° C. for 2-6 h. The reaction mixture was concentrated under reduced pressure and the residue was partitioned between ethyl acetate and water. The organic layer was separated, washed with saturated aqueous sodium chloride solution, dried over anhydrous sodium sulfate, filtered and concentrated under reduced pressure. The crude product was purified either by column chromatography (silica gel, eluent: cyclohexane/ethyl acetate mixtures or dichloromethane/methanol mixtures) or preparative HPLC (reversed phase, eluent: water/acetonitrile gradients or water/methanol gradients) or used in the subsequent reaction without further purification.

### General Method 6: Pyridinone Formation Using Ethanethiol and Sodium Hydride

The respective 2-methoxypyridine derivative (1.0 eq.) was dissolved in N,N-dimethylformamide (0.1-0.25 M) followed by the addition of ethanethiol (6.0-10.0 eq.). The mixture was cooled to 0° C., sodium hydride (60% dispersion in mineral oil, 3.0-5.0 eq.) was added, stirring was continued at 0° C. for 10-20 min and heated to 80-100° C. overnight. Subsequently, water was added, the mixture was neutralized to pH 7 by slow addition of aqueous hydrochloric acid (1 N) and extracted with ethyl acetate. The combined organic phases were dried over anhydrous magnesium sulfate, filtered and concentrated under reduced pressure. The crude product was purified by preparative HPLC (reversed phase, eluent: water/acetonitrile gradients or water/methanol gradients).

General Method 7: Alkylation of Pyridinone Derivatives with α-Bromo-Ester and α-Bromo-Amide Derivatives

The respective pyridinone derivative (1.0 eq.) was dissolved in a 4:1 mixture of 2-propanol and acetone (0.05-0.15 M) and 1,1,3,3-tetramethylguanidine (3.0-5.0 eq.) was added at RT. After stirring or shaking for 15 min, the respective α-bromo-ester or α-bromo-amide derivative (1.0-2.5 eq.) was added and stirring or shaking was continued overnight at RT. The crude mixture was directly purified by preparative HPLC or concentrated under reduced pressure and the crude mixture was purified by preparative HPLC (reversed phase, eluent: water/acetonitrile gradients or water/methanol gradients).

### General Method 8: Synthesis of Triflate Derivatives

A solution of trifluoromethanesulfonic anhydride (1.1-1.3 eq.) in dichloromethane (0.1-0.25 M) under argon atmosphere was cooled to −78° C. and a solution of the corresponding alcohol (1.0 eq.) and trimethylamine or pyridine (1.1-1.5 eq.) in dichloromethane (0.75-1.0 M) was added slowly. Stirring was continued at −78° C. for 0.5-2 h before the mixture was warmed to RT, diluted with methyl tert-butyl ether and washed with a 3:1 mixture of a saturated aqueous sodium chloride solution and aqueous hydrochloric acid (1 N). The organic phase was dried over anhydrous sodium sulfate, filtered and concentrated under reduced pressure to obtain the crude product, which was used in the subsequent reaction without further purification.

General Method 9: α-Alkylation of N-Substituted Pyridinone Derivatives with Triflate or Bromide Derivatives

Under argon atmosphere, the respective N-substituted pyridinone derivative (1.0 eq.) was dissolved in tetrahydrofuran (0.1-0.2 M). At −78° C., a solution of sodium bis(trimethylsilyl)amide in tetrahydrofuran (1 M, 1.15-1.5 eq.) was added dropwise. After 20-30 min, the respective triflate or bromide derivative (1.15-1.5 eq.) dissolved in tetrahydrofuran (0.2-0.4 M) was added slowly. The mixture was stirred at −78° C. for 15-30 min and for 0.5-1.0 h at RT. Then the mixture was cooled to −78° C. before glacial acetic acid (1.5-3.0 eq.) was added. The reaction mixture was concentrated under reduced pressure and the crude product was either purified by column chromatography (silica gel, eluent: cyclohexane/ethyl acetate mixtures or dichloromethane/methanol mixtures) or preparative HPLC (reversed phase, eluent: water/acetonitrile gradients or water/methanol gradients) or used in the subsequent reaction without further purification.

### General Method 10: Ester Cleavage Using Lithium Hydroxide

The respective ester (1.0 eq.) was dissolved in tetrahydrofuran (0.05-0.15 M) and an aqueous solution of lithium hydroxide or sodium hydroxide (0.3-1.0 M, 5.0-10.0 eq.) was added. The reaction mixture was stirred at RT for 2 h up to overnight and acidified by addition of aqueous solution of aqueous hydrochloric acid (1 N). The resulting crude product was filtered and subsequently washed with water and cyclohexane or directly concentrated under reduced pressure. The residue was purified by preparative HPLC (reversed phase, eluent: water/acetonitrile gradients or water/methanol gradients) or used in the subsequent reaction without further purification.

### General Method 11: Amide Coupling Using T3P/Pyridine

To a solution of the respective carboxylic acid (1 eq.) and the respective amine (1.1-1.5 eq.) in pyridine (about 0.1 M) was added T3P (50% solution in N,N-dimethylformamide or in ethyl acetate, 1.5-4.0 eq.) at RT and the mixture was stirred at RT or heated to 50-80° C. Alternatively, to a solution of the respective carboxylic acid (1.0 eq.) in pyridine (about 0.1 M) was added T3P (50% solution in N,N-dimethylformamide or in ethyl acetate, 1.5-4 eq.) and the solution was stirred for 1 to 10 min at RT. The respective amine (1.1-1.5 eq.) was added and the reaction mixture was stirred at RT or heated to 50-80° C. After stirring at the respective temperature for 1-48 h, the reaction mixture was cooled to RT and either directly concentrated under reduced pressure or diluted with water, extracted with ethyl acetate, dried over anhydrous sodium sulfate, filtered and concentrated under reduced pressure. The crude product was purified either by column chromatography (cyclohexane/ethyl acetate mixtures or dichloromethane/methanol mixtures) or preparative HPLC (reversed phase, eluent: water/acetonitrile gradients or water/methanol gradients).

### Starting Compounds

### Example 1.1A

### 4-{[(2R)-2-Bromobutanoyl]amino}-2-fluorobenzamide (Single Stereoisomer)

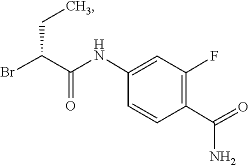 [SMILES: CC[C@@H](Br)C(=O)Nc1ccc(C(N)=O)c(F)c1]

(2R)-2-Bromobutanoic acid (single stereoisomer) (3.5 g, 21.2 mmol, 1.1 eq.), pyridine (1.7 ml, 21.2 mmol, 1.1 eq.) and T3P (17.2 ml, 50% solution in ethyl acetate, 28.9 mmol, 1.5 eq.) were added under argon atmosphere at 0-5° C. to a suspension of 4-amino-2-fluorobenzamide (3.0 g, 19.3 mmol) in tetrahydrofuran (30 ml). The reaction mixture was allowed to warm to RT and stirred for 30 min. The reaction mixture was cooled to 10° C., mixed dropwise with water (35 ml), stirred for 15 min, followed by the addition of further water (25 ml), and stirred for 30 min. The forming precipitate was filtered, washed with water and dried in vacuo. Yield: 5.23 g (90% of theory). From the combined filtrates, further precipitate formed which was filtered, washed with water and dried in vacuo. Yield: 0.5 g (9% of theory).

both batches: LC-MS (method 4): Rₜ=1.25 min; MS (ESIpos): m/z=303 [M+H]⁺

### Example 1.2A

### 4-{[(2R)-2-Bromobutanoyl]amino}benzamide (Single Stereoisomer)

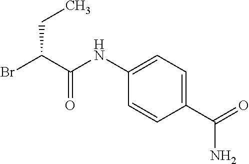 [SMILES: CC[C@@H](Br)C(=O)Nc1ccc(C(N)=O)cc1]

(2R)-2-Bromobutanoic acid (single stereoisomer) (3.97 g, 23.8 mmol, 1.1 eq.) was added slowly under argon atmosphere to an ice-cooled suspension of 4-aminobenzamide (3.00 g, 21.6 mmol) in tetrahydrofuran (22 ml), followed by the addition of pyridine (1.9 ml, 23.8 mmol, 1.1 eq.) and T3P (19 ml, 50% solution in ethyl acetate, 32.4 mmol, 1.5 eq.) at a temperature of 0-5° C. The reaction mixture was allowed to warm to RT and stirred at RT for 30 min. Water (35 ml) was added dropwise at 10° C. The reaction mixture was stirred for 15 min, followed by addition of further water (25 ml), and stirred for another 30 min. The forming precipitate was filtered, washed with water and dried in vacuo. The precipitate was suspended in a mixture of dichloromethane/ethyl acetate, filtered (the filtrate was discarded), suspended in tetrahydrofuran, filtered and dried in vacuo. Yield: 5.72 g (93% of theory). Further crystallization from the second filtrate yielded another 210 mg (3% of theory).

main batch: LC-MS (method 4): Rₜ=1.12 min; MS (ESIpos): m/z=285 [M+H]⁺

main batch: ¹H-NMR (400 MHz, DMSO-d₆): δ [ppm]=10.52 (s, 1H), 7.94-7.81 (m, 3H), 7.66 (d, 2H), 7.26 (br s, 1H), 4.48 (t, 1H), 2.17-1.88 (m, 2H), 0.96 (t, 3H).

### Example 1.3A

### (2R)-2-Bromo-N-(2-methyl-2H-benzotriazol-5-yl)butanamide (Single Stereoisomer)

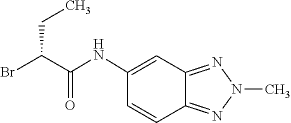 [SMILES: CC[C@@H](Br)C(=O)Nc1ccc2nn(C)nc2c1]

(2R)-2-Bromobutanoic acid (single stereoisomer) (695 mg, 4.16 mmol, 1.1 eq.) was added slowly under argon atmosphere to an ice-cooled suspension of 2-methyl-2H-benzotriazol-5-amine (590 mg, 3.78 mmol) in tetrahydrofuran (3.8 ml), followed by the addition of pyridine (337 μl, 4.16 mmol, 1.1 eq.) and T3P (3.38 ml, 50% solution in ethyl acetate, 5.67 mmol, 1.5 eq.) at a temperature of 0-5° C. The reaction mixture was allowed to warm to RT and stirred at RT for 30 min. Water (25 ml) was added dropwise at 10° C. The reaction mixture was stirred for 15 min, followed by addition of ethyl acetate (15 ml). After phase separation, the aqueous phase was extracted with ethyl acetate. The combined organic phases were washed with brine (15 ml), dried over anhydrous magnesium sulfate, filtered and concentrated under reduced pressure. Yield: 980 mg (85% of theory).

LC-MS (method 1): Rₜ=0.82 min; MS (ESIpos): m/z=297 [M+H]⁺

¹H-NMR (400 MHz, DMSO-d₆): δ [ppm]=10.55 (s, 1H), 8.36 (d, 1H), 7.89 (d, 1H), 7.43 (dd, 1H), 4.51 (t, 1H), 4.46 (s, 3H), 2.19-1.91 (m, 2H), 0.98 (t, 3H).

### Example 1.4A

### Tert-Butyl 3-(tetrahydro-2H-pyran-2-yl)propanoate (Racemate)

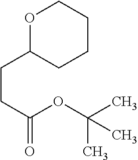

Tetrahydro-2H-pyran-2-ylmethanol (racemate) (20.0 g, 172 mmol, 1.0 eq.), tert-butyl acetate (230 ml, 1.7 mol, 10.0 eq.), bis(1,5-cyclooctadiene)diiridium(I) dichloride (5.78 g, 8.61 mmol, 0.1 eq.), triphenylphosphine (6.77 g, 25.8 mmol, 0.2 eq.) and potassium tert-butanolate (38.6 g, 344 mmol, 2.0 eq.) were dissolved in degassed tert-butanol (200 ml) and stirred at 100° C. for 6 days. The reaction mixture was filtered and washed repeatedly with ethyl acetate. After removal of the volatiles under reduced pressure, the crude mixture was purified by column chromatography (silica gel, eluent: cyclohexane/ethyl acetate with 0.1% N,N-diisopropylethylamine: 100:0 to 98:2 to 96:4). Yield: 8.40 g (23% of theory).

¹H-NMR (400 MHz, DMSO-d₆): δ [ppm]=3.88-3.80 (m, 1H), 3.32-3.24 (m, 1H), 3.20-3.13 (m, 1H), 2.29-2.13 (m, 2H), 1.78-1.67 (m, 1H), 1.63-1.49 (m, 3H), 1.47-1.35 (m, 3H), 1.39 (s, 9H), 1.18-1.06 (m, 1H).

### Example 1.4B

### tert-Butyl 2-bromo-3-(tetrahydro-2H-pyran-2-yl)propanoate (Mixture of Stereoisomers)

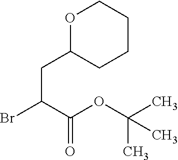 [SMILES: CC(C)(C)OC(=O)C(Br)CC1CCCCO1]

N,N-Diisopropylamine (7.7 ml, 55 mmol, 1.4 eq.) was dissolved in tetrahydrofuran (120 ml) and cooled to 0° C. before a solution of n-butyllithium (19 ml, 2.5 M in hexane, 47 mmol, 1.2 eq.) was added. The mixture was stirred at 0° C. for 30 min, cooled to −78° C., followed by the dropwise addition of tert-butyl 3-(tetrahydro-2H-pyran-2-yl)propanoate (racemate) (8.40 g, 39.2 mmol, 1.0 eq.) in tetrahydrofuran (62 ml). Stirring was continued at −78° C. for 45 min, 1,2-dibromo-1,1,2,2-tetrafluoroethane (12.2 g, 47.0 mmol, 1.2 eq.) in tetrahydrofuran (62 ml) was added dropwise and the mixture was warmed to RT over 1 h. After addition of saturated aqueous solution of ammonium chloride, the aqueous phase was extracted with dichloromethane. The combined organic phases were dried over sodium sulfate, filtered and concentrated under reduced pressure. The crude product was purified by column chromatography (silica gel, eluent: cyclohexane/ethyl acetate with 0.1% N,N-diisopropylethylamine: 100:0 to 96:4 to 70:30). Yield: 7.00 g (61% of theory).

GC-MS (method 6): Rₜ=5.38/5.50 min; MS (APCIpos): m/z=239 [M−tBu+H]⁺

### Example 1.4C

### 2-Bromo-3-(tetrahydro-2H-pyran-2-yl)propanoic Acid (Mixture of Stereoisomers)

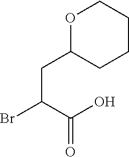 [SMILES: O=C(O)C(Br)CC1CCCCO1]

tert-Butyl 2-bromo-3-(tetrahydro-2H-pyran-2-yl)propanoate (mixture of stereoisomers) (7.00 g, 23.9 mmol, 1.0 eq.) was dissolved in trifluoroacetic acid (140 ml) and stirred at RT for 2 h. The reaction mixture was concentrated under reduced pressure and coevaporated three times with toluene. The crude product was used in the subsequent reaction without further purification. Yield: 5.65 g (quantitative of theory).

GC-MS (method 6): Rₜ=5.53 min; MS (APCIpos): m/z=239 [M+H]⁺

### Example 1.4D

### 4-({(2R)-2-Bromo-3-[(2S)-tetrahydro-2H-pyran-2-yl]propanoyl}amino)-2-fluorobenzamide (Single Stereoisomer)

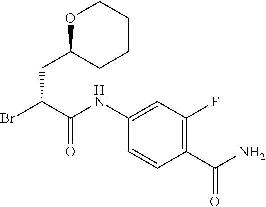 [SMILES: NC(=O)c1ccc(NC(=O)[C@H](Br)C[C@@H]2CCCCO2)cc1F]

General Method 11 was carried out with 4-amino-2-fluorobenzamide (3.68 g, 23.9 mmol, 1.0 eq.), 2-bromo-3-(tetrahydro-2H-pyran-2-yl)propanoic acid (mixture of stereoisomers) (5.66 g, 23.9 mmol, 1.0 eq.), pyridine (2.1 ml, 26 mmol, 1.1 eq.) and T3P (21 ml, 50% solution in ethyl acetate, 36 mmol, 1.5 eq.) in tetrahydrofuran (43 ml) including the following variations of the procedure: After stirring at RT overnight, the reaction mixture was worked up by addition of water, extracted with ethyl acetate, washed with brine, dried and concentrated under reduced pressure. Stereoisomer separation of the crude mixture gave:
single stereoisomer 1 (chiral SFC: Rₜ=1.52 min): 1.87 g,
single stereoisomer 2 (the title compound 1.4D) (chiral SFC: Rₜ=1.66 min, 99% de): 1.90 g (21% of theory),
single stereoisomer 3 (chiral SFC: Rₜ=1.30 min): 1.18 g,
single stereoisomer 4 (chiral SFC: Rₜ=1.34 min): 1.15 g.

Separation method 1: SFC: column: Daicel Chiralpak AD 20 μm, 450 mm×50 mm; eluent: 75% carbon dioxide/25% 2-propanol; temperature: 40° C.; flow rate: 400 m/min; UV detection: 210 nm.

Separation method 2: Single stereoisomer 3 and single stereoisomer 4 eluted as a mixture in the first separation. This mixture was then separated according to the following conditions: SFC: column: Daicel Chiralpak AD 20 μm, 450 mm×50 mm; eluent: 20% carbon dioxide/80% 2-propanol; temperature: 40° C.; flow rate: 400 ml/min; UV detection: 210 nm.

Analysis method: SFC: column: Daicel Chiralpak AD-H 5 μm, 250 mm×4.6 mm; eluent: 80% carbon dioxide/20% methanol; flow rate: 3.0 ml/min; UV detection: 210 nm.

LC-MS (method 1): Rₜ=0.82 min; MS (ESIpos): m/z=373 [M+H]⁺

¹H-NMR (500 MHz, DMSO-d₆): δ [ppm]=10.71 (s, 1H), 7.72-7.66 (m, 1H), 7.65-7.59 (m, 1H), 7.57-7.46 (m, 2H), 7.43-7.26 (m, 1H), 4.80-4.66 (m, 1H), 3.88 (d, 1H), 3.54-3.41 (m, 1H), 3.40-3.33 (m, 1H), 2.11-1.95 (m, 2H), 1.88-1.69 (m, 1H), 1.61 (d, 1H), 1.53-1.38 (m, 3H), 1.31-1.21 (m, 1H).

### Example 1.5A

### 4-({(2R)-2-Bromo-3-[(2S)-tetrahydro-2H-pyran-2-yl]propanoyl}amino)benzamide (Single Stereoisomer)

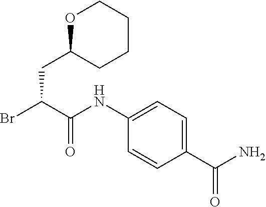 [SMILES: NC(=O)c1ccc(NC(=O)[C@H](Br)C[C@@H]2CCCCO2)cc1]

General Method 11 was carried out with 4-aminobenzamide (5.95 g, 43.7 mmol, 1.0 eq.), 2-bromo-3-(tetrahydro-2H-pyran-2-yl)propanoic acid (mixture of stereoisomers) (11.4 g, 48.1 mmol, 1.1 eq.), pyridine (3.9 ml, 48.1 mmol, 1.1 eq.) and T3P (39 ml, 50% solution in ethyl acetate, 66 mmol, 1.5 eq.) in tetrahydrofuran (79 ml) including the following variations of the procedure: After stirring at RT overnight, the reaction mixture was worked up by addition of water, extracted with ethyl acetate, washed with brine, dried and concentrated under reduced pressure. Stereoisomer separation of the crude mixture gave:
single stereoisomer 1 (the title compound 1.5A) (chiral SFC: Rₜ=1.52 min, 99% de): 2.71 g (17% of theory),
single stereoisomer 2 (chiral SFC: Rₜ=1.66 min): 3.29 g,
single stereoisomer 3 (chiral SFC: Rₜ=1.30 min): 1.43 g,
single stereoisomer 4 (chiral SFC: Rₜ=1.34 min): 1.45 g.

Separation method 1: SFC: column: Daicel Chiralpak AD 20 μm, 450 mm×50 mm; eluent: 75% carbon dioxide/25% 2-propanol; temperature: 40° C.; flow rate: 400 m/min; UV detection: 210 nm.

Separation method 2: Single stereoisomer 3 and single stereoisomer 4 eluted as a mixture in the first separation. This mixture was then separated according to the following conditions: SFC: column: Daicel Chiralpak AD 20 μm, 450 mm×50 mm; eluent: 20% carbon dioxide/80% 2-propanol; temperature: 40° C.; flow rate: 400 ml/min; UV detection: 210 nm.

Analysis method: SFC: column: Daicel Chiralpak AD-H 5 μm, 250 mm×4.6 mm; eluent: 80% carbon dioxide/20% methanol; flow rate: 3.0 ml/min; UV detection: 210 nm.

LC-MS (method 1): Rₜ=0.75 min; MS (ESIpos): m/z=355 [M+H]⁺

¹H-NMR (600 MHz, DMSO-d₆): δ [ppm]=10.58 (s, 1H), 7.95-7.82 (m, 3H), 7.71-7.63 (m, 2H), 7.29 (br s, 1H), 4.81 (dd, 1H), 3.92 (d, 1H), 3.58-3.43 (m, 1H), 3.42-3.37 (m, 1H), 2.12-1.99 (m, 2H), 1.80 (d, 1H), 1.64 (d, 1H), 1.57-1.41 (m, 4H), 1.37-1.22 (m, 1H).

### Example 1.6A

### Tert-Butyl (4S)-4-hydroxypentanoate (Single Stereoisomer)

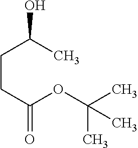

tert-Butyl acetate (4.00 g, 34.4 mmol, 2.0 eq.) was added dropwise at −78° C. to a stirred solution of lithium diisopropylamide (17.2 ml, 34.4 mmol, 2 M in tetrahydrofuran/n-hexane, 2.0 eq.) in tetrahydrofuran (50 ml). After stirring for 0.5 h, the mixture was warmed to −40° C. Diethylaluminum chloride (34.4 ml, 34.4 mmol, 1.0 M in n-hexane, 2.0 eq.) was added over a period of 5 min and stirring was continued for further 15 min. Subsequently, (2S)-2-methyloxirane (single stereoisomer) (1.00 g, 17.2 mmol, 1.0 eq.) was added and the mixture was stirred at −40° C. for 5 h, followed by the addition of saturated aqueous solution of ammonium chloride and ice in aqueous hydrochloric acid (6 N) at −20° C. The mixture was extracted with diethyl ether. The combined organic layers were washed with saturated aqueous solution of sodium bicarbonate and brine, dried over anhydrous sodium sulfate, filtered and concentrated under reduced pressure. The residue was purified by column chromatography (silica gel, eluent: n-hexane/ethyl acetate 4:1). Yield: 1.50 g (90% purity, 45% of theory).

¹H-NMR (400 MHz, DMSO-d₆): δ [ppm]=4.45 (d, 1H), 3.64-3.46 (m, 1H), 2.34-2.12 (m, 2H), 1.61-1.45 (m, 2H), 1.40 (s, 9H), 1.04 (d, 3H).

### Example 1.6B

### Tert-Butyl (4S)-4-methoxypentanoate (Single Stereoisomer)

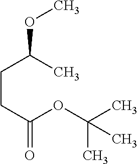

Iodomethane (61.3 g, 432 mmol, 10.0 eq.) was added at 0° C. to a suspension of tert-butyl (4S)-4-hydroxypentanoate (single stereoisomer) (8.00 g, 43.2 mmol, 1.0 eq.) and freshly prepared silver(I) oxide (30.0 g, 130 mmol, 3.0 eq.) in 1,2-dichloroethane (150 ml). After stirring at 40° C. for 72 h, the reaction mixture was filtered through a pad of Celite® and the filtrate was concentrated under reduced pressure. The residue was purified by column chromatography (silica gel, eluent: n-hexane/ethyl acetate 8:1 to 1:1). Yield: 2.20 g (26% of theory).

¹H-NMR (400 MHz, DMSO-d₆): δ [ppm]=3.28-3.21 (m, 1H), 3.18 (s, 3H), 2.20 (t, 2H), 1.68-1.54 (m, 2H), 1.39 (s, 9H), 1.04 (d, 3H).

### Example 1.6C

### Tert-Butyl (4S)-2-bromo-4-methoxypentanoate (Mixture of Two Diastereomers)

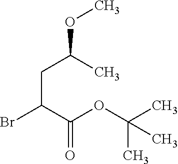

Lithium diisopropylamide (5.7 ml, 11.4 mmol, 2.0 M in tetrahydrofuran, 1.5 eq.) was added at −78° C. to a solution of Tert-butyl (4S)-4-methoxypentanoate (single stereoisomer) (1.50 g, 7.6 mmol, 1.0 eq.) in tetrahydrofuran (60 ml). After stirring at −78° C. for 15 min, a solution of 1,2-dibromo-1,1,2,2-tetrachloroethane (6.16 g, 18.9 mmol, 2.5 eq.) in tetrahydrofuran (8 ml) was added dropwise. The resulting mixture was slowly allowed to warm to RT over a period of 1 h and stirred for further 2 h before water was added. The aqueous phase was extracted with ethyl acetate. The combined organic layers were washed with water and brine, dried over anhydrous sodium sulfate, filtered and concentrated under reduced pressure. The residue was purified by preparative thin-layer chromatography (silica gel, eluent: petroleum ether/ethyl acetate 8:1). Yield: 0.94 g (85% purity, 40% of theory).

¹H-NMR (400 MHz, DMSO-d₆): δ [ppm]=4.41-4.28 (m, 1H), 3.47-3.36/3.35-3.25 (2m, 1H), 3.21/3.18 (2s, 3H), 2.21-1.85 (m, 2H), 1.43/1.42 (2s, 9H), 1.15-1.05 (m, 3H).

### Example 16D

### (4S)-2-Bromo-4-methoxypentanoic Acid (Mixture of Two Diastereomers)

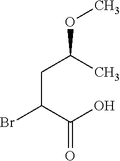

Trifluoroacetic acid (13.3 ml, 172.6 mmol, 20 eq.) was added dropwise under argon atmosphere to an ice-cooled solution of tert-butyl (4S)-2-bromo-4-methoxypentanoate (mixture of two diastereomers) (2.7 g, 85% purity, 8.6 mmol) in dichloromethane (50 ml). The reaction mixture was stirred at RT for 2 h, followed by the addition of further trifluoroacetic acid (3.3 ml, 43.2 mmol, 5.0 eq.). After stirring for another 1 h, the reaction mixture was concentrated in vacuo and coevaporated with dichloromethane. The residue was purified by column chromatography (silica gel, eluent: cyclohexane/ethyl acetate gradient). Yield: 1.37 g (75% of theory).

¹H-NMR (600 MHz, DMSO-d₆): δ [ppm]=4.41-4.36 (m, 1H), 3.48-3.40/3.35-3.28 (2m, 1H), 3.23/3.19 (2s, 3H), 2.17-2.02/1.96-1.89 (2m, 2H), 1.12/1.10 (2d, 3H).

### Example 1.6E

### 4-{[(2R,4S)-2-Bromo-4-methoxypentanoyl]amino}benzamide (Single Stereoisomer)

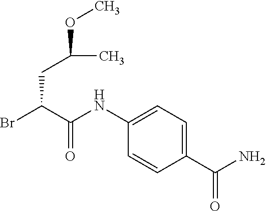 [SMILES: CO[C@@H](C)C[C@@H](Br)C(=O)Nc1ccc(C(N)=O)cc1]

(4S)-2-Bromo-4-methoxypentanoic acid (mixture of two diastereomers) (1.37 g, 6.5 mmol), pyridine (579 μl, 7.2 mmol, 1.1 eq.) and T3P (5.7 ml, 50% solution in ethyl acetate, 9.8 mmol, 1.5 eq.) were added under argon atmosphere at RT to a solution of 4-aminobenzamide (975 mg, 7.2 mmol, 1.1 eq.) in tetrahydrofuran (25 ml). The reaction mixture was stirred at RT for 2 h, quenched with water and diluted with ethyl acetate. After phase separation, the aqueous phase was extracted with ethyl acetate. The combined organic phases were washed with brine, dried over anhydrous sodium sulfate, filtered and concentrated under reduced pressure. The residue was purified by column chromatography (silica gel, eluent: dichloromethane/methanol gradient). Yield: 1.91 g (86% of theory).

LC-MS (method 4): Rₜ=1.18 min; MS (ESIpos): m/z=329 [M+H]⁺

Diastereomer separation of 1910 mg of 4-{[(4S)-2-bromo-4-methoxypentanoyl]amino}benzamide (mixture of two diastereomers) gave
single stereoisomer 1 (the title compound Example 1.6E) (chiral HPLC: Rₜ=11.7 min, >99% de): 901 mg,
single stereoisomer 2 (chiral HPLC: Rₜ=14.0 min, 89% de): 576 mg.

Separation method: HPLC: column: Daicel Chiralpak IE 5 μm, 250 mm×20 mm; eluent: 80% n-heptane/20% ethanol; temperature: 40° C.; flow rate: 15 ml/min; UV detection: 220 nm.

Analysis method: HPLC: column: Daicel Chiralpak IE 5 μm, 250 mm×4.6 mm; eluent: 80% iso-hexane/20% ethanol; temperature: 40° C.; flow rate: 1 ml/min; UV detection: 220 nm.

¹H-NMR (400 MHz, DMSO-d₆): δ [ppm]=10.56 (s, 1H), 7.92-7.81 (m, 3H), 7.65 (d, 2H), 7.25 (br s, 1H), 4.75 (t, 1H), 3.54-3.44 (m, 1H), 3.26 (s, 3H), 2.10-2.02 (m, 2H), 1.15 (d, 3H).

### Example 1.7A

### Tert-Butyl 4-oxobutanoate

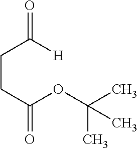

3,3,3-Triacetoxy-3-iodophthalide (123.7 g, 291.6 mmol, 2.0 eq.) was added in portions at 0° C. to a mixture of tert-butyl 4-hydroxybutanoate (23.4 g, 145.8 mmol, 1.0 eq.) and sodium bicarbonate (24.5 g, 291.6 mmol, 2.0 eq.) in dichloromethane (500 ml). After stirring at RT for 2 h, the reaction mixture was quenched by the addition of a mixture of saturated aqueous solution of sodium carbonate and sodium thiosulfate (1:1), stirred for further 30 min and extracted with dichloromethane. The combined organic phases were washed with water, brine, dried over anhydrous sodium sulfate, filtered and concentrated under reduced pressure. The residue was purified by column chromatography (silica gel, eluent: petroleum ether/ethyl acetate 95:5). Yield: 15.4 g (63% of theory).

¹H-NMR (300 MHz, CDCl₃): δ [ppm]=9.79 (s, 1H), 2.76-2.68 (m, 2H), 2.59-2.50 (m, 2H), 1.43 (s, 9H).

### Example 1.7B

### tert-Butyl 4-cyclopropyl-4-hydroxybutanoate (Racemate)

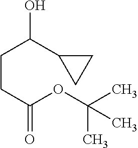

Cyclopropylmagnesium bromide (185.0 ml, 1.0 M in tetrahydrofuran, 185.0 mmol, 2.0 eq.) was added under argon atmosphere at −10° C. to a stirred solution of tert-butyl 4-oxobutanoate (15.4 g, 92.5 mmol) in tetrahydrofuran (300 ml). The reaction mixture was allowed to warm to RT within a period of 1 h, stirred for another 1 h, quenched with aqueous solution of aqueous hydrochloric acid (2 N), diluted with ethyl acetate and washed with water and brine. The organic phase was dried over anhydrous sodium sulfate, filtered and concentrated under reduced pressure. The residue was purified by column chromatography (silica gel, eluent: petroleum ether/ethyl acetate 90:10). Yield: 5.88 g (85% purity, 27% of theory).

¹H-NMR (300 MHz, DMSO-d₆): δ [ppm]=4.55-4.30 (m, 1H), 2.89-2.72 (m, 1H), 2.39-2.13 (m, 2H), 1.82-1.52 (m, 2H), 1.39 (s, 9H), 0.88-0.67 (m, 1H), 0.45-0.05 (m, 4H).

### Example 1.7C

### tert-Butyl 4-cyclopropyl-4-methoxybutanoate (Racemate)

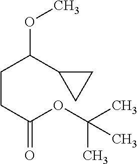

Iodomethane (34.82 g, 245.3 mmol, 10.0 eq.) was added under argon atmosphere at RT to a mixture of tert-butyl 4-cyclopropyl-4-hydroxybutanoate (racemate) (5.78 g, 85% purity, 24.5 mmol) and freshly prepared silver(I) oxide (17.05 g, 73.6 mmol, 3.0 eq.) in 1,2-dichloroethane (100 ml). After stirring at 45° C. for 24 h, the resulting mixture was filtered through Celite®. The filtrate was concentrated under reduced pressure. The residue was re-dissolved in 1,2-dichloroethane (100 ml), followed by the addition of silver(I) oxide (8.53 g, 36.8 mmol, 1.5 eq.) and iodomethane (17.41 g, 122.7 mmol, 5.0 eq.) under argon atmosphere at RT. The resulting mixture was stirred at 45° C. for 48 h and filtered through Celite®. The filtrate was concentrated under reduced pressure. The residue was purified by column chromatography (silica gel, eluent: petroleum ether/ethyl acetate 99:1 to 80:20). Yield: 1.44 g (27% of theory).

¹H-NMR (300 MHz, CDCl₃): δ [ppm]=3.38 (s, 3H), 2.53-2.42 (m, 1H), 2.42-2.28 (m, 2H), 1.97-1.77 (m, 2H), 1.44 (s, 9H), 0.84-0.69 (m, 1H), 0.68-0.54 (m, 1H), 0.53-0.31 (m, 2H), 0.14-0.02 (m, 1H).

### Example 1.7D

### tert-Butyl 2-bromo-4-cyclopropyl-4-methoxybutanoate (Mixture of Stereoisomers)

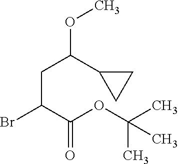 [SMILES: COC(CC(Br)C(=O)OC(C)(C)C)C1CC1]

Lithium diisopropylamide (4.9 ml, 2.0 M in tetrahydrofuran, 9.9 mmol, 1.5 eq.) was added under argon atmosphere at −78° C. to a solution of ter-butyl 4-cyclopropyl-4-methoxybutanoate (racemate) (1.44 g, 6.6 mmol) in tetrahydrofuran (20 ml). The resulting mixture was stirred at −78° C. for 1 h, followed by the dropwise addition of a solution of 1,2-dibromo-1,1,2,2-tetrachloroethane (2.57 g, 7.9 mmol, 1.2 eq.) in tetrahydrofuran (10 ml) at −78° C. The resulting mixture was allowed to warm to RT, stirred further for 2 h, quenched with saturated aqueous solution of ammonium chloride and extracted with ethyl acetate. The combined organic phases were washed with water and brine, dried over anhydrous sodium sulfate, filtered and concentrated in vacuo. The residue was purified by preparative thin-layer chromatography (silica gel, eluent: petroleum ether/ethyl acetate 80:20). Yield: 1.52 g (80% purity, 63% of theory).

¹H-NMR (400 MHz, CDCl₃): δ [ppm]=4.42-4.33 (m, 1H), 3.43/3.36 (2s, 3H), 2.74-2.61 (m, 1H), 2.57-2.49/2.44-2.34 (2m, 1H), 2.29-2.19 (m, 1H), 1.47 (s, 9H), 0.87-0.72 (m, 1H), 0.71-0.60 (m, 1H), 0.53-0.36 (m, 2H), 0.19-0.03 (m, 1H).

### Example 1.7E

### 2-Bromo-4-cyclopropyl-4-methoxybutanoic Acid (Mixture of Stereoisomers)

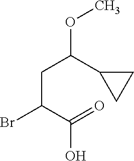

Trifluoroacetic acid (6.2 ml, 80.2 mmol, 20 eq.) was added dropwise under argon atmosphere to an ice-cooled solution of tert-butyl 2-bromo-4-cyclopropyl-4-methoxybutanoate (mixture of stereoisomers) (1.47 g, 80% purity, 4.01 mmol) in dichloromethane (40 ml). The reaction mixture was stirred at RT for 1.5 h, concentrated in vacuo and coevaporated two times with dichloromethane. The residue was purified by column chromatography (silica gel, eluent: dichloromethane/methanol gradient). Yield: 641 mg (67% of theory).

¹H-NMR (600 MHz, DMSO-d₆): δ [ppm]=13.15 (br s, 1H), 4.44-4.34 (m, 1H), 3.32/3.27 (2s, 3H), 3.04-2.97/2.85-2.79/2.72-2.62 (3m, 1H), 2.35-2.28/2.28-2.21 (2m, 1H), 2.19-2.11/2.07-2.00 (2m, 1H), 0.88-0.73 (m, 1H), 0.64-0.53 (m, 1H), 0.50-0.36 (m, 2H), 0.12-0.0 (m, 1H).

### Example 1.7F

### 4-{[(2R,4R)-2-Bromo-4-cyclopropyl-4-methoxybutanoyl]amino}benzamide (Single Stereoisomer)

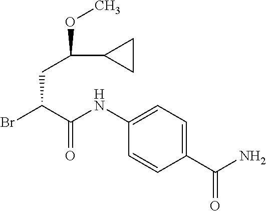 [SMILES: CO[C@H](C[C@@H](Br)C(=O)Nc1ccc(C(N)=O)cc1)C1CC1]

2-Bromo-4-cyclopropyl-4-methoxybutanoic acid (mixture of stereoisomers) (641 mg, 2.70 mmol), pyridine (241 μl, 2.97 mmol, 1.1 eq.) and T3P (2.37 ml, 50% solution in ethyl acetate, 4.06 mmol, 1.5 eq.) were added under argon atmosphere at RT to a solution of 4-aminobenzamide (368 mg, 2.70 mmol, 1.0 eq.) in tetrahydrofuran (15 ml). The reaction mixture was stirred at RT for 1.5 h before additional 4-aminobenzamide (110 mg, 0.81 mmol, 0.3 eq.) and T3P (316 μl, 50% solution in ethyl acetate, 0.54 mmol, 0.2 eq.) were added and stirred for another 1 h. The reaction mixture was quenched with water and diluted with ethyl acetate. After phase separation, the aqueous phase was extracted with ethyl acetate. The combined organic phases were washed with brine, dried over anhydrous sodium sulfate, filtered and concentrated under reduced pressure. Yield: 941 mg.

Stereoisomer separation of 941 mg of 4-(2-bromo-4-cyclopropyl-4-methoxybutan-amido)benzamide (mixture of stereoisomers) gave
mixture of two stereoisomers 1+2 (chiral SFC: Rₜ=2.81 min): 197 mg,
single stereoisomer 3 (chiral SFC: Rₜ=3.25 min, 98% ee): 134 mg,
single stereoisomer 4 (the title compound Example 1.7F) (chiral SFC: Rₜ=3.58 min, 98% ee): 123 mg.

Separation method: SFC: column: Daicel OJ-H, 250 mm×20 mm; eluent: 89% carbon dioxide/11% ethanol; temperature: 40° C.; flow rate: 80 ml/min; UV detection: 210 nm.

Analysis method: SFC: column: Daicel OJ-3, 50 mm×4.6 mm; eluent: 90% carbon dioxide/10% methanol; temperature: 40° C.; flow rate: 3 ml/min; UV detection: 210 nm.

LC-MS (method 4): Rₜ=1.38 min; MS (ESIneg): m/z=353 [M−H]⁻

¹H-NMR (600 MHz, DMSO-d₆): δ [ppm]=10.55 (s, 1H), 7.89-7.81 (m, 3H), 7.65 (d, 2H), 7.23 (br s, 1H), 4.74 (dd, 1H), 3.36 (s, 3H), 2.70 (dt, 1H), 2.26-2.13 (m, 2H), 0.84-0.76 (m, 1H), 0.65-0.57 (m, 1H), 0.49-0.41 (m, 2H), 0.06-0.0 (m, 1H).

### Example 1.8A

### Tert-Butyl 4-(difluoromethoxy)butanoate

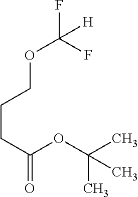

[Bromo(difluoro)methyl](trimethyl)silane (5.28 ml, 29.6 mmol, 2.0 eq.) was added dropwise under argon atmosphere at RT to a solution of tert-butyl 4-hydroxybutanoate (2.50 g, 14.8 mmol) and potassium acetate (5.82 g, 59.3 mmol, 4.0 eq.) in a mixture of dichloromethane (8.7 ml) and water (8.7 ml). The reaction mixture was stirred overnight and diluted with dichloromethane and water. After phase separation, the aqueous phase was extracted with dichloromethane. The combined organic phases were dried over anhydrous sodium sulfate, filtered and concentrated under reduced pressure. The crude product was used without further purification. Yield: 3.82 g.

¹H-NMR (600 MHz, DMSO-d₆): δ [ppm]=6.63 (t, 1H), 3.82 (t, 2H), 2.27 (t, 2H), 1.83-1.75 (m, 2H), 1.40 (s, 9H).

### Example 1.8B

### Tert-Butyl 2-bromo-4-(difluoromethoxy)butanoate (Racemate)

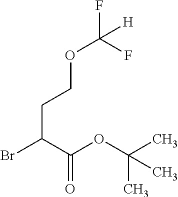

A solution of n-butyllithium (12.2 ml, 1.6 M in hexane, 19.5 mmol, 1.2 eq.) was added dropwise under argon atmosphere at 0° C. to a solution of diisopropylamine (3.2 ml, 22.8 mmol, 1.4 eq.) in tetrahydrofuran (25 ml). The reaction mixture was stirred at 0° C. for 30 min and cooled to −78° C. A solution of tert-butyl 4-(difluoromethoxy)butanoate (3.8 g, 90% purity, 16.3 mmol) in tetrahydrofuran (12.5 ml) was added dropwise and stirred at −78° C. for 30 min. A solution of 1,2-dibromo-1,1,2,2-tetrafluoroethane (5.1 g, 19.5 mmol, 1.2 eq.) was added dropwise and stirred at −78° C. for 10 min. The reaction mixture was allowed to warm to RT, stirred for another 1 h and quenched with saturated aqueous solution of ammonium chloride. After phase separation, the aqueous phase was extracted with ethyl acetate. The combined organic phases were washed with brine, dried over anhydrous sodium sulfate, filtered and concentrated under reduced pressure. The residue was purified by column chromatography (silica gel, eluent: cyclohexane/ethyl acetate gradient). Yield: 1.7 g (36% of theory).

¹H-NMR (600 MHz, DMSO-d₆): δ [ppm]=6.59 (t, 1H), 4.35-4.30 (m, 1H), 3.90-3.80 (m, 2H), 2.30-2.22 (m, 1H), 2.10-2.02 (m, 1H).

### Example 1.9A

### tert-Butyl (4S)-4-(difluoromethoxy)pentanoate (Single Stereoisomer)

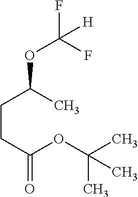

A mixture of tert-butyl (4S)-4-hydroxypentanoate (single stereoisomer) (3.00 g, 16.2 mmol, 1.0 eq.) in dichloromethane (10 ml) and water (10 ml) was added into a plastic bottle, followed by the addition of [bromo(difluoro)methyl](trimethyl)silane (9.86 g, 48.6 mmol, 3.0 eq.) and potassium hydrogen difluoride (7.58 g, 97.1 mmol, 6.0 eq.) at RT. After stirring at RT for 10 h, the reaction mixture was diluted with dichloromethane, washed with water, dried over anhydrous magnesium sulfate and filtered. The filtrate was concentrated under reduced pressure and used in the subsequent reaction without further purification. Yield: 3.50 g (92% of theory).

¹H-NMR (400 MHz, CDCl₃): δ [ppm]=6.47-5.97 (m, 1H), 4.39-4.18 (m, 1H), 2.39-2.25 (m, 2H), 1.90-1.72 (m, 2H), 1.45 (s, 9H), 1.30 (d, 3H).

### Example 1.9B

### Tert-Butyl (4S)-2-bromo-4-(difluoromethoxy)pentanoate (Mixture of Two Diastereomers)

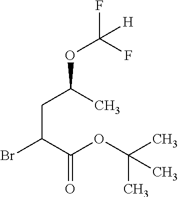 [SMILES: C[C@@H](CC(Br)C(=O)OC(C)(C)C)OC(F)F]

Lithium diisopropylamide (8.9 ml, 2.0 M in tetrahydrofuran, 17.8 mmol, 1.2 eq.) was added at −78° C. to a solution of tert-butyl (4S)-4-(difluoromethoxy)pentanoate (single stereoisomer) (3.50 g, 14.8 mmol, 1.0 eq.) in tetrahydrofuran (40 ml). After stirring at −78° C. for 1 h, 1,2-dibromo-1,1,2,2-tetrachloroethane (5.79 g, 17.8 mmol, 1.2 eq.) in tetrahydrofuran (15 ml) was added. The resulting mixture was allowed to warm to RT and stirred for further 4 h. The reaction mixture was quenched with saturated aqueous solution of ammonium chloride and extracted with ethyl acetate. The combined organic layers were washed with water and brine, dried over anhydrous sodium sulfate, filtered and concentrated under reduced pressure. The crude product was purified preparative thin-layer chromatography (silica gel, eluent: petroleum ether/ethyl acetate 91:9). Yield: 2.09 g (45% of theory).

¹H-NMR (400 MHz, CDCl₃): δ [ppm]=6.48-5.92 (m, 1H), 4.52-4.44 (m, 1H), 4.36-4.22 (m, 1H), 2.44-1.98 (m, 2H), 1.49/1.49 (2s, 9H), 1.36/1.32 (2d, 3H).

### Example 1.10A

### Ethyl 5,5-difluoro-4-oxopentanoate

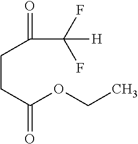

[Bromo(difluoro)methyl](trimethyl)silane (74.04 g, 364.5 mmol, 1.5 eq.), triphenylphosphine (70.12 g, 267.3 mmol, 1.1 eq.) and 1,3-dimethyl-3,4,5,6-tetrahydro-2(1H)-pyrimidinone (62.30 g, 486.1 mmol, 2.0 eq.) were added under argon atmosphere at RT to a solution of ethyl 4-chloro-4-oxobutanoate (40.0 g, 243.0 mmol, 1.0 eq.) in acetonitrile (240 ml). After stirring for 5 h at RT, the resulting mixture was quenched with water (200 ml) and pyridine (76.90 g, 972.1 mmol, 4.0 eq.). After stirring for further 1.5 h at 80° C., the reaction mixture was cooled to RT and concentrated under reduced pressure. The residue was diluted with water and extracted with methyl tert-butyl ether. The combined organic phases were washed with water and brine, dried over anhydrous sodium sulfate, filtered and concentrated under reduced pressure. The residue was purified by column chromatography (silica, gel, eluent: petroleum ether/ethyl acetate 95:5). Yield: 15.20 g (70% purity, 22% of theory).

¹H-NMR (400 MHz, CDCl₃): δ [ppm]=5.94-5.60 (m, 1H), 4.23-4.07 (m, 2H), 2.98-2.94 (m, 1H), 2.71-2.64 (m, 2H), 2.14-2.06 (m, 1H), 1.30-1.22 (m, 3H).

¹⁹F-NMR (376 MHz, CDCl₃): δ [ppm]=−127.60 (s, 2F).

### Example 1.10B

### Ethyl 5,5-difluoro-4-hydroxypentanoate (Racemate)

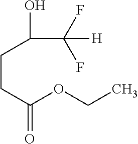

Sodium borohydride (2.23 g, 59.1 mmol, 1.0 eq.) was added at 0° C. to a solution of ethyl 5,5-difluoro-4-oxopentanoate (15.20 g, 70% purity, 59.1 mmol) in tetrahydrofuran (140 ml) and ethanol (20 ml). After stirring at 0° C. for 2 h, the reaction mixture was quenched with aqueous hydrochloric acid (3 N) at 0° C. and concentrated under reduced pressure. The residue was dissolved in ethyl acetate and washed with water and brine. The organic phase was dried over anhydrous sodium sulfate, filtered and concentrated under reduced pressure. The residue was purified by column chromatography (silica gel, eluent: petroleum ether/ethyl acetate 90:10). Yield: 5.40 g (48% of theory).

¹H-NMR (400 MHz, DMSO-d₆): δ [ppm]=5.96-5.67 (m, 1H), 5.53 (d, 1H), 4.08-4.00 (m, 2H), 3.69-3.53 (m, 1H), 2.48-2.32 (m, 2H), 1.82-1.69 (m, 1H), 1.64-1.51 (m, 1H), 1.18 (t, 3H).

¹⁹F-NMR (376 MHz, DMSO-d₆): δ [ppm]=−128.80 (q, 2F).

### Example 1.10C

### Ethyl 5,5-difluoro-4-methoxypentanoate (Racemate)

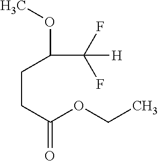

Iodomethane (40.39 g, 284.6 mmol, 10.0 eq.) was added under argon atmosphere at RT to a mixture of ethyl 5,5-difluoro-4-hydroxypentanoate (racemate) (5.40 g, 28.5 mmol, 1.0 eq.) and freshly prepared silver(I) oxide (19.78 g, 85.4 mmol, 3.0 eq.) in dichloroethane (80 ml). After stirring at 45° C. for 72 h, the resulting mixture was filtered through Celite®. The filtrate was concentrated under reduced pressure. The residue was purified by column chromatography (silica gel, eluent: petroleum ether/ethyl acetate 95:5). Yield: 2.40 g (90% purity, 39% of theory).

¹H-NMR (300 MHz, CDCl₃): δ [ppm]=5.84-5.45 (m, 1H), 4.17-4.09 (m, 2H), 3.47 (s, 3H), 3.42-3.36 (m, 1H), 2.54-2.40 (m, 2H), 2.03-1.89 (m, 1H), 1.88-1.74 (m, 1H), 1.28 (t, 3H).

¹⁹F-NMR (282 MHz, CDCl₃): δ [ppm]=−126.70 (d, 2F).

### Example 1.10D

### Ethyl 2-bromo-5,5-difluoro-4-methoxypentanoate (Mixture of Stereoisomers)

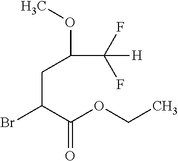

Lithium diisopropylamide solution (8.0 ml, 2.0 M in tetrahydrofuran, 16.0 mmol, 1.2 eq.) was added under argon atmosphere at −78° C. to a solution of ethyl 5,5-difluoro-4-methoxypentanoate (racemate) (2.90 g, 90% purity, 13.3 mmol, 1.0 eq.) in tetrahydrofuran (70 ml). The resulting mixture was stirred at −78° C. for 1 h, followed by the addition of a solution of 1,2-dibromo-1,1,2,2-tetrachloroethane (5.20 g, 16.0 mmol, 1.2 eq.) in tetrahydrofuran (20 ml) dropwise at the same temperature. After warming to RT and stirring for further 2 h, the reaction mixture was quenched with saturated aqueous solution of ammonium chloride and extracted with ethyl acetate. The combined organic phases were washed with water and brine, dried over anhydrous sodium sulfate, filtered and concentrated under reduced pressure. The residue was purified by preparative thin-layer chromatography (silica gel, eluent: petroleum ether/ethyl acetate 80:20). Yield: 1.85 g (85% purity, 43% of theory).

¹H-NMR (400 MHz, CDCl₃): δ [ppm]=5.87-5.70 (m, 1H), 4.50-4.22 (m, 3H), 3.66-3.42 (m, 4H), 2.23-2.16 (m, 2H), 1.35-1.26 (m, 3H).

¹⁹F-NMR (376 MHz, CDCl₃): δ [ppm]=−126.60 (s, 2F).

### Example 1.10E

### 2-Bromo-5,5-difluoro-4-methoxypentanoic Acid (Mixture of Stereoisomers)

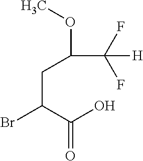

Lithium hydroxide (255 mg, 10.6 mmol, 2.0 eq.) was added at RT to a solution of ethyl 2-bromo-5,5-difluoro-4-methoxypentanoate (mixture of stereoisomers) (1.72 g, 85% purity, 5.3 mmol) in a mixture of tetrahydrofuran and water (3:1, 20 ml). The reaction mixture was stirred at RT for 100 min and acidified with aqueous hydrochloric acid (1 N). After removing all volatiles under reduced pressure, the resulting mixture was extracted with ethyl acetate. The combined organic phases were washed with brine, dried over anhydrous sodium sulfate, filtered and concentrated under reduced pressure (at <30° C. and >100 mbar). The crude product was used without further purification. Yield: 1.65 g.

### Example 1.10F

### 4-{[(2R,4R)-2-Bromo-5,5-difluoro-4-methoxypentanoyl]amino}benzamide (Single Stereoisomer)

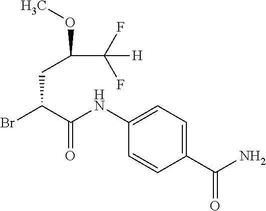 [SMILES: CO[C@H](C[C@@H](Br)C(=O)Nc1ccc(C(N)=O)cc1)C(F)F]

2-Bromo-5,5-difluoro-4-methoxypentanoic acid (mixture of stereoisomers) (1.65 g, 90% assumed purity of crude material, 6.0 mmol), pyridine (0.53 ml, 6.6 mmol, 1.1 eq.) and T3P (5.3 ml, 50% solution in ethyl acetate, 9.0 mmol, 1.5 eq.) were added under argon atmosphere at RT to a mixture of 4-aminobenzamide (817 mg, 6.0 mmol, 1.0 eq.) in tetrahydrofuran (20 ml). The reaction mixture was stirred at RT for 1 h, mixed with water, stirred for additional 15 min and mixed with additional water. After addition of ethyl acetate and phase separation, the aqueous phase was extracted with ethyl acetate. The combined organic phases were washed with brine, dried over anhydrous sodium sulfate, filtered and concentrated under reduced pressure. The residue was crystallized with dichloromethane, filtered and dried in vacuo. Yield: 993 mg (94% purity, 43% of theory). The combined mother liquids were concentrated under reduced pressure. The residue was purified by column chromatography (silica gel, eluent: dichloromethane/methanol gradient). Yield: 297 mg (13% of theory).

LC-MS (method 4): Rₜ=1.26 min; MS (ESIpos): m/z=365 [M+H]⁺

Stereoisomer separation of 1410 mg of 4-[(2-bromo-5,5-difluoro-4-methoxypentanoyl)amino]-benzamide (mixture of stereoisomers) gave
mixture of two stereoisomers 1+2 (chiral SFC 1: Rₜ=1.34/1.45 min): 290 mg,
single stereoisomer 3 (the title compound Example 1.10F) (chiral SFC 1: Rₜ=1.18 min, >98% ee): 330 mg,
single stereoisomer: (chiral SFC 2: Rₜ=1.40 min): 410 mg.

Separation method: SFC: column: Chiralpak AD-H 5 μm, 250 mm×20 mm; eluent: carbon dioxide/ethanol, gradient: 0 min 80% carbon dioxide, 5.00-8.43 min 70% carbon dioxide, 8.53-11.31 min 80% carbon dioxide; temperature: 40° C.; flow rate: 100 ml/min; UV detection: 210 nm.

Analysis method: SFC 1: column: Chiralpak AD-H, 50 mm×4.6 mm; eluent: 80% carbon dioxide/20% ethanol; temperature: 40° C.; flow rate: 3 ml/min; UV detection: 210 nm. SFC 2: column: Chiralpak AD-H, 50 mm×4.6 mm; eluent: 60% carbon dioxide/40% ethanol; temperature: 40° C.; flow rate: 3 ml/min; UV detection: 210 nm.

¹H-NMR (600 MHz, DMSO-d₆): δ [ppm]=10.61 (s, 1H), 7.90-7.81 (m, 3H), 7.65 (d, 2H), 7.24 (br s, 1H), 6.17 (dt, 1H), 4.76 (dd, 1H), 3.71-3.62 (m, 1H), 3.49 (s, 3H), 2.21-2.11 (m, 2H).

### Example 1.11A

### 4-{[(2R)-2-Bromopropanoyl]amino}benzamide (Single Stereoisomer)

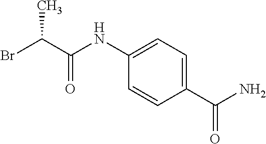

(2R)-2-Bromopropanoic acid (single stereoisomer) (1.00 g, 6.54 mmol), pyridine (0.58 ml, 7.19 mmol, 1.1 eq.) and T3P (5.73 ml, 50% solution in ethyl acetate, 9.81 mmol, 1.5 eq.) were added under argon atmosphere at RT to a mixture of 4-aminobenzamide (890 mg, 6.54 mmol, 1.0 eq.) in tetrahydrofuran (15 ml). The reaction mixture was stirred at RT for 30 min. After removing all volatiles under reduced pressure, the residue was mixed with additional water. The forming precipitate was filtered, washed with water and dried in vacuo. Yield: 1.77 g (quantitative of theory).

LC-MS (method 10): Rₜ=0.97 min; MS (ESIpos): m/z=271 [M+H]⁺

¹H-NMR (600 MHz, DMSO-d₆): δ [ppm]=10.52 (s, 1H), 7.87 (br s, 1H), 7.86 (d, 2H), 7.66 (d, 2H), 7.26 (br s, 1H), 4.71 (q, 1H), 1.76 (d, 3H).

### Example 2.1A

### 2-(2-Bromo-4-chlorophenyl)-N-methoxy-N-methylacetamide

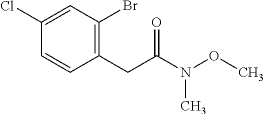

(2-Bromo-4-chlorophenyl)acetic acid (5.00 g, 20.0 mmol, 1.0 eq.) was dissolved in acetonitrile (100 ml). N-Methoxymethanamine hydrochloride (2.35 g, 24.0 mmol, 1.2 eq.), 1-(3-dimethylaminopropyl)-3-ethylcarbodiimide hydrochloride (4.61 g, 24.0 mmol, 1.2 eq.) and N-hydroxybenzotriazole (3.25 g, 24.0 mmol, 1.2 eq.) were added and the mixture was stirred at RT for 6 h. Subsequently, triethylamine (6.9 ml, 50 mmol, 2.5 eq.) was added and stirring was continued for 6 h. The suspension was concentrated and the residue was dissolved in ethyl acetate. The organic phase was washed with aqueous saturated sodium bicarbonate solution, water and brine, dried over anhydrous sodium sulfate, filtered and concentrated under reduced pressure. The crude product was purified by column chromatography (silica gel, eluent: cyclohexane/ethyl acetate 100:0 to 40:60). Yield: 4.00 g (66% of theory).

LC-MS (method 1): Rₜ=0.94 min; MS (ESIpos): m/z=294 [M+H]⁺

### Example 2.1B

### 1-(2-Bromo-4-chlorophenyl)acetone

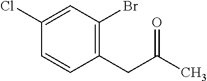

Methylmagnesium bromide solution (14 ml, 3.0 M in diethyl ether, 41 mmol, 3.0 eq.) was added to a solution of 2-(2-bromo-4-chlorophenyl)-N-methoxy-N-methylacetamide (4.00 g, 13.7 mmol, 1.0 eq.) in tetrahydrofuran (60 ml) at 0° C. under argon atmosphere. The reaction mixture was stirred at RT for 3 h. A saturated aqueous ammonium chloride solution was added under cooling, followed by addition of ethyl acetate. After phase separation, the organic phase was washed with brine, dried and concentrated. The crude product was purified by column chromatography (silica gel, eluent: cyclohexane/ethyl acetate 100:0 to 65:35). Yield: 1.60 g (47% of theory).

¹H-NMR (400 MHz, DMSO-d₆): δ [ppm]=7.73 (d, 1H), 7.43 (dd, 1H), 7.34 (d, 1H), 3.95 (s, 2H), 2.19 (s, 3H).

### Example 2.1C

### 1-(2-Bromo-4-chlorophenyl)propan-2-ol (Racemate)

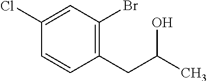

1-(2-Bromo-4-chlorophenyl)acetone (1.50 g, 6.06 mmol, 1.0 eq.) was dissolved in methanol (22 ml) and cooled to 0° C. Sodium borohydride (459 mg, 12.1 mmol, 2.0 eq.) was added and the reaction mixture was stirred for 10 min at 0° C. and for 1 h at RT. Acetone (2.9 ml) was added and stirring was continued for 5 min. After addition of saturated aqueous ammonium chloride solution, the mixture was concentrated under reduced pressure and ethyl acetate and water were added to the residue. After phase separation, the organic phase was washed with aqueous sodium chloride solution, dried over anhydrous sodium sulfate and concentrated. The crude product was purified by column chromatography (eluent: cyclohexane/ethyl acetate 100:0 to 60:40). Yield: 1.40 g (93% of theory).

¹H-NMR (600 MHz, DMSO-d₆): δ [ppm]=7.68 (d, 1H), 7.40-7.37 (m, 1H), 7.36-7.33 (m, 1H), 4.68 (d, 1H), 3.90-3.83 (m, 1H), 2.78-2.70 (m, 2H), 1.07 (d, 3H).

### Example 2.2A

### (2R)-1-(2-Bromo-4-chlorophenyl)propan-2-ol (Single Stereoisomer)

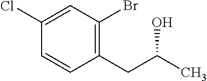

2-Bromo-4-chloro-1-iodobenzene (3.00 g, 9.45 mmol, 1.0 eq.) was dissolved in tetrahydrofuran (60 ml) and cooled to −40° C., followed by the dropwise addition of isopropylmagnesium chloride lithium chloride complex solution (8.7 ml, 1.3 M in tetrahydrofuran, 11 mmol, 1.2 eq.). The reaction mixture was stirred at −40° C. for 20 min, copper(I) chloride (234 mg, 2.36 mmol, 0.25 eq.) and (2R)-2-methyloxirane (single stereoisomer) (1.3 ml, 19 mmol, 2.0 eq.) were added and stirring was continued at RT overnight. After addition of saturated aqueous ammonium chloride solution, the mixture was concentrated under reduced pressure, dissolved in acetonitrile, filtered through diatomaceous earth and concentrated under reduced pressure. The residue was purified by column chromatography (silica gel, eluent: cyclohexane/ethyl acetate 99:1 to 3:2 followed by dichloromethane/methanol 4:1). Yield: 1.02 g (>99% ee, 43% of theory).

¹H-NMR (500 MHz, DMSO-d₆): δ [ppm]=7.68 (d, 1H), 7.40-7.33 (m, 2H), 4.66 (br s, 1H), 3.91-3.83 (m, 1H), 2.79-2.69 (m, 2H), 1.08 (d, 3H).

### Example 2.3A

### (2R)-1-(4-Chloro-3-fluorophenyl)propan-2-ol (Single Stereoisomer)

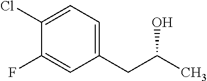

1-Chloro-2-fluoro-4-iodobenzene (2.5 ml, 19 mmol, 1.0 eq.) was dissolved in tetrahydrofuran (50 ml) and cooled to −40° C., followed by the dropwise addition of isopropylmagnesium chloride lithium chloride complex solution (18 ml, 1.3 M in tetrahydrofuran, 23 mmol, 1.2 eq.). The reaction mixture was stirred at −40° C. for 20 min, copper(I) chloride (483 mg, 4.87 mmol, 0.25 eq.) and (2R)-2-methyloxirane (single stereoisomer) (2.7 ml, 39 mmol, 2.0 eq.) were added and stirring was continued at RT overnight. After addition of saturated aqueous ammonium chloride solution, the mixture was concentrated under reduced pressure, dissolved in ethyl acetate and water and extracted with ethyl acetate. The combined organic phases were dried over anhydrous magnesium sulfate, filtered and concentrated under reduced pressure. The residue was purified by preparative HPLC (reversed phase, eluent: water with 0.05% formic acid/acetonitrile 80:20 to 5:95). Yield: 2.35 g (64% of theory).

¹H-NMR (600 MHz, DMSO-d₆): δ [ppm]=7.45 (dd, 1H), 7.24 (dd, 1H), 7.07 (d, 1H), 4.59 (d, 1H), 3.86-3.79 (m, 1H), 2.64 (d, 2H), 1.05 (d, 3H).

### Example 2.4A

### 2-Bromo-4-chloro-3-fluorobenzoic Acid

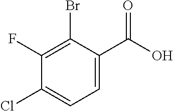 [SMILES: O=C(O)c1ccc(Cl)c(F)c1Br]

Lithium diisopropylamide (66 ml, 2.0 M in tetrahydrofuran, 132 mmol, 2.3 eq.) was added to a solution of 4-chloro-3-fluorobenzoic acid (10.0 g, 57.3 mmol, 1.0 eq.) in tetrahydrofuran (500 ml) dropwise at −78° C. under nitrogen atmosphere. The resulting mixture was stirred for 8 h at −78° C., followed by the addition of a solution of 1,2-dibromo-1,1,2,2-tetrachloroethane (37.3 g, 114.5 mmol, 2.0 eq.) in tetrahydrofuran (50 ml) dropwise over a period of 1 h. After stirring for 2 h at −78° C. and further 15 h at RT, the reaction mixture was worked up by addition of water and extracted with diethyl ether. The aqueous layer was adjusted to pH 3 by addition of aqueous hydrochloric acid (4 N) and extracted with ethyl acetate. The combined organic layers were washed with brine, dried over anhydrous sodium sulfate and filtered. The filtrate was concentrated under reduced pressure. The crude product was used in the subsequent reaction without further purification. Yield: 13.85 g (90% purity, 86% of theory).

¹H-NMR (400 MHz, DMSO-d₆): δ [ppm]=13.79 (br s, 1H), 7.75-7.69 (m, 1H), 7.67-7.60 (m, 1H).

### Example 2.4B

### 1-(2-Bromo-4-chloro-3-fluorophenyl)-2-diazoethan-1-one

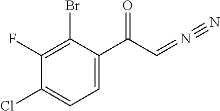 [SMILES: [N-]=[N+]=CC(=O)c1ccc(Cl)c(F)c1Br]

2-Bromo-4-chloro-3-fluorobenzoic acid (13.85 g, 90% purity, 49.2 mmol, 1.0 eq.) was dissolved in thionyl chloride (100 ml) and stirred for 2 h at 80° C. The reaction mixture was concentrated under reduced pressure, dissolved in tetrahydrofuran (270 ml), cooled to −5° C. and a solution of (trimethylsilyl)diazomethane (60 ml, 2.0 M in diethyl ether, 120 mmol, 2.5 eq.) was added. The resulting mixture was allowed to warm to RT during 2 h and concentrated under reduced pressure. The residue was purified by column chromatography (silica gel, eluent: petroleum ether/ethyl acetate 5:1). Yield: 13.0 g (90% purity, 87% of theory).

### Example 2.4C

### Methyl (2-bromo-4-chloro-3-fluorophenyl)acetate

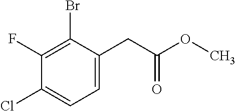

Silver(I) benzoate (4.83 g, 21.1 mmol, 0.5 eq.) was added to a solution of 1-(2-bromo-4-chloro-3-fluorophenyl)-2-diazoethan-1-one (13.0 g, 90% purity, 42.2 mmol, 1.0 eq.) in methanol (283 ml), followed by the addition of triethylamine (58.7 ml, 422 mmol, 10 eq.) dropwise at −5° C. After stirring for 15 h at RT, the reaction mixture was filtered through a pad of Celite®. The filtrate was concentrated under reduced pressure. The residue was dissolved in ethyl acetate and washed with saturated aqueous sodium carbonate solution, water and brine. The organic layer was dried over anhydrous sodium sulfate, filtered and concentrated under reduced pressure. The residue was purified by column chromatography (silica gel, eluent: petroleum ether/ethyl acetate 97:3). Yield: 9.00 g (73% of theory).

¹H-NMR (400 MHz, DMSO-d₆): δ [ppm]=7.68-7.58 (m, 1H), 7.38-7.25 (m, 1H), 3.91 (s, 2H), 3.64 (s, 3H).

### Example 2.4D

### (2-Bromo-4-chloro-3-fluorophenyl)acetic Acid

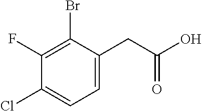 [SMILES: O=C(O)Cc1ccc(Cl)c(F)c1Br]

An aqueous solution of sodium hydroxide (84.3 ml, 1.0 M, 84.3 mmol, 4.0 eq.) was added to a solution of methyl 2-(2-bromo-4-chloro-3-fluorophenyl)acetate (6.11 g, 20.8 mmol, 1.0 eq.) in ethanol (80 ml). After stirring for 15 h at RT, the reaction mixture was concentrated under reduced pressure, adjusted to pH 3 by addition of aqueous hydrochloric acid (3 N) and extracted with ethyl acetate. The combined organic layers were washed with brine, dried over anhydrous sodium sulfate and filtered. The filtrate was concentrated under reduced pressure and the crude product was used in the subsequent step without further purification. Yield: 5.55 g (99% of theory).

¹H-NMR (400 MHz, DMSO-d₆): δ [ppm]=12.66 (br s, 1H), 7.61 (dd, 1H), 7.32 (d, 1H), 3.82 (s, 2H).

### Example 2.4E

### 2-(2-Bromo-4-chloro-3-fluorophenyl)-N-methoxy-N-methylacetamide

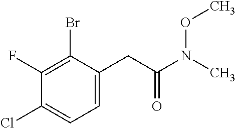 [SMILES: CON(C)C(=O)Cc1ccc(Cl)c(F)c1Br]

(2-Bromo-4-chloro-3-fluorophenyl)acetic acid (5.55 g, 20.5 mmol, 1.0 eq.) was dissolved in dichloromethane (110 ml) and N,O-dimethylhydroxylamine hydrochloride (3.00 g, 30.8 mmol, 1.5 eq.), 4-dimethylaminopyridine (2.51 g, 20.5 mmol, 1.0 eq.), 1-(3-dimethylaminopropyl)-3-ethylcarbodiimide hydrochloride (5.91 g, 30.8 mmol, 1.5 eq.) and triethylamine (11.4 ml, 82.2 mmol, 4.0 eq.) were added. After stirring for 15 h at RT, the reaction mixture was diluted with ethyl acetate, washed with saturated aqueous sodium bicarbonate solution, aqueous hydrochloric acid (2.0 N), water and brine. The organic layer was dried over anhydrous sodium sulfate and filtered. The filtrate was concentrated under reduced pressure and used in the subsequent step without further purification. Yield: 6.25 g (95% of theory).

¹H-NMR (300 MHz, DMSO-d₆): δ [ppm]=7.66-7.50 (m, 1H), 7.26 (dd, 1H), 3.98 (br s, 2H), 3.76 (s, 3H), 3.13 (br s, 3H).

### Example 2.4F

### 1-(2-Bromo-4-chloro-3-fluorophenyl)propan-2-one

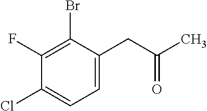

Methylmagnesium bromide solution (26.0 ml, 3.0 M in ethyl ether, 78.1 mmol, 4.0 eq.) was added to a solution of 2-(2-bromo-4-chloro-3-fluorophenyl)-N-methoxy-N-methylacetamide (6.25 g, 19.5 mmol, 1.0 eq.) in tetrahydrofuran (120 ml) at 0° C. under nitrogen atmosphere. After stirring for 4 h at RT, the reaction mixture was treated with aqueous hydrochloric acid (1 N) and extracted with ethyl acetate. The combined organic layers were washed with water and brine, dried over anhydrous sodium sulfate and filtered. The filtrate was concentrated under reduced pressure and used in the subsequent step without further purification. Yield 5.00 g (94% purity, 91% of theory).

¹H-NMR (300 MHz, DMSO-d₆): δ [ppm]=7.65-7.55 (m, 1H), 7.25-7.18 (m, 1H), 4.04 (s, 2H), 2.22 (s, 3H).

### Example 2.4G

### 1-(2-Bromo-4-chloro-3-fluorophenyl)propan-2-ol (Racemate)

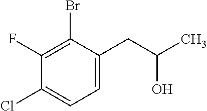

Sodium borohydride (1.01 g, 26.6 mmol, 1.5 eq.) was added portionwise to a solution of 1-(2-bromo-4-chloro-3-fluorophenyl)propan-2-one (5.00 g, 94% purity, 17.7 mmol, 1.0 eq.) in ethanol (100 ml) at 0° C. under nitrogen atmosphere. After stirring for 2 h at RT, the reaction mixture was treated with saturated aqueous ammonium chloride solution and extracted with ethyl acetate. The combined organic layers were washed with water and brine, dried over anhydrous sodium sulfate, filtered and concentrated under reduced pressure. The residue was purified by column chromatography (silica gel, eluent: petroleum ether/ethyl acetate 9:1). Yield: 4.00 g (95% purity, 80% of theory).

¹H-NMR (300 MHz, DMSO-d₆): δ [ppm]=7.54 (dd, 1H), 7.22 (dd, 1H), 4.70 (d, 1H), 3.95-3.82 (m, 1H), 2.80 (d, 2H), 1.09 (d, 3H).

### Example 2.5A

### 1-(2-Bromo-4-chlorophenyl)butan-2-one

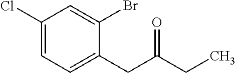

2-(2-Bromo-4-chlorophenyl)-N-methoxy-N-methylacetamide (24.0 g, 82.0 mmol, 1.0 eq.) was dissolved in tetrahydrofuran (360 ml) and ethyl magnesium bromide solution (137 ml, 3.0 M in diethyl ether, 410 mmol, 5.0 eq.) was added dropwise at −30° C. under nitrogen atmosphere. After stirring for 15 h at RT, the reaction mixture was treated slowly with aqueous hydrochloric acid (200 ml, 1 N) at −10° C., stirred for further 10 min, and extracted with ethyl acetate. The combined organic layers were washed with saturated aqueous sodium carbonate solution, water and with brine, dried over anhydrous sodium sulfate, filtered and concentrated under reduced pressure. The residue was purified by column chromatography (silica gel, eluent: petroleum ether/ethyl acetate 20:1). Yield: 8.00 g (95% purity, 35% of theory).

¹H-NMR (400 MHz, DMSO-d₆): δ [ppm]=7.44 (d, 1H), 7.45-7.42 (m, 1H), 7.37-7.33 (m, 1H), 3.94 (s, 2H), 2.58-2.52 (m, 2H), 0.97 (t, 3H).

### Example 2.5B

### 1-(2-Bromo-4-chlorophenyl)butan-2-ol (Racemate)

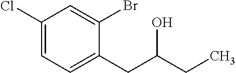

1-(2-Bromo-4-chlorophenyl)butan-2-one (8.00 g, 95% purity, 29.1 mmol, 1.0 eq.) was dissolved in ethanol (160 ml), cooled to 0° C. and sodium borohydride (1.32 g, 34.9 mmol, 1.2 eq.) was added. After stirring for 2 h at RT, the reaction mixture was treated with aqueous hydrochloric acid (1 N) and extracted with ethyl acetate. The combined organic layers were washed with water and brine, dried over anhydrous sodium sulfate and filtered. The filtrate was concentrated under reduced pressure and used in the subsequent reaction without further purification. Yield: 7.50 g (90% purity, 88% of theory).

¹H-NMR (400 MHz, DMSO-d₆): δ [ppm]=7.69 (d, 1H), 7.41-7.32 (m, 2H), 4.58 (d, 1H), 3.63-3.52 (m, 1H), 2.84-2.79 (m, 1H), 2.70-2.59 (m, 1H), 1.47-1.25 (m, 2H), 0.90 (t, 3H).

### Example 2.6A

### Ethyl 3-(2-bromo-4-chlorophenyl)acrylate (Mixture of E/Z Stereoisomers)

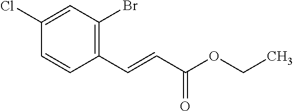

2-Bromo-4-chlorobenzaldehyde (12.0 g, 54.7 mmol, 1.0 eq.) was dissolved in dichloromethane (120 ml) and (ethoxycarbonylmethylene)-triphenylphosphorane (38.1 g, 109.4 mmol, 2.0 eq.) was added. After stirring for 1.5 h at RT, the reaction mixture was concentrated under reduced pressure. The residue was purified by column chromatography (silica gel, eluent: petroleum ether/ethyl acetate 10:1). Yield: 13.60 g (79% purity, 82% of theory).

¹H-NMR (400 MHz, DMSO-d₆): δ [ppm]=7.97 (d, 1H), 7.88-7.80 (m, 2H), 7.57-7.42 (m, 1H), 6.71/6.19 (2d, 1H), 4.22/4.04 (2q, 2H), 1.27/1.11 (2t, 3H).

### Example 2.6B

### 3-(2-Bromo-4-chlorophenyl)prop-2-en-1-ol (Mixture of E/Z Stereoisomers)

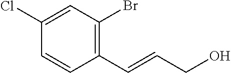 [SMILES: OC/C=C/c1ccc(Cl)cc1Br]

Ethyl 3-(2-bromo-4-chlorophenyl)acrylate (mixture of E/Z stereoisomers) (3.60 g, 12.4 mmol, 1.0 eq.) was dissolved in dichloromethane (75 ml) and diisobutylaluminum hydride solution (3.54 g, 1.0 M in toluene, 24.9 mmol, 2.0 eq.) was added dropwise at −70° C. After stirring for 2 h at RT, the reaction mixture was treated with sodium sulfate decahydrate (3.0 g) and stirring was continued for 20 min. After filtration through a pad of Celite®, the filtrate was diluted with dichloromethane and washed with water and brine, dried over anhydrous sodium sulfate, filtered and concentrated under reduced pressure. The resulting residue was purified by column chromatography (silica gel, eluent: petroleum ether/ethyl acetate 10:1). Yield: 2.90 g (91% of theory).

LC-MS (method 8): Rₜ=1.12 min; MS (ESIpos): m/z=229 [M+H−H₂O]⁺

¹H-NMR (300 MHz, DMSO-d₆): δ [ppm]=7.79/7.73 (2d, 1H), 7.70-7.67 (m, 1H), 7.49-7.26 (m, 1H), 6.83-6.77 (m, 1H), 6.50-6.39/6.00-5.90 (2m, 1H), 5.03/4.92 (2t, 1H), 4.19-4.10/4.09-4.01 (2m, 2H).

### Example 2.6C

### (3-(2-Bromo-4-chlorophenyl)oxiran-2-yl)methanol (Racemate)

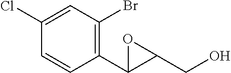 [SMILES: OCC1OC1c1ccc(Cl)cc1Br]

3-(2-Bromo-4-chlorophenyl)prop-2-en-1-ol (mixture of E/Z stereoisomers) (13.0 g, 49.9 mmol, 1.0 eq.) was dissolved in dichloromethane (250 ml), cooled to 0° C. and 3-chloroperoxybenzoic acid (17.2 g, 74.8 mmol, 75% purity, 1.5 eq.) was added. After stirring for 2 h at RT, the reaction mixture was diluted with dichloromethane and washed with saturated aqueous sodium bisulfite solution, saturated aqueous sodium bicarbonate solution, water and brine. The organic layer was dried over anhydrous sodium sulfate, filtered and concentrated under reduced pressure. The residue was purified by column chromatography (silica gel, eluent: petroleum ether/ethyl acetate 6:1). Yield: 12.0 g (85% purity, 78% of theory).

¹H-NMR (300 MHz, DMSO-d₆): δ [ppm]=7.79-7.69 (m, 1H), 7.59-7.43 (m, 1H), 7.25/7.20 (2d, 1H), 5.04/4.92 (2t, 1H), 4.13/4.03 (2d, 1H), 3.83-3.77 (m, 1H), 3.61-3.39 (m, 1H), 3.09-2.96 (m, 1H).

### Example 2.6D

### 3-(2-Bromo-4-chlorophenyl)propane-1,2-diol (Racemate)

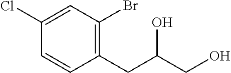 [SMILES: OCC(O)Cc1ccc(Cl)cc1Br]

(3-(2-Bromo-4-chlorophenyl)oxiran-2-yl)methanol (racemate) (12.0 g, 85% purity, 38.7 mmol, 1.0 eq.) was dissolved in dichloromethane (120 ml), cooled to 0° C. and diisobutylaluminum hydride solution (77.4 ml, 1.0 M in in toluene, 77.4 mmol, 2.0 eq.) was added. The reaction mixture was allowed to warm to RT and stirred for 2 h, followed by the addition of sodium sulfate decahydrate (75 g). Stirring was continued for 1 h at the same temperature. The resulting mixture was diluted with dichloromethane and filtered through a pad of Celite®. The filtrate was washed with water and brine, dried over anhydrous sodium sulfate, filtered and concentrated under reduced pressure. The residue was purified by column chromatography (silica gel, eluent: petroleum ether/ethyl acetate 1:1). Yield: 7.00 g (62% of theory).

¹H-NMR (300 MHz, DMSO-d₆): δ [ppm]=7.70-7.68 (m, 1H), 7.43-7.38 (m, 2H), 4.66-4.64 (m, 2H), 3.71-3.61 (m, 2H), 3.36-3.34 (m, 1H), 2.96-2.90 (m, 1H), 2.62-2.58 (m, 1H).

### Example 2.6E

### 1-(2-Bromo-4-chlorophenyl)-3-{[tert-butyl(diphenyl)silyl]oxy}propan-2-ol (Racemate)

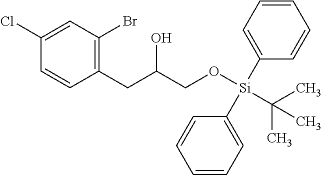 [SMILES: CC(C)(C)[Si](OCC(O)Cc1ccc(Cl)cc1Br)(c1ccccc1)c1ccccc1]

3-(2-Bromo-4-chlorophenyl)propane-1,2-diol (racemate) (10.0 g, 37.7 mmol, 1.0 eq.) was dissolved in N,N-dimethylformamide (150 ml), cooled to 0° C. and imidazole (5.64 g, 82.9 mmol, 2.2 eq.) and tert-butylchlorodiphenylsilane (15.5 g, 56.5 mmol, 1.5 eq.) were added. After stirring for 15 h at RT, the reaction mixture was treated with water and stirred for further 30 min, followed by extraction with ethyl acetate. The combined organic layers were washed with brine, dried over anhydrous sodium sulfate, filtered and concentrated under reduced pressure. The residue was purified by column chromatography (silica gel, eluent: petroleum ether/ethyl acetate 20:1) and further purified by column chromatography (silica gel, eluent: petroleum ether/dichloromethane 15:1). Yield: 5.50 g (91% purity) and 10.68 g (81% purity) (combined 72% of theory).

LC-MS (method 9): Rₜ=3.49 min; MS (ESIpos): m/z=527 [M+Na]⁺

¹H-NMR (300 MHz, DMSO-d₆): δ [ppm]=7.70-7.62 (m, 5H), 7.50-7.37 (m, 8H), 5.00-4.70 (m, 1H), 3.92-3.78 (m, 1H), 3.66-3.55 (m, 1H), 3.53-3.37 (m, 1H), 3.16-3.09 (m, 1H), 2.67-2.60 (m, 1H), 1.01 (s, 9H).

### Example 2.7A

### 1-(2-Bromo-4-chlorophenyl)-3-methylbutan-2-ol (Racemate)

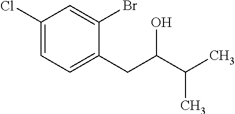

A solution of isopropylmagnesium bromide (19.3 ml, 3.0 M in 2-methyltetrahydrofuran, 58.0 mmol, 2.3 eq.) under argon atmosphere was cooled to −40° C. and tetrahydrofuran (40 ml) was added. A solution of (2-bromo-4-chlorophenyl)acetaldehyde (5.89 g, 25.2 mmol, 1.0 eq.) in tetrahydrofuran (20 ml) was added slowly. The mixture was stirred for 30 min at −40° C. and 1 h at RT. After cooling to 0° C., the reaction mixture was treated with a mixture of acetic acid and water (1:1, 7.2 ml) and combined with a mixture resulting from a similar experiment [with (2-bromo-4-chlorophenyl)acetaldehyde (1.0 g, 4.28 mmol, 1.0 eq) and isopropylmagnesium bromide solution (3.0 ml, 3.0 M in 2-methyltetrahydrofuran, 9 mmol, 2.1 eq.) in tetrahydrofuran (15 ml)]. Water was added and the mixture was extracted with ethyl acetate. The combined organic phases were washed with saturated aqueous sodium bicarbonate solution and brine, dried over anhydrous magnesium sulfate, filtered and concentrated under reduced pressure. The residue was purified by column chromatography (silica gel, eluent: cyclohexane/ethyl acetate 100:0 to 9:1). Yield: 3.20 g (80% purity, 31% of theory for both experiments).

¹H-NMR (400 MHz, DMSO-d₆): δ [ppm]=7.70-7.65 (m, 1H), 7.41-7.33 (m, 2H), 4.46 (d, 1H), 3.48-3.40 (m, 1H), 2.90-2.83 (m, 1H), 2.60-2.54 (m, 1H, partially concealed), 1.69-1.56 (m, 1H), 0.92 (d, 6H).

### Example 2.8A

### 3-(2-Bromo-4-chlorophenyl)-1,1-difluoropropan-2-ol (Racemate)

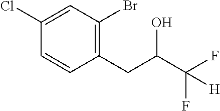 [SMILES: OC(Cc1ccc(Cl)cc1Br)C(F)F]

Trimethylsilyl chloride (11.7 g, 107 mmol, 1.5 eq.) was added to a solution of (2-bromo-4-chlorophenyl)acetaldehyde (16.7 g, 71.5 mmol, 1.0 eq.) and difluoro(triphenylphosphonio)acetate (33.2 g, 93.2 mmol, 1.3 eq.) in dichloromethane (250 ml). The resulting mixture was heated to 55° C. and stirred at this temperature for 3.5 h. After cooling to 5° C., the mixture was treated with a solution of potassium fluoride (12.5 g, 215 mmol, 3.0 eq.) in water (250 ml) and stirring was continued at 20° C. for 18 h. After phase separation, the aqueous phase was extracted with methyl tert-butyl ether. The combined organic phases were dried over anhydrous sodium sulfate, filtered, concentrated under reduced pressure and purified by column chromatography (silica gel, eluent: petroleum ether/ethyl acetate 1:1 to pure ethyl acetate). The obtained crude product was further purified by recrystallization from n-hexane. Yield: 3.70 g (18% of theory).

¹H-NMR (400 MHz, DMSO-d₆): δ [ppm]=7.73 (d, 1H), 7.44-7.38 (m, 2H), 6.19-5.74 (m, 1H), 5.64 (d, 1H), 3.93-3.81 (m, 1H), 2.97 (dd, 1H), 2.75 (dd, 1H).

### Example 2.9A

### 2-(2-Bromo-4-chlorophenyl)ethanol

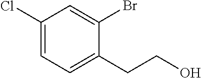 [SMILES: OCCc1ccc(Cl)cc1Br]

Methyl (2-bromo-4-chlorophenyl)acetate (10.70 g, 40.6 mmol, 1.0 eq.) under nitrogen atmosphere was dissolved in dichloromethane (100 ml) and cooled to −20° C. Diisobutylaluminum hydride solution (81.2 ml, 1.0 M in toluene, 81.2 mmol, 2.0 eq.) was added dropwise. The reaction mixture was stirred for 2 h at RT, treated with sodium sulfate decahydrate (60.0 g) and stirred for 20 min. After filtration through a pad of Celite®, the filtrate was concentrated under reduced pressure. The residue was purified by column chromatography (silica gel, eluent: petroleum ether/ethyl acetate 5:1). Yield: 8.80 g (92% of theory).

¹H-NMR (300 MHz, DMSO-d₆): δ [ppm]=7.69 (d, 1H), 7.41-7.35 (m, 2H), 4.76 (t, 1H), 3.61-3.54 (m, 2H), 2.83 (t, 2H).

### Example 2.9B

### 2-(2-Bromo-4-chlorophenyl)acetaldehyde

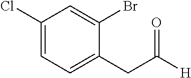 [SMILES: O=CCc1ccc(Cl)cc1Br]

2-(2-Bromo-4-chlorophenyl)ethanol (0.90 g, 2.5 mmol, 1.0 eq.) was dissolved in dichloromethane (5.0 ml) and Dess-Martin periodinane (2.14 g, 5.0 mmol, 2.0 eq.) was added. The reaction mixture was stirred at RT for 1 h. The mixture was concentrated under reduced pressure and the residue was purified by column chromatography (silica gel, eluent: petroleum ether/ethyl acetate 5:1). Yield: 0.20 g (85% purity, 28% of theory).

¹H-NMR (400 MHz, DMSO-d₆): δ [ppm]=9.69 (s, 1H), 7.81-7.78 (m, 1H), 7.49-7.39 (m, 2H), 3.97 (s, 2H).

### Example 2.9C

### 3-(2-Bromo-4-chlorophenyl)-1,1,1-trifluoropropan-2-ol (Racemate)

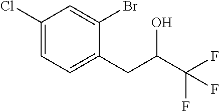 [SMILES: OC(Cc1ccc(Cl)cc1Br)C(F)(F)F]

Tetra-N-butylammonium fluoride solution (30.0 ml, 1.0 M in tetrahydrofuran, 30.0 mmol, 2.0 eq.) was added to a mixture of (2-bromo-4-chlorophenyl)acetaldehyde (7.00 g, 50% purity, 15.0 mmol, 1.0 eq.) and trimethyl(trifluoromethyl)silane (10.66 g, 74.9 mmol, 5.0 eq.) in tetrahydrofuran (70.0 ml) at 0° C. under nitrogen atmosphere. The reaction mixture was stirred at 0° C. for 2 h, followed by the addition of aqueous hydrochloric acid (1 N). The mixture was concentrated under reduced pressure and extracted with ethyl acetate. The combined organic layers were washed with brine, dried over anhydrous sodium sulfate and concentrated under reduced pressure. The residue was purified by column chromatography (silica gel, eluent: petroleum ether/ethyl acetate 5:1). Yield: 2.80 g (94% purity, 59% of theory).

¹H-NMR (300 MHz, DMSO-d₆): δ [ppm]=7.79-7.76 (m, 1H), 7.48-7.44 (m, 2H), 6.33 (d, 1H), 4.18-4.16 (m, 1H), 3.09-3.04 (m, 1H), 2.90-2.81 (m, 1H).

### Example 2.10A

### (2S)-3-(4-Chloro-3-fluorophenyl)-1,1,1-trifluoropropan-2-ol (Single Stereoisomer)

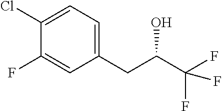 [SMILES: O[C@@H](Cc1ccc(Cl)c(F)c1)C(F)(F)F]

1-Chloro-2-fluoro-4-iodobenzene (3.4 ml, 27 mmol, 1.0 eq.) was dissolved in tetrahydrofuran (50 ml) and cooled to −40° C., followed by the dropwise addition of isopropylmagnesium chloride lithium chloride complex solution (26 ml, 1.3 M in tetrahydrofuran, 33 mmol, 1.25 eq.). The reaction mixture was stirred at −40° C. for 20 min, copper(I) chloride (663 mg, 6.69 mmol, 0.25 eq.) and (2S)-2-(trifluoromethyl)oxirane (single stereoisomer) (2.3 ml, 27 mmol, 1.0 eq.) were added and stirring was continued at RT overnight. After addition of saturated aqueous ammonium chloride solution, the mixture was concentrated under reduced pressure, dissolved in ethyl acetate and water and extracted with ethyl acetate. The combined organic phases were washed with brine, dried over anhydrous magnesium sulfate, filtered and concentrated under reduced pressure. The residue was purified by column chromatography (silica gel, eluent: cyclohexane/ethyl acetate mixture). Yield: 4.12 g (63% of theory).

¹H-NMR (600 MHz, DMSO-d₆): δ [ppm]=7.53-7.48 (m, 1H), 7.42-7.36 (m, 1H), 7.20-7.16 (m, 1H), 6.25 (d, 1H), 4.25-4.18 (m, 1H), 2.96 (dd, 1H), 2.75 (dd, 1H).

### Example 2.11A

### (2S)-3-(3,4-Dichlorophenyl)-1,1,1-trifluoropropan-2-ol (Single Stereoisomer)

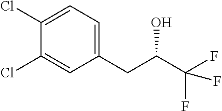 [SMILES: O[C@@H](Cc1ccc(Cl)c(Cl)c1)C(F)(F)F]

1,2-Dichloro-4-iodobenzene (7.06 g, 25.9 mmol, 1.0 eq.) was dissolved in tetrahydrofuran (50 ml) and cooled to −40° C., followed by the dropwise addition of isopropylmagnesium chloride lithium chloride complex solution (24 ml, 1.3 M in tetrahydrofuran, 31 mmol, 1.2 eq.). The reaction mixture was stirred at −40° C. for 20 min, copper(I) chloride (640 mg, 6.47 mmol, 0.25 eq.) and (2S)-2-(trifluoromethyl)oxirane (single stereoisomer) (2.8 ml, 32 mmol, 1.25 eq.) were added and stirring was continued at RT overnight. After addition of saturated aqueous ammonium chloride solution, the mixture was concentrated under reduced pressure, dissolved in ethyl acetate and water and extracted with ethyl acetate. The combined organic phases were washed with brine, dried over anhydrous magnesium sulfate, filtered and concentrated under reduced pressure. The residue was purified by preparative HPLC (reversed phase, eluent: water with 0.05% formic acid/acetonitrile 80:20 to 5:95). Yield: 4.53 g (68% of theory).

¹H-NMR (600 MHz, DMSO-d₆): δ [ppm]=7.61 (d, 1H), 7.56 (d, 1H), 7.31 (dd, 1H), 6.25 (d, 1H), 4.25-4.18 (m, 1H), 2.95 (dd, 1H), 2.74 (dd, 1H).

### Example 3.1A

### 4-Iodo-2-methoxy-5-methylpyridine

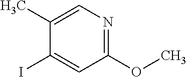

Sodium methoxide (144 g, 2.53 mol, 4.0 eq.) was dissolved in methanol (2.01) and 2-fluoro-4-iodo-5-methylpyridine (150 g, 633 mmol, 1.0 eq.) was added. The reaction mixture was heated to 60° C. and stirred at this temperature for 16 h. Subsequently, water and ethyl acetate were added, the resulting precipitate was filtered and the aqueous phase was extracted with ethyl acetate. The combined organic phases were washed with brine, dried over anhydrous sodium sulfate, filtered and concentrated under reduced pressure. The crude product was used in the subsequent reaction without further purification. Yield: 148 g (94% of theory).

LC-MS (method 1): Rₜ=0.99 min; MS (ESIpos): m/z=250 [M+H]⁺

¹H-NMR (400 MHz, DMSO-d₆): δ [ppm]=7.98 (s, 1H), 7.38 (s, 1H), 3.80 (s, 3H), 2.25 (s, 3H).

### Example 3.1B

### 5-(Bromomethyl)-4-iodo-2-methoxypyridine

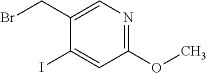

4-Iodo-2-methoxy-5-methylpyridine (25.0 g, 100 mmol, 1.0 eq.) was dissolved in chloroform (780 ml), followed by the addition of N-bromosuccinimide (26.8 g, 151 mmol, 1.5 eq.) and dibenzoyl peroxide (1.62 g, 75% in water, 5.02 mmol, 0.1 eq.). The resulting mixture was slowly heated to reflux, stirred at this temperature for 16 h and cooled down to RT. Diatomaceous earth was added and the volatiles were removed under reduced pressure. The crude product was purified by column chromatography (silica gel, eluent: cyclohexane/ethyl acetate 98:2 to 92:8). Yield: 22.5 g (90% purity, 62% of theory).

LC-MS (method 1): Rₜ=1.02 min; MS (ESIpos): m/z=328 [M+H]⁺

### Example 3.2A

### 4-Iodo-6-methoxynicotinaldehyde

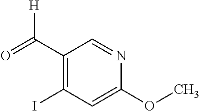

N-Methylmorpholine N-oxide (16.5 g, 122 mmol, 1.0 eq.) and activated 4 Å molecular sieve (17.0 g) were suspended in acetonitrile (480 ml) and cooled to 0° C. 5-(Bromomethyl)-4-iodo-2-methoxypyridine (20.0 g, 61.0 mmol, 2.0 eq.) was added and the reaction mixture was stirred for 2.5 h at RT. The reaction mixture was filtered through a pad of Celite®, washed with acetonitrile and concentrated under reduced pressure. The residue was purified by column chromatography (silica gel, eluent: cyclohexane/ethyl acetate 95:5 to 9:1 to 4:1). Yield: 12.0 g (91% purity, 68% of theory).

LC-MS (method 1): Rₜ=0.84 min; MS (ESIpos): m/z=264 [M+H]⁺

¹H-NMR (500 MHz, DMSO-d₆): δ [ppm]=9.88 (s, 1H), 8.51 (s, 1H), 7.55 (s, 1H), 3.94 (s, 3H).

### Example 3.2B

### (4-Iodo-6-methoxypyridin-3-yl)methanol

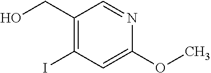

4-Iodo-6-methoxynicotinaldehyde (16.5 g, 60.1 mmol, 1.0 eq.) was dissolved in methanol (320 ml), cooled to 0° C. and sodium borohydride (2.73 g, 72.1 mmol, 1.2 eq.) was added. After stirring at RT for 2 h, the reaction mixture was concentrated under reduced pressure. The residue was dissolved in ethyl acetate and washed with saturated aqueous sodium carbonate solution, saturated aqueous of ammonium chloride solution, water and brine. The organic layer was dried over anhydrous sodium sulfate, filtered and concentrated under reduced pressure. The residue was purified by column chromatography (silica gel, eluent: petroleum ether/ethyl acetate 5:1). Yield: 14.8 g (91% of theory).

¹H-NMR (300 MHz, DMSO-d₆): δ [ppm]=8.06 (s, 1H), 7.34 (s, 1H), 5.31 (t, 1H), 4.41 (d, 2H), 3.82 (s, 3H).

### Example 3.3A

### 1-(4-Iodo-6-methoxypyridin-3-yl)ethanol (Racemate)

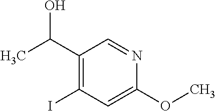

4-Iodo-6-methoxypyridine-3-carbaldehyde (13.5 g, 51.3 mmol, 1.0 eq.) was dissolved in tetrahydrofuran (140 ml) and cooled to −78° C., followed by the dropwise addition of methylmagnesium bromide (27 ml, 3.0 M in diethyl ether, 82 mmol, 1.6 eq.). The reaction mixture was stirred at −78° C. for 1 h, followed by addition of saturated aqueous ammonium chloride solution. The aqueous phase was extracted with ethyl acetate and the combined organic phases were dried over anhydrous sodium sulfate, filtered and concentrated under reduced pressure. The residue was purified by column chromatography (silica gel, eluent: cyclohexane/ethyl acetate 9:1 to 1:1). Yield: 11.8 g (82% of theory).

LC-MS (method 4): Rₜ=1.36 min; MS (ESIpos): m/z=280 [M+H]⁺

¹H-NMR (500 MHz, DMSO-d₆): δ [ppm]=8.15 (s, 1H), 7.29 (s, 1H), 5.41 (d, 1H), 4.79-4.72 (m, 1H), 3.32 (s, 3H), 1.31 (d, 3H).

### Example 4.1A

### Ethyl 1-(2,4-dimethoxybenzyl)-4-hydroxy-6-oxo-1,6-dihydropyridine-3-carboxylate

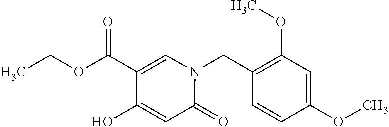 [SMILES: CCOC(=O)c1cn(Cc2ccc(OC)cc2OC)c(=O)cc1O]

A mixture of diethyl 3-oxopentanedioate (75.5 g, 359 mmol, 1.0 eq.) and diethoxymethyl acetate (61.1 g, 377 mmol, 1.05 eq.) was heated to 125° C. and stirred at this temperature for 2 h. For three times, the crude intermediate was treated with toluene and concentrated under reduced pressure. The residue was dissolved in tetrahydrofuran (150 ml) under argon atmosphere, 1-(2,4-dimethoxyphenyl)methanamine (68.0 g, 394 mmol, 1.1 eq.) was added and the resulting reaction mixture was stirred for 2 days at RT. After addition of tetrahydrofuran (100 ml) and cooling to 0° C., sodium hydride (10.0 g, 60% dispersion in mineral oil, 251 mmol, 0.7 eq.) was added in several portions within 0.5 h and the reaction mixture was stirred overnight. Aqueous hydrochloric acid (286 ml, 1 N) was added at 0° C. and the mixture was extracted with ethyl acetate. The combined organic phases were washed with brine, dried over anhydrous sodium sulfate, filtered and the filtrate was concentrated under reduced pressure. The crude product was used in the subsequent reaction without further purification. Yield: 143 g (81% purity, 97% of theory).

LC-MS (method 4): Rₜ=1.70 min; MS (ESIpos): m/z=334 [M+H]⁺

¹H-NMR (400 MHz, DMSO-d₆): δ [ppm]=10.71 (br s, 1H), 8.43 (s, 1H), 7.07 (d, 1H), 6.59 (d, 1H), 6.49 (dd, 1H), 5.66 (s, 1H), 4.95 (s, 2H), 4.27 (q, 2H), 3.81 (s, 3H), 3.75 (s, 3H), 1.29 (t, 3H).

### Example 4.1B

### Ethyl 1-(2,4-dimethoxybenzyl)-6-oxo-4-{[(trifluoromethyl)sulfonyl]oxy}-1,6-dihydropyridine-3-carboxylate

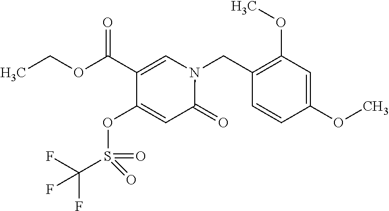 [SMILES: CCOC(=O)c1cn(Cc2ccc(OC)cc2OC)c(=O)cc1OS(=O)(=O)C(F)(F)F]

A solution of ethyl 1-(2,4-dimethoxybenzyl)-4-hydroxy-6-oxo-1,6-dihydropyridine-3-carboxylate (11.9 g, 35.0 mmol, 1.0 eq.) in dichloromethane (100 ml) under argon atmosphere was cooled to −78° C. and triethylamine (7.31 ml, 52.4 mmol, 1.5 eq.) was added. After addition of N-(5-chloropyridin-2-yl)-1,1,1-trifluoro-N-[(trifluoromethyl)sulfonyl]methanesulfonamide (19.2 g, 49.0 mmol, 1.4 eq.), the mixture was stirred at −78° C. for 0.5 h. The cooling bath was removed and stirring was continued for 2 h at RT. The mixture was concentrated under reduced pressure and the residue was purified by column chromatography (silica gel, eluent: cyclohexane/ethyl acetate 85:15 to 0:100). Yield: 14.35 g (88% of theory).

LC-MS (method 1): Rₜ=1.13 min; MS (ESIpos): m/z=466 [M+H]⁺

¹H-NMR (400 MHz, DMSO-d₆): δ [ppm]=8.68 (s, 1H), 7.19 (d, 1H), 6.61-6.51 (m, 2H), 6.55-6.50 (m, 1H), 5.05 (s, 2H), 4.29 (q, 2H), 3.80 (s, 3H), 3.76 (s, 3H), 1.29 (t, 3H).

### Example 4.1C

### Ethyl 1-(2,4-dimethoxybenzyl)-6-oxo-4-(4,4,5,5-tetramethyl-1,3,2-dioxaborolan-2-yl)-1,6-dihydropyridine-3-carboxylate

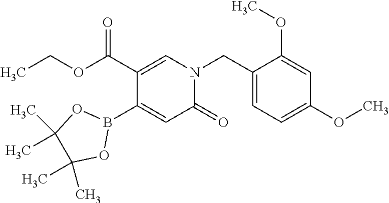 [SMILES: CCOC(=O)c1cn(Cc2ccc(OC)cc2OC)c(=O)cc1B1OC(C)(C)C(C)(C)O1]

Ethyl 1-(2,4-dimethoxybenzyl)-6-oxo-4-{[(trifluoromethyl)sulfonyl]oxy}-1,6-dihydropyridine-3-carboxylate (40.0 g, 80% purity, 68.8 mmol, 1.0 eq.) was dissolved in 1,4-dioxane (800 ml). Bis(pinacolato)diboron (26.2 g, 103 mmol, 1.5 eq.), potassium acetate (20.2 g, 206 mmol, 3.0 eq.) and [1,1′-bis(diphenylphosphino)ferrocene]palladium(II)dichloride dichloromethane complex (5.62 g, 6.88 mmol, 0.1 eq.) were added. Nitrogen was passed through the mixture for 15 min and the mixture was stirred at 80° C. for 15 h under nitrogen atmosphere. After cooled to RT, the reaction mixture was filtered through a pad of Celite® and washed with 1,4-dioxane. The filtrate was concentrated under reduced pressure. The crude product was used in the subsequent reaction without further purification. Yield: 52.0 g (58% purity, quantitative of theory).

LC-MS (method 16): Rₜ=1.13 min; MS (ESIpos): m/z=444 [M+H]⁺

### Example 4.1D

### Ethyl 4-[5-chloro-2-(2-hydroxypropyl)phenyl]-1-(2,4-dimethoxybenzyl)-6-oxo-1,6-dihydropyridine-3-carboxylate (Racemate)

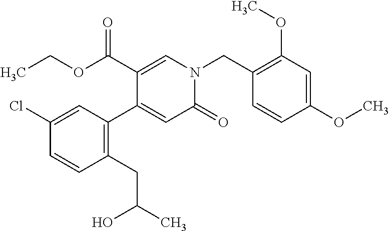 [SMILES: CCOC(=O)c1cn(Cc2ccc(OC)cc2OC)c(=O)cc1-c1cc(Cl)ccc1CC(C)O]

Ethyl 1-(2,4-dimethoxybenzyl)-6-oxo-4-{[(trifluoromethyl)-sulfonyl]oxy}-1,6-dihydropyridine-3-carboxylate (14.2 g, 30.5 mmol, 1.0 eq.) under argon atmosphere was dissolved in 1,4-dioxane (130 ml), bis(pinacolato)diboron (9.30 g, 36.6 mmol, 1.2 eq.), potassium acetate (8.99 g, 91.6 mmol, 3.0 eq.) and [1,1′-bis(diphenylphosphino)ferrocene]dichloropalladium(II) dichloromethane complex (300 mg, 0.37 mmol, 0.0125 eq.) were added and argon was passed through the resulting suspension for 10 min. The reaction mixture was heated to 100° C. and stirred at this temperature overnight. 1-(2-Bromo-4-chlorophenyl)propan-2-ol (racemate) (9.52 g, 38.2 mmol, 1.25 eq.), aqueous sodium carbonate solution (30.5 ml, 2.0 M, 61.0 mmol, 2.0 eq.) and [1,1′-bis(diphenylphosphino)ferrocene]dichloropalladium(II) dichloromethane complex (1.57 g, 1.92 mmol, 0.0625 eq.) were added and the mixture was stirred at 120° C. for 3 h. After cooling to RT, water and ethyl acetate were added and the aqueous phase was extracted with ethyl acetate. The combined organic phases were washed with brine, dried over anhydrous sodium sulfate, filtered over silica gel and concentrated under reduced pressure. The crude product was purified by column chromatography (silica gel, eluent: cyclohexane/ethyl acetate 92:8 to 34:66). Yield: 5.50 g (37% of theory).

LC-MS (method 1): Rₜ=1.04 min; MS (ESIpos): m/z=486 [M+H]⁺

¹H-NMR (400 MHz, DMSO-d₆): δ [ppm]=8.49 (d, 1H), 7.37-7.24 (m, 2H), 7.18 (dd, 1H), 7.12 (dd, 1H), 6.63 (d, 1H), 6.57-6.48 (m, 1H), 6.18 (d, 1H), 5.14-4.96 (m, 2H), 4.49 (dd, 1H), 3.97-3.87 (m, 2H), 3.83 (s, 3H), 3.77 (s, 3H), 3.75-3.54 (m, 1H), 2.63-2.43 (m, 1H), 2.40-2.21 (m, 1H), 0.91 (t, 3H), 0.93-0.82 (m, 3H).

### Example 4.1E

### 4-[5-Chloro-2-(2-hydroxypropyl)phenyl]-1-(2,4-dimethoxybenzyl)-5-(hydroxymethyl)pyridin-2(1H)-one (Racemate)

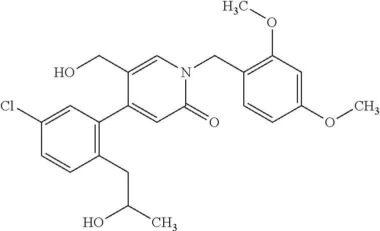 [SMILES: COc1ccc(Cn2cc(CO)c(-c3cc(Cl)ccc3CC(C)O)cc2=O)c(OC)c1]

Ethyl 4-[5-chloro-2-(2-hydroxypropyl)phenyl]-1-(2,4-dimethoxybenzyl)-6-oxo-1,6-dihydropyridine-3-carboxylate (racemate) (5.50 g, 11.3 mmol, 1.0 eq.) under argon atmosphere was dissolved in dichloromethane (200 ml) and cooled to −78° C. A solution of diisobutylaluminum hydride solution (56.6 ml, 1.0 M in dichloromethane, 56.6 mmol, 5.0 eq.) was added dropwise and the mixture was stirred at −78° C. for 0.5 h. Aqueous hydrochloric acid (1 N) was added, followed by the addition of saturated aqueous potassium and sodium tartrate solution and water. After warming to RT, stirring was continued for 15 min. The phases were separated and the aqueous phase was extracted with ethyl acetate. The combined organic phases were washed with brine, dried over anhydrous sodium sulfate and concentrated under reduced pressure. The crude product was purified by preparative HPLC (reversed phase, eluent: water with 0.05% formic acid/acetonitrile 90:10 to 5:95). Yield: 2.62 g (50% of theory).

LC-MS (method 5): Rₜ=1.09 min; MS (ESIpos): m/z=444 [M+H]⁺

¹H-NMR (400 MHz, DMSO-d₆): δ [ppm]=7.61 (s, 1H), 7.40-7.33 (m, 2H), 7.18 (s, 1H), 7.05 (dd, 1H), 6.62 (d, 1H), 6.50 (dd, 1H), 6.15 (d, 1H), 5.09-4.86 (m, 3H), 4.61-4.46 (m, 1H), 3.94-3.86 (m, 2H), 3.83 (s, 3H), 3.76 (s, 3H), 3.75-3.63 (m, 1H), 2.58 (dd, 1H), 2.49-2.42 (m, 1H), 2.33 (dd, 1H), 0.93 (dd, 3H).

### Example 4.1F

### 11-Chloro-3-(2,4-dimethoxybenzyl)-7-methyl-3,5,7,8-tetrahydro-2H-[3]benzoxocino[5,6-c]pyridin-2-one (Racemate)

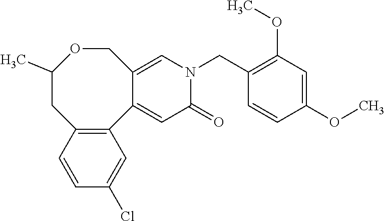

4-[5-Chloro-2-(2-hydroxypropyl)phenyl]-1-(2,4-dimethoxybenzyl)-5-(hydroxymethyl)pyridin-2(1H)-one (racemate) (2.61 g, 5.58 mmol, 1.0 eq.) under argon atmosphere was dissolved in dichloromethane (150 ml). Triethylamine (1.95 ml, 14.0 mmol, 2.5 eq.) was added and the solution was cooled to 0° C. At this temperature, methanesulfonyl chloride (0.52 ml, 6.70 mmol, 1.2 eq.) was added and the reaction mixture was stirred at RT overnight and at 40° C. for 3 h. Further amounts of triethylamine (1.01 ml, 7.26 mmol, 1.3 eq.) and methanesulfonyl chloride (0.26 ml, 3.35 mmol, 0.6 eq.) were added and stirring was continued at 40° C. for 2 h. The reaction mixture was concentrated under reduced pressure and the crude mixture was purified by column chromatography (silica gel, eluent: cyclohexane/ethyl acetate 80:20 to 50:50). Yield: 1.80 g (74% of theory).

LC-MS (method 5): Rₜ=1.42 min; MS (ESIpos): m/z=426 [M+H]⁺

¹H-NMR (400 MHz, DMSO-d₆): δ [ppm]=7.79 (s, 1H), 7.53-7.46 (m, 1H), 7.42-7.31 (m, 2H), 7.07 (d, 1H), 6.60 (d, 1H), 6.51 (dd, 1H), 6.35 (s, 1H), 5.09-4.85 (m, 2H), 4.39 (d, 1H), 3.83 (s, 3H), 3.75 (s, 3H), 3.61-3.42 (m, 2H), 2.80 (d, 1H), 2.29 (dd, 1H), 1.23 (d, 3H).

### Example 4.1G

### 11-Chloro-7-methyl-3,5,7,8-tetrahydro-2H-[3]benzoxocino[5,6-c]pyridin-2-one (Racemate)

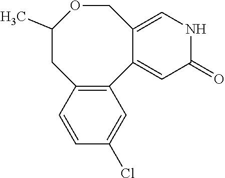 [SMILES: CC1Cc2ccc(Cl)cc2-c2cc(=O)[nH]cc2CO1]

11-Chloro-3-(2,4-dimethoxybenzyl)-7-methyl-3,5,7,8-tetrahydro-2H-[3]benzoxocino[5,6-c]pyridin-2-one (racemate) (1.80 g, 4.14 mmol, 1.0 eq.) was dissolved in trifluoroacetic acid (50 ml). The reaction mixture was heated to 65° C. and stirred at this temperature for 2 h. The volatiles were removed under reduced pressure and for three times, the crude mixture was treated with dichloromethane and subsequently concentrated under reduced pressure. The resulting residue was purified by preparative HPLC (reversed phase, eluent: water with 0.05% formic acid/acetonitrile: 90:10 to 5:95). Yield: 0.54 g (47% of theory).

LC-MS (method 5): Rₜ=1.03 min; MS (ESIpos): m/z=276 [M+H]⁺

¹H-NMR (400 MHz, DMSO-d₆): δ [ppm]=11.80 (br s, 1H), 7.56 (s, 1H), 7.53-7.44 (m, 1H), 7.41-7.33 (m, 1H), 7.32-7.25 (m, 1H), 6.27 (s, 1H), 4.43 (d, 1H), 3.58-3.50 (m, 1H), 3.48 (d, 1H), 3.41-3.34 (m, 1H), 2.84-2.74 (m, 1H), 2.33-2.19 (m, 1H), 1.23 (d, 3H).

### Example 4.2A

### 5-({[(2R)-1-(2-Bromo-4-chlorophenyl)propan-2-yl]oxy}methyl)-4-iodo-2-methoxypyridine (Single Stereoisomer)

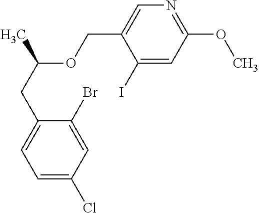 [SMILES: COc1cc(I)c(CO[C@H](C)Cc2ccc(Cl)cc2Br)cn1]

Sodium hydride (264 mg, 60% dispersion in mineral oil, 6.59 mmol, 2.5 eq.) was added under argon atmosphere at 0° C. to a mixture of (2R)-1-(2-bromo-4-chlorophenyl)propan-2-ol (single stereoisomer) (658 mg, 2.64 mmol) and 5-(bromomethyl)-4-iodo-2-methoxypyridine (1.00 g, 2.90 mmol, 1.1 eq.) in tetrahydrofuran (15 ml). The reaction mixture was stirred at 0° C. for 1 h, allowed to warm to RT, stirred at RT for 3 days and diluted with ethyl acetate (15 ml). The organic phase was washed with saturated aqueous ammonium chloride solution, water and brine, dried over anhydrous sodium sulfate, filtered and concentrated under reduced pressure. The residue was purified by column chromatography (silica gel, eluent: cyclohexane/ethyl acetate gradient). Yield: 1.08 g (82% of theory).

LC-MS (method 4): Rₜ=2.85 min; MS (ESIpos): m/z=496 [M+H]⁺

### Example 4.2B

### (7R)-11-Chloro-2-methoxy-7-methyl-7,8-dihydro-5H-[3]benzoxocino[5,6-c]pyridine (Single Stereoisomer)

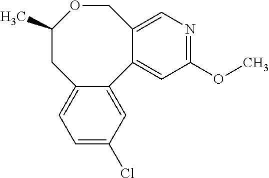

In a flame dried flask, 5-({[(2R)-1-(2-bromo-4-chlorophenyl)propan-2-yl]oxy}methyl)-4-iodo-2-methoxypyridine (single stereoisomer) (968 mg, 1.95 mmol) was dissolved under argon atmosphere in N,N-dimethylformamide (18 ml). Bis(pinacolato)diboron (743 mg, 2.92 mmol, 1.5 eq.), palladium(II) acetate (87.5 mg, 390 μmol, 0.2 eq.), tricyclohexylphosphine (82.0 mg, 292 μmol, 0.15 eq.) and potassium acetate (574 mg, 5.85 mmol, 3.0 eq.) were added, and argon was passed through the reaction mixture for 15 min. The reaction mixture was stirred at 80° C. for 3.5 h, potassium carbonate (538 mg, 3.90 mmol, 2.0 eq.) was added, the reaction mixture was stirred at 80° C. for 2.5 h, water (2 drops) was added and the reaction mixture was stirred at 80° C. for 4 h. After storage overnight at RT, the mixture was filtered through Celite®. The filter cake was washed several times with N,N-dimethylformamide. The combined filtrates were concentrated under reduced pressure. The residue was diluted with water and extracted with ethyl acetate. The combined organic phases were washed with brine, dried over anhydrous sodium sulfate, filtered and concentrated under reduced pressure. The residue was purified by column chromatography (silica gel, eluent: cyclohexane/ethyl acetate gradient). Yield: 295 mg (50% of theory).

LC-MS (method 4): Rₜ=2.23 min; MS (ESIpos): m/z=290 [M+H]⁺

### Example 4.2C

### (7R)-11-Chloro-7-methyl-3,5,7,8-tetrahydro-2H-[3]benzoxocino[5,6-c]pyridin-2-one (Single Stereoisomer)

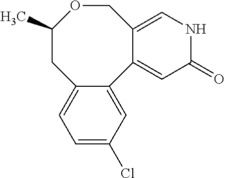 [SMILES: C[C@@H]1Cc2ccc(Cl)cc2-c2cc(=O)[nH]cc2CO1]

Sodium iodide (293 mg, 1.95 mmol, 2.0 eq.) was added under argon atmosphere to a solution of (7R)-11-chloro-2-methoxy-7-methyl-7,8-dihydro-5H-[3]benzoxocino[5,6-c]pyridine (single stereoisomer) (295 mg, 0.98 mmol) in glacial acetic acid (12 ml). The reaction mixture was stirred at 80° C. for 4 h. Additional sodium iodide (73 mg, 0.49 mmol, 0.5 eq.) was added and the reaction mixture stirred for 1 h. After cooling to RT, the reaction mixture was diluted with dichloromethane and neutralized carefully with aqueous sodium hydrogen carbonate solution. After phase separation, the aqueous phase was extracted with dichloromethane. The combined organic phases were washed with brine, dried over anhydrous sodium sulfate, filtered and concentrated under reduced pressure. The residue was purified by preparative HPLC (reversed phase, eluent: acetonitrile/water gradient). Yield: 110 mg (41% of theory).

LC-MS (method 4): Rₜ=1.43 min; MS (ESIpos): m/z=276 [M+H]⁺

### Alternative Synthetic Route:

Enantiomer separation of 574 mg of 11-chloro-7-methyl-3,5,7,8-tetrahydro-2H-[3]benzoxocino[5,6-c]pyridin-2-one (racemate), Example 4.1G gave
single stereoisomer 1 (chiral SFC: Rₜ=2.37 min, 99% ee): 227 mg,
single stereoisomer 2 (the title compound Example 4.2C) (chiral SFC: Rₜ=3.77 min, 99% ee): 216 mg.

Separation method: SFC: column: Daicel Chiralpak AZ-H 5 μm, 250 mm×20 mm; eluent: 80% carbon dioxide/20% ethanol; temperature: 40° C.; flow rate: 114 ml/min; UV detection: 210 nm.

Analysis method: SFC: column: Daicel Chiralpak AZ-H 3 μm, 100 mm×4.6 mm; eluent: 80% carbon dioxide/20% ethanol; temperature: 40° C.; flow rate: 3.0 ml/min; UV detection: 210 nm.

### Example 4.3A

### 5-({[1-(4-Chlorophenyl)propan-2-yl]oxy}methyl)-4-iodo-2-methoxypyridine (Racemate)

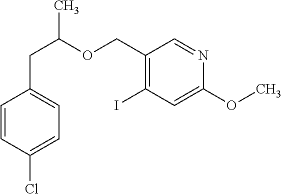 [SMILES: COc1cc(I)c(COC(C)Cc2ccc(Cl)cc2)cn1]

General Method 1 was carried out with 1-(4-chlorophenyl)propan-2-ol (racemate) (1.00 g, 80% purity, 4.69 mmol, 1.0 eq.), 5-(bromomethyl)-4-iodo-2-methoxypyridine (2.11 g, 80% purity, 5.16 mmol, 1.0 eq.) and sodium hydride (469 mg, 60% dispersion in mineral oil, 11.7 mmol, 2.5 eq.) in tetrahydrofuran (8.0 ml) for 1 h at −78° C. and overnight at RT. The crude product was purified by preparative HPLC (reversed phase, eluent: water with 0.05% formic acid/acetonitrile 90:10 to 5:95). Yield: 900 mg (42% of theory).

LC-MS (method 1): Rₜ=1.39 min; MS (ESIpos): m/z=418 [M+H]⁺

### Example 4.3B

### 11-Chloro-2-methoxy-7-methyl-7,8-dihydro-5H-[3]benzoxocino[5,6-c]pyridine (Racemate)

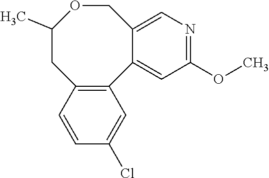

General Method 3b was carried out with 5-({[1-(4-chlorophenyl)propan-2-yl]oxy}methyl)-4-iodo-2-methoxypyridine (racemate) (50 mg, 120 μmol, 1.0 eq.), tetrakis(triphenylphosphine)-palladium(0) (13.8 mg, 12 μmol, 0.1 eq.) and potassium acetate (47 mg, 479 μmol, 4.0 eq.) in N,N-dimethylacetamide (2.0 ml) for 64 h at 120° C. The crude mixture was extracted with ethyl acetate, dried over anhydrous magnesium sulfate, filtered and concentrated under reduced pressure. The residue was purified by preparative HPLC (reversed phase, eluent: water with 0.05% formic acid/acetonitrile 90:10 to 5:95). Yield: 12.5 mg (36% of theory).

LC-MS (method 1): Rₜ=1.15 min; MS (ESIpos): m/z=290 [M+H]⁺

### Example 4.4A

### 5-({[1-(2-Bromo-4-chloro-3-fluorophenyl)propan-2-yl]oxy}methyl)-4-iodo-2-methoxypyridine (Racemate)

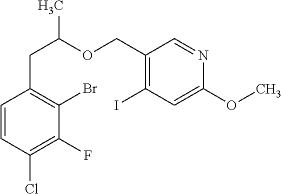 [SMILES: COc1cc(I)c(COC(C)Cc2ccc(Cl)c(F)c2Br)cn1]

(4-Iodo-6-methoxypyridin-3-yl)methanol (0.68 g, 2.51 mmol, 1.0 eq.) and scandium(III) trifluoromethanesulfonate (1.24 g, 2.51 mmol, 1.0 eq.) were added to a solution of 1-(2-bromo-4-chloro-3-fluorophenyl)propan-2-ol (racemate) (0.80 g, 84% purity, 2.51 mmol, 1.0 eq.) in nitromethane (13 ml) under nitrogen atmosphere. After stirring for 5.5 h at 90° C., the reaction mixture was diluted with ethyl acetate and washed with saturated aqueous sodium bicarbonate solution, water and brine. The organic layer was dried over anhydrous sodium sulfate, filtered and concentrated under reduced pressure. The residue was purified by preparative thin-layer chromatography (silica gel, eluent: petroleum ether/ethyl acetate 4:1). Yield: 0.45 g (80% purity, 28% of theory).

¹H-NMR (400 MHz, CDCl₃): δ [ppm]=7.92 (s, 1H), 7.28-7.22 (m, 2H), 7.05-6.98 (m, 1H), 4.55-4.45 (m, 1H), 4.33-4.23 (m, 1H), 3.93 (s, 3H), 3.90-3.82 (m, 1H), 3.07-2.98 (m, 1H), 2.92-2.85 (m, 1H), 1.29 (d, 3H).

### Example 4.4B

### 11-Chloro-12-fluoro-2-methoxy-7-methyl-7,8-dihydro-5H-[3]benzoxocino[5,6-c]pyridine (Racemate)

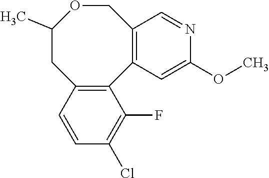

5-({[1-(2-Bromo-4-chloro-3-fluorophenyl)propan-2-yl]oxy}methyl)-4-iodo-2-methoxypyridine (racemate) (1.50 g, 94% purity, 2.74 mmol, 1.0 eq.) was dissolved in N,N-dimethylformamide (40 ml), followed by the addition of bis(pinacolato)diboron (1.39 g, 5.48 mmol, 2.0 eq.), palladium(II) acetate (62 mg, 0.27 mmol, 0.1 eq.), tricyclohexylphosphine (0.12 g, 0.41 mmol, 0.15 eq.) and potassium acetate (807 mg, 8.22 mmol, 3.0 eq.). Nitrogen was passed through the mixture for 5 min and the mixture was stirred at 80° C. for 15 h under nitrogen atmosphere. After cooling to RT, saturated aqueous sodium bicarbonate solution (5.0 ml) was added, nitrogen was passed through the mixture for 5 min and stirring was continued for further 4 h at 80° C. After cooling to RT, the reaction mixture was diluted with ethyl acetate and filtered through a pad of Celite®. The filtrate was washed with water and brine, dried over anhydrous sodium sulfate, filtered and concentrated under reduced pressure. The residue was purified by column chromatography (silica gel, eluent: petroleum ether/ethyl acetate 9:1). Yield: 200 mg (85% purity, 20% of theory).

¹H-NMR (400 MHz, CDCl₃): δ [ppm]=8.38 (s, 1H), 7.44 (dd, 1H), 7.03 (dd, 1H), 6.82 (d, 1H), 4.70 (d, 1H), 4.02 (s, 3H), 3.83 (d, 1H), 3.70-3.60 (m, 1H), 2.70 (d, 1H), 2.40-2.30 (m, 1H), 1.33 (d, 3H).

### Example 4.4C

### 11-Chloro-12-fluoro-7-methyl-3,5,7,8-tetrahydro-2H-[3]benzoxocino[5,6-c]pyridin-2-one (Racemate)

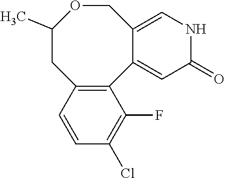 [SMILES: CC1Cc2ccc(Cl)c(F)c2-c2cc(=O)[nH]cc2CO1]

11-Chloro-12-fluoro-2-methoxy-7-methyl-7,8-dihydro-5H-[3]benzoxocino[5,6-c]pyridine (racemate) (0.47 g, 80% purity, 1.22 mmol, 1.0 eq.) was dissolved in 1-butanol (14 ml) before 4-toluenesulfonic acid monohydrate (0.46 g, 2.44 mmol, 2.0 eq.) and anhydrous lithium iodide (0.82 g, 6.1 mmol, 5.0 eq.) were added. After stirring for 3 h at 80° C., the reaction mixture was diluted with ethyl acetate and washed with saturated aqueous sodium carbonate solution, water and brine. The organic layer was dried over anhydrous sodium sulfate, filtered and concentrated under reduced pressure. The residue was purified by column chromatography (silica gel, eluent: dichloromethane/methanol 95:5). Yield: 160 mg (80% purity, 36% of theory).

¹H-NMR (400 MHz, DMSO-d₆): δ [ppm]=11.86 (br s, 1H), 7.69-7.57 (m, 2H), 7.25 (d, 1H), 6.36 (s, 1H), 4.42 (d, 1H), 3.56-3.44 (m, 2H), 2.83 (br d, 1H), 2.30-2.18 (m, 1H), 1.22 (d, 3H).

### Example 4.5A

### 5-({[(2R)-1-(4-Chloro-3-fluorophenyl)propan-2-yl]oxy}methyl)-4-iodo-2-methoxypyridine (Single Stereoisomer)

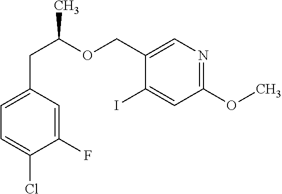 [SMILES: COc1cc(I)c(CO[C@H](C)Cc2ccc(Cl)c(F)c2)cn1]

General Method 1 was carried out with (2R)-1-(4-chloro-3-fluorophenyl)propan-2-ol (single stereoisomer) (1.00 g, 5.30 mmol, 1.0 eq.), 5-(bromomethyl)-4-iodo-2-methoxypyridine (2.41 g, 72% purity, 5.30 mmol, 1.0 eq.) and sodium hydride (530 mg, 60% dispersion in mineral oil, 13.3 mmol, 2.5 eq.) in tetrahydrofuran (20 ml) for 1 h at 0° C. and 48 h at RT. The crude product was purified by column chromatography (silica gel, eluent: cyclohexane/ethyl acetate). Yield: 1.56 g (92% purity, 62% of theory).

LC-MS (method 1): Rₜ=1.36 min; MS (ESIpos): m/z=436 [M+H]⁺

### Example 45B

### (7R)-11-Chloro-12-fluoro-2-methoxy-7-methyl-7,8-dihydro-5H-[3]benzoxocino[5,6-c]pyridine (Single Stereoisomer)

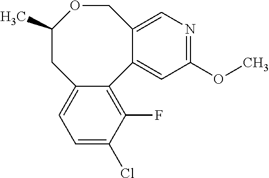

General Method 3b was carried out with 5-({[(2R)-1-(4-chloro-3-fluorophenyl)propan-2-yl]oxy}methyl)-4-iodo-2-methoxypyridine (single stereoisomer) (750 mg, 1.72 mmol, 1.0 eq.), tetrakis(triphenylphosphine)palladium(0) (199 mg, 172 μmol, 0.1 eq.) and potassium pivalate (966 mg, 6.89 mmol, 4.0 eq.) in N,N-dimethylacetamide (59 ml) for 4 h at 150° C. The crude mixture was extracted with methyl tert-butyl ether, dried over anhydrous magnesium sulfate, filtered and concentrated under reduced pressure. The residue was purified by column chromatography (silica gel, eluent: cyclohexane/ethyl acetate 98:2 to 80:20). Yield: 497 mg (86% purity, 81% of theory).

LC-MS (method 1): Rₜ=1.09 min; MS (ESIpos): m/z=308 [M+H]⁺

### Example 4.5C

### (7R)-11-Chloro-12-fluoro-7-methyl-3,5,7,8-tetrahydro-2H-[3]benzoxocino[5,6-c]pyridin-2-one (Single Stereoisomer)

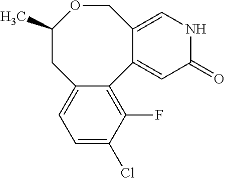 [SMILES: C[C@@H]1Cc2ccc(Cl)c(F)c2-c2cc(=O)[nH]cc2CO1]

General Method 4 was carried out with (7R)-11-chloro-12-fluoro-2-methoxy-7-methyl-7,8-dihydro-5H-[3]benzoxocino[5,6-c]pyridine (single stereoisomer) (498 mg, 1.62 mmol, 1.0 eq.) and sodium iodide (970 mg, 6.47 mmol, 4.0 eq.) in acetic acid (45 ml) for 3 h at 100° C. including the following variations of the procedure: The reaction mixture was concentrated under reduced pressure, dissolved in ethyl acetate and saturated aqueous sodium bicarbonate solution and washed with aqueous sodium thiosulfate solution (10%) and water. The aqueous phase was extracted with ethyl acetate, the combined organic phases were dried over anhydrous magnesium sulfate, filtered and concentrated under reduced pressure. The crude product was purified by column chromatography (silica gel, eluent: dichloromethane/methanol 99:1 to 90:10). Yield: 151 mg (100% purity) and 160 mg (73% purity) (combined 57% of theory).

LC-MS (method 1): Rₜ=0.70 min; MS (ESIpos): m/z=294 [M+H]⁺

### Example 4.6A

### 5-(((1-(2-Bromo-4-chlorophenyl)butan-2-yl)oxy)methyl)-4-iodo-2-methoxypyridine (Racemate)

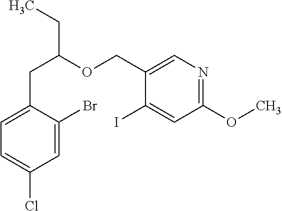 [SMILES: CCC(Cc1ccc(Cl)cc1Br)OCc1cnc(OC)cc1I]

1-(2-Bromo-4-chlorophenyl)butan-2-ol (racemate) (2.70 g, 90% purity, 9.2 mmol, 1.1 eq.) was dissolved in N,N-dimethylformamide (50 ml), cooled to 0° C. and sodium hydride (0.40 g, 60% dispersion in mineral oil, 10.1 mmol, 1.2 eq.) was added in portions. The resulting mixture was stirred for 1 h at the same temperature, followed by the addition of 5-(bromomethyl)-4-iodo-2-methoxypyridine (3.16 g, 87% purity, 8.4 mmol, 1.0 eq.). After stirring for further 3 h at RT, the mixture was treated with water and extracted with ethyl acetate. The organic layer was washed with water and brine, dried over anhydrous sodium sulfate, filtered and concentrated under reduced pressure. The residue was purified by column chromatography (silica gel, eluent: petroleum ether/ethyl acetate 12:1). Yield: 3.00 g (95% purity, 66% of theory).

LC-MS (method 11): Rₜ=1.82 min; MS (ESIpos): m/z=510 [M+H]⁺

¹H-NMR (300 MHz, DMSO-d₆): δ [ppm]=7.94 (s, 1H), 7.64 (s, 1H), 7.36-7.30 (m, 3H), 4.43-4.30 (m, 2H), 3.83 (s, 3H), 3.65-3.60 (m, 1H), 2.92-2.85 (m, 2H), 1.55-1.50 (m, 2H), 0.92 (t, 3H).

### Example 4.6B

### 11-Chloro-7-ethyl-2-methoxy-7,8-dihydro-5H-[3]benzoxocino[5,6-c]pyridine (Racemate)

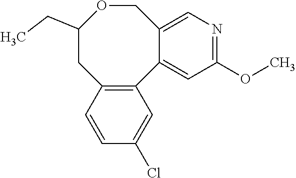 [SMILES: CCC1Cc2ccc(Cl)cc2-c2cc(OC)ncc2CO1]

5-(((1-(2-Bromo-4-chlorophenyl)butan-2-yl)oxy)methyl)-4-iodo-2-methoxypyridine (racemate) (3.00 g, 95% purity, 5.6 mmol, 1.0 eq.) was dissolved in N,N-dimethylformamide (60 ml). Bis(pinacolato)diboron (2.84 g, 11.2 mmol, 2.0 eq.), palladium(II)acetate (0.13 g, 0.6 mmol, 0.1 eq.), tricyclohexylphosphine (0.24 g, 0.8 mmol, 0.15 eq.) and potassium acetate (1.64 g, 16.7 mmol, 3.0 eq.) were added. Nitrogen was passed through the mixture for 5 min and the mixture was stirred for 15 h at 80° C. under nitrogen atmosphere. After cooling to RT, the resulting mixture was treated with saturated aqueous sodium bicarbonate solution (15 ml) and stirred for further 4 h at 80° C. Subsequently, the reaction mixture was cooled to RT, diluted with ethyl acetate and filtered through a pad of Celite®. The filtrate was washed with water and brine, dried over anhydrous sodium sulfate, filtered and concentrated under reduced pressure. The residue was purified by column chromatography (silica gel, eluent: petroleum ether/ethyl acetate 50:1) twice. Yield: 1.50 g (90% purity, 79% of theory).

LC-MS (method 12): Rₜ=1.27 min; MS (ESIpos): m/z=304 [M+H]⁺

¹H-NMR (300 MHz, DMSO-d₆): δ [ppm]=8.31 (s, 1H), 7.54-7.50 (m, 1H), 7.42-7.39 (m, 2H), 6.85 (s, 1H), 4.70 (d, 1H), 3.92 (s, 3H), 3.66 (d, 1H), 3.33-3.29 (m, 1H), 2.83-2.78 (m, 1H), 2.14-2.00 (m, 1H), 1.62-1.52 (m, 2H), 0.98-0.88 (m, 3H).

### Example 4.6C

### 11-Chloro-7-ethyl-3,5,7,8-tetrahydro-2H-[3]benzoxocino[5,6-c]pyridin-2-one (Racemate)

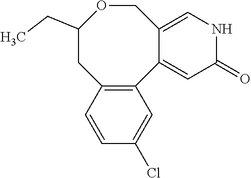 [SMILES: CCC1Cc2ccc(Cl)cc2-c2cc(=O)[nH]cc2CO1]

11-Chloro-7-ethyl-2-methoxy-7,8-dihydro-5H-[3]benzoxocino[5,6-c]pyridine (racemate) (1.50 g, 90% purity, 4.4 mmol, 1.0 eq.) was dissolved in 1-butanol (30 ml). 4-Toluenesulfonic acid monohydrate (1.52 g, 8.0 mmol, 1.8 eq.) and lithium iodide (2.97 g, 22.2 mmol, 5.0 eq.) were added. After stirring for 3 h at 80° C., the reaction mixture was concentrated under reduced pressure. The residue was dissolved in ethyl acetate and water. The organic layer was separated, washed with brine, dried over anhydrous sodium sulfate, filtered and concentrated under reduced pressure. The residue was purified by column chromatography (silica gel, eluent: dichloromethane/methanol 20:1). Yield: 0.65 g (76% purity, 38% of theory).

LC-MS (method 13): Rₜ=0.95 min; MS (ESIpos): m/z=290 [M+H]⁺

### Example 4.7A

### 5-({[(−1-(2-Bromo-4-chlorophenyl)-3-{[tert-butyl(diphenyl)silyl]oxy}propan-2-yl]oxy}methyl)-4-iodo-2-methoxypyridine (Racemate)

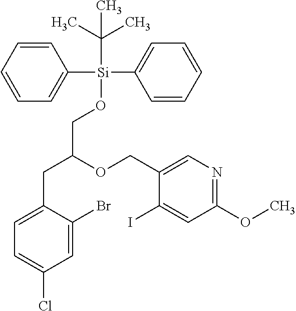 [SMILES: COc1cc(I)c(COC(CO[Si](c2ccccc2)(c2ccccc2)C(C)(C)C)Cc2ccc(Cl)cc2Br)cn1]

5-(Bromomethyl)-4-iodo-2-methoxypyridine (4.00 g, 11.1 mmol, 1.0 eq.) and 1-(2-bromo-4-chlorophenyl)-3-{[tert-butyl(diphenyl)silyl]oxy}propan-2-ol (racemate) (6.34 g, 81% purity, 12.2 mmol, 1.1 eq.) were dissolved in tetrahydrofuran (80 ml) and cooled to 0° C. Sodium hydride (1.11 g, 60% dispersion in mineral oil, 27.7 mmol, 2.5 eq.) was added and stirring was continued for 1 h at 0° C. and overnight at RT. The reaction mixture was diluted with ethyl acetate and saturated aqueous ammonium chloride solution, washed with water and brine, dried over anhydrous sodium sulfate and filtered. The filtrate was concentrated under reduced pressure and the crude product was purified by column chromatography (silica gel, eluent: cyclohexane/ethyl acetate 98:2 to 70:30). Yield: 8.74 g (89% purity, 93% of theory).

LC-MS (method 3): Rₜ=6.73 min; MS (ESIpos): m/z=750 [M+H]⁺

¹H-NMR (500 MHz, DMSO-d₆): δ [ppm]=7.89 (s, 1H), 7.68-7.56 (m, 4H), 7.49-7.32 (m, 7H), 7.32-7.25 (m, 3H), 4.48 (d, 1H), 4.31 (d, 1H), 3.87-3.79 (m, 1H), 3.83 (s, 3H), 3.74 (dd, 2H), 2.99 (dd, 1H), 2.91 (dd, 1H), 0.99 (s, 9H).

### Example 4.7B

### 7-({[Tert-Butyl(diphenyl)silyl]oxy}methyl)-11-chloro-2-methoxy-7,8-dihydro-5H-[3]benzoxocino[5,6-c]pyridine (Racemate)

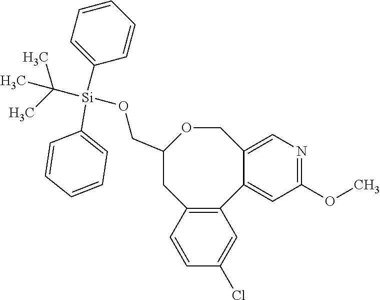 [SMILES: COc1cc2c(cn1)COC(CO[Si](c1ccccc1)(c1ccccc1)C(C)(C)C)Cc1ccc(Cl)cc1-2]

5-({[−1-(2-Bromo-4-chlorophenyl)-3-{[tert-butyl(diphenyl)silyl]oxy}propan-2-yl]oxy}methyl)-4-iodo-2-methoxypyridine (racemate) (8.74 g, 89% purity, 11.6 mmol, 1.0 eq.), bis(pinacolato)diboron (4.14 g, 16.3 mmol, 1.4 eq.), palladium(II) acetate (78.4 mg, 0.35 mmol, 0.03 eq.) and potassium acetate (3.43 g, 34.9 mmol, 3.0 eq.) were suspended in N,N-dimethylformamide (150 ml) and argon was passed through the resulting suspension for 10 min. The reaction mixture was heated to 80° C. and stirred at this temperature overnight, followed by the addition of saturated aqueous sodium bicarbonate solution (70 ml) and tetrakis(triphenylphosphine)palladium(0) (405 mg, 0.35 mmol, 0.03 eq.). Stirring was continued at 85° C. for additional 2 h and the reaction mixture was filtered over silica gel and eluted with dichloromethane. The filtrate was concentrated under reduced pressure and the crude product was purified by column chromatography (silica gel, eluent: cyclohexane/ethyl acetate 100:0 to 80:20). Yield: 3.02 g (94% purity, 45% of theory).

LC-MS (method 3): Rₜ=6.19 min; MS (ESIpos): m/z=544 [M+H]⁺

### Example 4.7C

### 7-({[Tert-Butyl(diphenyl)silyl]oxy}methyl)-11-chloro-3,5,7,8-tetrahydro-2H-[3]benzoxocino[5,6-c]pyridin-2-one (Racemate)

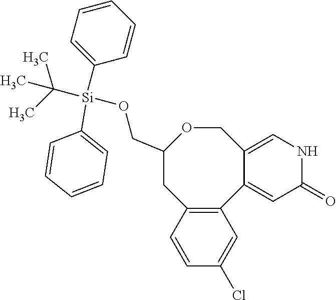 [SMILES: CC(C)(C)[Si](OCC1Cc2ccc(Cl)cc2-c2cc(=O)[nH]cc2CO1)(c1ccccc1)c1ccccc1]

7-({[tert-butyl(diphenyl)silyl]oxy}methyl)-11-chloro-2-methoxy-7,8-dihydro-5H-[3]benzoxocino[5,6-c]pyridine (racemate) (1.50 g, 94% purity, 1.59 mmol, 1.0 eq.) was dissolved in acetic acid (30 ml). Sodium iodide (777 mg, 5.18 mmol, 2.0 eq.) was added and the resulting mixture was stirred at 100° C. for 2 h. Dichloromethane and saturated aqueous sodium bicarbonate solution were added and the aqueous phase was extracted with dichloromethane. The combined organic phases were washed with brine, dried over anhydrous sodium sulfate, filtered and concentrated under reduced pressure. The crude product was used in the subsequent reaction without further purification. Yield: 1.46 g (89% purity, 95% of theory).

LC-MS (method 3): Rₜ=4.86 min; MS (ESIpos): m/z=530 [M+H]⁺

¹H-NMR (600 MHz, DMSO-d₆): δ [ppm]=11.86 (br s, 1H), 7.70-7.59 (m, 4H), 7.56-7.41 (m, 8H), 7.34 (d, 1H), 7.25 (d, 1H), 6.28 (s, 1H), 4.47 (d, 1H), 3.77 (dd, 1H), 3.59 (dd, 1H), 3.53-3.45 (m, 2H), 3.04 (d, 1H), 2.28 (dd, 1H), 1.03 (s, 9H).

### Example 4.8A

### Ethyl 4-[5-chloro-2-(2-hydroxy-3-methylbutyl)phenyl]-1-(2,4-dimethoxybenzyl)-6-oxo-1,6-dihydropyridine-3-carboxylate (Racemate)

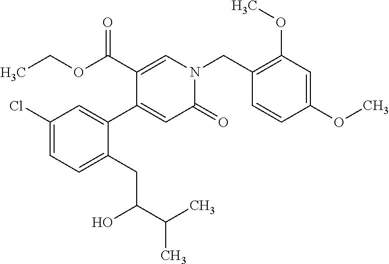 [SMILES: CCOC(=O)c1cn(Cc2ccc(OC)cc2OC)c(=O)cc1-c1cc(Cl)ccc1CC(O)C(C)C]

Ethyl 1-(2,4-dimethoxybenzyl)-6-oxo-4-{[(trifluoromethyl)sulfonyl]oxy}-1,6-dihydropyridine-3-carboxylate (2.77 g, 5.95 mmol, 1.0 eq.) under argon atmosphere was dissolved in 1,4-dioxane (13.9 ml). Bis(pinacolato)diboron (1.74 g, 6.85 mmol, 1.15 eq.), potassium acetate (1.75 g, 17.9 mmol, 3.0 eq.) and [1,1′-bis(diphenylphosphino)ferrocene]dichloropalladium(II) dichloromethane complex (250 mg, 0.32 mmol, 0.05 eq.) were added. Argon was passed through the resulting suspension for 5 min. The reaction mixture was heated to 100° C. overnight. 1-(2-Bromo-4-chlorophenyl)-3-methylbutan-2-ol (racemate) (1.90 g, 6.85 mmol, 1.15 eq.), aqueous sodium carbonate solution (5.95 ml, 2.0 M, 11.9 mmol, 2.0 eq.) and [1,1′-bis(diphenylphosphino)ferrocene]dichloropalladium(II) dichloromethane complex (100 mg, 0.13 mmol, 0.025 eq.) were added and the reaction mixture was heated at 120° C. for 3 h. After cooling to RT, water was added and the aqueous phase was extracted with ethyl acetate. The combined organic phases were washed with brine, dried over anhydrous magnesium sulfate, filtered and concentrated under reduced pressure. The crude product was purified by column chromatography (silica gel, eluent: cyclohexane/ethyl acetate 100:0 to 3:2). Yield: 1.84 g (60% of theory).

LC-MS (method 4): Rₜ=2.19 min; MS (ESIpos): m/z=514 [M+H]⁺

¹H-NMR (400 MHz, DMSO-d₆): δ [ppm]=8.55-8.45 (m, 1H), 7.42-7.21 (m, 2H), 7.20-7.07 (m, 2H), 6.65-6.58 (m, 1H), 6.54-6.46 (m, 1H), 6.18 (d, 1H), 5.16-4.96 (m, 2H), 4.38-4.27 (m, 1H), 3.99-3.86 (m, 2H), 3.84-3.70 (m, 6H), 3.37-3.06 (m, 1H, partially concealed), 2.48-2.23 (m, 2H, partially concealed), 1.50-1.29 (m, 1H), 0.93 (t, 3H), 0.73-0.65 (m, 3H), 0.63-0.53 (m, 3H).

### Example 4.8B

### 4-[5-Chloro-2-(2-hydroxy-3-methylbutyl)phenyl]-1-(2,4-dimethoxybenzyl)-5-(hydroxymethyl)-pyridin-2(1H)-one (Racemate)

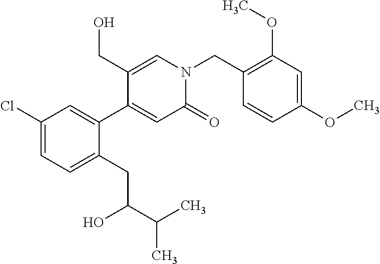 [SMILES: COc1ccc(Cn2cc(CO)c(-c3cc(Cl)ccc3CC(O)C(C)C)cc2=O)c(OC)c1]

Ethyl 4-[5-chloro-2-(2-hydroxy-3-methylbutyl)-phenyl]-1-(2,4-dimethoxybenzyl)-6-oxo-1,6-dihydropyridine-3-carboxylate (racemate) (1.8 g, 3.5 mmol, 1.0 eq.) under argon atmosphere was dissolved in dichloromethane (68 ml) and cooled to −78° C. A diisobutylaluminum hydride solution (17.5 ml, 1.0 M in dichloromethane, 17.5 mmol, 5.0 eq.) was added dropwise and the reaction mixture was stirred at −78° C. for 30 min. Subsequently, the mixture was treated with aqueous hydrochloric acid (1 N) and saturated aqueous potassium sodium tartrate solution was added. Stirring was continued for 15 min, followed by extraction with dichloromethane. The organic phases were washed with brine, dried over anhydrous magnesium sulfate, filtered and concentrated under reduced pressure. The crude product was purified by column chromatography (silica gel, eluent: cyclohexane/ethyl acetate 100:0 to 3:1). Yield: 985 mg (60% of theory).

LC-MS (method 4): Rₜ=1.70 min; MS (ESIpos): m/z=472 [M+H]⁺

¹H-NMR (400 MHz, DMSO-d₆): δ [ppm]=7.61 (s, 1H), 7.45-7.30 (m, 2H), 7.28-7.12 (m, 1H), 7.07-6.96 (m, 1H), 6.65-6.57 (m, 1H), 6.50-6.37 (m, 1H), 6.27-6.06 (m, 1H), 5.14-4.84 (m, 3H), 4.47-4.26 (m, 1H), 3.97-3.84 (m, 2H), 3.83-3.73 (m, 6H), 3.25-3.12 (m, 1H), 2.69-2.21 (m, 2H, partially concealed), 1.50-1.34 (m, 1H), 0.75-0.60 (m, 6H).

### Example 4.8C

### 11-Chloro-3-(2,4-dimethoxybenzyl)-7-isopropyl-3,5,7,8-tetrahydro-2H-[3]benzoxocino[5,6-c]pyridin-2-one (Racemate)

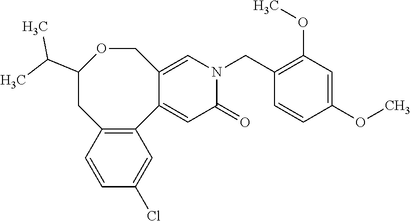 [SMILES: COc1ccc(Cn2cc3c(cc2=O)-c2cc(Cl)ccc2CC(C(C)C)OC3)c(OC)c1]

4-[5-Chloro-2-(2-hydroxy-3-methylbutyl)phenyl]-1-(2,4-dimethoxybenzyl)-5-(hydroxymethyl)pyridin-2(1H)-one (racemate) (980 mg, 2.1 mmol, 1.0 eq.) under argon atmosphere was dissolved in dichloromethane (23 ml), triethylamine (0.69 ml, 4.98 mmol, 2.4 eq.) and methanesulfonyl chloride (0.21 ml, 2.70 mmol, 1.3 eq.) were added and the reaction mixture was stirred overnight. The reaction mixture was concentrated under reduced pressure and combined with another mixture resulting from a similar experiment [4-[5-chloro-2-(2-hydroxy-3-methylbutyl)phenyl]-1-(2,4-dimethoxybenzyl)-5-(hydroxyl-methyl)pyridin-2(1H)-one (racemate) (45 mg, 0.01 mmol, 1.0 eq.), triethylamine (31 μl, 0.2 mmol, 20 eq.) and methanesulfonyl chloride (9 μl, 0.1 mmol, 10 eq.) in dichloromethane (1 ml)]. The combined crude material was purified by column chromatography (silica gel, eluent: cyclohexane/ethyl acetate 100:0 to 1:1). Yield: 863 mg (88% of theory for both experiments).

LC-MS (method 4): Rₜ=2.45 min; MS (ESIpos): m/z=454 [M+H]⁺

¹H-NMR (400 MHz, DMSO-d₆): δ [ppm]=7.83 (s, 1H), 7.55-7.44 (m, 1H), 7.37-7.33 (m, 1H), 7.29 (d, 1H), 7.10-7.04 (m, 1H), 6.60 (d, 1H), 6.52-6.44 (m, 1H), 6.35 (s, 1H), 5.07-4.90 (m, 2H), 4.46 (d, 1H), 3.85-3.72 (m, 6H), 3.50 (d, 1H), 3.19-3.11 (m, 1H), 2.78 (d, 1H), 2.26-2.17 (m, 1H), 1.92-1.77 (m, 1H), 0.96-0.87 (m, 6H).

### Example 4.8D

### 11-Chloro-7-isopropyl-3,5,7,8-tetrahydro-2H-[3]benzoxocino[5,6-c]pyridin-2-one (Racemate)

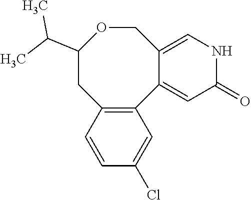 [SMILES: CC(C)C1Cc2ccc(Cl)cc2-c2cc(=O)[nH]cc2CO1]

11-Chloro-3-(2,4-dimethoxybenzyl)-7-isopropyl-3,5,7,8-tetrahydro-2H-[3]benzoxocino[5,6-c]pyridin-2-one (racemate) (1.05 g, 2.31 mmol, 1.0 eq.) was dissolved in trifluoroacetic acid (30 ml). The mixture was heated to 65° C. for 2 h. Further amounts of trifluoroacetic acid (20 ml) were added and stirring was continued for 1 h at 65° C. and at RT overnight. The reaction mixture was concentrated under reduced pressure. For three times, the residue was dissolved in dichloromethane and concentrated under reduced pressure. The crude product was purified by column chromatography (silica gel, eluent: dichloromethane/methanol 100:0 to 25:1). Yield: 549 mg (74% of theory).

LC-MS (method 5): Rₜ=1.25 min; MS (ESIpos): m/z=304 [M+H]⁺

¹H-NMR (400 MHz, DMSO-d₆): δ [ppm]=11.81 (br s, 1H), 7.59 (s, 1H), 7.51-7.46 (m, 1H), 7.34-7.27 (m, 2H), 6.27 (s, 1H), 4.50 (d, 1H), 3.47 (d, 1H), 3.19-3.12 (m, 1H), 2.81-2.74 (m, 1H), 2.26-2.16 (m, 1H), 1.91-1.78 (m, 1H), 0.97-0.86 (m, 6H).

### Example 4.9A

### 5-({[3-(2-Bromo-4-chlorophenyl)-1,1-difluoropropan-2-yl]oxy}methyl)-4-iodo-2-methoxypyridine (Racemate)

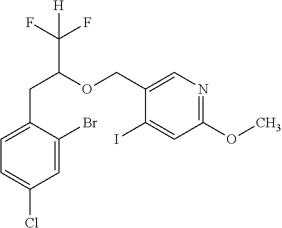 [SMILES: COc1cc(I)c(COC(Cc2ccc(Cl)cc2Br)C(F)F)cn1]

General Method 1 was carried out with 5-(bromomethyl)-4-iodo-2-methoxypyridine (1.12 g, 93% purity, 3.18 mmol, 1.0 eq.), 3-(2-bromo-4-chlorophenyl)-1,1-difluoropropan-2-ol (racemate) (1.00 g, 3.50 mmol, 1.1 eq.), sodium hydride (318 mg, 60% in mineral oil, 7.96 mmol, 2.5 eq.) in tetrahydrofuran (20 ml) for 1 h at 0° C. and at RT overnight and the crude mixture was purified by preparative HPLC (reversed phase, eluent: water with 0.05% formic acid/acetonitrile 90:10 to 5:95). Yield: 1.01 g (58% of theory).

LC-MS (method 1): Rₜ=1.39 min; MS (ESIpos): m/z=532 [M+H]⁺

### Example 4.9B

### 11-Chloro-7-(difluoromethyl)-2-methoxy-7,8-dihydro-5H-[3]benzoxocino[5,6-c]pyridine (Racemate)

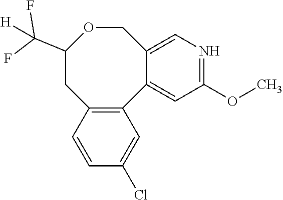 [SMILES: COc1cc2c(cn1)COC(C(F)F)Cc1ccc(Cl)cc1-2]

General Method 3a was carried out with 5-({[3-(2-bromo-4-chlorophenyl)-1,1-difluoropropan-2-yl]oxy}methyl)-4-iodo-2-methoxypyridine (racemate) (900 mg, 1.64 mmol, 1.0 eq.), bis(pinacolato)diboron (583 mg, 2.29 mmol, 1.4 eq.), palladium(II) acetate (11.0 mg, 49.2 μmol, 0.03 eq.) and potassium acetate (483 mg, 4.92 mmol, 3.0 eq.) in N,N-dimethylformamide (20 ml) including the following variations of the procedure: The reaction mixture was filtered through a pad of silica gel and anhydrous sodium sulfate and eluted with a mixture of cyclohexane and ethyl acetate (1:1). The filtrate was concentrated under reduced pressure and the crude mixture was purified by preparative HPLC (reversed phase, eluent: water with 0.05% formic acid/acetonitrile 90:10 to 5:95). Yield: 356 mg (67% of theory).

LC-MS (method 1): Rₜ=1.16 min; MS (ESIpos): m/z=326 [M+H]⁺

### Example 4.9C

### 11-Chloro-7-(difluoromethyl)-3,5,7,8-tetrahydro-2H-[3]benzoxocino[5,6-c]pyridin-2-one (Racemate)

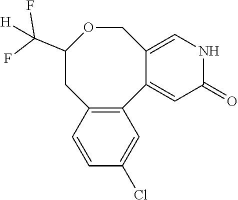 [SMILES: O=c1cc2c(c[nH]1)COC(C(F)F)Cc1ccc(Cl)cc1-2]

General Method 4 was carried out with 11-chloro-7-(difluoromethyl)-2-methoxy-7,8-dihydro-5H-[3]benzoxocino[5,6-c]pyridine (racemate) (350 mg, 1.07 mmol, 1.0 eq.), sodium iodide (483 mg, 3.22 mmol, 3.0 eq.) in acetic acid (20 ml) at 100° C. for 2 h including the following variations of the procedure: The crude mixture was used in the subsequent reaction without further purification. Yield: 450 mg (74% purity, 99% of theory).

LC-MS (method 1): Rₜ=0.81 min; MS (ESIpos): m/z=312 [M+H]⁺

### Example 4.10A

### Ethyl 4-[5-chloro-2-(3,3,3-trifluoro-2-hydroxypropyl)phenyl]-1-(2,4-dimethoxybenzyl)-6-oxo-1,6-dihydropyridine-3-carboxylate (Racemate)

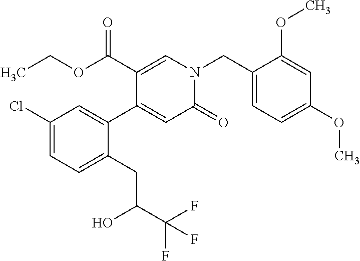 [SMILES: CCOC(=O)c1cn(Cc2ccc(OC)cc2OC)c(=O)cc1-c1cc(Cl)ccc1CC(O)C(F)(F)F]

Ethyl 1-(2,4-dimethoxybenzyl)-6-oxo-4-(4,4,5,5-tetramethyl-1,3,2-dioxaborolan-2-yl)-1,6-dihydropyridine-3-carboxylate (83.5 g, 58% purity, 109.2 mmol, 1.7 eq.) was dissolved in 1,4-dioxane (600 ml) and water (30 ml). Subsequently, 3-(2-bromo-4-chlorophenyl)-1,1,1-trifluoropropan-2-ol (racemate) (26.0 g, 75% purity, 64.2 mmol, 1.0 eq.), sodium carbonate (20.43 g, 192.7 mmol, 3.0 eq.) and [1,1′-bis(diphenylphosphino)ferrocene]dichloropalladium(II) dichloromethane complex (5.2 g, 6.4 mmol, 0.1 eq.) were added. Nitrogen was passed through the mixture for 15 min and stirring was continued for 4 h at 100° C. under nitrogen atmosphere. After cooling to RT, the reaction mixture was filtered through a pad of Celite® and the filtrate was concentrated under reduced pressure. The residue was dissolved in ethyl acetate, washed with water and brine, dried over anhydrous sodium sulfate, filtered and concentrated under reduced pressure. The residue was purified by column chromatography (silica gel, eluent: petroleum ether/ethyl acetate 10:1). Yield: 24.0 g (68% purity, 47% of theory).

LC-MS (method 15): Rₜ=2.38 min; MS (ESIpos): m/z=540 [M+H]⁺

### Example 4.10B

### 4-[5-Chloro-2-(3,3,3-trifluoro-2-hydroxypropyl)phenyl]-1-(2,4-dimethoxybenzyl)-5-(hydroxymethyl)pyridin-2(11)-one (Racemate)

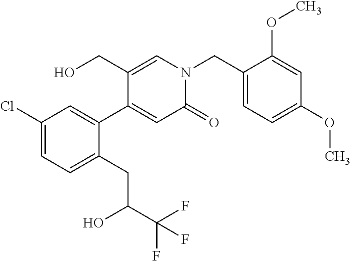 [SMILES: COc1ccc(Cn2cc(CO)c(-c3cc(Cl)ccc3CC(O)C(F)(F)F)cc2=O)c(OC)c1]

Ethyl 4-[5-chloro-2-(3,3,3-trifluoro-2-hydroxypropyl)phenyl]-1-(2,4-dimethoxybenzyl)-6-oxo-1,6-dihydropyridine-3-carboxylate (racemate) (24.0 g, 68% purity, 30.2 mmol, 1.0 eq.) was dissolved in dichloromethane (400 ml) and diisobutylaluminum hydride solution (212 ml, 1.0 M in toluene, 212 mmol, 7.0 eq.) was added dropwise at −30° C. under nitrogen atmosphere. After warming to RT and stirring for 2 h, the reaction mixture was treated with sodium sulfate decahydrate (50 g) and stirred for further 20 min. The resulting mixture was diluted with dichloromethane and filtered through a pad of Celite®. The filtrate was concentrated under reduced pressure. The residue was purified by column chromatography (silica gel, eluent: petroleum ether/ethyl acetate 3:2). Yield: 3.80 g (83% purity, 21% of theory).

¹H-NMR (300 MHz, DMSO-d₆): δ [ppm]=7.66-7.61 (m, 1H), 7.50-7.41 (m, 2H), 7.26-7.24 (m, 1H), 7.00-6.95 (m, 1H), 6.64-6.59 (m, 1H), 6.48-6.44 (m, 1H), 6.26-6.18 (m, 2H), 5.08-4.99 (m, 2H), 4.92 (t, 1H), 4.04-3.99 (m, 1H), 3.96-3.88 (m, 2H), 3.82 (s, 3H), 3.76 (s, 3H), 2.85-2.51 (m, 2H, partially concealed).

### Example 4.10C

### 11-Chloro-3-(2,4-dimethoxybenzyl)-7-(trifluoromethyl)-3,5,7,8-tetrahydro-2H-[3]benzoxocino[5,6-c]pyridin-2-one (Racemate)

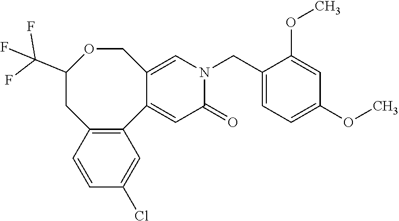 [SMILES: COc1ccc(Cn2cc3c(cc2=O)-c2cc(Cl)ccc2CC(C(F)(F)F)OC3)c(OC)c1]

4-[5-Chloro-2-(3,3,3-trifluoro-2-hydroxypropyl)phenyl]-1-(2,4-dimethoxybenzyl)-5-(hydroxymethyl)-pyridin-2(1H)-one (racemate) (3.80 g, 83% purity, 6.34 mmol, 1.0 eq.) was dissolved in dichloromethane (100 ml). Methanesulfonyl chloride (0.80 g, 7.0 mmol, 1.1 eq.) and triethylamine (1.41 g, 13.9 mmol, 2.2 eq.) were added. After stirring for 3 h at RT, the reaction mixture was concentrated under reduced pressure. The residue was purified by column chromatography (silica gel, eluent: petroleum ether/ethyl acetate 10:1). Yield: 1.80 g (56% of theory).

¹H-NMR (300 MHz, DMSO-d₆): δ [ppm]=7.94 (s, 1H), 7.58-7.54 (m, 1H), 7.43-7.39 (m, 2H), 7.09 (d, 1H), 6.61 (d, 1H), 6.52-6.49 (m, 1H), 6.38 (s, 1H), 5.08-4.92 (m, 2H), 4.63 (d, 1H), 4.21-4.11 (m, 1H), 3.82 (s, 3H), 3.76 (s, 3H), 3.72 (d, 1H), 3.09 (d, 1H), 2.46-2.41 (m, 1H, partially concealed).

### Example 4.10D

### 11-Chloro-7-(trifluoromethyl)-3,5,7,8-tetrahydro-2H-[3]benzoxocino[5,6-c]pyridin-2-one (Racemate)

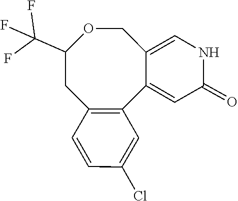 [SMILES: O=c1cc2c(c[nH]1)COC(C(F)(F)F)Cc1ccc(Cl)cc1-2]

A solution of 11-chloro-3-(2,4-dimethoxybenzyl)-7-(trifluoromethyl)-3,5,7,8-tetrahydro-2H-[3]benzoxocino[5,6-c]pyridin-2-one (racemate) (1.75 g, 3.46 mmol, 1.0 eq.) in trifluoroacetic acid (20 ml) was stirred for 3 h at 65° C. After being cooled to RT, the reaction mixture was concentrated under reduced pressure. The residue was treated with a mixture of N,N-dimethylformamide (15 ml) and water (30 ml) and the resulting solid was collected by filtration. The filtrate was adjusted to pH 8 by addition of sodium carbonate and extracted with dichloromethane. The combined organic layers were washed with water and brine, dried over anhydrous sodium sulfate, filtered and concentrated under reduced pressure. The collected solid was dissolved in dichloromethane, dried over anhydrous sodium sulfate, filtered and concentrated under reduced pressure. The residues were combined and purified by column chromatography (silica gel, eluent: dichloromethane/methanol 50:1). Yield: 1.10 g (84% purity, 81% of theory).

LC-MS (method 14): Rₜ=1.33 min; MS (ESIpos): m/z=330 [M+H]⁺

Alternatively, General Method 4 was carried out with 11-chloro-2-methoxy-7-(trifluoromethyl)-7,8-dihydro-5H-[3]benzoxocino[5,6-c]pyridine (racemate) (940.0 mg, 84% purity, 2.30 mmol, 1.0 eq.) and sodium iodide (689 mg, 4.59 mmol, 2.0 eq.) in acetic acid (35 ml) for 2 h at 100° C. The crude product was purified by column chromatography (silica gel, eluent: cyclohexane/ethyl acetate 100:0 to 0:100). Yield: 420 mg (85% purity, 47% of theory).

LC-MS (method 3): Rₜ=2.60 min; MS (ESIpos): m/z=330 [M+H]⁺

### Example 4.10E

### (7S)-11-Chloro-7-(trifluoromethyl)-3,5,7,8-tetrahydro-2H-[3]benzoxocino[5,6-c]pyridin-2-one (Single Stereoisomer)

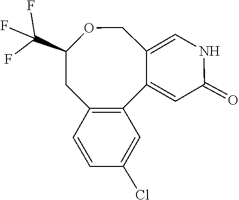 [SMILES: O=c1cc2c(c[nH]1)CO[C@H](C(F)(F)F)Cc1ccc(Cl)cc1-2]

Enantiomer separation of 420 mg of 11-chloro-7-(trifluoromethyl)-3,5,7,8-tetrahydro-2H-[3]benzoxocino[5,6-c]pyridin-2-one (racemate), Example 4.10D gave
single stereoisomer 1 (chiral SFC: Rₜ=1.13 min): 105 mg,
single stereoisomer 2 (the title compound Example 4.10E) (chiral SFC: Rₜ=1.53 min, 99% ee): 104 mg.

Separation method: SFC: column: Daicel Chiralpak AZ-H 5 μm, 250 mm×20 mm; eluent: 80% carbon dioxide/20% methanol; temperature: 40° C.; flow rate: 80 ml/min; UV detection: 210 nm.

Analysis method: SFC: column: Daicel Chiralpak AZ-H 5 μm, 250 mm×4.6 mm; eluent: 80% carbon dioxide/20% methanol; temperature: 40° C.; flow rate: 3.0 ml/min; UV detection: 210 nm.

LC-MS (method 5): Rₜ=1.15 min; MS (ESIpos): m/z=330 [M+H]⁺

### Example 4.11A

### 5-({[3-(2-Bromo-4-chlorophenyl)-1,1,1-trifluoropropan-2-yl]oxy}methyl)-4-iodo-2-methoxypyridine (Racemate)

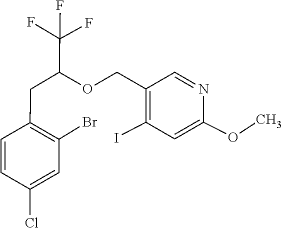 [SMILES: COc1cc(I)c(COC(Cc2ccc(Cl)cc2Br)C(F)(F)F)cn1]

General Method 1 was carried out with 5-(bromomethyl)-4-iodo-2-methoxypyridine (4.30 g, 13.1 mmol, 1.0 eq.), 3-(2-bromo-4-chlorophenyl)-1,1,1-trifluoropropan-2-ol (racemate)(4.38 g, 14.4 mmol, 1.1. eq.) and sodium hydride (1.31 g, 60% dispersion in mineral oil, 32.8 mmol, 2.5 eq.) in tetrahydrofuran (93 ml) for 1 h at 0° C. and 16 h at RT. The crude product was purified by column chromatography (silica gel, eluent: cyclohexane/ethyl acetate 9:1). Yield: 4.22 g (88% purity, 51% of theory).

LC-MS (method 1): Rₜ=1.43 min; MS (ESIpos): m/z=550 [M+H]⁺

### Example 4.11B

### 11-Chloro-2-methoxy-7-(trifluoromethyl)-7,8-dihydro-5H-[3]benzoxocino[5,6-c]pyridine (Racemate)

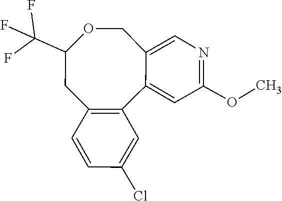 [SMILES: COc1cc2c(cn1)COC(C(F)(F)F)Cc1ccc(Cl)cc1-2]

General Method 3a was carried out with 5-({[3-(2-bromo-4-chlorophenyl)-1,1,1-trifluoropropan-2-yl]oxy}methyl)-4-iodo-2-methoxypyridine (racemate) (4.20 g, 88% purity, 6.71 mmol, 1.0 eq.), bis(pinacolato)diboron (2.13 g, 8.39 mmol, 1.25 eq.), palladium(II) acetate (85.6 mg, 381 μmol, 0.057 eq.), potassium acetate (2.25 g, 22.9 mmol, 3.45 eq.) and tricyclohexylphosphine (321 mg, 1.14 mmol, 0.17 eq.) in N,N-dimethylformamide (91 ml) for 16 h at 80° C. including the following variations of the procedure: After 16 h, aqueous sodium bicarbonate solution (46 ml) was added and stirring was continued at 85° C. for 16 h. The mixture was concentrated and the crude product was purified by column chromatography (silica gel, eluent: cyclohexane/ethyl acetate 9:1 to 1:1). Yield: 1.04 g (84% purity, 38% of theory).

LC-MS (method 3): Rₜ=3.97 min; MS (ESIpos): m/z=344 [M+H]⁺

### Example 4.12A

### 5-({[(2S)-3-(4-Chloro-3-fluorophenyl)-1,1,1-trifluoropropan-2-yl]oxy}methyl)-4-iodo-2-methoxypyridine (Single Stereoisomer)

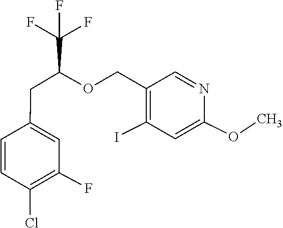 [SMILES: COc1cc(I)c(CO[C@@H](Cc2ccc(Cl)c(F)c2)C(F)(F)F)cn1]

General Method 1 was carried out with (2S)-3-(4-chloro-3-fluorophenyl)-1,1,1-trifluoropropan-2-ol (single stereoisomer) (1.68 g, 6.94 mmol, 1.0 eq.), 5-(bromomethyl)-4-iodo-2-methoxypyridine (3.45 g, 66% purity, 6.94 mmol, 1.0 eq.) and sodium hydride (416 mg, 17.3 mmol, 2.5 eq.) in tetrahydrofuran (25 ml). The crude mixture was purified by column chromatography (silica gel, eluent: cyclohexane/ethyl acetate 98:2 to 83:17). Yield: 1.77 g (52% of theory).

LC-MS (method 1): Rₜ=1.36 min; MS (ESIpos): m/z=490 [M+H]⁺

### Example 4.12B

### (7S)-11-Chloro-12-fluoro-2-methoxy-7-(trifluoromethyl)-7,8-dihydro-5H-[3]benzoxocino[5,6-c]pyridine (Single Stereoisomer)

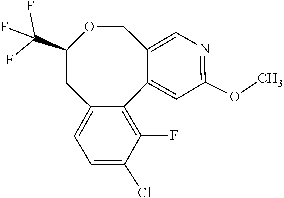 [SMILES: COc1cc2c(cn1)CO[C@H](C(F)(F)F)Cc1ccc(Cl)c(F)c1-2]

5-({[(2S)-3-(4-Chloro-3-fluorophenyl)-1,1,1-trifluoropropan-2-yl]oxy}methyl)-4-iodo-2-methoxypyridine (single stereoisomer) (1.77 g, 3.61 mmol, 1.0 eq.) and potassium pivalate (2.03 g, 14.5 mmol, 4.0 eq.) were dissolved in N,N-dimethylacetamide (150 ml). Argon was passed through the mixture for 10 min, tetrakis(triphenylphosphine)palladium(0) (418 mg, 361 μmol, 0.1 eq.) was added and the mixture was stirred for 48 h at 120° C. After addition of saturated aqueous ammonium chloride solution, the mixture was concentrated under reduced pressure at 60° C. The crude product was diluted with water and extracted with diethyl ether. The combined organic phases were washed with brine, dried over anhydrous magnesium sulfate, filtered and concentrated under reduced pressure. The crude mixture was purified by preparative HPLC (reversed phase, eluent: water with 0.05% formic acid/acetonitrile 80:20 to 5:95). Yield: 1.08 g (82% of theory).

LC-MS (method 1): Rₜ=1.23 min; MS (ESIpos): m/z=362 [M+H]⁺

### Example 4.12C

### (7S)-11-Chloro-12-fluoro-7-(trifluoromethyl)-3,5,7,8-tetrahydro-2H-[3]benzoxocino[5,6-c]pyridin-2-one (Single Stereoisomer)

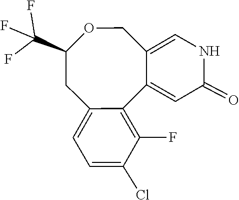 [SMILES: O=c1cc2c(c[nH]1)CO[C@H](C(F)(F)F)Cc1ccc(Cl)c(F)c1-2]

General Method 4 was carried out with (7S)-11-chloro-12-fluoro-2-methoxy-7-(trifluoromethyl)-7,8-dihydro-5H-[3]benzoxocino[5,6-c]pyridine (single stereoisomer) (1.08 g, 2.98 mmol, 1.0 eq.) and sodium iodide (1.79 g, 11.9 mmol, 4.0 eq.) in acetic acid (90 ml) at 100° C. for 2 h including the following variations of the procedure: The reaction mixture was concentrated under reduced pressure, dissolved in ethyl acetate and saturated aqueous sodium bicarbonate solution and washed with aqueous sodium thiosulfate solution (10%) and water. The aqueous phase was extracted with ethyl acetate, the combined organic phases were dried over anhydrous magnesium sulfate, filtered and concentrated under reduced pressure. The crude mixture was used in the subsequent step without further purification. Yield: 774 mg (75% of theory).

LC-MS (method 1): Rₜ=0.88 min; MS (ESIpos): m/z=348 [M+H]⁺

### Example 4.13A

### 5-({[(2S)-3-(3,4-Dichlorophenyl)-1,1,1-trifluoropropan-2-yl]oxy}methyl)-4-iodo-2-methoxypyridine (Single Stereoisomer)

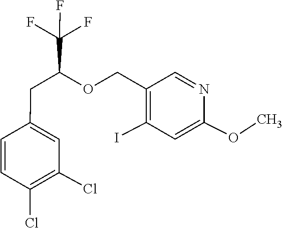 [SMILES: COc1cc(I)c(CO[C@@H](Cc2ccc(Cl)c(Cl)c2)C(F)(F)F)cn1]

General Method 1 was carried out with (2S)-3-(3,4-dichlorophenyl)-1,1,1-trifluoropropan-2-ol (single stereoisomer) (199 mg, 768 μmol, 1.0 eq.), 5-(bromomethyl)-4-iodo-2-methoxypyridine (462 mg, 60% purity, 845 μmol, 1.1 eq.) and sodium hydride (76.8 mg, 60% in mineral oil, 1.92 mmol, 2.5 eq.) in tetrahydrofuran (2.0 ml). The crude mixture was purified by preparative HPLC (reversed phase, eluent: water with 0.05% formic acid/acetonitrile 80:20 to 5:95). Yield: 313 mg (81% of theory).

LC-MS (method 1): Rₜ=1.40 min; MS (ESIpos): m/z=506 [M+H]⁺

### Example 4.13B

### (7S)-11,12-Dichloro-2-methoxy-7-(trifluoromethyl)-7,8-dihydro-5H-[3]benzoxocino[5,6-c]pyridine (Single Stereoisomer)

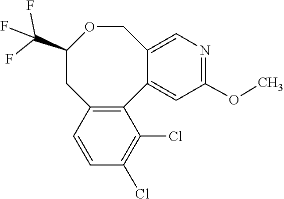 [SMILES: COc1cc2c(cn1)CO[C@H](C(F)(F)F)Cc1ccc(Cl)c(Cl)c1-2]

5-({[(2S)-3-(3,4-Dichlorophenyl)-1,1,1-trifluoropropan-2-yl]oxy}methyl)-4-iodo-2-methoxypyridine (single stereoisomer) (313 mg, 618 μmol, 1.0 eq.) and potassium acetate (243 mg, 2.47 mmol, 4.0 eq.) were dissolved in N,N-dimethylacetamide (50 ml) and argon was passed through the mixture for 10 min. Subsequently, tetrakis(triphenylphosphine)palladium(0) (71.5 mg, 61.8 μmol, 0.1 eq.) was added and the mixture was stirred for 48 h at 120° C. After addition of saturated aqueous ammonium chloride solution, the mixture was concentrated under reduced pressure at 60° C. The crude product was diluted with water and extracted with diethyl ether. The combined organic phases were washed with brine, dried over anhydrous magnesium sulfate, filtered and concentrated under reduced pressure. The crude mixture was purified by preparative HPLC (reversed phase, eluent: water with 0.05% formic acid/acetonitrile 80:20 to 5:95). Yield: 94.0 mg (78% purity, 31% of theory). In addition, the regioisomeric ring closure product (7S)-10,11-dichloro-2-methoxy-7-(trifluoromethyl)-7,8-dihydro-5H-[3]benzoxocino[5,6-c]pyridine (single stereoisomer) could be isolated. Yield: 142 mg (61% of theory).

LC-MS (method 3): Rₜ=4.19 min; MS (ESIpos): m/z=506 [M+H]⁺

¹H-NMR (400 MHz, DMSO-d₆): δ [ppm]=8.46/8.30 (2s, 1H), 7.79/7.67 (2d, 1H), 7.44/7.28 (2d, 1H), 6.91/6.74 (2s, 1H), 4.83/4.71 (2d, 1H), 4.21-4.11 (m, 1H), 3.93 (s, 3H), 3.92-3.88 (m, 1H), 3.11 (d, 1H), 2.23-2.15 (m, 1H). Additional signals of minor rotamers were also detected.

### Example 4.13C

### (7S)-11,12-Dichloro-7-(trifluoromethyl)-3,5,7,8-tetrahydro-2H-[3]benzoxocino[5,6-c]pyridin-2-one (Single Stereoisomer)

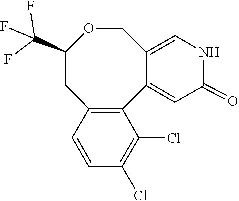 [SMILES: O=c1cc2c(c[nH]1)CO[C@H](C(F)(F)F)Cc1ccc(Cl)c(Cl)c1-2]

General Method 4 was carried out with (7S)-11,12-dichloro-2-methoxy-7-(trifluoromethyl)-7,8-dihydro-5H-[3]benzoxocino[5,6-c]pyridine (single stereoisomer) (65.0 mg, 78% purity, 134 μmol, 1.0 eq.), sodium iodide (70.3 mg, 469 μmol, 4.0 eq.) in acetic acid (2.9 ml) at 100° C. for 4 h including the following variations of the procedure: The reaction mixture was concentrated under reduced pressure, dissolved in ethyl acetate and saturated aqueous sodium bicarbonate solution and washed with aqueous sodium thiosulfate solution (10%) and water. The aqueous phase was extracted with ethyl acetate, the combined organic phases were dried over anhydrous magnesium sulfate, filtered and concentrated under reduced pressure. The crude mixture was used in the subsequent step without further purification. Yield: 75 mg (81% purity).

LC-MS (method 1): Rₜ=0.90 min; MS (ESIpos): m/z=364 [M+H]⁺

### Example 4.13D

### (7S)-10,11-Dichloro-2-methoxy-7-(trifluoromethyl)-7,8-dihydro-5H-[3]benzoxocino[5,6-c]pyridine (Single Stereoisomer)

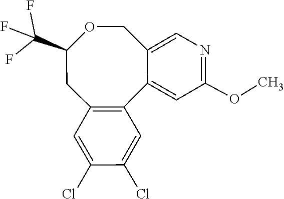 [SMILES: COc1cc2c(cn1)CO[C@H](C(F)(F)F)Cc1cc(Cl)c(Cl)cc1-2]

Regioisomer (7S)-10,11-dichloro-2-methoxy-7-(trifluoromethyl)-7,8-dihydro-5H-[3]benzoxocino[5,6-c]pyridine (single stereoisomer) of (7S)-11,12-dichloro-2-methoxy-7-(trifluoromethyl)-7,8-dihydro-5H-[3]benzoxocino[5,6-c]pyridine (single stereoisomer) could be isolated as an additional product of the synthetic procedure for Example 4.13B. Yield: 142 mg (61% of theory).

LC-MS (method 3): Rₜ=4.37 min; MS (ESIpos): m/z=378 [M+H]⁺

¹H-NMR (400 MHz, DMSO-d₆): δ [ppm]=8.38 (s, 1H), 7.76 (s, 1H), 7.67 (s, 1H), 6.90 (s, 1H), 4.85 (d, 1H), 4.33-4.22 (m, 1H), 3.92 (s, 3H), 3.91-3.86 (m, 1H), 3.14 (d, 1H), 2.24 (dd, 1H). Additional signals of minor rotamers were also detected.

### Example 4.14A

### 5-(1-{[(2R)-1-(2-Bromo-4-chlorophenyl)propan-2-yl]oxy}ethyl)-4-iodo-2-methoxypyridine (Mixture of Two Diastereomers)

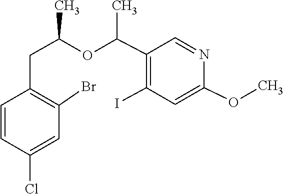 [SMILES: COc1cc(I)c(C(C)O[C@H](C)Cc2ccc(Cl)cc2Br)cn1]

1-(4-Iodo-6-methoxypyridin-3-yl)ethanol (racemate) (687 mg, 2.46 mmol, 1.0 eq.), (2R)-1-(2-bromo-4-chlorophenyl)propan-2-ol (single stereoisomer) (640 mg, 96% purity, 2.46 mmol, 1.0 eq.) and scandium(III) trifluoromethanesulfonate (1.21 g, 2.46 mmol, 1.0 eq.) were dissolved in nitromethane (12 ml), equally distributed into two vials, sealed and stirred at 90° C. for 4.5 h. The two reaction mixtures were combined, followed by addition of saturated aqueous solution of sodium bicarbonate and ethyl acetate. The organic phase was washed with water and brine and the aqueous phase was extracted with ethyl acetate. The combined organic phases were dried over anhydrous magnesium sulfate, filtered, concentrated under reduced pressure and purified by column chromatography (silica gel, eluent: cyclohexane/ethyl acetate 100:0 to 40:60). Yield: 706 mg (56% of theory).

LC-MS (method 3): Rₜ=5.31/5.35 min; MS (ESIpos): m/z=512 [M+H]⁺

### Example 4.14B

### (5S,7R)-11-Chloro-2-methoxy-5,7-dimethyl-7,8-dihydro-5H-[3]benzoxocino[5,6-c]pyridine (Single Stereoisomer)

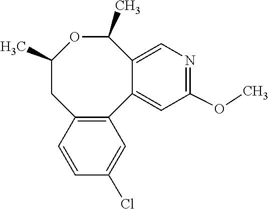

General Method 3a was carried out with 5-(1-{[(2R)-1-(2-bromo-4-chlorophenyl)propan-2-yl]oxy}ethyl)-4-iodo-2-methoxypyridine (mixture of two diastereomers) (1.60 g, 3.13 mmol, 1.0 eq.), bis(pinacolato)diboron (1.19 g, 4.70 mmol, 1.5 eq.), palladium(II) acetate (70.4 mg, 313 μmol, 0.1 eq.), potassium acetate (923 mg, 9.40 mmol, 3.0 eq.) and tricyclohexylphosphine (87.9 mg, 313 μmol, 0.1 eq.) in N,N-dimethylformamide (33 ml) including the following variations of the procedure: After stirring at 100° C. overnight, tetrakis(triphenylphosphine)palladium(0) (181 mg, 157 μmol, 0.05 eq.), sodium bicarbonate (1.32 g, 15.7 mmol, 5.0 eq.) and water (0.4 ml) were added to the reaction mixture. Stirring was continued at 100° C. overnight. The volatiles were removed at 60° C. under reduced pressure and the residue was dissolved in water and ethyl acetate, extracted with ethyl acetate, dried over anhydrous magnesium sulfate, filtered and concentrated again under reduced pressure. The crude mixture was purified by column chromatography allowing also a separation of the two diastereomers (silica gel, eluent: cyclohexane/ethyl acetate 100:0 to 40:60). Yield: 210 mg (81% purity, 18% of theory for the title diastereomer).

LC-MS (method 1): Rₜ=1.23 min; MS (ESIpos): m/z=304 [M+H]⁺

### Example 4.14C

### (5S,7R)-11-Chloro-5,7-dimethyl-3,5,7,8-tetrahydro-2H-[3]benzoxocino[5,6-c]pyridin-2-one (Single Stereoisomer)

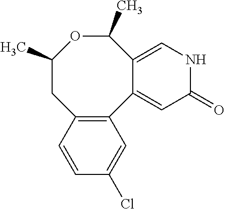 [SMILES: C[C@@H]1Cc2ccc(Cl)cc2-c2cc(=O)[nH]cc2[C@H](C)O1]

General Method 6 was carried out with (5S,7R)-11-chloro-2-methoxy-5,7-dimethyl-7,8-dihydro-5H-[3]benzoxocino[5,6-c]pyridine (single stereoisomer) (75.0 mg, 81% purity, 200 μmol, 1.0 eq.), ethanethiol (89 μl, 1.2 mmol, 6.0 eq.) and sodium hydride (24.0 mg, 60% in mineral oil, 600 μmol, 3.0 eq.) in N,N-dimethylformamide (2.2 ml) including the following variations of the procedure: The crude mixture was purified by preparative HPLC (reversed phase, eluent: water with 0.05% formic acid/acetonitrile 90:10 to 1:99). Yield: 52.0 mg (90% of theory).

LC-MS (method 3): Rₜ=2.45 min; MS (ESIpos): m/z=290 [M+H]⁺

¹H-NMR (400 MHz, DMSO-d₆): δ [ppm]=11.79 (br s, 1H), 7.95/7.53 (2s, 1H), 7.50-7.44 (m, 1H), 7.41-7.07 (m, 2H), 6.23/5.98 (2s, 1H), 4.64-4.53/3.90-3.77 (2m, 1H), 3.68-3.54 (m, 1H), 2.90-2.71 (m, 1H), 2.43-2.17 (m, 1H), 1.29/1.06 (2d, 3H), 1.20/1.01 (2d, 3H). Additional signals of minor rotamers were also detected.

### Example 4.15A

### 5-(1-{[(2R)-1-(4-Chloro-3-fluorophenyl)propan-2-yl]oxy}ethyl)-4-iodo-2-methoxypyridine (Mixture of Two Diastereomers)

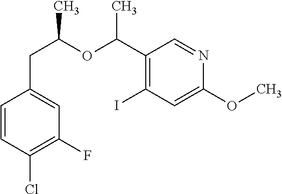 [SMILES: COc1cc(I)c(C(C)O[C@H](C)Cc2ccc(Cl)c(F)c2)cn1]

1-(4-Iodo-6-methoxypyridin-3-yl)ethanol (racemate) (592 mg, 2.12 mmol, 1.0 eq.), (2R)-1-(4-chloro-3-fluorophenyl)propan-2-ol (single stereoisomer) (400 mg, 2.12 mmol, 1.0 eq.) and scandium(III) trifluoromethanesulfonate (1.04 g, 2.12 mmol, 1.0 eq.) were dissolved in nitromethane (6.4 ml), the mixture was equally distributed into two vials, sealed and stirred at 90° C. for 4.5 h. The two reaction mixtures were combined, concentrated under reduced pressure and purified by column chromatography (silica gel, eluent: cyclohexane/ethyl acetate 94:6 to 50:50). The product was isolated in several batches: Yield: 157 mg (85% purity, 14% of theory); 122 mg (80% purity, 10% of theory); 655 mg (64% purity, 44% of theory).

LC-MS (method 1): Rₜ=1.46 min; MS (ESIpos): m/z=450 [M+H]⁺

### Example 4.15B

### (7R)-11-Chloro-12-fluoro-2-methoxy-5,7-dimethyl-7,8-dihydro-5H-[3]benzoxocino[5,6-c]pyridine (Mixture of Two Diastereomers)

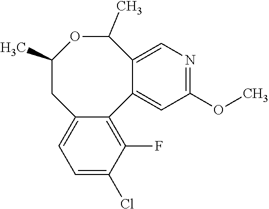

5-(1-{[(2R)-1-(4-Chloro-3-fluorophenyl)propan-2-yl]oxy}ethyl)-4-iodo-2-methoxypyridine (mixture of two diastereomers) (157 mg, 85% purity, 297 μmol, 1.0 eq.) and potassium acetate (116 mg, 1.19 mmol, 4.0 eq.) were dissolved in N,N-dimethylacetamide (10 ml) and argon was passed through the mixture for 10 min. Subsequently, tetrakis(triphenylphosphine)palladium(0) (34.3 mg, 29.7 μmol, 0.1 eq.) was added and the mixture was stirred overnight at 120° C. The crude mixture was purified by preparative HPLC (reversed phase, eluent: water with 0.05% formic acid/acetonitrile 80:20 to 5:95). Yield: 25.6 mg (78% purity, 31% of theory).

LC-MS (method 1): Rₜ=1.18 min; MS (ESIpos): m/z=322 [M+H]⁺

### Example 4.15C

### (7R)-11-Chloro-12-fluoro-5,7-dimethyl-3,5,7,8-tetrahydro-2H-[3]benzoxocino[5,6-c]pyridin-2-one (Mixture of Two Diastereomers)

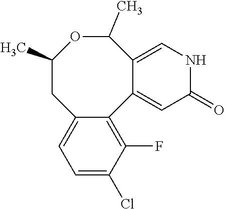 [SMILES: CC1O[C@H](C)Cc2ccc(Cl)c(F)c2-c2cc(=O)[nH]cc21]

General Method 4 was carried out with (7R)-11-chloro-12-fluoro-2-methoxy-5,7-dimethyl-7,8-dihydro-5H-[3]benzoxocino[5,6-c]pyridine (mixture of two diastereomers) (42.0 mg, 131 μmol, 1.0 eq.) and sodium iodide (68.5 mg, 457 μmol, 3.5 eq.) in acetic acid (3.0 ml) at 100° C. for 5 h including the following variations of the procedure: The reaction mixture was concentrated under reduced pressure and purified by preparative HPLC (reversed phase, eluent: water with 0.05% formic acid/acetonitrile 80:20 to 5:95). Yield: 16.2 mg (40% of theory).

LC-MS (method 1): Rₜ=0.81/0.84 min; MS (ESIpos): m/z=308 [M+H]⁺

### Example 5.1A

### 2-(Cyclopropyloxy)ethyl Trifluoromethanesulfonate

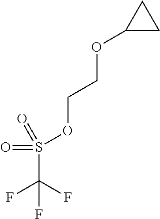 [SMILES: O=S(=O)(OCCOC1CC1)C(F)(F)F]

2,6-Dimethylpyridine (171 μl, 1.47 mmol, 1.5 eq.) and trifluoromethanesulfonic anhydride (249 μl, 1.47 mmol, 1.5 eq.) were added dropwise under argon atmosphere at 0° C. to a solution of 2-(cyclopropyloxy)ethan-1-ol (100 mg, 0.98 mmol) in dichloromethane (4 ml). The reaction mixture was stirred at 0° C. for 45 min, diluted with tert-butyl methyl ether and extracted with a mixture (3:1) of saturated aqueous solution of sodium chloride and aqueous hydrochloric acid (1 N). The combined organic phases were dried over anhydrous sodium sulfate, filtered and concentrated under reduced pressure. The crude product was used without further purification. Yield: 262 mg.

¹H-NMR (600 MHz, DMSO-d₆): δ [ppm]=4.44-4.37 (m, 2H), 3.72-3.65 (m, 2H), 3.45-3.38 (m, 1H), 0.55-0.45 (m, 4H).

### Example 6.1A

### Tert-Butyl (11-chloro-7-methyl-2-oxo-7,8-dihydro-2H-[3]benzoxocino[5,6-c]pyridin-3(5H)-yl)-acetate (Racemate)

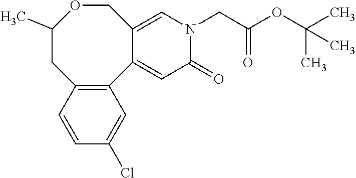 [SMILES: CC1Cc2ccc(Cl)cc2-c2cc(=O)n(CC(=O)OC(C)(C)C)cc2CO1]

11-Chloro-7-methyl-3,5,7,8-tetrahydro-2H-[3]benzoxocino[5,6-c]pyridin-2-one (racemate) (495 mg, 1.77 mmol, 1.0 eq.) and tert-butyl bromoacetate (416 mg, 2.13 mmol, 1.2 eq.) were dissolved in N,N-dimethylformamide (5 ml) and potassium carbonate (368 mg, 2.66 mmol, 1.5 eq.) was added. The reaction mixture was heated to 100° C. and stirred at this temperature for 1 h. After concentrating under reduced pressure, the crude product was dissolved in dichloromethane and purified by column chromatography (silica gel, eluent: cyclohexane/ethyl acetate 100:0 to 50:50). Yield: 582 mg (84% of theory).

LC-MS (method 5): Rₜ=1.37 min; MS (ESIpos): m/z=390 [M+H]⁺

¹H-NMR (400 MHz, DMSO-d₆): δ [ppm]=7.87 (s, 1H), 7.55-7.47 (m, 1H), 7.43-7.37 (m, 1H), 7.34 (d, 1H), 6.39 (s, 1H), 4.63 (s, 2H), 4.38 (d, 1H), 3.61-3.48 (m, 2H), 2.82 (d, 1H), 2.29 (dd, 1H), 1.51-1.39 (s, 9H), 1.29-1.19 (d, 3H).

### Example 6.1B

### Tert-Butyl [(7R)-11-chloro-7-methyl-2-oxo-7,8-dihydro-2H-[3]benzoxocino[5,6-c]pyridin-3(5H)-yl]acetate (Single Stereoisomer)

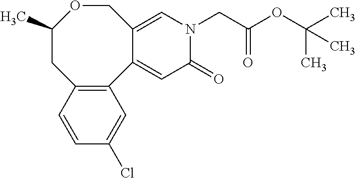 [SMILES: C[C@@H]1Cc2ccc(Cl)cc2-c2cc(=O)n(CC(=O)OC(C)(C)C)cc2CO1]

Enantiomer separation of 782 mg of tert-butyl (11-chloro-7-methyl-2-oxo-7,8-dihydro-2H-[3]benzoxocino[5,6-c]pyridin-3(5H)-yl)acetate (racemate), Example 6.1A gave
single stereoisomer 1 (the title compound Example 6.1B) (chiral SFC: Rₜ=1.20 min, >99% ee): 356 mg,
single stereoisomer 2 (chiral SFC: Rₜ=1.72 min, 94% ee): 350 mg.

Separation method: SFC: column: Daicel Chiralpak AD-H 5 μm, 250 mm×30 mm; eluent: 82% carbon dioxide/18% 2-propanol; temperature: 40° C.; flow rate: 114 ml/min; UV detection: 210 nm.

Analysis method: SFC: column: Daicel Chiralpak AD-H 3 μm, 100 mm×4.6 mm; eluent: 80% carbon dioxide/20% 2-propanol; temperature: 40° C.; flow rate: 3.0 ml/min; UV detection: 210 nm.

### Example 6.2A

### Tert-Butyl 2-(11-chloro-7-methyl-2-oxo-7,8-dihydro-2H-[3]benzoxocino[5,6-c]pyridin-3(5H)-yl)-3-[(2S)-tetrahydro-2H-pyran-2-yl]propanoate (Mixture of Stereoisomers)

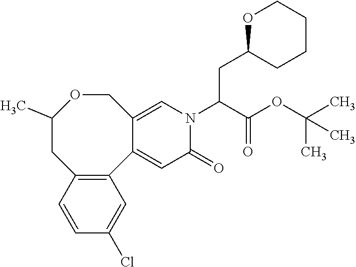 [SMILES: CC1Cc2ccc(Cl)cc2-c2cc(=O)n(C(C[C@@H]3CCCCO3)C(=O)OC(C)(C)C)cc2CO1]

tert-Butyl (1-chloro-7-methyl-2-oxo-7,8-dihydro-2H-[3]benzoxocino[5,6-c]pyridin-3(5H)-yl)acetate (racemate) (100 mg, 0.26 mmol, 1.0 eq.) under argon atmosphere was dissolved in tetrahydrofuran (4 ml) and the mixture was cooled to −78° C. Sodium bis(trimethylsilyl)amide solution (0.32 ml, 1.0 M in tetrahydrofuran, 0.32 mmol, 1.25 eq.) was added dropwise. After 10 min, a solution of (2S)-tetrahydro-2H-pyran-2-ylmethyl trifluoromethanesulfonate (single stereoisomer) (prepared according to WO 2017/005725, example 3.4C) (82 mg, 0.32 mmol, 1.25 eq.) in tetrahydrofuran (2 ml) was added slowly. The mixture was stirred at −78° C. for 15 min and at RT for 30 min. The mixture was cooled to −78° C., acetic acid (22 μl, 0.39 mmol, 1.5 eq.) was added and the mixture was allowed to warm to RT. The mixture was combined with another mixture resulting from a similar experiment [tert-butyl (11-chloro-7-methyl-2-oxo-7,8-dihydro-2H-[3]benzoxocino[5,6-c]pyridin-3(5H)-yl)acetate (racemate) (50 mg, 0.13 mmol, 1.0 eq.) in tetrahydrofuran (2 ml), sodium bis(trimethylsilyl)amide solution (0.15 ml, 1.0 M in tetrahydrofuran, 0.15 mmol, 1.2 eq.) and (2S)-tetrahydro-2H-pyran-2-ylmethyl trifluoromethanesulfonate (single stereoisomer) (39 mg, 0.15 mmol, 1.2 eq.)]. The combined reaction mixtures were concentrated under reduced pressure. The residue was purified by column chromatography (silica gel, eluent: cyclohexane/ethyl acetate 100:0 to 1:1). Yield: 157 mg (83% of theory for both experiments).

LC-MS (method 4): Rₜ=2.44/2.47 min; MS (ESIpos): m/z=488 [M+H]⁺

¹H-NMR (400 MHz, DMSO-d₆): δ [ppm]=7.91-7.67 (m, 1H), 7.56-7.30 (m, 3H), 6.40-6.18 (m, 1H), 5.31-4.98 (m, 1H), 4.51-4.29 (m, 1H), 3.92-3.71 (m, 1H), 3.61-3.44 (m, 2H), 3.41-3.06 (m, 2H, partially concealed), 2.92-2.73 (m, 2H), 2.37-2.19 (m, 2H), 2.17-1.96 (m, 1H), 1.79-1.55 (m, 1H), 1.54-1.34 (m, 13H), 1.28-1.20 (m, 3H).

### Example 6.2B

### 2-(11-Chloro-7-methyl-2-oxo-7,8-dihydro-2H-[3]benzoxocino[5,6-c]pyridin-3(5H)-yl)-3-[(2S)-tetrahydro-2H-pyran-2-yl]propanoic Acid (Mixture of Stereoisomers)

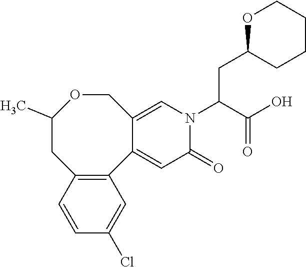 [SMILES: CC1Cc2ccc(Cl)cc2-c2cc(=O)n(C(C[C@@H]3CCCCO3)C(=O)O)cc2CO1]

tert-Butyl 2-(11-chloro-7-methyl-2-oxo-7,8-dihydro-2H-[3]benzoxocino[5,6-c]pyridin-3(5H)-yl)-3-[(2S)-tetrahydro-2H-pyran-2-yl]propanoate (mixture of stereoisomers) (155 mg, 0.32 mmol, 1.0 eq.) was dissolved in dichloromethane (2.0 ml) and trifluoroacetic acid (4.0 ml) was added. The reaction mixture was stirred for 45 min and concentrated under reduced pressure. For three times, the residue was dissolved in dichloromethane and concentrated under reduced pressure. The crude product was used in the subsequent reaction without further purification. Yield: 162 mg.

LC-MS (method 5): Rₜ=1.26/1.28 min; MS (ESIpos): m/z=432 [M+H]⁺

### Example 6.3A

### Tert-Butyl 2-(11-chloro-7-methyl-2-oxo-7,8-dihydro-2H-[3]benzoxocino[5,6-c]pyridin-3(5H)-yl)-3-[(2R)-1,4-dioxan-2-yl]propanoate (Mixture of Stereoisomers)

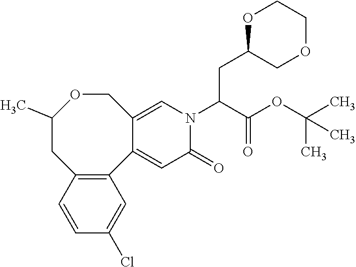 [SMILES: CC1Cc2ccc(Cl)cc2-c2cc(=O)n(C(C[C@@H]3COCCO3)C(=O)OC(C)(C)C)cc2CO1]

tert-Butyl (11-chloro-7-methyl-2-oxo-7,8-dihydro-2H-[3]benzoxocino[5,6-c]pyridin-3(5H)-yl)acetate (racemate) (405 mg, 1.04 mmol, 1.0 eq.) under argon atmosphere was dissolved in tetrahydrofuran (12 ml), 4 Å molecular sieves (1.0 g) was added and the mixture was stirred at RT for 15 min. The mixture was cooled to −78° C. and sodium bis(trimethylsilyl)amide solution (1.30 ml, 1.0 M in tetrahydrofuran, 1.30 mmol, 1.25 eq.) was added dropwise. After 20 min, a solution of (2S)-1,4-dioxan-2-ylmethyl trifluoromethanesulfonate (single stereoisomer) (prepared according to WO 2017/005725, example 3.5A) (335 mg, 1.30 mmol, 1.25 eq.) in tetrahydrofuran (3.0 ml) was added slowly. The mixture was stirred at −78° C. for 2 h, treated with acetic acid (90 μl, 1.56 mmol, 1.5 eq.) and concentrated under reduced pressure. The residue was purified by column chromatography (silica gel, eluent: cyclohexane/ethyl acetate 100:0 to 1:1). Yield: 557 mg (quantitative of theory).

LC-MS (method 4): Rₜ=2.12/2.15 min; MS (ESIpos): m/z=490 [M+H]⁺

¹H-NMR (400 MHz, DMSO-d₆): δ [ppm]=7.94-7.71 (m, 1H), 7.55-7.44 (m, 1H), 7.44-7.27 (m, 2H), 6.44-6.25 (m, 1H), 5.30-4.94 (m, 1H), 4.48-4.38 (m, 1H), 3.76-3.35 (m, 7H), 3.27-3.19 (m, 1H), 3.19-3.02 (m, 1H), 2.89-2.74 (m, 1H), 2.36-2.18 (m, 2H), 2.14-1.95 (m, 1H), 1.48-1.34 (m, 9H), 1.27-1.20 (m, 3H).

### Example 6.3B

### 2-(11-Chloro-7-methyl-2-oxo-7,8-dihydro-2H-[3]benzoxocino[5,6-c]pyridin-3(5H)-yl)-3-[(2R)-1,4-dioxan-2-yl]propanoic Acid (Mixture of Stereoisomers)

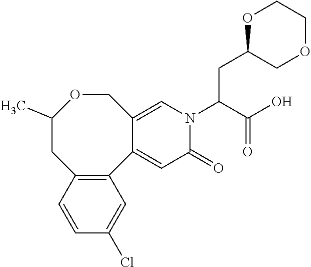 [SMILES: CC1Cc2ccc(Cl)cc2-c2cc(=O)n(C(C[C@@H]3COCCO3)C(=O)O)cc2CO1]

tert-Butyl 2-(11-chloro-7-methyl-2-oxo-7,8-dihydro-2H-[3]benzoxocino[5,6-c]pyridin-3(5H)-yl)-3-[(2R)-1,4-dioxan-2-yl]propanoate (mixture of stereoisomers) (555 mg, 1.13 mmol, 1.0 eq.) was dissolved in dichloromethane (10 ml) and trifluoroacetic acid (10 ml) was added. The reaction mixture was stirred for 45 min and concentrated under reduced pressure. For three times, the residue was dissolved in dichloromethane and concentrated under reduced pressure. The crude product was used in the subsequent reaction without further purification. Yield: 537 mg.

LC-MS (method 1): Rₜ=0.82 min; MS (ESIpos): m/z=434 [M+H]⁺

### Example 6.4A

### Tert-Butyl (2ξ)-2-[(7R)-11-chloro-7-methyl-2-oxo-7,8-dihydro-2H-[3]benzoxocino[5,6-c]pyridin-3(5H)-yl]-2,3,5-trideoxy-4-O-methyl-L-glycero-pentonate (Mixture of Two Diastereomers)

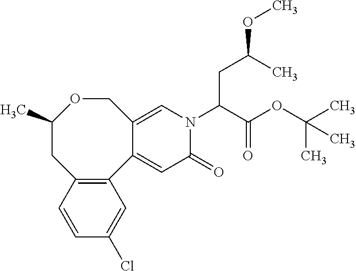 [SMILES: CO[C@@H](C)CC(C(=O)OC(C)(C)C)n1cc2c(cc1=O)-c1cc(Cl)ccc1C[C@@H](C)OC2]

A solution of lithium bis(trimethylsilyl)amide (0.46 ml, 1.0 M in tetrahydrofuran, 0.46 mmol, 1.2 eq.) was added under argon atmosphere at −78° C. to a solution of tert-butyl [(7R)-11-chloro-7-methyl-2-oxo-7,8-dihydro-2H-[3]benzoxocino[5,6-c]pyridin-3(5H)-yl]acetate (single stereoisomer) (150 mg, 0.39 mmol) in tetrahydrofuran (5 ml). After stirring at −78° C. for 15 min, (2S)-2-methoxypropyl trifluoromethanesulfonate (single stereoisomer) (145 mg, 0.65 mmol, 1.7 eq.) (prepared according to WO 2014/154794, example 28.1A) was added dropwise. The reaction mixture was stirred at −78° C. for 15 min, allowed to warm to 0° C. and stirred at 0° C. for 1 h. The reaction mixture was quenched with saturated aqueous solution of ammonium chloride. After addition of ethyl acetate and phase separation, the aqueous phase was extracted with ethyl acetate. The combined organic phases were washed with brine, dried over anhydrous sodium sulfate, filtered and concentrated under reduced pressure. The residue was purified by column chromatography (silica gel, eluent: cyclohexane/ethyl acetate gradient). Yield: 123 mg (92% purity, 64% of theory).

LC-MS (method 4): Rₜ=2.41 min; MS (ESIpos): m/z=462 [M+H]⁺

### Example 6.4B

### (2ξ)-2-[(7R)-11-Chloro-7-methyl-2-oxo-7,8-dihydro-2H-[3]benzoxocino[5,6-c]pyridin-3(5H)-yl]-2,3,5-trideoxy-4-O-methyl-L-glycero-pentonic Acid (Mixture of Two Diastereomers)

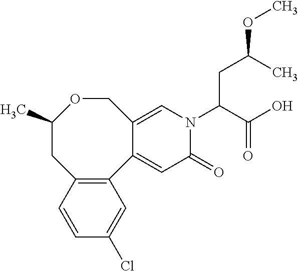

Lithium hydroxide (12 mg, 0.49 mmol, 2.0 eq.) was added at RT to a solution of tert-butyl (2ξ)-2-[(7R)-11-chloro-7-methyl-2-oxo-7,8-dihydro-2H-[3]benzoxocino[5,6-c]pyridin-3(5H)-yl]-2,3,5-trideoxy-4-O-methyl-L-glycero-pentonate (mixture of two diastereomers) (123 mg, 92% purity, 0.25 mmol) in a mixture of tetrahydrofuran and water (3:1, 5 ml). The reaction mixture was stirred at RT overnight and acidified with aqueous hydrochloric acid (1 N). After removing all volatiles under reduced pressure, the aqueous residue was extracted with ethyl acetate. The combined organic phases were washed with brine, dried over anhydrous sodium sulfate, filtered and concentrated under reduced pressure. The residue was purified by preparative HPLC (reversed phase, eluent: acetonitrile/water gradient). Yield: 58 mg (58% of theory).

LC-MS (method 4): Rₜ=1.66/1.69 min; MS (ESIpos): m/z=406 [M+H]⁺

### Example 6.5A

### Tert-Butyl 2-[(7R)-11-chloro-7-methyl-2-oxo-7,8-dihydro-2H-[3]benzoxocino[5,6-c]pyridin-3(5H)-yl]-4-(difluoromethoxy)butanoate (Mixture of Two Diastereomers)

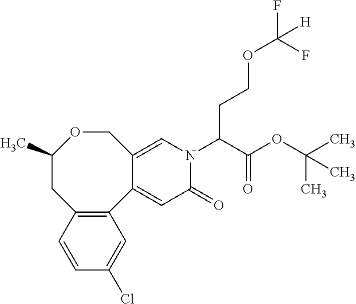 [SMILES: C[C@@H]1Cc2ccc(Cl)cc2-c2cc(=O)n(C(CCOC(F)F)C(=O)OC(C)(C)C)cc2CO1]

1,1,3,3-Tetramethylguanidine (143 μl, 1.14 mmol, 3.0 eq.) was added under argon atmosphere at RT to a solution of (7R)-11-chloro-7-methyl-3,5,7,8-tetrahydro-2H-[3]benzoxocino[5,6-c]pyridin-2-one (single stereoisomer) (105 mg, 0.38 mmol) in 2-propanol (1.5 ml) and acetone (0.4 ml). The mixture was stirred at RT for 15 min, followed by addition of tert-butyl 2-bromo-4-(difluoromethoxy)butanoate (racemate) (135 mg, 90% purity, 0.42 mmol, 1.1 eq.) and of further 2-propanol (1.5 ml) and acetone (0.4 ml). The reaction mixture was stirred at RT for 3 days and concentrated under reduced pressure. The residue was purified by preparative HPLC (reversed phase, eluent: acetonitrile/water gradient). Yield: 169 mg (92% of theory).

LC-MS (method 3): Rₜ=3.94/3.97 min; MS (ESIpos): m/z=484 [M+H]⁺

### Example 6.5B

### 2-[(7R)-11-Chloro-7-methyl-2-oxo-7,8-dihydro-2H-[3]benzoxocino[5,6-c]pyridin-3(5H)-yl]-4-(difluoromethoxy)butanoic Acid (Mixture of Two Diastereomers)

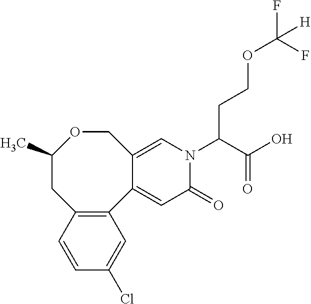 [SMILES: C[C@@H]1Cc2ccc(Cl)cc2-c2cc(=O)n(C(CCOC(F)F)C(=O)O)cc2CO1]

Lithium hydroxide (9 mg, 0.39 mmol, 2.0 eq.) was added at RT to a solution of tert-butyl 2-[(7R)-11-chloro-7-methyl-2-oxo-7,8-dihydro-2H-[3]benzoxocino[5,6-c]pyridin-3(5H)-yl]-4-(difluoromethoxy)-butanoate (mixture of two diastereomers) (93 mg, 0.20 mmol) in a mixture of tetrahydrofuran and water (3:1, 5 ml). The reaction mixture was stirred at RT overnight and acidified with aqueous hydrochloric acid (1 N). After removing all volatiles under reduced pressure, the residue was crystallized with water, filtered and dried in vacuo. Yield: 72 mg (88% of theory).

LC-MS (method 4): Rₜ=1.71 min; MS (ESIpos): m/z=428 [M+H]⁺

### Example 6.6A

### Tert-Butyl (2ξ)-2-[(7R)-11-chloro-7-methyl-2-oxo-7,8-dihydro-2H-[3]benzoxocino[5,6-c]pyridin-3(5H)-yl]-2,3,5-trideoxy-4-O-(difluoromethyl)-L-glycero-pentonate (Mixture of Two Diastereomers)

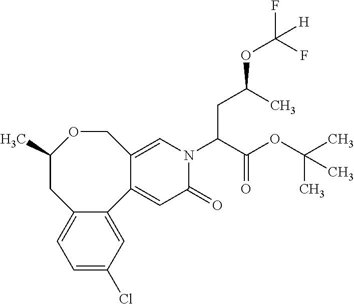 [SMILES: C[C@@H]1Cc2ccc(Cl)cc2-c2cc(=O)n(C(C[C@H](C)OC(F)F)C(=O)OC(C)(C)C)cc2CO1]

1,1,3,3-Tetramethylguanidine (151 μl, 1.20 mmol, 3.0 eq.) was added under argon atmosphere at RT to a solution of (7R)-11-chloro-7-methyl-3,5,7,8-tetrahydro-2H-[3]benzoxocino[5,6-c]pyridin-2-one (single stereoisomer) (110 mg, 0.40 mmol) in 2-propanol/acetone (4:1, 2.5 ml). The mixture was stirred at RT for 15 min, followed by addition of tert-butyl (4S)-2-bromo-4-(difluoromethoxy)-pentanoate (mixture of two diastereomers) (138 mg, 0.44 mmol, 1.1 eq.) and of further 2-propanol/acetone (4:1, 2.5 ml). The reaction mixture was stirred at RT for 3 days and concentrated under reduced pressure. The residue was purified by preparative HPLC (reversed phase, eluent: acetonitrile/water gradient). Yield: 132 mg (66% of theory).

LC-MS (method 4): Rₜ=2.33/2.35 min; MS (ESIpos): m/z=498 [M+H]⁺

### Example 6.6B

### (2ξ)-2-[(7R)-11-Chloro-7-methyl-2-oxo-7,8-dihydro-2H-[3]benzoxocino[5,6-c]pyridin-3(5H)-yl]-2,3,5-trideoxy-4-O-(difluoromethyl)-L-glycero-pentonic Acid (Mixture of Two Diastereomers)

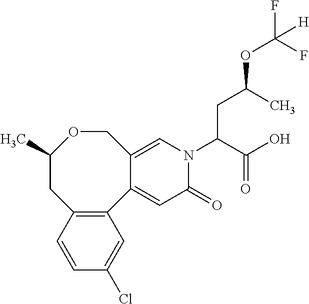 [SMILES: C[C@@H]1Cc2ccc(Cl)cc2-c2cc(=O)n(C(C[C@H](C)OC(F)F)C(=O)O)cc2CO1]

Lithium hydroxide (13 mg, 0.53 mmol, 2.0 eq.) was added at RT to a solution of tert-butyl (2ξ)-2-[(7R)-1-chloro-7-methyl-2-oxo-7,8-dihydro-2H-[3]benzoxocino[5,6-c]pyridin-3(5H)-yl]-2,3,5-trideoxy-4-O-(difluoromethyl)-L-glycero-pentonate (mixture of two diastereomers) (132 mg, 0.27 mmol) in a mixture of tetrahydrofuran and water (3:1, 5 ml). The reaction mixture was stirred at RT overnight and acidified with aqueous hydrochloric acid (1 N). After removing all volatiles under reduced pressure, the residue was crystallized with water, filtered and dried in vacuo. Yield: 95 mg (81% of theory).

LC-MS (method 4): Rₜ=1.84 min; MS (ESIpos): m/z=442 [M+H]⁺

### Example 6.7A

### Tert-Butyl 2-[(7R)-11-chloro-7-methyl-2-oxo-7,8-dihydro-2H-[3]benzoxocino[5,6-c]pyridin-3(5H)-yl]-4-isopropoxybutanoate (Mixture of Two Diastereomers)

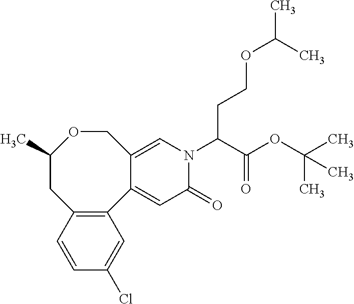 [SMILES: CC(C)OCCC(C(=O)OC(C)(C)C)n1cc2c(cc1=O)-c1cc(Cl)ccc1C[C@@H](C)OC2]

A solution of lithium bis(trimethylsilyl)amide (0.46 ml, 1.0 M in tetrahydrofuran, 0.46 mmol, 1.2 eq.) was added under argon atmosphere at −78° C. to a solution of tert-butyl [(7R)-11-chloro-7-methyl-2-oxo-7,8-dihydro-2H-[3]benzoxocino[5,6-c]pyridin-3(5H)-yl]acetate (single stereoisomer) (150 mg, 0.39 mmol) in tetrahydrofuran (5 ml). After stirring at −78° C. for 15 min, 2-[(propan-2-yl)oxy]ethyl trifluoromethanesulfonate (136 mg, 0.58 mmol, 1.5 eq.) (prepared according to WO 2016/146606, example 2.1A) was added dropwise. The resulting reaction mixture was stirred at −78° C. for 15 min, allowed to warm to RT and stirred at RT for 1.5 h. The reaction mixture was quenched with saturated aqueous solution of ammonium chloride at 0° C. After addition of ethyl acetate and phase separation, the aqueous phase was extracted with ethyl acetate. The combined organic phases were washed with brine, dried over anhydrous sodium sulfate, filtered and concentrated under reduced pressure. The residue was purified by column chromatography (silica gel, eluent: cyclohexane/ethyl acetate gradient). Yield: 93 mg (51% of theory).

LC-MS (method 4): Rₜ=2.41/2.45 min; MS (ESIpos): m/z=476 [M+H]⁺

### Example 6.7B

### 2-[(7R)-11-Chloro-7-methyl-2-oxo-7,8-dihydro-2H-[3]benzoxocino[5,6-c]pyridin-3(5H)-yl]-4-isopropoxybutanoic Acid (Mixture of Two Diastereomers)

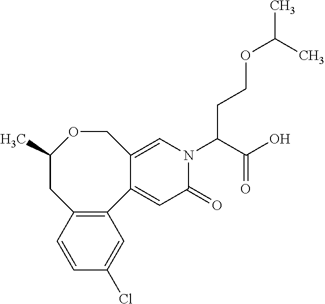 [SMILES: CC(C)OCCC(C(=O)O)n1cc2c(cc1=O)-c1cc(Cl)ccc1C[C@@H](C)OC2]

Lithium hydroxide (9 mg, 0.39 mmol, 2.0 eq.) was added at RT to a solution of tert-butyl 2-[(7R)-11-chloro-7-methyl-2-oxo-7,8-dihydro-2H-[3]benzoxocino[5,6-c]pyridin-3(5H)-yl]-4-isopropoxybutanoate (mixture of two diastereomers) (93 mg, 0.20 mmol) in a mixture of tetrahydrofuran and water (3:1, 5 ml). The reaction mixture was stirred at RT overnight and acidified with aqueous hydrochloric acid (1 N). After removing all volatiles under reduced pressure, the residue was crystallized with water, filtered and dried in vacuo. Yield: 72 mg (88% of theory).

LC-MS (method 4): Rₜ=1.82/1.85 min; MS (ESIpos): m/z=420 [M+H]⁺

### Example 6.5A

### Tert-Butyl 4-tert-butoxy-2-(11-chloro-7-methyl-2-oxo-7,8-dihydro-2H-[3]benzoxocino[5,6-c]-pyridin-3(5H)-yl)butanoate (Mixture of Stereoisomers)

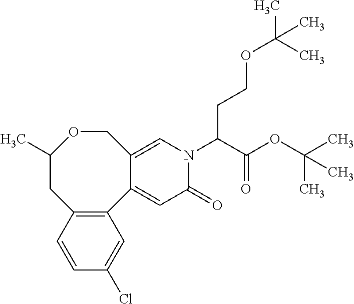 [SMILES: CC1Cc2ccc(Cl)cc2-c2cc(=O)n(C(CCOC(C)(C)C)C(=O)OC(C)(C)C)cc2CO1]

tert-Butyl (11-chloro-7-methyl-2-oxo-7,8-dihydro-2H-[3]benzoxocino[5,6-c]pyridin-3(5H)-yl)acetate (racemate) (75 mg, 0.19 mmol, 1.0 eq.) under argon atmosphere was dissolved in tetrahydrofuran (2 ml) and cooled to −78° C. Sodium bis(trimethylsilyl)amide solution (240 μl, 1.0 M in tetrahydrofuran, 0.24 mmol, 1.25 eq.) was added. After stirring for 20 min, a solution of 2-ten-butoxyethyl trifluoromethanesulfonate (prepared according to WO 2016/146606, example 2.3A) (60 mg, 0.24 mmol, 1.25 eq.) in tetrahydrofuran (1 ml) was added dropwise and the reaction mixture was treated with acetic acid (17 μl, 0.29 mmol, 1.5 eq.) at −78° C. The volatiles were removed under reduced pressure and the residue was purified by column chromatography (silica gel, eluent: cyclohexane/ethyl acetate 92:8 to 33:66). Yield: 36 mg (38% of theory).

LC-MS (method 4): Rₜ=2.52/2.57 min; MS (ESIpos): m/z=490 [M+H]⁺

¹H-NMR (400 MHz, DMSO-d₆): δ [ppm]=7.85/7.82 (2s, 1H), 7.50 (dd, 1H), 7.39 (d, 1H), 7.35-7.28 (m, 1H), 6.36 (s, 1H), 5.24-5.07 (m, 1H), 4.51-4.36 (m, 1H), 3.62-3.49 (m, 2H), 3.43-3.32 (m, 1H), 3.28-2.99 (m, 1H), 2.87-2.76 (m, 1H), 2.39-2.17 (m, 3H), 1.46-1.37 (m, 9H), 1.26-1.19 (m, 3H), 1.07/1.03 (2s, 9H).

### Example 6.8B

### 4-tert-Butoxy-2-(11-chloro-7-methyl-2-oxo-7,8-dihydro-2H-[3]benzoxocino[5,6-c]pyridin-3(5H)-yl)butanoic Acid (Mixture of Stereoisomers)

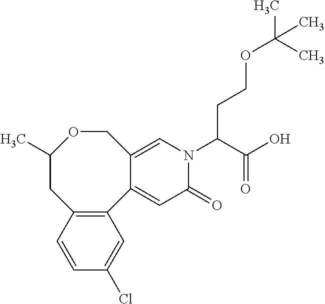 [SMILES: CC1Cc2ccc(Cl)cc2-c2cc(=O)n(C(CCOC(C)(C)C)C(=O)O)cc2CO1]

tert-Butyl 4-tert-butoxy-2-(11-chloro-7-methyl-2-oxo-7,8-dihydro-2H-[3]benzoxocino[5,6-c]pyridin-3(5H)-yl)butanoate (mixture of stereoisomers) (417 mg, 0.85 mmol, 1.0 eq.) was dissolved in tetrahydrofuran (20 ml) and a solution of lithium hydroxide (102 mg, 4.25 mmol, 5.0 eq.) in water (10 ml) was added. The reaction mixture was stirred at RT overnight and acidified by addition of aqueous hydrochloric acid (1 N). The resulting crude product was filtered and subsequently washed with water and cyclohexane. The residue was purified by preparative HPLC (reversed phase, eluent: water with 0.05% formic acid/acetonitrile 95:5 to 40:60). Yield: 307 mg (79% of theory).

LC-MS (method 5): Rₜ=1.33/1.34 min; MS (ESIneg): m/z=432 [M−H]⁻

¹H-NMR (400 MHz, DMSO-d₆): δ [ppm]=12.98 (br s, 1H), 7.86/7.84 (2s, 1H), 7.50 (m, 1H), 7.40/7.38 (2s, 1H), 7.36-7.28 (m, 1H), 6.36/6.35 (2s, 1H), 5.30-5.22 (m, 1H), 5.09 (m, 1H), 4.53-4.36 (m, 1H), 3.63-3.46 (m, 2H), 3.41-3.34 (m, 1H), 3.22-3.02 (m, 1H), 2.88-2.76 (m, 1H), 2.43-2.19 (m, 3H), 1.27-1.17 (m, 3H), 1.08/1.02 (2s, 9H).

### Example 6.9A

### Tert-Butyl 2-[(7R)-11-chloro-7-methyl-2-oxo-7,8-dihydro-2H-[3]benzoxocino[5,6-c]pyridin-3(5H)-yl]-4-(cyclopropyloxy)butanoate (Mixture of Two Diastereomers)

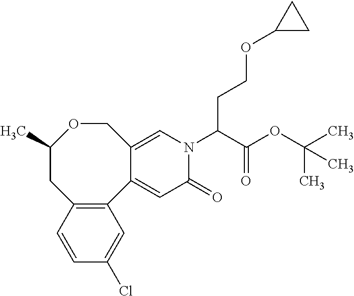 [SMILES: C[C@@H]1Cc2ccc(Cl)cc2-c2cc(=O)n(C(CCOC3CC3)C(=O)OC(C)(C)C)cc2CO1]

A solution of lithium bis(trimethylsilyl)amide (0.42 ml, 1.0 M in tetrahydrofuran, 0.42 mmol, 1.2 eq.) was added under argon atmosphere at −78° C. to a solution of tert-butyl [(7R)-11-chloro-7-methyl-2-oxo-7,8-dihydro-2H-[3]benzoxocino[5,6-c]pyridin-3(5H)-yl]acetate (single stereoisomer) (137 mg, 0.35 mmol) in tetrahydrofuran (5 ml). After stirring at −78° C. for 15 min, 2-(cyclopropyloxy)ethyl trifluoromethanesulfonate (155 mg, 90% purity, 0.60 mmol, 1.7 eq.) was added dropwise. The reaction mixture was stirred at −78° C. for 15 min, allowed to slowly warm to 0° C. and stirred at 0° C. for 1 h. The reaction mixture was quenched with saturated aqueous solution of ammonium chloride. After addition of ethyl acetate and phase separation, the aqueous phase was extracted with ethyl acetate. The combined organic phases were washed with brine, dried over anhydrous sodium sulfate, filtered and concentrated under reduced pressure. The residue was purified by preparative HPLC (reversed phase, eluent: acetonitrile/water gradient). Yield: 51 mg (31% of theory).

LC-MS (method 4): Rₜ=2.34/2.37 min; MS (ESIpos): m/z=474 [M+H]⁺

### Example 6.9B

### 2-[(7R)-11-Chloro-7-methyl-2-oxo-7,8-dihydro-2H-[3]benzoxocino[5,6-c]pyridin-3(5H)-yl]-4-(cyclopropyloxy)butanoic Acid (Mixture of Two Diastereomers)

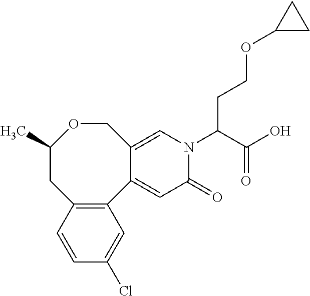 [SMILES: C[C@@H]1Cc2ccc(Cl)cc2-c2cc(=O)n(C(CCOC3CC3)C(=O)O)cc2CO1]

Lithium hydroxide (5 mg, 0.22 mmol, 2.0 eq.) was added at RT to a solution of tert-butyl 2-[(7R)-11-chloro-7-methyl-2-oxo-7,8-dihydro-2H-[3]benzoxocino[5,6-c]pyridin-3(5H)-yl]-4-(cyclopropyloxy)butanoate (mixture of two diastereomers) (51 mg, 0.11 mmol) in a mixture of tetrahydrofuran and water (3:1, 4 ml). The reaction mixture was stirred at RT overnight and acidified with aqueous hydrochloric acid (1 N). After removing all volatiles under reduced pressure, the residue was diluted and crystallized with water, filtered and dried in vacuo. Yield: 46 mg (quantitative of theory).

LC-MS (method 4): Rₜ=1.77/1.79 min; MS (ESIpos): m/z=418 [M+H]⁺

### Example 6.10A

### Tert-Butyl (11-chloro-7-ethyl-2-oxo-7,8-dihydro-2H-[3]benzoxocino[5,6-c]pyridin-3(5H)-yl)acetate (Racemate)

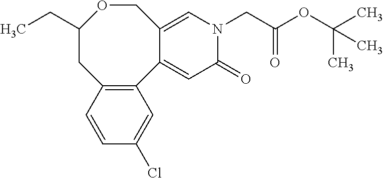 [SMILES: CCC1Cc2ccc(Cl)cc2-c2cc(=O)n(CC(=O)OC(C)(C)C)cc2CO1]

11-Chloro-7-ethyl-3,5,7,8-tetrahydro-2H-[3]benzoxocino[5,6-c]pyridin-2-one (racemate) (0.65 g, 76% purity, 1.7 mmol, 1.0 eq.) was dissolved in N,N-dimethylformamide (7 ml). tert-Butyl bromoacetate (0.50 g, 2.6 mmol, 1.5 eq.) and potassium carbonate (0.47 g, 3.4 mmol, 2.0 eq.) were added. After stirring for 1 h at 100° C., the reaction mixture was cooled to RT, diluted with ethyl acetate and washed with water and brine. The organic layer was dried over anhydrous sodium sulfate, filtered and concentrated under reduced pressure. The residue was purified by column chromatography (silica gel, eluent: petroleum ether/ethyl acetate 50:1 to 10:1). Yield: 0.37 g (95% purity, 51% of theory).

LC-MS (method 14): Rₜ=1.74 min; MS (ESIpos): m/z=404 [M+H]⁺

¹H-NMR (400 MHz, DMSO-d₆): δ [ppm]=7.89 (s, 1H), 7.52-7.49 (m, 1H), 7.37-7.34 (m, 2H), 6.39 (s, 1H), 4.63 (s, 2H), 4.43 (d, 1H), 3.54 (d, 1H), 3.33-3.26 (m, 1H), 2.82 (d, 1H), 2.29-2.23 (m, 1H), 1.60-1.52 (m, 2H), 1.44 (s, 9H), 0.96-0.88 (m, 3H).

### Example 6.10B

### tert-Butyl [(7R)-11-chloro-7-ethyl-2-oxo-7,8-dihydro-2H-[3]benzoxocino[5,6-c]pyridin-3(5H)-yl]acetate (Single Stereoisomer)

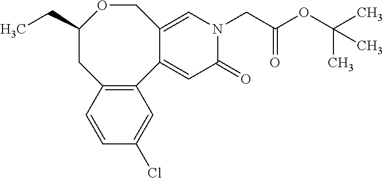 [SMILES: CC[C@@H]1Cc2ccc(Cl)cc2-c2cc(=O)n(CC(=O)OC(C)(C)C)cc2CO1]

Enantiomer separation of 0.37 g of tert-butyl (11-chloro-7-ethyl-2-oxo-7,8-dihydro-2H-[3]benzoxocino[5,6-c]pyridin-3(5H)-yl)acetate (racemate) Example 6.10A gave
single stereoisomer 1 (the title compound Example 6.10B) (chiral SFC: Rₜ=0.87 min, 99% ee): 123 mg,
single stereoisomer 2 (chiral SFC: Rₜ=0.99 min): 125 mg.

Separation method: SFC: column: Daicel Chiralcel OJ-H 5 μm, 250 mm×20 mm; eluent: carbon dioxide/methanol 94:6; temperature: 40° C.; flow rate: 80 ml/min; UV detection: 210 nm.

Analysis method: SFC: column: Daicel Chiralcel OJ-H 5 μm, 250 mm×4.6 mm; eluent: carbon dioxide/methanol 90:10; temperature: 40° C.; flow rate: 3.0 ml/min; UV detection: 210 nm.

### Example 6.11A

### Tert-Butyl 2-[(7R)-11-chloro-7-ethyl-2-oxo-7,8-dihydro-2H-[3]benzoxocino[5,6-c]pyridin-3(5H)-yl]-3-[(2S)-tetrahydro-2H-pyran-2-yl]propanoate (Mixture of Two Diastereomers)

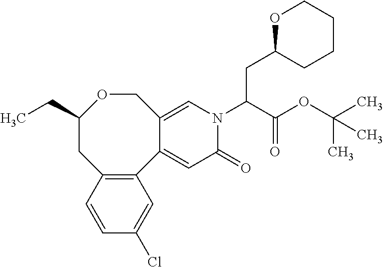 [SMILES: CC[C@@H]1Cc2ccc(Cl)cc2-c2cc(=O)n(C(C[C@@H]3CCCCO3)C(=O)OC(C)(C)C)cc2CO1]

tert-Butyl [(7R)-11-chloro-7-ethyl-2-oxo-7,8-dihydro-2H-[3]benzoxocino[5,6-c]pyridin-3(5H)-yl]acetate (single stereoisomer) (123 mg, 0.29 mmol, 1.0 eq.) under argon atmosphere was dissolved in tetrahydrofuran (6.0 ml), cooled to −78° C. and sodium bis(trimethylsilyl)amide solution (0.37 ml, 1.0 M in tetrahydrofuran, 0.37 mmol, 1.25 eq.) was added dropwise. After 20 min, a solution of (2S)-tetrahydro-2H-pyran-2-ylmethyl trifluoromethanesulfonate (single stereoisomer) (prepared according to WO 2017/005725, example 3.4C) (96 mg, 0.37 mmol, 1.25 eq.) in tetrahydrofuran (3.0 ml) was added slowly. The mixture was stirred at −78° C. for 15 min and at RT for 30 min. The mixture was cooled to −78° C., acetic acid (25 μl, 0.44 mmol, 1.5 eq.) was added and the mixture was concentrated under reduced pressure. The residue was purified by column chromatography (silica gel, eluent: cyclohexane/ethyl acetate 100:0 to 1:1). Yield: 77 mg (51% of theory).

LC-MS (method 4): Rₜ=2.60/2.61 min; MS (ESIpos): m/z=502 [M+H]⁺

### Example 6.11B

### 2-[(7R)-11-Chloro-7-ethyl-2-oxo-7,8-dihydro-2H-[3]benzoxocino[5,6-c]pyridin-3(5H)-yl]-3-[(2S)-tetrahydro-2H-pyran-2-yl]propanoic Acid (Mixture of Two Diastereomers)

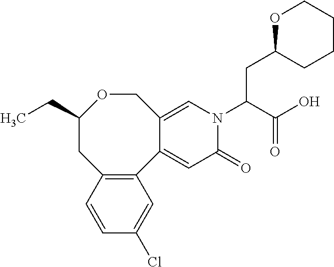 [SMILES: CC[C@@H]1Cc2ccc(Cl)cc2-c2cc(=O)n(C(C[C@@H]3CCCCO3)C(=O)O)cc2CO1]

tert-Butyl 2-[(7R)-11-chloro-7-ethyl-2-oxo-7,8-dihydro-2H-[3]benzoxocino[5,6-c]pyridin-3(5H)-yl]-3-[(2S)-tetrahydro-2H-pyran-2-yl]propanoate (mixture of two diastereomers) (75 mg, 0.15 mmol, 1.0 eq.) was dissolved in a mixture of tetrahydrofuran and water (2:1, 4.5 ml) and lithium hydroxide (72 mg, 3.0 mmol, 20 eq.) was added. The reaction mixture was stirred at 40° C. overnight and for additional 8 h at 40° C. The reaction mixture was concentrated under reduced pressure and the aqueous phase was adjusted to pH 2 using aqueous hydrochloric acid (1 N). The aqueous phase was extracted with ethyl acetate, the combined organic phases dried over anhydrous sodium sulfate, filtered and concentrated under reduced pressure. The crude product was used in the subsequent reaction without further purification. Yield: 64 mg (95% of theory).

LC-MS (method 4): Rₜ=2.01/2.05 min; MS (ESIpos): m/z=446 [M+H]⁺

### Example 6.12A

### Tert-Butyl [7-({[tert-butyl(diphenyl)silyl]oxy}methyl)-11-chloro-2-oxo-7,8-dihydro-2H-[3]benzoxocino[5,6-c]pyridin-3(5H)-yl]acetate (Racemate)

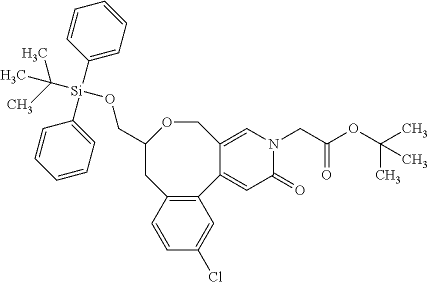 [SMILES: CC(C)(C)OC(=O)Cn1cc2c(cc1=O)-c1cc(Cl)ccc1CC(CO[Si](c1ccccc1)(c1ccccc1)C(C)(C)C)OC2]

7-({[tert-Butyl(diphenyl)silyl]oxy}methyl)-11-chloro-3,5,7,8-tetrahydro-2H-[3]benzoxocino[5,6-c]pyridin-2-one (racemate) (1.10 g, 89% purity, 1.85 mmol, 1.0 eq.) and tert-butyl bromoacetate (540 mg, 2.77 mmol, 1.5 eq.) were dissolved in N,N-dimethylformamide (22 ml) and potassium carbonate (638 mg, 2.77 mmol, 2.5 eq.) was added. The resulting mixture was heated to 100° C. overnight, followed by the addition of further amounts of tert-butyl bromoacetate (540 mg, 2.77 mmol, 1.5 eq.). Stirring was continued at 100° C. for additional 3.5 h. After filtration, the crude mixture was purified by preparative HPLC (reversed phase, eluent: water with 0.05% formic acid/acetonitrile 90:10 to 5:95). Yield: 426 mg (82% purity), 344 mg (97% purity) (combined: 57% of theory).

LC-MS (method 3): Rₜ=5.66 min; MS (ESIpos): m/z=644 [M+H]⁺

### Example 6.13A

### Tert-Butyl 2-[7-({[tert-butyl(diphenyl)silyl]oxy}methyl)-11-chloro-2-oxo-7,8-dihydro-2H-[3]benzoxocino[5,6-c]pyridin-3(5H)-yl]-3-[(2S)-tetrahydro-2H-pyran-2-yl]propanoate (Mixture of Stereoisomers)

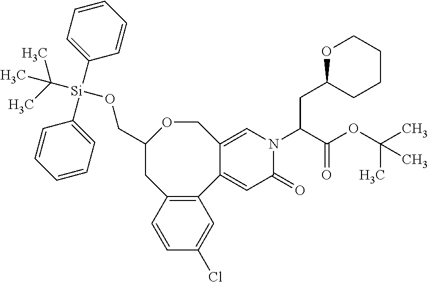 [SMILES: CC(C)(C)OC(=O)C(C[C@@H]1CCCCO1)n1cc2c(cc1=O)-c1cc(Cl)ccc1CC(CO[Si](c1ccccc1)(c1ccccc1)C(C)(C)C)OC2]

[7-({[tert-Butyl(diphenyl)silyl]oxy}methyl)-11-chloro-2-oxo-7,8-dihydro-2H-[3]benzoxocino[5,6-c]pyridin-3(5H)-yl]acetate (racemate) (820 mg, 1.24 mmol, 1.0 eq.) was dissolved in tetrahydrofuran (20 ml), cooled to −78° C. and sodium bis(trimethylsilyl)amide solution (1.54 ml, 1.0 M in tetrahydrofuran, 1.54 mmol, 1.25 eq.) was added dropwise. After 20 min, a solution of (2S)-tetrahydro-2H-pyran-2-ylmethyl trifluoromethanesulfonate (single stereoisomer) (prepared according to WO 2017/005725, example 3.4C) (435 mg, 88% purity, 1.54 mmol, 1.25 eq.) dissolved in tetrahydrofuran (10 ml) was added slowly. The mixture was stirred at −78° C. for 50 min, followed by the addition of acetic acid (106 μl, 1.85 mmol, 1.5 eq.). The reaction mixture was concentrated under reduced pressure and used in the subsequent reaction without further purification. Yield: 1.28 g (78% purity, quantitative of theory).

LC-MS (method 3): Rₜ=6.26/6.31 min; MS (ESIpos): m/z=742 [M+H]⁺

### Example 6.13B

### Tert-Butyl 2-[11-chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanoate (Mixture of Stereoisomers)

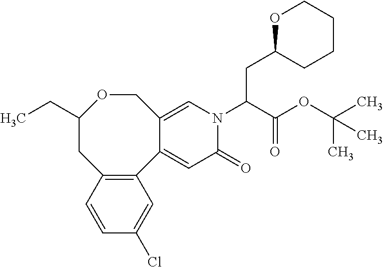 [SMILES: CCC1Cc2ccc(Cl)cc2-c2cc(=O)n(C(C[C@@H]3CCCCO3)C(=O)OC(C)(C)C)cc2CO1]

tert-Butyl 2-[7-({[tert-butyl(diphenyl)silyl]oxy}methyl)-11-chloro-2-oxo-7,8-dihydro-2H-[3]benzoxocino[5,6-c]pyridin-3(5H)-yl]-3-[(2S)-tetrahydro-2H-pyran-2-yl]propanoate (mixture of stereoisomers) (1.28 g, 78% purity, 1.87 mmol, 1.0 eq.) was dissolved in tetrahydrofuran (50 ml), followed by the addition of tetra-N-butylammonium fluoride solution (3.36 ml, 1.0 M in tetrahydrofuran, 3.36 mmol, 2.5 eq.). The reaction mixture was stirred at RT overnight, concentrated under reduced pressure and the crude product was purified by preparative HPLC (reversed phase, eluent: water with 0.05% formic acid/acetonitrile 90:10 to 5:95). Yield: 340 mg (49% of theory).

LC-MS (method 1): Rₜ=1.06 min; MS (ESIpos): m/z=504 [M+H]⁺

### Example 6.13C

### 2-[11-Chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanoic Acid (Mixture of Stereoisomers)

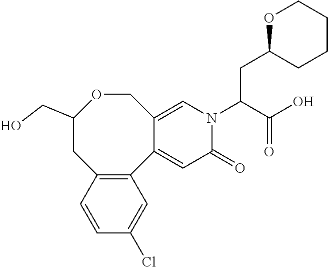 [SMILES: O=C(O)C(C[C@@H]1CCCCO1)n1cc2c(cc1=O)-c1cc(Cl)ccc1CC(CO)OC2]

tert-Butyl 2-[11-chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanoate (mixture of stereoisomers) (240 mg, 0.46 mmol, 1.0 eq.) was dissolved in a mixture of tetrahydrofuran and water (2:1, 15 ml), followed by the addition of lithium hydroxide (55.3 mg, 2.31 mmol, 5.0 eq.). The reaction mixture was stirred at RT overnight, subsequently treated with aqueous hydrochloric acid (1 N) and concentrated under reduced pressure. The crude product was used in the subsequent reaction without further purification. Yield: 261 mg (quantitative of theory).

LC-MS (method 4): Rₜ=2.25/2.37 min; MS (ESIpos): m/z=448 [M+H]⁺

### Example 6.14A

### Tert-Butyl 2-[7-({[tert-butyl(diphenyl)silyl]oxy}methyl)-11-chloro-2-oxo-7,8-dihydro-2H-[3]benzoxocino[5,6-c]pyridin-3(5H)-yl]-3-[(2R)-1,4-dioxan-2-yl]propanoate (Mixture of Stereoisomers)

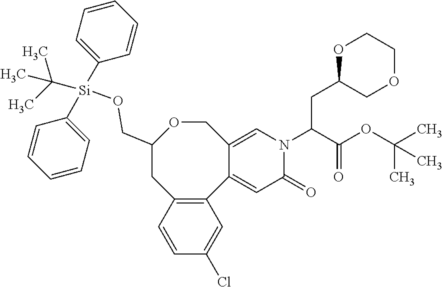 [SMILES: CC(C)(C)OC(=O)C(C[C@@H]1COCCO1)n1cc2c(cc1=O)-c1cc(Cl)ccc1CC(CO[Si](c1ccccc1)(c1ccccc1)C(C)(C)C)OC2]

[7-({[tert-Butyl(diphenyl)silyl]oxy}methyl)-11-chloro-2-oxo-7,8-dihydro-2H-[3]benzoxocino[5,6-c]pyridin-3(5H)-yl]acetate (racemate) (274 mg, 0.41 mmol, 1.0 eq.) was dissolved in tetrahydrofuran (7 ml), cooled to −78° C. and sodium bis(trimethylsilyl)amide solution (0.52 ml, 1.0 M in tetrahydrofuran, 0.52 mmol, 1.25 eq.) was added dropwise. After 20 min, (2S)-1,4-dioxan-2-ylmethyl trifluoromethanesulfonate (single stereoisomer) (prepared according to WO 2017/005725, example 3.5A) (129 mg, 0.52 mmol, 1.25 eq.) dissolved in tetrahydrofuran (3 ml) was added slowly. The reaction mixture was stirred at −78° C. for 50 min, followed by the addition of acetic acid (35 μl, 0.62 mmol, 1.5 eq.). After concentration under reduced pressure, the crude product was purified by preparative HPLC (reversed phase, eluent: water with 0.05% formic acid/acetonitrile 90:10 to 5:95). Yield: 248 mg (85% purity, 69% of theory).

LC-MS (method 3): Rₜ=5.80/5.84 min; MS (ESIpos): m/z=744 [M+H]⁺

### Example 6.14B

### 2-[7-({[tert-Butyl(diphenyl)silyl]oxy}methyl)-11-chloro-2-oxo-7,8-dihydro-2H-[3]benzoxocino[5,6-c]pyridin-3(5H)-yl]-3-[(2R)-1,4-dioxan-2-yl]propanoic Acid (Mixture of Stereoisomers)

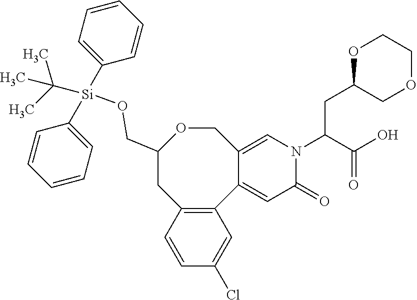 [SMILES: CC(C)(C)[Si](OCC1Cc2ccc(Cl)cc2-c2cc(=O)n(C(C[C@@H]3COCCO3)C(=O)O)cc2CO1)(c1ccccc1)c1ccccc1]

tert-Butyl 2-[7-({[tert-butyl(diphenyl)silyl]-oxy}methyl)-11-chloro-2-oxo-7,8-dihydro-2H-[3]benzoxocino[5,6-c]pyridin-3(5H)-yl]-3-[(2R)-1,4-dioxan-2-yl]propanoate (mixture of stereoisomers) (243 mg, 85% purity, 0.28 mmol, 1.0 eq.) was dissolved in a mixture of tetrahydrofuran and water (2:1, 10 ml), followed by the addition of lithium hydroxide (66.5 mg, 2.76 mmol, 10 eq.). The reaction mixture was stirred at 40° C. overnight, subsequently treated with aqueous hydrochloric acid (1 N) and extracted with ethyl acetate. The combined organic phases were washed with brine, dried over anhydrous sodium sulfate, filtered and concentrated under reduced pressure. The crude product was used in the subsequent reaction without further purification. Yield: 216 mg (63% purity, 71% of theory).

LC-MS (method 3): Rₜ=4.89 min; MS (ESIneg): m/z=686 [M−H]

### Example 6.14C

### 2-[11-Chloro-7-(hydroxymethyl)-2-oxo-7,8-dihydro-2H-[3]benzoxocino[5,6-c]pyridin-3(5H)-yl]-3-[(2R)-1,4-dioxan-2-yl]propanoic Acid (Mixture of Stereoisomers)

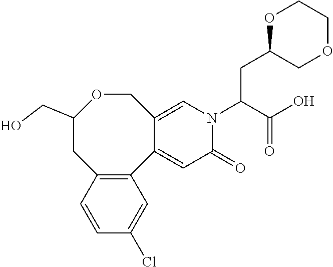 [SMILES: O=C(O)C(C[C@@H]1COCCO1)n1cc2c(cc1=O)-c1cc(Cl)ccc1CC(CO)OC2]

2-[7-({[tert-Butyl(diphenyl)silyl]oxy}methyl)-11-chloro-2-oxo-7,8-dihydro-2H-[3]benzoxocino[5,6-c]pyridin-3(5H)-yl]-3-[(2R)-1,4-dioxan-2-yl]propanoic acid (mixture of stereoisomers) (215 mg, 63% purity, 0.20 mmol, 1.0 eq.) was dissolved in tetrahydrofuran (8.0 ml), followed by the addition of tetra-N-butylammonium fluoride solution (394 μl, 1.0 M in tetrahydrofuran, 0.39 mmol, 2.0 eq.). The reaction mixture was stirred at RT overnight, concentrated under reduced pressure and purified by preparative HPLC (reversed phase, eluent: water with 0.05% formic acid/acetonitrile 10:90 to 95:5). Yield: 110 mg (92% purity, quantitative of theory).

LC-MS (method 3): Rₜ=1.80 min; MS (ESIpos): m/z=450 [M+H]⁺

### Example 6.15A

### Tert-Butyl (11-chloro-7-isopropyl-2-oxo-7,8-dihydro-2H-[3]benzoxocino[5,6-c]pyridin-3(5H)-yl)acetate (Racemate)

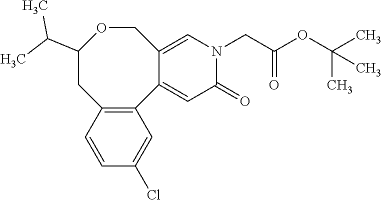 [SMILES: CC(C)C1Cc2ccc(Cl)cc2-c2cc(=O)n(CC(=O)OC(C)(C)C)cc2CO1]

11-Chloro-7-isopropyl-3,5,7,8-tetrahydro-2H-[3]benzoxocino[5,6-c]pyridin-2-one (racemate) (540 mg, 1.78 mmol, 1.0 eq.) and tert-butyl bromoacetate (416 mg, 2.13 mmol, 1.2 eq.) were dissolved in N,N-dimethylformamide (5 ml) and potassium carbonate (369 mg, 2.67 mmol, 1.5 eq.) was added. The mixture was heated to 100° C. for 1 h. Then the reaction mixture was concentrated under reduced pressure. The crude product was purified by column chromatography (silica gel, eluent: cyclohexane/ethyl acetate 100:0 to 7:3). Yield: 649 mg (87% of theory).

LC-MS (method 4): Rₜ=2.36 min; MS (ESIpos): m/z=418 [M+H]⁺

¹H-NMR (400 MHz, DMSO-d₆): δ [ppm]=7.91 (s, 1H), 7.54-7.47 (m, 1H), 7.37-7.27 (m, 2H), 6.39 (s, 1H), 4.63 (s, 2H), 4.46 (d, 1H), 3.53 (d, 1H), 3.21-3.13 (m, 1H), 2.81 (d, 1H), 2.27-2.18 (m, 1H), 1.92-1.78 (m, 1H), 1.48-1.39 (m, 9H), 0.97-0.85 (m, 6H).

### Example 6.16A

### Tert-Butyl 2-(11-chloro-7-isopropyl-2-oxo-7,8-dihydro-2H-[3]benzoxocino[5,6-c]pyridin-3(5H)-yl)-3-[(2S)-tetrahydro-2H-pyran-2-yl]propanoate (Mixture of Stereoisomers)

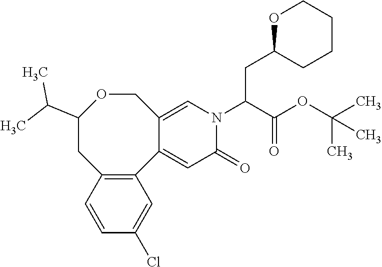 [SMILES: CC(C)C1Cc2ccc(Cl)cc2-c2cc(=O)n(C(C[C@@H]3CCCCO3)C(=O)OC(C)(C)C)cc2CO1]

tert-Butyl (11-chloro-7-isopropyl-2-oxo-7,8-dihydro-2H-[3]benzoxocino[5,6-c]pyridin-3(5H)-yl)acetate (racemate) (250 mg, 0.60 mmol, 1.0 eq.) under argon atmosphere was dissolved in tetrahydrofuran (6 ml). At −78° C., sodium bis(trimethylsilyl)amide solution (0.75 ml, 1.0 M in tetrahydrofuran, 0.75 mmol, 1.25 eq.) was added dropwise. After 20 min, a solution of (2S)-tetrahydro-2H-pyran-2-ylmethyl trifluoromethanesulfonate (single stereoisomer) (prepared according to WO 2017/005725, example 3.4C) (191 mg, 0.75 mmol, 1.25 eq.) in tetrahydrofuran (2 ml) was added slowly. The mixture was stirred at −78° C. for 15 min, at RT for 30 min and cooled to −78° C. before acetic acid (51 μl, 0.90 mmol, 1.5 eq.) was added. The reaction mixture was concentrated under reduced pressure and the residue was purified by column chromatography (silica gel, eluent: cyclohexane/ethyl acetate 100:0 to 5:1). Yield: 228 mg (74% of theory).

LC-MS (method 4): Rₜ=2.74/2.78 min; MS (ESIpos): m/z=516 [M+H]⁺

¹H-NMR (400 MHz, DMSO-d₆): δ [ppm]=7.92-7.78 (m, 1H), 7.54-7.44 (m, 1H), 7.43-7.28 (m, 2H), 6.42-6.28 (m, 1H), 5.32-5.04 (m, 1H), 4.58-4.36 (m, 1H), 3.92-3.72 (m, 1H), 3.62-3.43 (m, 1H), 3.29-2.72 (m, 4H, partially concealed), 2.44-2.00 (m, 3H), 1.92-1.82 (m, 1H), 1.77-1.57 (m, 1H), 1.53-1.05 (m, 14H), 0.96-0.83 (m, 6H).

### Example 6.16B

### 2-(11-Chloro-7-isopropyl-2-oxo-7,8-dihydro-2H-[3]benzoxocino[5,6-c]pyridin-3(5H)-yl)-3-[(2S)-tetrahydro-2H-pyran-2-yl]propanoic Acid (Mixture of Stereoisomers)

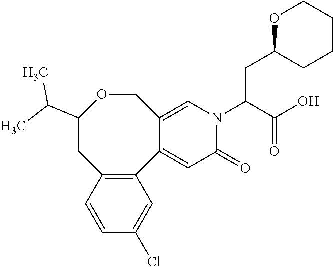 [SMILES: CC(C)C1Cc2ccc(Cl)cc2-c2cc(=O)n(C(C[C@@H]3CCCCO3)C(=O)O)cc2CO1]

tert-Butyl 2-(11-chloro-7-isopropyl-2-oxo-7,8-dihydro-2H-[3]benzoxocino[5,6-c]pyridin-3(5H)-yl)-3-[(2S)-tetrahydro-2H-pyran-2-yl]propanoate (mixture of stereoisomers) (220 mg, 0.43 mmol, 1.0 eq.) was dissolved in dichloromethane (5.0 ml) and trifluoroacetic acid (5.0 ml) was added. The reaction mixture was stirred for 45 min and concentrated under reduced pressure. For three times, the residue was dissolved in dichloromethane and concentrated under reduced pressure. The crude product was used in the subsequent reaction without further purification. Yield: 228 mg (86% purity).

LC-MS (method 5): Rₜ=1.44/1.46/1.48 min; MS (ESIpos): m/z=460 [M+H]⁺

### Example 6.17A

### Tert-Butyl 2-(11-chloro-7-isopropyl-2-oxo-7,8-dihydro-2H-[3]benzoxocino[5,6-c]pyridin-3(5H)-yl)-3-[(2R)-1,4-dioxan-2-yl]propanoate (Mixture of Stereoisomers)

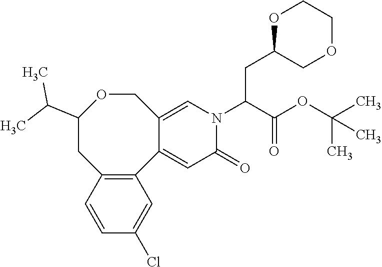 [SMILES: CC(C)C1Cc2ccc(Cl)cc2-c2cc(=O)n(C(C[C@@H]3COCCO3)C(=O)OC(C)(C)C)cc2CO1]

tert-Butyl (11-chloro-7-isopropyl-2-oxo-7,8-dihydro-2H-[3]benzoxocino[5,6-c]pyridin-3(5H)-yl)acetate (racemate) (250 mg, 0.60 mmol, 1.0 eq.) under argon atmosphere was dissolved in tetrahydrofuran (6 ml), 4 Å molecular sieves (0.5 g) was added and the mixture was stirred at RT for 10 min. The mixture was cooled to −78° C. and sodium bis(trimethylsilyl)amide solution (0.75 ml, 1.0 M in tetrahydrofuran, 0.75 mmol, 1.25 eq.) was added dropwise. After 20 min, a solution of (2S)-1,4-dioxan-2-ylmethyl trifluoromethanesulfonate (single stereoisomer) (prepared according to WO 2017/005725, example 3.5A) (197 mg, 0.75 mmol, 1.25 eq.) in tetrahydrofuran (2 ml) was added slowly. The mixture was stirred at −78° C. for 2 h and acetic acid (51 μl, 0.90 mmol, 1.5 eq.) was added. At this stage, the mixture was combined with another mixture resulting from a previous experiment [tert-butyl (11-chloro-7-isopropyl-2-oxo-7,8-dihydro-2H-[3]benzoxocino[5,6-c]pyridin-3(5H)-yl)acetate (racemate) (50 mg, 0.12 mmol, 1.0 eq.) in tetrahydrofuran (2 ml), sodium bis(trimethylsilyl)amide solution (0.15 ml, 1.0 M in tetrahydrofuran, 0.15 mmol, 1.25 eq.), (2S)-1,4-dioxan-2-ylmethyl trifluoromethanesulfonate (single stereoisomer) (39 mg, 0.15 mmol, 1.25 eq.) in tetrahydrofuran (1 ml)]. The combined reaction mixtures were concentrated under reduced pressure. The residue was purified by column chromatography (silica gel, eluent: cyclohexane/ethyl acetate 100:0 to 1:1). Yield: 351 mg (95% of theory for both experiments).

LC-MS (method 4): Rₜ=2.47/2.49 min; MS (ESIpos): m/z=518 [M+H]⁺

¹H-NMR (400 MHz, DMSO-d₆): δ [ppm]=7.95-7.79 (m, 1H), 7.54-7.46 (m, 1H), 7.41-7.23 (m, 2H), 6.41-6.31 (m, 1H), 5.24-5.03 (m, 1H), 4.57-4.44 (m, 1H), 3.80-3.34 (m, 6H), 3.27-3.11 (m, 3H), 2.80 (d, 1H), 2.35-2.15 (m, 2H), 2.12-1.99 (m, 1H), 1.92-1.76 (m, 1H), 1.46-1.31 (m, 9H), 0.98-0.84 (m, 6H).

### Example 6.17B

### 2-(11-Chloro-7-isopropyl-2-oxo-7,8-dihydro-2H-[3]benzoxocino[5,6-c]pyridin-3(5H)-yl)-3-[(2R)-1,4-dioxan-2-yl]propanoic Acid (Mixture of Stereoisomers)

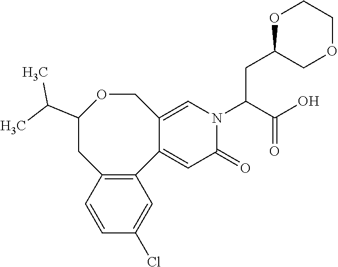 [SMILES: CC(C)C1Cc2ccc(Cl)cc2-c2cc(=O)n(C(C[C@@H]3COCCO3)C(=O)O)cc2CO1]

tert-Butyl 2-(11-chloro-7-isopropyl-2-oxo-7,8-dihydro-2H-[3]benzoxocino[5,6-c]pyridin-3(5H)-yl)-3-[(2R)-1,4-dioxan-2-yl]propanoate (mixture of stereoisomers) (350 mg, 0.68 mmol, 1.0 eq.) was dissolved in dichloromethane (8.0 ml) and trifluoroacetic acid (8.0 ml) was added. The reaction mixture was stirred for 45 min and concentrated under reduced pressure. For three times, the residue was dissolved in dichloromethane and concentrated under reduced pressure. The crude product was used in the subsequent reaction without further purification. Yield: 377 mg.

LC-MS (method 1): Rₜ=0.99 min; MS (ESIpos): m/z=462 [M+H]⁺

### Example 6.18A

### Tert-Butyl [11-chloro-2-oxo-7-(trifluoromethyl)-7,8-dihydro-2H-[3]benzoxocino[5,6-c]pyridin-3(5H)-yl]acetate (Racemate)

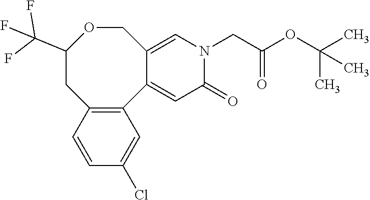 [SMILES: CC(C)(C)OC(=O)Cn1cc2c(cc1=O)-c1cc(Cl)ccc1CC(C(F)(F)F)OC2]

11-Chloro-7-(trifluoromethyl)-3,5,7,8-tetrahydro-2H-[3]benzoxocino[5,6-c]pyridin-2-one (racemate) (1.10 g, 84% purity, 2.80 mmol, 1.0 eq.) was dissolved in N,N-dimethylformamide (20 ml). tert-Butyl bromoacetate (0.82 g, 4.20 mmol, 1.5 eq.) and potassium carbonate (0.78 g, 5.61 mmol, 2.0 eq.) were added. After stirring for 1 h at 100° C., the reaction mixture was cooled to RT, diluted with ethyl acetate and washed with a saturated aqueous sodium carbonate solution, water and brine. The organic layer was dried over anhydrous sodium sulfate, filtered and concentrated under reduced pressure. The residue was purified by column chromatography (silica gel, eluent: petroleum ether/ethyl acetate 50:1 to 10:1). Yield: 0.66 g (53% of theory).

¹H-NMR (300 MHz, DMSO-d₆): δ [ppm]=7.98 (s, 1H), 7.59-7.55 (m, 1H), 7.43-7.40 (m, 2H), 6.43 (s, 1H), 4.64 (s, 2H), 4.62-4.57 (m, 1H), 4.21-4.15 (m, 1H), 3.77-3.72 (m, 1H), 3.16-3.09 (m, 1H), 2.49-2.40 (m, 1H, partially concealed), 1.44 (s, 9H).

### Example 6.18B

### Tert-Butyl [(7S)-11-chloro-2-oxo-7-(trifluoromethyl)-7,8-dihydro-2H-[3]benzoxocino[5,6-c]pyridin-3(5H)-yl]acetate (Single Stereoisomer)

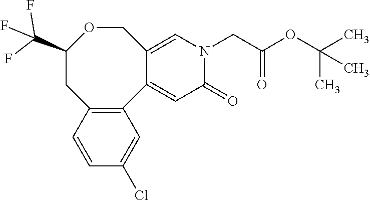 [SMILES: CC(C)(C)OC(=O)Cn1cc2c(cc1=O)-c1cc(Cl)ccc1C[C@@H](C(F)(F)F)OC2]

Enantiomer separation of 506 mg of tert-butyl [11-chloro-2-oxo-7-(trifluoromethyl)-7,8-dihydro-2H-[3]benzoxocino[5,6-c]pyridin-3(5H)-yl]acetate (racemate), Example 6.18A gave
single stereoisomer 1 (chiral SFC: Rₜ=1.52 min, >99% ee): 33 mg,
single stereoisomer 2 (the title compound Example 6.18B) (chiral SFC: Rₜ=4.01 min, >99% ee): 339 mg.

Separation method: SFC: column: Chiralcel IC CSP 20 μm, 450 mm×50 mm; eluent: 70% carbon dioxide/30% methanol; temperature: 30° C.; flow rate: 400 ml/min; UV detection: 210 nm.

Analysis method: SFC: column: Chiralcel IC, 100 mm×4.6 mm; eluent: 80% carbon dioxide/20% methanol; temperature: 40° C.; flow rate: 3.0 ml/min; UV detection: 210 nm.

LC-MS (method 4): Rₜ=2.14 min; MS (ESIpos): m/z=444 [M+H]⁺

### Example 6.19A

### Tert-Butyl 2-[11-chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoate (Mixture of Stereoisomers)

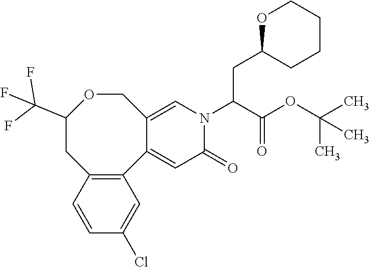 [SMILES: CC(C)(C)OC(=O)C(C[C@@H]1CCCCO1)n1cc2c(cc1=O)-c1cc(Cl)ccc1CC(C(F)(F)F)OC2]

tert-Butyl [11-chloro-2-oxo-7-(trifluoromethyl)-7,8-dihydro-2H-[3]benzoxocino[5,6-c]pyridin-3(5H)-yl]acetate (racemate) (430 mg, 0.94 mmol, 1.0 eq.) under argon atmosphere was dissolved in tetrahydrofuran (14 ml), cooled to −78° C. and sodium bis(trimethylsilyl)amide solution (1.2 ml, 1.0 M in tetrahydrofuran, 1.2 mmol, 1.25 eq.) was added dropwise. After 20 min, a solution of (2S)-tetrahydro-2H-pyran-2-ylmethyl trifluoromethanesulfonate (single stereoisomer) (prepared according to WO 2017/005725, example 3.4C) (306 mg, 1.17 mmol, 1.25 eq.) in tetrahydrofuran (7 ml) was added slowly. The mixture was stirred at −78° C. for 15 min and at RT for 30 min. After cooling to −78° C., acetic acid (80 μl, 1.4 mmol, 1.5 eq.) was added and the reaction mixture was concentrated under reduced pressure. The residue was purified by column chromatography (silica gel, eluent: cyclohexane/ethyl acetate 100:0 to 1:1). Yield: 381 mg (74% of theory).

LC-MS (method 4): Rₜ=2.47/2.49 min; MS (ESIpos): m/z=542 [M+H]⁺

### Example 6.19B

### 2-[11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoic Acid (Mixture of Stereoisomers)

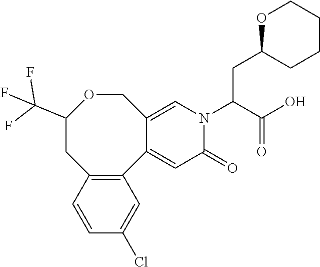 [SMILES: O=C(O)C(C[C@@H]1CCCCO1)n1cc2c(cc1=O)-c1cc(Cl)ccc1CC(C(F)(F)F)OC2]

tert-Butyl 2-[11-chlor-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoate (mixture of stereoisomers) (380 mg, 0.70 mmol, 1.0 eq.) was dissolved in tetrahydrofuran (14 ml) and aqueous lithium hydroxide solution (7.0 ml, 1.0 M, 7.0 mmol, 10 eq.) was added. The reaction mixture was stirred at RT overnight. Additional amounts of aqueous lithium hydroxide solution (7.0 ml, 1.0 M, 7.0 mmol, 10 eq.) were added and the reaction mixture was stirred for 23 h at 40° C. After concentration under reduced pressure, the aqueous phase was adjusted to pH 2 using aqueous hydrochloric acid (1 N). The aqueous phase was extracted with ethyl acetate and the combined organic phases were dried over anhydrous sodium sulfate, filtered and concentrated under reduced pressure. The crude product was used in the subsequent reaction without further purification. Yield: 301 mg (84% of theory).

LC-MS (method 4): Rₜ=1.95/1.98 min; MS (ESIpos): m/z=486 [M+H]⁺

### Example 6.20A

### Tert-Butyl 2-[11-chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoate (Mixture of Stereoisomers)

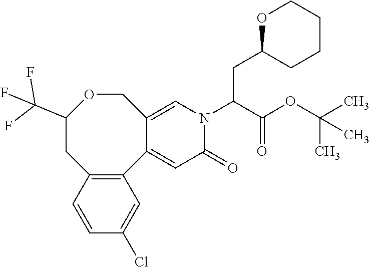 [SMILES: CC(C)(C)OC(=O)C(C[C@@H]1CCCCO1)n1cc2c(cc1=O)-c1cc(Cl)ccc1CC(C(F)(F)F)OC2]

Tert-Butyl [11-chloro-2-oxo-7-(trifluoromethyl)-7,8-dihydro-2H-[3]benzoxocino[5,6-c]pyridin-3(5H)-yl]acetate (racemate) (400 mg, 0.89 mmol, 1.0 eq.) under argon atmosphere was dissolved in tetrahydrofuran (14 ml), cooled to −78° C. and sodium bis(trimethylsilyl)amide solution (1.1 ml, 1.0 M in tetrahydrofuran, 1.12 mmol, 1.25 eq.) was added dropwise. After 20 min, a solution of (2S)-1,4-dioxan-2-ylmethyl trifluoromethanesulfonate (single stereoisomer) (prepared according to WO 2017/005725, example 3.5A) (294 mg, 1.12 mmol, 1.25 eq.) in tetrahydrofuran (7 ml) was added slowly. The mixture was stirred at −78° C. for 30 min, followed by the addition of acetic acid (77 μl, 1.3 mmol, 1.5 eq.) and concentration under reduced pressure. The residue was purified by column chromatography (silica gel, eluent: cyclohexane/ethyl acetate 100:0 to 3:2). Yield: 446 mg (85% of theory).

LC-MS (method 4): Rₜ=2.21/2.22 min; MS (ESIpos): m/z=544 [M+H]⁺

### Example 6.20B

### 2-[11-Chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoic Acid (Mixture of Stereoisomers)

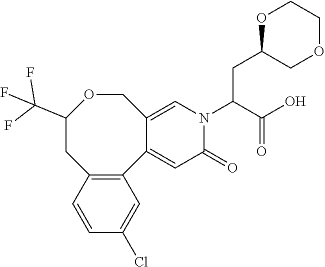 [SMILES: O=C(O)C(C[C@@H]1COCCO1)n1cc2c(cc1=O)-c1cc(Cl)ccc1CC(C(F)(F)F)OC2]

tert-Butyl 2-[1-chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoate (mixture of stereoisomers) (445 mg, 0.82 mmol, 1.0 eq.) was dissolved in a mixture of tetrahydrofuran and water (2:1, 24 ml) and lithium hydroxide (392 mg, 16.4 mmol, 20 eq.) was added. The reaction mixture was stirred at 40° C. overnight. The pH of the reaction mixture was adjusted to 2 using aqueous hydrochloric acid (1 N). Ethyl acetate was added and the phases were separated. The aqueous phase was extracted with ethyl acetate and the combined organic phases were washed with brine, dried over anhydrous sodium sulfate, filtered and concentrated under reduced pressure. The crude product was used in the subsequent reaction without further purification. Yield: 392 mg (97% of theory).

LC-MS (method 4): Rₜ=1.68 min; MS (ESIpos): m/z=488 [M+H]⁺

### Example 6.21A

### Tert-Butyl N-(diphenylmethylene)-O-methylhomoserinate (Racemate)

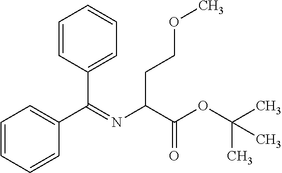 [SMILES: COCCC(N=C(c1ccccc1)c1ccccc1)C(=O)OC(C)(C)C]

tert-Butyl N-(diphenylmethylene)glycinate (4.43 g, 15.0 mmol, 1.0 eq.) under argon atmosphere was dissolved in tetrahydrofuran (150 ml), cooled to −78° C., subsequently followed by the dropwise addition of lithium bis(trimethylsilyl)amide solution (19.5 ml, 1.0 M in tetrahydrofuran, 19.5 mmol, 1.3 eq.). The reaction mixture was stirred for 15 min and 2-methoxyethyl trifluoromethanesulfonate (5.85 g, 80% purity, 22.5 mmol, 1.5 eq.) was added dropwise. The reaction mixture was stirred for 15 min at −78° C., allowed to warm to RT and stirred for 30 min. Subsequently, water and ethyl acetate were added, the aqueous phase was extracted with ethyl acetate. The combined organic phases were washed with brine, dried over anhydrous sodium sulfate, filtered and concentrated under reduced pressure. The residue was purified by column chromatography (silica gel, eluent: cyclohexane/ethyl acetate mixture). Yield: 4.61 g (87% of theory).

LC-MS (method 1): Rₜ=1.17 min; MS (ESIpos): m/z=354 [M+H]⁺

¹H-NMR (400 MHz, DMSO-d₆): δ [ppm]=7.56-7.35 (m, 8H), 7.18-7.11 (m, 2H), 3.92 (dd, 1H), 3.38-3.32 (m, 1H), 3.26-3.17 (m, 1H), 3.12 (s, 3H), 2.10-1.91 (m, 2H), 1.37 (s, 9H).

### Example 6.21B

### Tert-Butyl O-methylhomoserinate (Racemate)

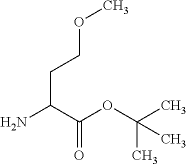

An aqueous citric acid solution (150 ml, 1.0 M) was added to a solution of tert-butyl N-(diphenylmethylene)-O-methylhomoserinate (racemate) (4.61 g, 13.0 mmol, 1.0 eq.) in tetrahydrofuran (150 ml) and the mixture was stirred at RT for 2 h. Subsequently, the volatiles were removed under reduced pressure. The resulting aqueous solution was carefully neutralized with solid sodium bicarbonate and extracted with dichloromethane. The combined organic phases were dried over anhydrous sodium sulfate, filtered and concentrated on under reduced pressure (>150 mbar at <25° C. water bath temperature). The residue was purified by column chromatography (silica gel, eluent: dichloromethane/methanol mixture). Yield: 2.58 g (quantitative of theory).

GC-MS (method 2): Rₜ=3.26 min; MS: m/z=189 [M]⁺

¹H-NMR (400 MHz, DMSO-d₆): δ [ppm]=3.48-3.39 (m, 1H), 3.39-3.33 (m, 1H), 3.25-3.14 (m, 1H), 3.21 (s, 3H), 1.84-1.73 (m, 1H), 1.66 (br s, 1H), 1.60-1.50 (m, 1H), 1.40 (s, 9H).

### Example 6.21C

### Ethyl 1-(1-tert-butoxy-4-methoxy-1-oxobutan-2-yl)-4-hydroxy-6-oxo-1,6-dihydropyridine-3-carboxylate (Racemate)

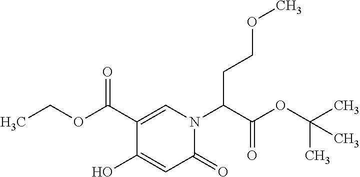 [SMILES: CCOC(=O)c1cn(C(CCOC)C(=O)OC(C)(C)C)c(=O)cc1O]

Diethyl 3-oxopentanedicarboxylate (2.35 ml, 90% purity, 11.7 mmol, 1.0 eq.) and diethoxymethyl acetate (2.67 ml 16.3 mmol, 1.4 eq.) were stirred for 2.5 h at 100° C. After cooling to RT, the mixture was coevaporated with toluene for three times. The residue dissolved in ethanol (70 ml), cooled to 0° C. and a solution of tert-butyl O-methylhomoserinate (racemate) (2.32 g, 12.2 mmol, 1.05 eq.) in ethanol (20 ml) was added. The mixture was stirred at RT for 1 h. Subsequently, a solution of sodium methoxide (4.36 ml, 21% in ethanol, 11.7 mmol, 1.0 eq., previously dried over activated 3 Å molecular sieves) was added. The mixture was stirred for 1 h at RT before saturated aqueous ammonium chloride solution was added. The precipitate was filtered, washed with ethyl acetate and discarded. After phase separation of the combined filtrates, the aqueous phase was extracted with ethyl acetate. The combined organic phases were washed with brine, dried over anhydrous sodium sulfate, filtered and concentrated under reduced pressure. The residue was purified by column chromatography (silica gel, eluent: cyclohexane/ethyl acetate mixture). Yield: 2.37 g (56% of theory).

LC-MS (method 1): Rₜ=0.98 min; MS (ESIneg): m/z=354 [M−H]⁻

¹H-NMR (400 MHz, DMSO-d₆): δ [ppm]=10.85 (s, 1H), 8.31 (s, 1H), 5.69 (s, 1H), 5.11 (dd, 1H), 4.36-4.23 (m, 2H), 3.38-3.32 (m, 1H), 3.15 (s, 3H), 3.14-3.07 (m, 1H), 2.31-2.14 (m, 2H), 1.37 (s, 9H), 1.29 (t, 3H).

### Example 6.21D

### Ethyl 1-(1-tert-butoxy-4-methoxy-1-oxobutan-2-yl)-6-oxo-4-{[(trifluoromethyl)sulfonyl]oxy}-1,6-dihydropyridine-3-carboxylate (Racemate)

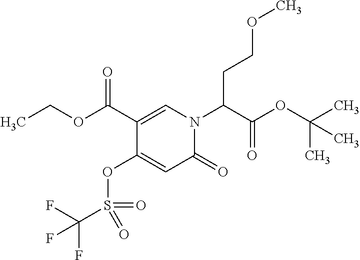 [SMILES: CCOC(=O)c1cn(C(CCOC)C(=O)OC(C)(C)C)c(=O)cc1OS(=O)(=O)C(F)(F)F]

Triethylamine (3.45 ml, 24.8 mmol, 2.0 eq.) was added to a solution of ethyl 1-(1-tert-butoxy-4-methoxy-1-oxobutan-2-yl)-4-hydroxy-6-oxo-1,6-dihydropyridine-3-carboxylate (racemate) (4.49 g, 12.4 mmol, 1.0 eq.) in dichloromethane (50 ml) at −78° C. under argon atmosphere. Subsequently, N-(4-tert-butylphenyl)-1,1,1-trifluoro-N-[(trifluoromethyl)sulfonyl]-methanesulfonamide (8.19 g, 19.8 mmol, 1.6 eq.) was added portionwise. The reaction mixture was allowed to warm to RT, stirred for 6 h at RT and concentrated under reduced pressure. The residue was purified by column chromatography (silica gel, eluent: cyclohexane/ethyl acetate mixture). Yield: 4.85 g (76% of theory).

LC-MS (method 1): Rₜ=1.23 min; MS (ESIpos): m/z=488 [M+H]⁺

¹H-NMR (400 MHz, DMSO-d₆): δ [ppm]=8.65 (s, 1H), 6.66 (s, 1H), 5.27 (t, 1H), 4.38-4.25 (m, 2H), 3.44-3.36 (m, 1H), 3.23-3.14 (m, 1H), 3.11 (s, 3H), 2.35-2.24 (m, 2H), 1.36 (s, 9H), 1.30 (t, 3H).

### Example 6.21E

### Ethyl 1-(1-tert-butoxy-4-methoxy-1-oxobutan-2-yl)-6-oxo-4-(4,4,5,5-tetramethyl-1,3,2-dioxaborolan-2-yl)-1,6-dihydropyridine-3-carboxylate (Racemate)

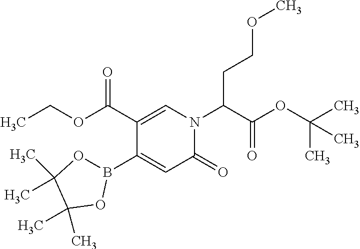 [SMILES: CCOC(=O)c1cn(C(CCOC)C(=O)OC(C)(C)C)c(=O)cc1B1OC(C)(C)C(C)(C)O1]

Ethyl 1-(1-ter-butoxy-4-methoxy-1-oxobutan-2-yl)-6-oxo-4-{[(trifluoromethyl)sulfonyl]oxy}-1,6-dihydropyridine-3-carboxylate (racemate) (1.00 g, 85% purity, 1.74 mmol), bis(pinacolato)diboron (487 mg, 1.92 mmol, 1.1 eq.) and potassium acetate (513 mg, 5.23 mmol, 3.0 eq.) were dissolved in 1,4-dioxane (18 ml) under argon atmosphere. [1,1′-Bis(diphenylphosphino)ferrocene]dichloropalladium(II) dichloromethane complex (42.7 mg, 0.05 mmol, 0.03 eq.) was added and the reaction mixture was stirred at 80° C. for 7 h. The reaction mixture was cooled, filtered through a pad of Celite® and washed with 1,4-dioxane. The filtrate was concentrated under reduced pressure, dried at 40° C. under high vacuum and used in the subsequent reaction without further purification. Yield: 980 mg (80% purity, 97% of theory).

¹H-NMR (400 MHz, DMSO-d₆): δ [ppm]=8.31 (s, 1H), 6.39 (s, 1H), 5.12 (dd, 1H), 4.34-4.20 (m, 2H), 3.38-3.33 (m, 1H), 3.16-3.04 (m, 4H), 2.34-2.17 (m, 2H), 1.36 (s, 9H), 1.33-1.25 (m, 15H).

### Example 6.21F

### Ethyl 1-(1-tert-butoxy-4-methoxy-1-oxobutan-2-yl)-4-[5-chloro-2-(3,3,3-trifluoro-2-hydroxy-propyl)phenyl]-6-oxo-1,6-dihydropyridine-3-carboxylate (Mixture of Stereoisomers)

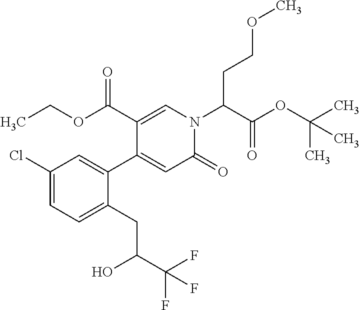 [SMILES: CCOC(=O)c1cn(C(CCOC)C(=O)OC(C)(C)C)c(=O)cc1-c1cc(Cl)ccc1CC(O)C(F)(F)F]

Ethyl 1-(1-ter-butoxy-4-methoxy-1-oxobutan-2-yl)-6-oxo-4-{[(trifluoromethyl)sulfonyl]oxy}-1,6-dihydropyridine-3-carboxylate (racemate) (1.00 g, 91% purity, 1.87 mmol, 1.0 eq.) was dissolved in 1,4-dioxane (19 ml), bis(pinacolato)diboron (569 mg, 2.24 mmol, 1.2 eq.) and potassium acetate (550 mg, 5.60 mmol, 3.0 eq.) were added and argon was passed through the resulting suspension for 5 min. [1,1-Bis(diphenylphosphino)ferrocene]dichloropalladium(II) dichloromethane complex (100 mg, 0.12 mmol, 0.066 eq.) was added and the reaction mixture was heated to 100° C. for 45 min. 3-(2-Bromo-4-chlorophenyl)-1,1,1-trifluoropropan-2-ol (racemate) (567 mg, 1.87 mmol, 1.0 eq.), aqueous sodium carbonate solution (1.9 ml, 2.0 M, 3.74 mmol, 2.0 eq.) and [1,1′-bis(diphenylphosphino)ferrocene]-dichloropalladium(II) dichloromethane complex (52 mg, 0.063 mmol, 0.033 eq.) were added and the reaction mixture was stirred at 100° C. overnight. The reaction mixture was cooled to RT and filtered through a pad of Celite®. The filtrate was concentrated under reduced pressure and the crude product was purified by column chromatography (silica gel, eluent: cyclohexane/ethyl acetate 100:0 to 1:9). Yield: 612 mg (58% of theory).

LC-MS (method 3): Rₜ=3.83 min; MS (ESIpos): m/z=562 [M+H]⁺

### Example 6.21G

### Tert-Butyl 2-{4-[5-chloro-2-(3,3,3-trifluoro-2-hydroxypropyl)phenyl]-5-(hydroxymethyl)-2-oxopyridin-1(2H)-yl}-4-methoxybutanoate (Mixture of Stereoisomers)

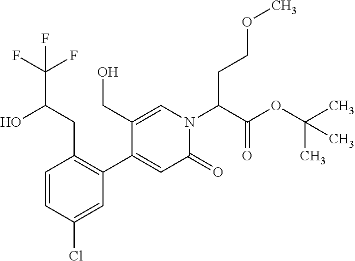 [SMILES: COCCC(C(=O)OC(C)(C)C)n1cc(CO)c(-c2cc(Cl)ccc2CC(O)C(F)(F)F)cc1=O]

Ethyl 1-(1-tert-butoxy-4-methoxy-1-oxobutan-2-yl)-4-[5-chloro-2-(3,3,3-trifluoro-2-hydroxypropyl)-phenyl]-6-oxo-1,6-dihydropyridine-3-carboxylate (mixture of stereoisomers) (4.00 g, 88% purity, 6.26 mmol, 1.0 eq.) was dissolved in dichloromethane (80 ml) and cooled to −78° C. Diisobutylaluminum hydride solution (20.9 ml, 1.2 M in toluene, 25.1 mmol, 4.0 eq.) was added dropwise and the reaction mixture was stirred at −78° C. for 90 min. Sodium sulfate decahydrate (20 g), a mixture of anhydrous sodium sulfate (3.55 g) and water (4.5 ml) were added and the mixture was warmed to RT over 20 min. The mixture was diluted with dichloromethane and Celite® was added. After stirring for 5 min, the suspension was filtered and the solids were washed with dichloromethane. The combined filtrates were concentrated under reduced pressure and dried under high vacuum overnight. The crude reaction mixture was dissolved in ethanol (96 ml) and cooled to 0° C. Sodium borohydride (474 mg, 12.5 mmol, 2.0 eq.) was added and the mixture was stirred for 1.5 h at 0-4° C. After addition of saturated aqueous ammonium chloride solution, stirring was continued for 15 min. The mixture was extracted with ethyl acetate and the combined organic phases were washed with brine, filtered and dried over anhydrous magnesium sulfate und concentrated under reduced pressure. The crude product was used in the subsequent reaction without further purification. Yield: 2.36 g (72% purity, 52% of theory).

LC-MS (method 1): Rₜ=0.94 min; MS (ESIpos): m/z=520 [M+H]⁺

### Example 6.21H

### Tert-Butyl 2-[11-chloro-2-oxo-7-(trifluoromethyl)-7,8-dihydro-2H-[3]benzoxocino[5,6-c]pyridin-3(5H)-yl]-4-methoxybutanoate (Mixture of Stereoisomers)

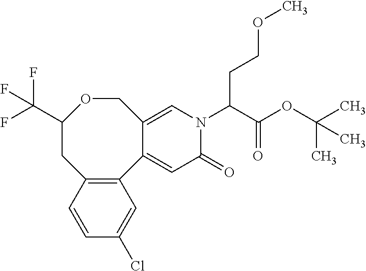 [SMILES: COCCC(C(=O)OC(C)(C)C)n1cc2c(cc1=O)-c1cc(Cl)ccc1CC(C(F)(F)F)OC2]

tert-Butyl 2-{4-[5-chloro-2-(3,3,3-trifluoro-2-hydroxypropyl)phenyl]-5-(hydroxymethyl)-2-oxopyridin-1(2H)-yl}-4-methoxybutanoate (mixture of stereoisomers) (2.36 g, 72% purity, 3.27 mmol, 1.0 eq.) was dissolved in dichloromethane (33 ml), triethylamine (1.23 ml, 8.82 mmol, 2.7 eq.) was added and the solution was cooled to 0° C. Methanesulfonyl chloride (0.35 ml, 4.58 mmol, 1.4 eq.) was added and the reaction mixture was heated to reflux overnight. After cooling to RT, the mixture was treated with dichloromethane and phosphate buffer (pH 7). The phases were separated and the organic phase was washed with phosphate buffer (pH 7). The combined aqueous phases were extracted with dichloromethane, dried over anhydrous magnesium sulfate, filtered and concentrated under reduced pressure. The crude product was purified by column chromatography (silica gel, cyclohexane/ethyl acetate 100:0 to 0:100). Yield: 277 mg (91% purity, 15% of theory).

LC-MS (method 1): Rₜ=1.20 min; MS (ESIpos): m/z=502 [M+H]⁺

### Example 6.21I

### 2-[11-Chloro-2-oxo-7-(trifluoromethyl)-7,8-dihydro-2H-[3]benzoxocino[5,6-c]pyridin-3(5H)-yl]-4-methoxybutanoic Acid (Mixture of Stereoisomers)

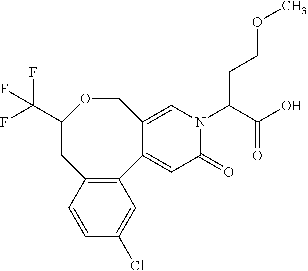 [SMILES: COCCC(C(=O)O)n1cc2c(cc1=O)-c1cc(Cl)ccc1CC(C(F)(F)F)OC2]

tert-Butyl 2-[11-chloro-2-oxo-7-(trifluoromethyl)-7,8-dihydro-2H-[3]benzoxocino[5,6-c]pyridin-3(5H)-yl]-4-methoxybutanoate (mixture of stereoisomers) (277 mg, 91% purity, 0.501 mmol, 1.0 eq.) was dissolved in tetrahydrofuran (2.0 ml) and an aqueous lithium hydroxide solution (5.0 ml, 1.0 M, 5.0 mmol, 10 eq.) was added. The reaction mixture was stirred at RT for 4 h. Additional amounts of aqueous lithium hydroxide solution (5.0 ml, 1.0 M, 5.0 mmol, 10 eq.) were added and the reaction mixture was stirred overnight. Further amounts of aqueous lithium hydroxide solution (5.0 ml, 1.0 M, 5.0 mmol, 10.0 eq.) were added and the reaction mixture was stirred for 72 h. After adding aqueous lithium hydroxide solution (5.0 ml, 1.0 M, 5.0 mmol, 10.0 eq.), stirring was continued at RT for 6 h. Additional amounts of lithium hydroxide (50 mg, 2.1 mmol, 0.4 eq.) were added and stirring was continued at 40° C. overnight. The reaction mixture was concentrated under reduced pressure and the pH was adjusted to 2 by addition of aqueous hydrochloric acid (1 N). The aqueous phase was extracted with ethyl acetate. The combined organic phases were dried over anhydrous magnesium sulfate, filtered and concentrated under reduced pressure. The crude product was used in the subsequent reaction without further purification. Yield: 213 mg (95% of theory).

LC-MS (method 4): Rₜ=1.77/1.79 min; MS (ESIpos): m/z=446 [M+H]⁺

### Example 6.22A

### Tert-Butyl (2ξ)-2-[(7S)-11-chloro-2-oxo-7-(trifluoromethyl)-7,8-dihydro-2H-[3]benzoxocino[5,6-c]pyridin-3(5H)-yl]-2,3,5-trideoxy-4-O-methyl-L-glycero-pentonate (Mixture of Two Diastereomers)

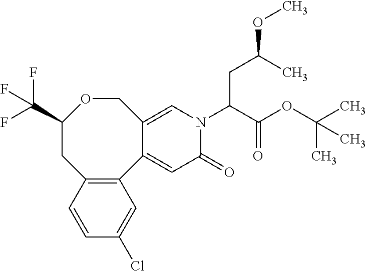 [SMILES: CO[C@@H](C)CC(C(=O)OC(C)(C)C)n1cc2c(cc1=O)-c1cc(Cl)ccc1C[C@@H](C(F)(F)F)OC2]

A solution of lithium bis(trimethylsilyl)amide (0.32 ml, 1.0 M in tetrahydrofuran, 0.32 mmol, 1.2 eq.) was added under argon atmosphere at −78° C. to a solution of tert-butyl [(7S)-11-chloro-2-oxo-7-(trifluoromethyl)-7,8-dihydro-2H-[3]benzoxocino[5,6-c]pyridin-3(5H)-yl]acetate (single stereoisomer) (120 mg, 0.27 mmol) in tetrahydrofuran (4 ml). After stirring at −78° C. for 15 min, (2S)-2-methoxypropyl trifluoromethanesulfonate (single stereoisomer) (120 mg, 90% purity, 0.49 mmol, 1.8 eq.) (prepared according to WO 2014/154794, example 28.1A) was added dropwise. The reaction mixture was stirred at −78° C. for 15 min, allowed to slowly warm to 0° C., stirred at 0° C. for 30 min, allowed to warm to RT and stirred at RT for 1.5 h. The reaction mixture was quenched with saturated aqueous solution of ammonium chloride. After addition of ethyl acetate and phase separation, the aqueous phase was extracted with ethyl acetate. The combined organic phases were washed with brine, dried over anhydrous sodium sulfate, filtered and concentrated under reduced pressure. The residue was purified by preparative HPLC (reversed phase, eluent: acetonitrile/water gradient). Yield: 60 mg (43% of theory).

LC-MS (method 1): Rₜ=1.25 min; MS (ESIpos): m/z=516 [M+H]⁺

### Example 6.22B

### (2ξ)-2-[(7S)-11-Chloro-2-oxo-7-(trifluoromethyl)-7,8-dihydro-2H-[3]benzoxocino[5,6-c]pyridin-3(5H)-yl]-2,3,5-trideoxy-4-O-methyl-L-glycero-pentonic Acid (Mixture of Two Diastereomers)

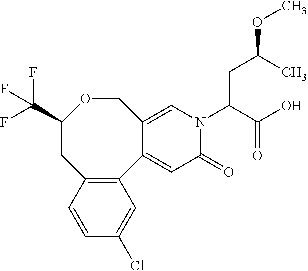 [SMILES: CO[C@@H](C)CC(C(=O)O)n1cc2c(cc1=O)-c1cc(Cl)ccc1C[C@@H](C(F)(F)F)OC2]

Lithium hydroxide (11 mg, 0.47 mmol, 2.0 eq.) was added at RT to a solution of tert-butyl (2ξ)-2-[(7S)-11-chloro-2-oxo-7-(trifluoromethyl)-7,8-dihydro-2H-[3]benzoxocino[5,6-c]pyridin-3(5H)-yl]-2,3,5-trideoxy-4-O-methyl-L-glycero-pentonate (mixture of two diastereomers) (120 mg, 0.23 mmol) in a mixture of tetrahydrofuran and water (3:1, 3 ml). The reaction mixture was stirred at RT for 2 days and acidified with aqueous hydrochloric acid (1 N). After addition of ethyl acetate and phase separation, the aqueous phase was extracted with ethyl acetate. The combined organic phases were washed with brine, dried over anhydrous sodium sulfate, filtered and concentrated under reduced pressure. Yield: 94 mg (88% of theory).

LC-MS (method 4): Rₜ=1.84/1.85 min; MS (ESIpos): m/z=460 [M+H]⁺

### Example 7.1A

### Tert-Butyl [1,2,4]triazolo[1,5-a]pyridin-7-ylcarbamate

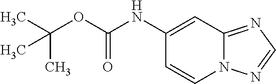 [SMILES: CC(C)(C)OC(=O)Nc1ccn2ncnc2c1]

A mixture of 7-bromo[1,2,4]triazolo[1,5-a]pyridine (18.5 g, 93.4 mmol, 1.0 eq.), tert-butyl carbamate (16.42 g, 140.1 mmol, 1.5 eq.), palladium(II) acetate (2.10 g, 9.3 mmol, 0.1 eq.), 2-dicyclohexyl-phosphino-2′,4′,6′-triisopropyl-1,1′-biphenyl (13.4 g, 28.0 mmol, 0.3 eq.) and caesium carbonate (60.9 g, 186.8 mmol, 2.0 eq.) in 1,4-dioxane (930 ml) was purged with nitrogen gas for 10 min. The reaction mixture was heated to 100° C. for 15 h, cooled to RT and filtered through Celite®. The filtrate was concentrated. The residue was purified by column chromatography (silica gel, eluent: petroleum ether/ethyl acetate 1:3). Yield: 17.0 g (77% of theory).

¹H-NMR (400 MHz, DMSO-d₆): δ [ppm]=9.99 (s, 1H), 8.77 (d, 1H), 8.30 (s, 1H), 7.87 (s, 1H), 7.19-7.12 (m, 1H), 1.49 (s, 9H).

### Example 7.1B

### [1,2,4]Triazolo[1,5-a]pyridin-7-amine Hydrochloride

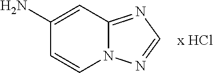

Hydrogen chloride solution (363 ml, 4 M in 1,4-dioxane) was added to a stirred solution of tert-butyl [1,2,4]triazolo[1,5-a]pyridin-7-ylcarbamate (17.00 g, 72.6 mmol) in dichloromethane (11). The reaction mixture was stirred at RT for 15 h and diluted with diethyl ether. The precipitate was collected by filtration, washed with diethyl ether and dried under reduced pressure. Yield: 10.45 g (84% of theory).

¹H-NMR (400 MHz, DMSO-d₆): δ [ppm]=8.97-8.90 (m, 1H), 8.67 (d, 1H), 7.40 (br s, 2H), 6.90-6.81 (m, 1H), 6.73-6.68 (m, 1H).

### WORKING EXAMPLES

### 4-({2-(11-Chloro-7-methyl-2-oxo-7,8-dihydro-2H-[3]benzoxocino[5,6-c]pyridin-3(5H)-yl)-3-[(2S)-tetrahydro-2H-pyran-2-yl]propanoyl}amino)-2-fluorobenzamide (Mixture of Stereoisomers)

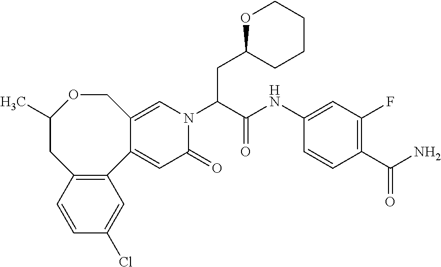 [SMILES: CC1Cc2ccc(Cl)cc2-c2cc(=O)n(C(C[C@@H]3CCCCO3)C(=O)Nc3ccc(C(N)=O)c(F)c3)cc2CO1]

2-(11-Chloro-7-methyl-2-oxo-7,8-dihydro-2H-[3]benzoxocino[5,6-c]pyridin-3(5H)-yl)-3-[(2S)-tetrahydro-2H-pyran-2-yl]propanoic acid (mixture of stereoisomers) (47 mg, 80% purity, 87 μmol) and 4-amino-2-fluorobenzamide (22 mg, 141 μmol, 1.63 eq.) were reacted according to General Method 11 including the following variations of the procedure: After stirring for 1 h at 50° C. and 1 h at 60° C., additional T3P (40 μl, 50% solution in ethyl acetate, 169 μmol) was added followed by stirring for 1 h at 60° C. The reaction mixture was diluted with acetonitrile (1 ml) and water (0.5 ml) and directly purified by preparative HPLC (eluent: water with 0.01% trifluoroacetic acid/acetonitrile 90:10 to 5:95). Three fractions were obtained:
mixture of stereoisomers 1 (the title compound Example 1) (LC-MS (method 4): Rₜ=1.97 min): 23 mg (90% purity, 41% of theory),
mixture of stereoisomers 2 (LC-MS (method 4): Rₜ=1.92 min): 8.5 mg (17% of theory),
mixture of stereoisomers 3 (LC-MS (method 4): Rₜ=1.92/1.96 min): 8.9 mg (18% of theory).

LC-MS (method 4): Rₜ=1.97 min; MS (ESIpos): m/z=568 [M+H]⁺

¹H-NMR (400 MHz, DMSO-d₆): δ [ppm]=10.80/10.78 (2s, 1H), 7.99/7.92 (2d, 1H), 7.72-7.62 (m, 2H), 7.57-7.37 (m, 5H), 7.37-7.32 (m, 1H), 6.39/6.39 (2s, 1H), 5.80-5.67 (m, 1H), 4.62-4.52 (m, 1H), 3.88-3.75 (m, 1H), 3.62-3.50 (m, 2H), 3.29-3.12 (m, 2H), 2.90-2.77 (m, 1H), 2.40-2.24 (m, 2H), 2.24-2.02 (m, 1H), 1.82-1.66 (m, 1H), 1.66-1.52 (m, 1H), 1.50-1.33 (m, 3H), 1.31-1.16 (m, 4H). Additional signals of a minor rotamer were also detected.

### Example 2

### 4-({(2S)-2-[(7R)-11-Chloro-7-methyl-2-oxo-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (Single Stereoisomer)

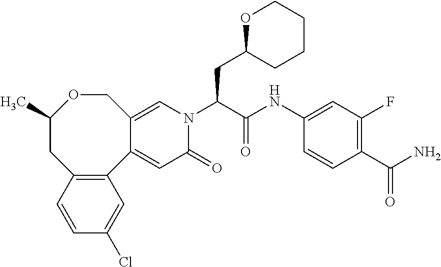 [SMILES: C[C@@H]1Cc2ccc(Cl)cc2-c2cc(=O)n([C@@H](C[C@@H]3CCCCO3)C(=O)Nc3ccc(C(N)=O)c(F)c3)cc2CO1]

Stereoisomer separation of 20 mg of 4-({2-(11-chloro-7-methyl-2-oxo-7,8-dihydro-2H-[3]benzoxocino[5,6-c]pyridin-3(5H)-yl)-3-[(2S)-tetrahydro-2H-pyran-2-yl]propanoyl}amino)-2-fluorobenzamide (mixture of stereoisomers 1 of Example 1) gave
single stereoisomer 1 (the title compound Example 2) (chiral SFC: Rₜ=1.24 min, >99% de): 8 mg,
single stereoisomer 2 (chiral SFC: Rₜ=1.76 min): 9 mg.

Separation method: SFC: column: Daicel OD-H 5 μm, 250 mm×20 mm; eluent: 70% carbon dioxide/30% ethanol; temperature: 40° C.; flow rate: 70 m/min; UV detection: 210 nm.

Analysis method: SFC: column: Daicel OD-H 3 μm, 10 mm×4.6 mm; eluent: 70% carbon dioxide/30% ethanol; flow rate: 3 ml/min; UV detection: 210 nm.

LC-MS (method 4): Rₜ=2.01 min; MS (ESIpos): m/z=568 [M+H]⁺

¹H-NMR (400 MHz, DMSO-d₆): δ [ppm]=10.80 (s, 1H), 8.00 (s, 1H), 7.73-7.63 (m, 2H), 7.58-7.44 (m, 4H), 7.43-7.36 (m, 1H), 7.36-7.31 (m, 1H), 6.39 (s, 1H), 5.82-5.68 (m, 1H), 4.56 (d, 1H), 3.88-3.80 (m, 1H), 3.61-3.50 (m, 2H), 3.30-3.17 (m, 2H, partially concealed), 2.85-2.77 (m, 1H), 2.38-2.23 (m, 2H), 2.12-2.02 (m, 1H), 1.82-1.70 (m, 1H), 1.64-1.56 (m, 1H), 1.46-1.37 (m, 3H), 1.28-1.22 (m, 4H). Additional signals of a minor rotamer were also detected.

### Example 3

### 5-{2-(11-Chloro-7-methyl-2-oxo-7,8-dihydro-2H-[3]benzoxocino[5,6-c]pyridin-3(5H)-yl)-3-[(2S)-tetrahydro-2H-pyran-2-yl]propanamido}pyridine-2-carboxamide (Mixture of Stereoisomers)

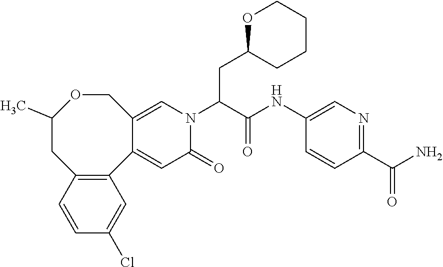 [SMILES: CC1Cc2ccc(Cl)cc2-c2cc(=O)n(C(C[C@@H]3CCCCO3)C(=O)Nc3ccc(C(N)=O)nc3)cc2CO1]

1-Chloro-N,N,2-trimethylprop-1-en-1-amine (20 μl, 0.15 mmol, 1.3 eq.) was added to a solution of 2-(11-chloro-7-methyl-2-oxo-7,8-dihydro-2H-[3]benzoxocino[5,6-c]pyridin-3(5H)-yl)-3-[(2S)-tetrahydro-2H-pyran-2-yl]propanoic acid (mixture of stereoisomers) (51 mg, 0.12 mmol, 1.0 eq.) in dichloromethane (3.6 ml). The reaction mixture was stirred at RT for 10 min, followed by the addition of 5-aminopyridine-2-carboxamide (21 mg, 0.15 mmol, 1.3 eq.). The reaction mixture was stirred at RT for 2 h, followed by evaporation of all volatiles under reduced pressure. The residue was purified by preparative HPLC (reversed phase, eluent: water with 0.05% formic acid/acetonitrile 95:5 to 10:90). Yield (mixture of stereoisomers 1): 18 mg (27% of theory); Yield (mixture of stereoisomers 2): 27 mg (38% of theory).

mixture of stereoisomers 1:

LC-MS (method 5): Rₜ=1.29 min; MS (ESIneg): m/z=549 [M−H]⁻

¹H-NMR (400 MHz, DMSO-d₆): δ [ppm]=10.93/10.88 (2s, 1H), 8.87 (dd, 1H), 8.25-8.15 (m, 1H), 8.04-7.95 (m, 3H), 7.55-7.46 (m, 2H), 7.42-7.31 (m, 2H), 6.41/6.39 (2s, 1H), 5.85-5.72 (m, 1H), 4.51 (d, 1H), 3.90-3.73 (m, 1H), 3.62-3.52 (m, 2H), 3.29-3.05 (m, 2H), 2.87-2.76 (m, 1H), 2.41-2.07 (m, 3H), 1.81-1.70 (m, 1H), 1.70-1.57 (m, 1H), 1.48-1.35 (m, 4H), 1.26 (d, 3H). Additional signals of a minor rotamer were also detected.
mixture of stereoisomers 2:

LC-MS (method 5): Rₜ=1.32 min; MS (ESIneg): m/z=549 [M−H]⁻

¹H-NMR (400 MHz, DMSO-d₆): δ [ppm]=10.89/10.85 (2s, 1H), 8.87 (dd, 1H), 8.28-8.16 (m, 1H), 8.04-7.96 (m, 3H), 7.55-7.46 (m, 2H), 7.42-7.31 (m, 2H), 6.40/6.39 (2s, 1H), 5.83-5.71 (m, 1H), 4.57 (dd, 1H), 3.89-3.76 (m, 1H), 3.61-3.52 (m, 2H), 3.29-3.14 (m, 2H), 2.82 (dd, 1H), 2.40-2.27 (m, 2H), 2.27-2.04 (m, 1H), 1.79-1.69 (m, 1H), 1.60 (t, 1H), 1.48-1.19 (m, 4H), 1.26 (t, 3H). Additional signals of a minor rotamer were also detected.

### Example 4

### 5-({(2S)-2-[(7R)-11-Chloro-7-methyl-2-oxo-7,8-dihydro-2H-[3]benzoxocino[5,6-c]pyridin-3(5H)-yl]-3-[(2S)-tetrahydro-2H-pyran-2-yl]propanoyl}amino)pyridine-2-carboxamide (Single Stereoisomer)

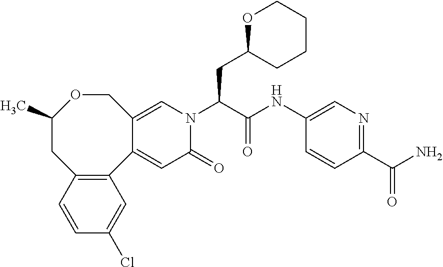 [SMILES: C[C@@H]1Cc2ccc(Cl)cc2-c2cc(=O)n([C@@H](C[C@@H]3CCCCO3)C(=O)Nc3ccc(C(N)=O)nc3)cc2CO1]

Stereoisomer separation of 27 mg of 5-{2-(11-chloro-7-methyl-2-oxo-7,8-dihydro-2H-[3]benzoxocino[5,6-c]pyridin-3(5H)-yl)-3-[(2S)-tetrahydro-2H-pyran-2-yl]propanamido}pyridine-2-carboxamide (mixture of stereoisomers 2 of Example 3) gave
single stereoisomer 3 (chiral HPLC: Rₜ=11.12 min): 8 mg,
single stereoisomer 4 (the title compound Example 4) (chiral HPLC: Rₜ=20.08 min, 99% de): 8 mg.

Separation method: HPLC: column: Daicel Chiralpak IF 5 μm, 250 mm×20 mm; eluent: 100% ethanol; temperature: 70° C.; flow rate: 15 ml/min; UV detection: 220 nm.

Analysis method: HPLC: column: Daicel Chiralpak IE 5 μm, 250 mm×4.6 mm; eluent: 100% ethanol; temperature: 60° C.; flow rate: 1 ml/min; UV detection: 220 nm.

LC-MS (method 3): Rₜ=3.15 min; MS (ESIneg): m/z=549 [M−H]⁻

¹H-NMR (600 MHz, DMSO-d₆): δ [ppm]=10.89 (s, 1H), 8.88 (d, 1H), 8.26 (dd, 1H), 8.04-7.94 (m, 3H), 7.55-7.45 (m, 2H), 7.39 (s, 1H), 7.35-7.33 (m, 1H), 6.40 (s, 1H), 5.77 (t, 1H), 4.56 (d, 1H), 3.89-3.82 (m, 1H), 3.60-3.52 (m, 2H), 3.30-3.22 (m, 2H), 2.81 (d, 1H), 2.54 (s, 1H), 2.37-2.27 (m, 2H), 2.09 (s, 1H), 1.75 (br s, 1H), 1.62 (br d, 1H), 1.48-1.20 (m, 4H), 1.25 (d, 3H). Additional signals of minor rotamers were also detected.

### Example 5

### 4-({2-(11-Chloro-7-methyl-2-oxo-7,8-dihydro-2H-[3]benzoxocino[5,6-c]pyridin-3(5H)-yl)-3-[(2S)-tetrahydro-2H-pyran-2-yl]propanoyl}amino)benzamide (Mixture of Stereoisomers)

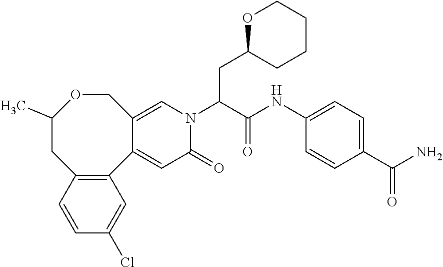 [SMILES: CC1Cc2ccc(Cl)cc2-c2cc(=O)n(C(C[C@@H]3CCCCO3)C(=O)Nc3ccc(C(N)=O)cc3)cc2CO1]

2-(11-Chloro-7-methyl-2-oxo-7,8-dihydro-2H-[3]benzoxocino[5,6-c]pyridin-3(5H)-yl)-3-[(2S)-tetrahydro-2H-pyran-2-yl]propanoic acid (mixture of stereoisomers) (110 mg, 0.242 mmol) and 4-aminobenzamide (49 mg, 0.36 mmol, 1.5 eq.) were reacted according to General Method 11. Yield: 113 mg (84% of theory).

LC-MS (method 4): Rₜ=1.81/1.87 min; MS (ESIpos): m/z=550 [M+H]⁺

¹H-NMR (400 MHz, DMSO-d₆): δ [ppm]=10.67/10.65/10.63 (3s, 1H), 8.03-7.92 (m, 1H), 7.91-7.81 (m, 3H), 7.75-7.65 (m, 2H), 7.53-7.47 (m, 1H), 7.43-7.33 (m, 2H), 7.27-7.19 (m, 1H), 6.41-6.37 (m, 1H), 5.85-5.72 (m, 1H), 4.62-4.45 (m, 1H), 3.90-3.75 (m, 1H), 3.63-3.49 (m, 2H), 3.29-2.98 (m, 2H), 2.88-2.76 (m, 1H), 2.38-2.01 (m, 3H), 1.80-1.68 (m, 1H), 1.66-1.55 (m, 1H), 1.48-1.33 (m, 3H), 1.30-1.15/1.10-1.04 (2m, 4H). Additional signals of minor rotamers were also detected.

### Example 6

### 4-({(2S)-2-[(7R)-11-Chloro-7-methyl-2-oxo-7,8-dihydro-2H-[3]benzoxocino[5,6-c]pyridin-3(5H)-yl]-3-[(2S)-tetrahydro-2H-pyran-2-yl]propanoyl}amino)benzamide (Single Stereoisomer)

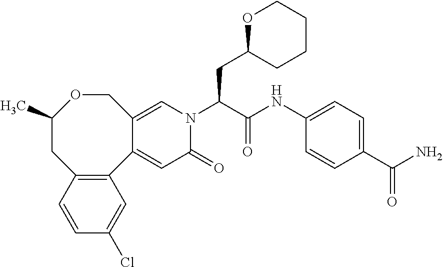 [SMILES: C[C@@H]1Cc2ccc(Cl)cc2-c2cc(=O)n([C@@H](C[C@@H]3CCCCO3)C(=O)Nc3ccc(C(N)=O)cc3)cc2CO1]

Stereoisomer separation of 110 mg of 4-({2-(11-chloro-7-methyl-2-oxo-7,8-dihydro-2H-[3]benzoxocino[5,6-c]pyridin-3(5H)-yl)-3-[(2S)-tetrahydro-2H-pyran-2-yl]propanoyl}amino)-benzamide (mixture of stereoisomers), Example 5 gave
single stereoisomer 1 (chiral HPLC: Rₜ=6.48 min): 41 mg,
single stereoisomer 2 (chiral HPLC: Rₜ=7.78 min): 23 mg,
single stereoisomer 3 (the title compound Example 6) (chiral HPLC: Rₜ=8.98 min, 99% de): 30 mg,
single stereoisomer 4 (chiral HPLC: Rₜ=13.82 min): 13 mg.

First separation method: HPLC: column: Daicel Chiralpak IG 5 μm, 250 mm×20 mm; eluent: 100% ethanol; temperature: 70° C.; flow rate: 15 ml/min; UV detection: 220 nm.

Second separation method: The fractions containing single stereoisomer 3 were combined and again purified with the following conditions: HPLC: column: Daicel Chiralpak IG 5 μm, 250 mm×20 mm; eluent: 100% ethanol; temperature: 70° C.; flow rate: 15 ml/min; UV detection: 220 nm.

Analysis method: HPLC: column: Daicel Chiralpak IG 5 μm, 250 mm×4.6 mm; eluent: 100% ethanol; temperature: 70° C.; flow rate: 1 ml/min; UV detection: 220 nm.

LC-MS (method 4): Rₜ=1.87 min; MS (ESIpos): m/z=550 [M+H]⁺

¹H-NMR (400 MHz, DMSO-d₆): δ [ppm]=10.65 (s, 1H), 8.01 (s, 1H), 7.89-7.81 (m, 3H), 7.75-7.70 (m, 2H), 7.52-7.48 (m, 1H), 7.41-7.37 (m, 1H), 7.35-7.33 (m, 1H), 7.27-7.22 (m, 1H), 6.39 (s, 1H), 5.82-5.75 (m, 1H), 4.56 (d, 1H), 3.89-3.81 (m, 1H), 3.59-3.51 (m, 2H), 3.30-3.19 (m, 2H), 2.81 (d, 1H), 2.38-2.23 (m, 2H), 2.11-2.02 (m, 1H), 1.78-1.70 (m, 1H), 1.65-1.57 (m, 1H), 1.47-1.39 (m, 3H), 1.30-1.21 (m, 4H). Additional signals of minor rotamers were also detected.

### Example 7

### 4-({2-(11-Chloro-7-methyl-2-oxo-7,8-dihydro-2H-[3]benzoxocino[5,6-c]pyridin-3(5H)-yl)-3-[(2R)-1,4-dioxan-2-yl]propanoyl}amino)-2-fluorobenzamide (mixture of stereoisomers)

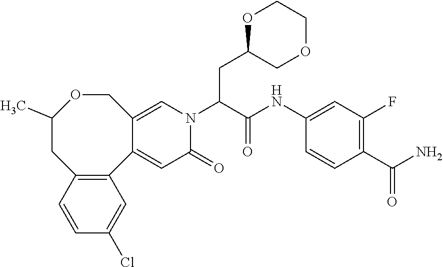 [SMILES: CC1Cc2ccc(Cl)cc2-c2cc(=O)n(C(C[C@@H]3COCCO3)C(=O)Nc3ccc(C(N)=O)c(F)c3)cc2CO1]

2-(11-Chloro-7-methyl-2-oxo-7,8-dihydro-2H-[3]benzoxocino[5,6-c]pyridin-3(5H)-yl)-3-[(2R)-1,4-dioxan-2-yl]propanoic acid (mixture of stereoisomers) (120 mg, 90% purity, 0.25 mmol) and 4-amino-2-fluorobenzamide (62 mg, 0.40 mmol, 1.63 eq.) were reacted according to General Method 11. Yield: 110 mg (74% of theory).

LC-MS (method 7): Rₜ=5.44/5.48/5.53 min; MS (ESIpos): m/z=570 [M+H]⁺

¹H-NMR (400 MHz, DMSO-d₆): δ [ppm]=10.84/10.78 (2s, 1H), 8.02-7.94 (m, 1H), 7.72-7.59 (m, 2H), 7.57-7.47 (m, 3H), 7.47-7.33 (m, 3H), 6.40/6.40 (2s, 1H), 5.80-5.65 (m, 1H), 4.64-4.47 (m, 1H), 3.75-3.48 (m, 7H, partially concealed), 3.35-3.18 (m, 2H), 2.88-2.76 (m, 1H), 2.38-2.20 (m, 2H), 2.19-1.99 (m, 1H), 1.30-1.22 (m, 3H). Additional signals of minor rotamers were also detected.

### Example 8

### 4-({(2S)-2-[(7R)-11-Chloro-7-methyl-2-oxo-7,8-dihydro-2H-[3]benzoxocino[5,6-c]pyridin-3(5H)-yl]-3-[(2R)-1,4-dioxan-2-yl]propanoyl}amino)-2-fluorobenzamide (Single Stereoisomer)

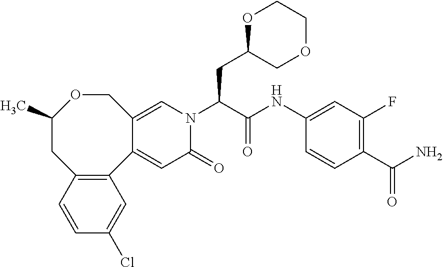 [SMILES: C[C@@H]1Cc2ccc(Cl)cc2-c2cc(=O)n([C@@H](C[C@@H]3COCCO3)C(=O)Nc3ccc(C(N)=O)c(F)c3)cc2CO1]

Stereoisomer separation of 105 mg of 4-({2-(11-chloro-7-methyl-2-oxo-7,8-dihydro-2H-[3]benzoxocino[5,6-c]pyridin-3(5H)-yl)-3-[(2R)-1,4-dioxan-2-yl]propanoyl}amino)-2-fluorobenzamide (mixture of stereoisomers), Example 7 gave
single stereoisomer 1 (chiral HPLC: %=5.89 min): 31 mg,
single stereoisomer 2 (the title compound Example 8) (chiral HPLC: Rₜ=5.50 min, 99% de): 12 mg,
single stereoisomer 3 (chiral HPLC: Rₜ=8.80 min): 6 mg,
single stereoisomer 4 (chiral HPLC: Rₜ=5.15 min): 11 mg.

First separation method: HPLC: column: Daicel Chiralpak IG 5 μm, 250 mm×20 mm; eluent: 100% ethanol with 0.1% water; temperature: 30° C.; flow rate: 15 ml/min; UV detection: 220 nm.

Second separation method: Single stereoisomer 2 and single stereoisomer 3 eluted as a mixture in the first separation. The mixture was purified according to the following conditions: HPLC: column: Daicel Chiralpak OZ-H 5 μm, 250 mm×20 mm; eluent: 100% ethanol with 1% water and 0.2% trifluoroacetic acid; temperature: 30° C.; flow rate: 15 ml/min; UV detection: 220 nm.

Third separation method: The four final fractions from separation methods 1 and 2 were each purified using the following conditions: HPLC: column: Chromatorex C18 10 μm, 125 mm×30 mm; eluent: acetonitrile/water with 0.01% trifluoroacetic acid, gradient: 10%-90% acetonitrile within 15 min; flow rate: 75 ml/min.

Analysis method: HPLC: column: Daicel Chiralcel OZ-H 5 μm, 250 mm×4.6 mm; eluent: 100% ethanol with 1% water and 0.2% trifluoroacetic acid; temperature: 50° C.; flow rate: 1 ml/min; UV detection: 220 nm.

LC-MS (method 5): Rₜ=1.21 min; MS (ESIpos): m/z=570 [M+H]⁺

¹H-NMR (400 MHz, DMSO-d₆): δ [ppm]=10.78 (s, 1H), 8.01 (s, 1H), 7.72-7.64 (m, 2H), 7.58-7.48 (m, 3H), 7.47-7.42 (m, 1H), 7.42-7.37 (m, 1H), 7.37-7.33 (m, 1H), 6.40 (s, 1H), 5.74-5.66 (m, 1H), 4.55 (d, 1H), 3.75-3.65 (m, 2H), 3.64-3.40 (m, 6H), 3.28-3.20 (m, 1H), 2.84-2.78 (m, 1H), 2.38-2.19 (m, 2H), 2.12-1.99 (m, 1H), 1.25 (d, 3H). Additional signals of a minor rotamer were also detected.

### Example 9

### 4-({2-(11-Chloro-7-methyl-2-oxo-7,8-dihydro-2H-[3]benzoxocino[5,6-c]pyridin-3(5H)-yl)-3-[(2R)-1,4-dioxan-2-yl]propanoyl}amino)benzamide (Mixture of Stereoisomers)

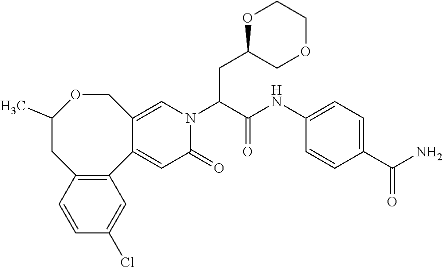 [SMILES: CC1Cc2ccc(Cl)cc2-c2cc(=O)n(C(C[C@@H]3COCCO3)C(=O)Nc3ccc(C(N)=O)cc3)cc2CO1]

2-(11-Chloro-7-methyl-2-oxo-7,8-dihydro-2H-[3]benzoxocino[5,6-c]pyridin-3(5H)-yl)-3-[(2R)-1,4-dioxan-2-yl]propanoic acid (mixture of stereoisomers) (120 mg, 90% purity, 0.25 mmol) and 4-aminobenzamide (55 mg, 0.40 mmol, 1.63 eq.) were reacted according to General Method 11. Yield: 113 mg (81% of theory).

LC-MS (method 5): Rₜ=1.13/1.16/1.17 min; MS (ESIneg): m/z=550 [M−H]⁻

### Example 10

### 4-({(2S)-2-[(7R)-11-Chloro-7-methyl-2-oxo-7,8-dihydro-2H-[3]benzoxocino[5,6-c]pyridin-3(5H)-yl]-3-[(2R)-1,4-dioxan-2-yl]propanoyl}amino)benzamide (Single Stereoisomer)

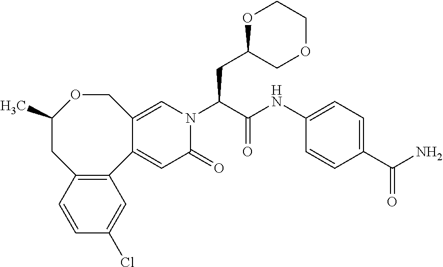 [SMILES: C[C@@H]1Cc2ccc(Cl)cc2-c2cc(=O)n([C@@H](C[C@@H]3COCCO3)C(=O)Nc3ccc(C(N)=O)cc3)cc2CO1]

Stereoisomer separation of 110 mg of 4-({2-(11-chloro-7-methyl-2-oxo-7,8-dihydro-2H-[3]benzoxocino[5,6-c]pyridin-3(5H)-yl)-3-[(2R)-1,4-dioxan-2-yl]propanoyl}amino)benzamide (mixture of stereoisomers), Example 9 gave
single stereoisomer 1 (the title compound Example 10) (chiral HPLC: Rₜ=5.23 min, 98% de): 21 mg,
single stereoisomer 2 (chiral HPLC: Rₜ=4.97 min): 13 mg,
single stereoisomer 3 (chiral HPLC: Rₜ=6.98 min): 8 mg,
single stereoisomer 4 (chiral HPLC: Rₜ=13.03 min): 18 mg.

First separation method: HPLC: column: Daicel Chiralpak OZ-H 5 μm, 250 mm×20 mm; eluent: 100% ethanol with 1% water and 0.2% trifluoroacetic acid; temperature: 30° C.; flow rate: 15 ml/min; UV detection: 220 nm.

Second separation method: Single stereoisomer 1 and single stereoisomer 2 eluted as a mixture in the first separation. The mixture was purified according to the following conditions: HPLC: column: Daicel Chiralpak IG 5 μm, 250 mm×20 mm; eluent: 100% ethanol with 0.2% trifluoroacetic acid; temperature: 30° C.; flow rate: 15 ml/min; UV detection: 220 nm.

Third separation method: The four final fractions from separation methods 1 and 2 were each purified using the following conditions: HPLC: column: Chromatorex C18 10 μm, 125 mm×30 mm, eluent: acetonitrile/water with 0.01% trifluoroacetic acid, gradient: 10%-90% acetonitrile within 15 min; flow rate: 75 ml/min.

Analysis method: HPLC: column: Daicel Chiralcel OZ-H 5 μm, 250 mm×4.6 mm; eluent: 100% ethanol with 1% water and 0.2% trifluoroacetic acid; temperature: 50° C.; flow rate: 1 ml/min; UV detection: 220 nm.

LC-MS (method 5): Rₜ=1.21 min; MS (ESIpos): m/z=552 [M+H]⁺

¹H-NMR (400 MHz, DMSO-d₆): δ [ppm]=10.65 (m, 1H), 8.02 (s, 1H), 7.90-7.82 (m, 3H), 7.74-7.66 (m, 2H), 7.53-7.47 (m, 1H), 7.42-7.33 (m, 2H), 7.29-7.21 (m, 1H), 6.41 (s, 1H), 5.79-5.71 (m, 1H), 4.55 (d, 1H), 3.76-3.65 (m, 2H), 3.64-3.39 (m, 6H), 3.29-3.21 (m, 1H), 2.85-2.76 (m, 1H), 2.37-2.18 (m, 2H), 2.13-2.01 (m, 1H), 1.25 (d, 3H). Additional signals of a minor rotamer were also detected.

### Example 11

### 2-(11-Chloro-7-methyl-2-oxo-7,8-dihydro-2H-[3]benzoxocino[5,6-c]pyridin-3(5H)-yl)-3-[(2R)-1,4-dioxan-2-yl]-N-(2-methyl-2H-benzotriazol-5-yl)propanamide (Mixture of Stereoisomers)

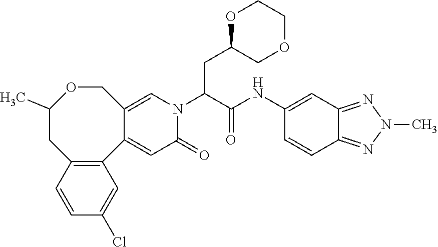

2-(11-Chloro-7-methyl-2-oxo-7,8-dihydro-2H-[3]benzoxocino[5,6-c]pyridin-3(5H)-yl)-3-[(2R)-1,4-dioxan-2-yl]propanoic acid (mixture of stereoisomers) (60 mg, 91% purity, 0.13 mmol) and 2-methyl-2H-benzotriazol-5-amine (30 mg, 0.20 mmol, 1.63 eq.) were reacted according to General Method 11. Yield: 59 mg (91% purity, 75% of theory).

LC-MS (method 4): Rₜ=1.80/1.82/1.85 min; MS (ESIpos): m/z=564 [M+H]⁺

¹H-NMR (400 MHz, DMSO-d₆): δ [ppm]=10.71/10.68 (2s, 1H), 8.38-8.30 (m, 1H), 8.07-7.95 (m, 1H), 7.91-7.84 (m, 1H), 7.56-7.47 (m, 2H), 7.43-7.33 (m, 2H), 6.41/6.41/6.40 (3s, 1H), 5.85-5.73 (m, 1H), 4.65-4.49 (m, 1H), 4.46/4.45/4.44 (3s, 3H), 3.81-3.39 (m, 8H, partially concealed), 3.34-3.17 (m, 1H), 2.86-2.76 (m, 1H), 2.38-2.22 (m, 2H), 2.20-2.02 (m, 1H), 1.31-1.22 (m, 3H).

Additional signals of minor rotamers were also detected.

### Example 12

### (2S)-2-[(7R)-11-Chloro-7-methyl-2-oxo-7,8-dihydro-2H-[3]benzoxocino[5,6-c]pyridin-3(5H)-yl]-3-[(2R)-1,4-dioxan-2-yl]-N-(2-methyl-2H-benzotriazol-5-yl)propanamide (Single Stereoisomer)

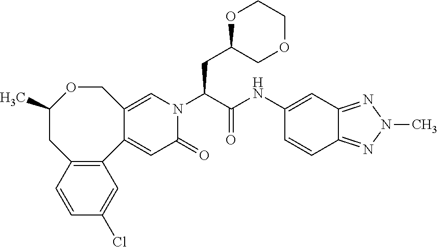

Stereoisomer separation of 55 mg of 2-(11-chloro-7-methyl-2-oxo-7,8-dihydro-2H-[3]benzoxocino[5,6-c]pyridin-3(5H)-yl)-3-[(2R)-1,4-dioxan-2-yl]-N-(2-methyl-2H-benzotriazol-5-yl)propanamide (mixture of stereoisomers), Example 11 gave
single stereoisomer 1 (chiral HPLC: Rₜ=10.74): 7 mg,
single stereoisomer 2 (chiral HPLC: Rₜ=11.75): 8 mg,
single stereoisomer 3 (the title compound Example 12) (chiral HPLC: Rₜ=15.74 min, >99% de): 15 mg,
single stereoisomer 4 (chiral HPLC: Rₜ=13.65 min): 17 mg.

Separation method 1: HPLC: column: Daicel Chiralpak IC 5 μm, 250 mm×20 mm; eluent: 40% n-heptane/60% 2-propanol; temperature: 55° C.; flow rate: 15 ml/min; UV detection: 220 nm.

Separation method 2: Single stereoisomer 2 and single stereoisomer 3 eluted as a mixture in the first separation. The mixture was purified according to the following conditions: HPLC: column: Daicel Chiralpak IC 5 μm, 250 mm×20 mm; eluent: 50% n-heptane/50% 2-propanol; temperature: 55° C.; flow rate: 15 ml/min; UV detection: 220 nm.

Analysis method: HPLC: column: Daicel Chiralpak IC 5 μm, 250 mm×4.6 mm; eluent: 50% n-heptane/50% 2-propanol with 1% water and 0.2% trifluoroacetic acid; temperature: 55° C.; flow rate: 1.0 ml/min; UV detection: 220 nm.

LC-MS (method 4): Rₜ=1.84 min; MS (ESIpos): m/z=564 [M+H]⁺

¹H-NMR (400 MHz, DMSO-d₆): δ [ppm]=10.68 (s, 1H), 8.38-8.32 (m, 1H), 8.05 (s, 1H), 7.88 (d, 1H), 7.56-7.48 (m, 2H), 7.42-7.35 (m, 2H), 6.41 (s, 1H), 5.79 (t, 1H), 4.56 (d, 1H), 4.46 (s, 3H), 3.78-3.65 (m, 2H), 3.65-3.41 (m, 6H), 3.30-3.22 (m, 1H, partially concealed), 2.81 (d, 1H), 2.38-2.23 (m, 2H), 2.13-2.03 (m, 1H), 1.25 (d, 3H). Additional signals of a minor rotamer were also detected.

### Example 13

### 4-({(2S,4S)-2-[(7R)-11-Chloro-7-methyl-2-oxo-7,8-dihydro-2H-[3]benzoxocino[5,6-c]pyridin-3(5H)-yl]-4-methoxypentanoyl}amino)benzamide (Single Stereoisomer)

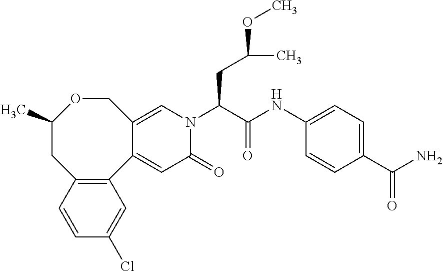 [SMILES: CO[C@@H](C)C[C@@H](C(=O)Nc1ccc(C(N)=O)cc1)n1cc2c(cc1=O)-c1cc(Cl)ccc1C[C@@H](C)OC2]

(2ξ)-2-[(7R)-11-Chloro-7-methyl-2-oxo-7,8-dihydro-2H-[3]benzoxocino[5,6-c]pyridin-3(5H)-yl]-2,3,5-trideoxy-4-O-methyl-L-glycero-pentonic acid (mixture of two diastereomers) (58 mg, 0.14 mmol), pyridine (13 μl, 0.16 mmol, 1.1 eq.) and T3P (125 μl, 50% solution in ethyl acetate, 0.21 mmol, 1.5 eq.) were added under argon atmosphere at RT to a solution of 4-aminobenzamide (21 mg, 0.16 mmol, 1.1 eq.) in tetrahydrofuran (4 ml). The reaction mixture was stirred at RT for 70 min before all volatiles were removed under reduced pressure. The residue was purified by preparative HPLC (reversed phase, eluent: acetonitrile/water gradient). Yield: 49 mg (diastereomeric mixture with 78% of the desired stereoisomer, 51% of theory).

Separation of 47 mg of this diastereomeric mixture gave
single stereoisomer 1 (chiral HPLC: Rₜ=7.15 min, 99% de): 11 mg,
single stereoisomer 2 (the title compound Example 13) (chiral HPLC: Rₜ=8.84 min, 99% de): 33 mg.

Separation method: HPLC: column: Daicel Chiralpak IF 5 μm, 250 mm×20 mm; eluent: 50% n-heptane/50% ethanol; temperature: 30° C.; flow rate: 15 ml/min; UV detection: 220 nm.

Analysis method: HPLC: column: Daicel Chiralpak IF 5 μm, 250 mm×4.6 mm; eluent: 50% iso-hexane/50% ethanol; temperature: 40° C.; flow rate: 1 ml/min; UV detection: 220 nm.

¹H-NMR (600 MHz, DMSO-d₆): δ [ppm]=10.71 (m, 1H), 8.06/7.97 (2s, 1H), 7.91-7.82 (m, 3H), 7.73/7.70 (2d, 2H), 7.50/7.46 (2dd, 1H), 7.40/7.32 (2d, 1H), 7.34 (d, 1H), 7.26 (br s, 1H), 6.40/6.34 (2s, 1H), 5.85-5.79 (m, 1H), 4.57/4.24/3.92/3.55 (4d, 2H), 4.16-4.10/3.60-3.53 (2m, 1H, partially concealed), 3.27-3.20 (m, 1H), 3.18/3.17 (2s, 3H), 2.85-2.76 (m, 1H), 2.36-2.30 (m, 1H), 2.26-2.15 (m, 2H), 1.26/1.16/1.14/1.06 (4d, 6H). Additional signals of minor rotamers were also detected.

### Example 14

### 4-({(2S,4R)-2-[(7R)-11-Chloro-7-methyl-2-oxo-7,8-dihydro-2H-[3]benzoxocino[5,6-c]pyridin-3(5H)-yl]-4-cyclopropyl-4-methoxybutanoyl}amino)benzamide (Single Stereoisomer)

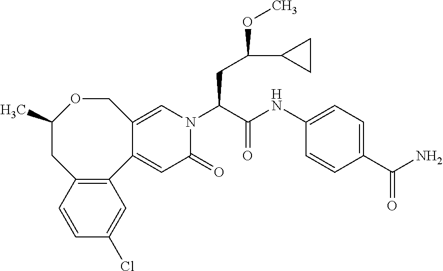 [SMILES: CO[C@H](C[C@@H](C(=O)Nc1ccc(C(N)=O)cc1)n1cc2c(cc1=O)-c1cc(Cl)ccc1C[C@@H](C)OC2)C1CC1]

1,1,3,3-Tetramethylguanidine (30 μl, 0.24 mmol, 3.0 eq.) was added under argon atmosphere at RT to a solution of (7R)-11-chloro-7-methyl-3,5,7,8-tetrahydro-2H-[3]benzoxocino[5,6-c]pyridin-2-one (single stereoisomer) (22 mg, 0.08 mmol) in 2-propanol/acetone (4:1, 1.0 ml). The mixture was stirred at RT for 15 min, followed by addition of 4-{[(2R,4R)-2-bromo-4-cyclopropyl-4-methoxybutanoyl]-amino}benzamide (single stereoisomer) (31 mg, 0.09 mmol, 1.1 eq.) and of further 2-propanol/acetone (4:1, 1.0 ml). The reaction mixture was stirred at RT for 5 days and concentrated under reduced pressure. The residue was purified by preparative HPLC (reversed phase, eluent: acetonitrile/water gradient). Yield: 32 mg (73% of theory).

LC-MS (method 4): Rₜ=1.95 min; MS (ESIpos): m/z=550 [M+H]⁺

¹H-NMR (600 MHz, DMSO-d₆): δ [ppm]=10.71-10.56 (m, 1H), 7.89-7.79 (m, 4H), 7.73-7.66 (m, 2H), 7.48-7.37/7.35-7.28 (2m, 3H), 7.22 (br s, 1H), 6.39-6.30 (m, 1H), 5.89-5.77 (m, 1H), 4.53-4.44/4.30-4.24/3.90-3.84/3.3-3.22 (4m, 2H, partially concealed), 3.27/3.21 (2s, 3H), 2.89-2.80/2.72-2.60/2.59-2.5 (3m, 2H), 2.41-2.18 (m, 3H), 2.10-1.95 (m, 1H), 0.94-0.78 (m, 4H), 0.61-0.53 (m, 1H), 0.44-0.34 (m, 2H). Additional signals of minor rotamers were also detected.

### Example 15

### 4-({(2S,4R)-2-[(7R)-11-Chloro-7-methyl-2-oxo-7,8-dihydro-2H-[3]benzoxocino[5,6-c]pyridin-3(5H)-yl]-5,5-difluoro-4-methoxypentanoyl}amino)benzamide (Single Stereoisomer)

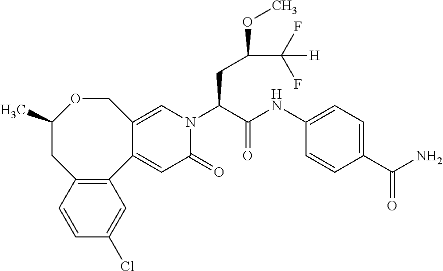 [SMILES: CO[C@H](C[C@@H](C(=O)Nc1ccc(C(N)=O)cc1)n1cc2c(cc1=O)-c1cc(Cl)ccc1C[C@@H](C)OC2)C(F)F]

1,1,3,3-Tetramethylguanidine (23 μl, 0.18 mmol, 3.0 eq.) was added under argon atmosphere at RT to a solution of (7R)-11-chloro-7-methyl-3,5,7,8-tetrahydro-2H-[3]benzoxocino[5,6-c]pyridin-2-one (single stereoisomer) (17 mg, 0.06 mmol) in 2-propanol/acetone (4:1, 1.0 ml). The mixture was stirred at RT for 15 min, followed by addition of 4-{[(2R,4R)-2-bromo-5,5-difluoro-4-methoxypentanoyl]amino}benzamide (single stereoisomer) (24 mg, 0.07 mmol, 1.1 eq.) and of further 2-propanol/acetone (4:1, 1.0 ml). The reaction mixture was stirred at RT for 2 days and concentrated under reduced pressure. The residue was purified by preparative HPLC (reversed phase, eluent: acetonitrile/water gradient). Yield: 25 mg (74% of theory).

LC-MS (method 4): Rₜ=1.87 min; MS (ESIpos): m/z=560 [M+H]⁺

¹H-NMR (600 MHz, DMSO-d₆): δ [ppm]=10.88-10.39 (m, 1H), 7.91-7.78 (m, 4H), 7.74-7.66 (m, 2H), 7.49-7.28 (m, 3H), 7.23 (br s, 1H), 6.44-6.33 (m, 1H), 6.24-6.00 (m, 1H), 5.91-5.75 (m, 1H), 4.53-4.44/4.32-4.23/3.92-3.85/3.59-3.46 (4m, 2H), 3.33 (s, 3H), 2.89-2.81/2.5-1.95 (2m, 5H, partially concealed), 2.74-2.64 (m, 1H), 0.95-0.82 (m, 3H). Additional signals of minor rotamers were also detected.

### Example 16

### 4-{2-[(7R)-11-Chlor-7-methyl-2-oxo-7,8-dihydro-2H-[3]benzoxocino[5,6-c]pyridin-3(5H)-yl]-4-(difluoromethoxy)butanamido}benzamide (Mixture of Two Diastereomers)

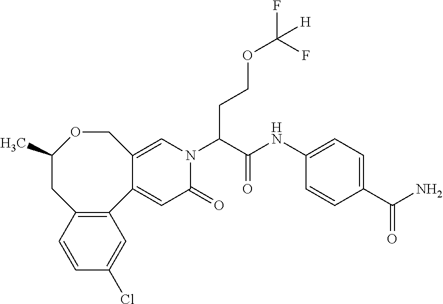 [SMILES: C[C@@H]1Cc2ccc(Cl)cc2-c2cc(=O)n(C(CCOC(F)F)C(=O)Nc3ccc(C(N)=O)cc3)cc2CO1]

2-[(7R)-11-Chloro-7-methyl-2-oxo-7,8-dihydro-2H-[3]benzoxocino[5,6-c]pyridin-3(5H)-yl]-4-(difluoromethoxy)butanoic acid (mixture of two diastereomers) (138 mg, 0.32 mmol), pyridine (29 μl, 0.36 mmol, 1.1 eq.) and T3P (283 μl, 50% solution in ethyl acetate, 0.48 mmol, 1.5 eq.) were added under argon atmosphere at RT to a solution of 4-aminobenzamide (48 mg, 0.36 mmol, 1.1 eq.) in tetrahydrofuran (8 ml). The reaction mixture was stirred at RT for 30 min before all volatiles were removed under reduced pressure. The residue was purified by preparative HPLC (reversed phase, eluent: acetonitrile/water gradient). Yield: 123 mg (70% of theory).

LC-MS (method 4): Rₜ=1.74/1.78 min; MS (ESIpos): m/z=546 [M+H]⁺

¹H-NMR (600 MHz, DMSO-d₆): δ [ppm]=10.78-10.68 (m, 1H), 8.04/8.02/8.00/7.97 (4s, 1H), 7.96-7.83 (m, 3H), 7.73/7.71 (2d, 2H), 7.58-7.48 (m, 1H), 7.47-7.33 (m, 2H), 7.26 (br s, 1H), 6.66/6.65 (2t, 1H), 6.45/6.44/6.39/6.37 (4s, 1H), 5.87-5.77 (m, 1H), 4.60/4.54/4.27/3.59 (4d, 2H), 4.22-4.14/4.00-3.75 (2m, 2H), 3.66-3.55 (m, 1H, partially concealed), 2.89-2.77 (m, 1H), 2.5-2.45 (m, 2H, partially concealed), 2.41-2.34 (m, 1H), 1.35-1.25/1.10 (m/d, 3H). Additional signals of minor rotamers were also detected.

### Example 17

### 4-{[(2S)-2-[(7R)-11-Chloro-7-methyl-2-oxo-7,8-dihydro-2H-[3]benzoxocino[5,6-c]pyridin-3(5H)-yl]-4-(difluoromethoxy)butanoyl]amino}benzamide (Single Stereoisomer)

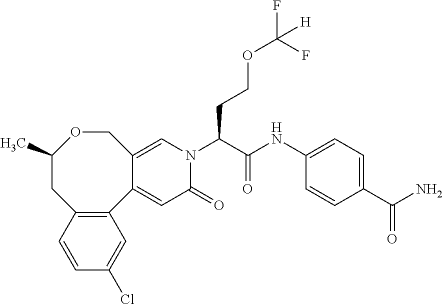 [SMILES: C[C@@H]1Cc2ccc(Cl)cc2-c2cc(=O)n([C@@H](CCOC(F)F)C(=O)Nc3ccc(C(N)=O)cc3)cc2CO1]

Diastereomer separation of 121 mg of 4-{2-[(7R)-11-chloro-7-methyl-2-oxo-7,8-dihydro-2H-[3]benzoxocino[5,6-c]pyridin-3(5H)-yl]-4-(difluoromethoxy)butanamido}benzamide (mixture of two diastereomers), Example 16 gave
single stereoisomer 1 (chiral SFC: Rₜ=1.55 min, 99% de): 28 mg,
single stereoisomer 2 (the title compound Example 17) (chiral SFC: Rₜ=2.05 min, >99% de): 76 mg.

Separation method: SFC: column: Maisch Daicel OJ-H, 250 mm×25 mm; eluent: 75% carbon dioxide/25% ethanol; temperature: 40° C.; flow rate: 80 ml/min; UV detection: 210 nm.

Analysis method: SFC: column: Maisch Daicel OJ-H, 50 mm×4.6 mm; eluent: 85% carbon dioxide/15% ethanol; temperature: 40° C.; flow rate: 3 ml/min; UV detection: 210 nm.

¹H-NMR (600 MHz, DMSO-d₆): δ [ppm]=10.73-10.67 (m, 1H), 8.01/7.93 (2s, 1H), 7.90-7.82 (m, 3H), 7.70/7.67 (2d, 2H), 7.50/7.46 (2dd, 1H), 7.42-7.37 (m, 1H), 7.34-7.30 (m, 1H), 7.23 (br s, 1H), 6.63/6.62 (2t, 1H), 6.41/6.33 (2s, 1H), 5.82-5.75 (m, 1H), 4.56/4.24/3.94/3.56 (4d, 2H), 4.17-4.11/3.94-3.72 (2m, 2H), 3.61-3.53 (m, 1H, partially concealed), 2.85-2.73 (m, 1H), 2.5-2.43 (m, 2H, partially concealed), 2.35 (dd, 1H), 1.26/1.06 (2d, 3H). Additional signals of minor rotamers were also detected.

### Example 18

### 4-{[(4S)-2-[(7R)-11-Chloro-7-methyl-2-oxo-7,8-dihydro-2H-[3]benzoxocino[5,6-c]pyridin-3(5H)-yl]-4-(difluoromethoxy)pentanoyl]amino}benzamide (Mixture of Two Diastereomers)

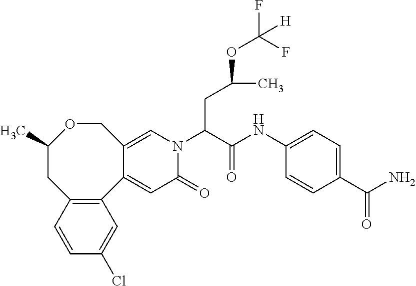 [SMILES: C[C@@H]1Cc2ccc(Cl)cc2-c2cc(=O)n(C(C[C@H](C)OC(F)F)C(=O)Nc3ccc(C(N)=O)cc3)cc2CO1]

(2ξ)-2-[(7R)-11-Chloro-7-methyl-2-oxo-7,8-dihydro-2H-[3]benzoxocino[5,6-c]pyridin-3(5H)-yl]-2,3,5-trideoxy-4-O-(difluoromethyl)-L-glycero-pentonic acid (mixture of two diastereomers) (93 mg, 0.21 mmol), pyridine (19 μl, 0.23 mmol, 1.1 eq.) and T3P (184 μl, 50% solution in ethyl acetate, 0.32 mmol, 1.5 eq.) were added under argon atmosphere at RT to a solution of 4-aminobenzamide (32 mg, 0.23 mmol, 1.1 eq.) in tetrahydrofuran (4.5 ml). The reaction mixture was stirred at RT for 30 min before all volatiles were removed under reduced pressure. The residue was purified by preparative HPLC (reversed phase, eluent: acetonitrile/water gradient). Yield: 85 mg (71% of theory).

LC-MS (method 4): Rₜ=1.84 min; MS (ESIpos): m/z=560 [M+H]⁺

¹H-NMR (600 MHz, DMSO-d₆): δ [ppm]=10.80-10.67 (m, 1H), 8.02/7.99/7.92 (3s, 1H), 7.91-7.81 (m, 3H), 7.74-7.64 (m, 2H), 7.53-7.45 (m, 1H), 7.42-7.37 (m, 1H), 7.37-7.29 (m, 1H), 7.29-7.22 (m, 1H), 6.64/6.63/6.59 (3t, 1H), 6.42/6.39/6.35/6.34 (4s, 1H), 5.85-5.77 (m, 1H), 4.55/4.50/4.28/4.23/3.93/3.86/3.58/3.53 (8d, 2H), 4.19-4.12 (m, 1H), 3.61-3.53 (m, 1H, partially concealed), 2.85-2.73 (m, 1H), 2.45-2.29 (m, 3H), 1.35-1.04 (m, 6H). Additional signals of minor rotamers were also detected.

### Example 19

### 4-{[(2S,4S)-2-[(7R)-11-Chloro-7-methyl-2-oxo-7,8-dihydro-2H-[3]benzoxocino[5,6-c]pyridin-3(5H)-yl]-4-(difluoromethoxy)pentanoyl]amino}benzamide (Single Stereoisomer)

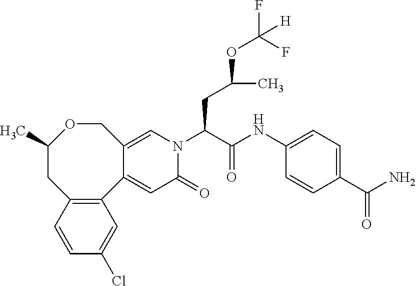 [SMILES: C[C@@H]1Cc2ccc(Cl)cc2-c2cc(=O)n([C@@H](C[C@H](C)OC(F)F)C(=O)Nc3ccc(C(N)=O)cc3)cc2CO1]

Diastereomer separation of 85 mg of 4-{[(4S)-2-[(7R)-11-chloro-7-methyl-2-oxo-7,8-dihydro-2H-[3]benzoxocino[5,6-c]pyridin-3(5H)-yl]-4-(difluoromethoxy)pentanoyl]amino}benzamide (mixture of two diastereomers), Example 18 gave
single stereoisomer 1 (chiral HPLC: Rₜ=5.38 min, 99% de): 35 mg,
single stereoisomer 2 (the title compound Example 19) (chiral HPLC: Rₜ=6.39 min, 99% de): 41 mg.

Separation method: HPLC: column: Daicel Chiralpak IF 5 μm, 250 mm×20 mm; eluent: 50% n-heptane/50% ethanol; temperature: 30° C.; flow rate: 15 ml/min; UV detection: 220 nm.

Analysis method: HPLC: column: Daicel Chiralpak IF 5 μm, 250 mm×4.6 mm; eluent: 50% iso-hexane/50% ethanol; temperature: 40° C.; flow rate: 1 ml/min; UV detection: 220 nm.

¹H-NMR (400 MHz, DMSO-d₆): δ [ppm]=10.80-10.67 (m, 1H), 8.02/7.92 (2s, 1H), 7.91-7.81 (m, 3H), 7.74-7.64 (m, 2H), 7.50/7.46 (2dd, 1H), 7.43-7.36 (m, 1H), 7.34-7.28 (m, 1H), 7.25 (br s, 1H), 6.64/6.63 (2t, 1H), 6.42/6.34 (2s, 1H), 5.82 (t, 1H), 4.55/4.23/3.93/3.53 (4d, 2H), 4.21-4.10 (m, 1H), 3.61-3.53 (m, 1H, partially concealed), 2.85-2.73 (m, 1H), 2.43-2.29 (m, 3H), 1.29/1.25/1.09/1.06 (4d, 6H). Additional signals of minor rotamers were also detected.

### Example 20

### 4-({(2S)-2-[(7R)-11-Chloro-7-methyl-2-oxo-7,8-dihydro-2H-[3]benzoxocino[5,6-c]pyridin-3(5H)-yl]-4-[(propan-2-yl)oxy]butanoyl}amino)benzamide (Single Stereoisomer)

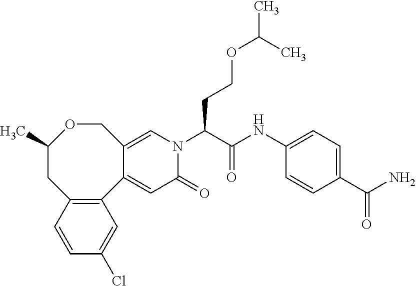 [SMILES: CC(C)OCC[C@@H](C(=O)Nc1ccc(C(N)=O)cc1)n1cc2c(cc1=O)-c1cc(Cl)ccc1C[C@@H](C)OC2]

2-[(7R)-11-Chloro-7-methyl-2-oxo-7,8-dihydro-2H-[3]benzoxocino[5,6-c]pyridin-3(5H)-yl]-4-isopropoxybutanoic acid (mixture of two diastereomers) (72 mg, 0.17 mmol), pyridine (15 μl, 0.19 mmol, 1.1 eq.) and T3P (150 μl, 50% solution in ethyl acetate, 0.26 mmol, 1.5 eq.) were added under argon atmosphere at RT to a solution of 4-aminobenzamide (26 mg, 0.19 mmol, 1.1 eq.) in tetrahydrofuran (4.5 ml). The reaction mixture was stirred at RT for 70 min before all volatiles were removed under reduced pressure. The residue was purified by preparative HPLC (reversed phase, eluent: acetonitrile/water gradient). Yield: 45 mg (diastereomeric mixture with 91% of the desired stereoisomer, 45% of theory).

Separation of 43 mg of this diastereomeric mixture gave
single stereoisomer 1 (the title compound Example 20) (chiral HPLC: Rₜ=1.77 min, 99% de): 25 mg,
single stereoisomer 2 (chiral HPLC: Rₜ=2.95 min, 99% de): 4 mg.

Separation method: HPLC: column: Daicel Chiralcel OX-H 5 μm, 250 mm×20 mm; eluent: 45% n-heptane/55% ethanol; temperature: 45° C.; flow rate: 25 ml/min; UV detection: 220 nm.

Analysis method: HPLC: column: Daicel Chiralpak OX 3 μm, 50 mm×4.6 mm; eluent: 50% n-hexane/50% ethanol with 0.2% diethylamine; temperature: 40° C.; flow rate: 1 ml/min; UV detection: 220 nm.

¹H-NMR (600 MHz, DMSO-d₆): δ [ppm]=10.66-10.59 (m, 1H), 7.99/7.92 (2s, 1H), 7.90-7.81 (m, 3H), 7.71/7.69 (2d, 2H), 7.50/7.46 (2dd, 1H), 7.42-7.37 (m, 1H), 7.31/7.28 (2d, 1H), 7.22 (br s, 1H), 6.39/6.32 (2s, 1H), 5.80-5.72 (m, 1H), 4.56/4.24/3.93/3.55 (4d, 2H), 3.60-3.53 (m, 1H, partially concealed), 3.50-3.39 (m, 1H), 3.3-3.22 (m, 1H, partially concealed), 2.85-2.76 (m, 1H), 2.44-2.25 (m, 3H), 1.29-1.22 (m, 3H), 1.10-0.93 (m, 6H). Additional signals of minor rotamers were also detected.

### Example 21

### 4-{[4-tert-Butoxy-2-(11-chloro-7-methyl-2-oxo-7,8-dihydro-2H-[3]benzoxocino[5,6-c]pyridin-3(5H)-yl)butanoyl]amino}-2-fluorobenzamide (Mixture of Stereoisomers)

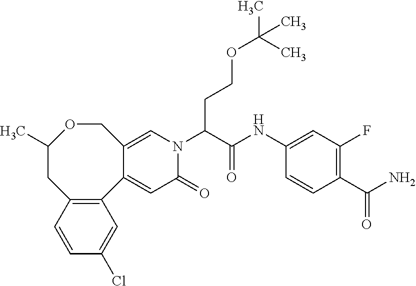 [SMILES: CC1Cc2ccc(Cl)cc2-c2cc(=O)n(C(CCOC(C)(C)C)C(=O)Nc3ccc(C(N)=O)c(F)c3)cc2CO1]

4-tert-Butoxy-2-(11-chloro-7-methyl-2-oxo-7,8-dihydro-2H-[3]benzoxocino[5,6-c]pyridin-3(5H)-yl)butanoic acid (mixture of stereoisomers) (100 mg, 0.22 mmol, 1.0 eq.) was dissolved in dichloromethane (8.0 ml) under argon atmosphere and 1-chloro-N,N,2-trimethylprop-1-en-1-amine (38 μl, 0.29 mmol, 1.3 eq.) was added. The reaction mixture was stirred at RT for 10 min, followed by addition of 4-amino-2-fluorobenzamide (44 mg, 0.29 mmol, 1.3 eq.). Stirring was continued for 3 h, followed by evaporation of all volatiles under reduced pressure and purification by preparative HPLC (reversed phase, eluent: water with 0.05% formic acid/acetonitrile 95:5 to 10:90). Yield: 129 mg (quantitative of theory).

LC-MS (method 5): Rₜ=1.36/1.39 min; MS (ESIneg): m/z=568 [M−H]⁻

¹H-NMR (500 MHz, DMSO-d₆): δ [ppm]=10.87/10.80 (2s, 1H), 7.98/7.97 (2s, 1H), 7.73-7.65 (m, 2H), 7.56-7.47 (m, 3H), 7.47-7.38 (m, 2H), 7.35/7.23 (2d, 1H), 6.40 (m, 1H), 5.81-5.68 (m, 1H), 4.57/4.51 (2d, 1H), 3.62-3.52 (m, 1H), 3.49-3.35 (m, 1H, partially concealed), 3.27-3.18 (m, 1H, partially concealed), 2.84/2.80 (2d, 1H), 2.45-2.26 (m, 3H), 1.26/1.23 (2d, 3H), 1.03/1.01 (2s, 9H). Additional signals of minor rotamers were also detected.

### Example 22

### 4-({(2S)-4-tert-Butoxy-2-[(7R)-11-chloro-7-methyl-2-oxo-7,8-dihydro-2H-[3]benzoxocino[5,6-c]pyridin-3(5H)-yl]butanoyl}amino)-2-fluorobenzamide (Single Stereoisomer)

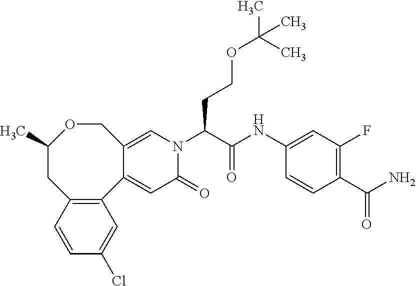 [SMILES: C[C@@H]1Cc2ccc(Cl)cc2-c2cc(=O)n([C@@H](CCOC(C)(C)C)C(=O)Nc3ccc(C(N)=O)c(F)c3)cc2CO1]

Stereoisomer separation of 129 mg of 4-{[4-tert-butoxy-2-(11-chloro-7-methyl-2-oxo-7,8-dihydro-2H-[3]benzoxocino[5,6-c]pyridin-3(5H)-yl)butanoyl]amino}-2-fluorobenzamide (mixture of stereoisomers), Example 21 gave
single stereoisomer 1 (chiral HPLC: Rₜ=5.81 min): 43 mg,
single stereoisomer 2 (the title compound Example 22) (chiral HPLC: Rₜ=7.39 min, 99% de): 36 mg,
single stereoisomer 3 (chiral HPLC: Rₜ=8.06 min): 17 mg,
single stereoisomer 4 (chiral HPLC: Rₜ=10.34 min): 34 mg.

First separation method: HPLC: column: Daicel Chiralpak ID 5 μm, 250 mm×20 mm; eluent: 50% n-heptane/50% 2-propanol with 1% water and 0.2% trifluoroacetic acid; temperature: 40° C.; flow rate: 15 m/min; UV detection: 220 nm.

Second separation method: Single stereoisomer 2 and single stereoisomer 3 eluted as a mixture in the first separation method. Their fractions were combined and the stereoisomers were separated with the following conditions: HPLC: column: Daicel Chiralcel OZ-H 5 μm, 250 mm×20 mm; eluent: 50% n-heptane/50% 2-propanol with 1% water and 0.2% trifluoroacetic acid; temperature: 40° C.; flow rate: 15 ml/min; UV detection: 220 nm.

Analysis method: HPLC: column: Daicel Chiralpak ID 5 μm, 250 mm×4.6 mm; eluent: 50% n-heptane/50% 2-propanol with 0.2% trifluoroacetic acid; temperature: 40° C.; flow rate: 1 ml/min; UV detection: 220 nm.

LC-MS (method 1): Rₜ=1.08 min; MS (ESIpos): m/z=570 [M+H]⁺

¹H-NMR (400 MHz, DMSO-d₆): δ [ppm]=10.79 (s, 1H), 7.98 (s, 1H), 7.73-7.64 (m, 2H), 7.54 (br s, 1H), 7.51 (dd, 2H), 7.45 (dd, 1H), 7.42-7.37 (m, 1H), 7.23 (d, 1H), 6.40 (s, 1H), 5.71 (dd, 1H), 4.56 (d, 1H), 3.61-3.51 (m, 2H), 3.49-3.34 (m, 1H), 3.26-3.19 (m, 1H), 2.83 (d, 1H), 2.41-2.27 (m, 3H), 1.26 (d, 3H), 1.03 (s, 9H). Additional signals of minor rotamers were also detected.

### Example 23

### 4-{[4-tert-Butoxy-2-(11-chloro-7-methyl-2-oxo-7,8-dihydro-2H-[3]benzoxocino[5,6-c]pyridin-3(5H)-yl)butanoyl]amino}benzamide (Mixture of Stereoisomers)

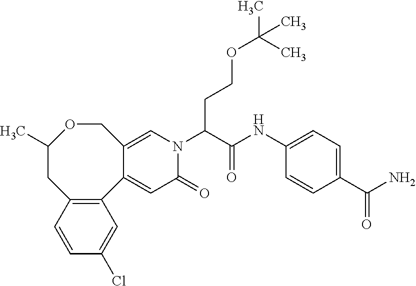 [SMILES: CC1Cc2ccc(Cl)cc2-c2cc(=O)n(C(CCOC(C)(C)C)C(=O)Nc3ccc(C(N)=O)cc3)cc2CO1]

4-tert-Butoxy-2-(11-chloro-7-methyl-2-oxo-7,8-dihydro-2H-[3]benzoxocino[5,6-c]pyridin-3(5H)-yl)butanoic acid (mixture of stereoisomers) (120 mg, 0.26 mmol, 1.0 eq.) was dissolved in dichloromethane (8.0 ml) under argon atmosphere and 1-chloro-N,N,2-trimethylprop-1-en-1-amine (45 μl, 0.34 mmol, 1.3 eq.) was added. The reaction mixture was stirred at RT for 10 min, followed by addition of 4-aminobenzamide (46 mg, 0.34 mmol, 1.3 eq.). Stirring was continued for 3 h, followed by evaporation of all volatiles under reduced pressure and purification by preparative HPLC (reversed phase, eluent: water with 0.05% formic acid/acetonitrile: 95:5 to 10:90). Yield: 117 mg (81% of theory).

LC-MS (method 5): Rₜ=1.31/1.35 min; MS (ESIneg): m/z=550 [M−H]⁻

¹H-NMR (500 MHz, DMSO-d₆): δ [ppm]=10.69/10.63 (2s, 1H), 8.04-7.93 (m, 1H), 7.89-7.80 (m, 3H), 7.75-7.66 (m, 2H), 7.56-7.47 (m, 1H), 7.41-7.36 (m, 1H), 7.35/7.23 (2d, 1H), 7.24 (m, 1H), 6.40/6.39 (2s, 1H), 5.85-5.60 (m, 1H), 4.57/4.51 (2d, 1H), 3.66-3.52 (m, 2H), 3.48-3.47 (m, 1H), 3.49-3.34 (m, 1H), 3.27-3.20 (m, 1H), 2.86-2.76 (m, 1H), 2.44-2.22 (m, 3H), 1.26/1.24 (2d, 3H), 1.03/1.02 (2s, 9H). Additional signals of minor rotamers were also detected.

### Example 24

### 4-({(2S)-4-tert-Butoxy-2-[(7R)-11-chloro-7-methyl-2-oxo-7,8-dihydro-2H-[3]benzoxocino[5,6-c]pyridin-3(5H)-yl]butanoyl}amino)benzamide (Single Stereoisomer)

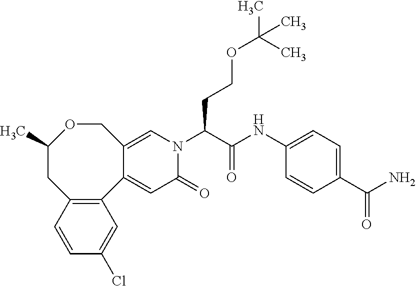 [SMILES: C[C@@H]1Cc2ccc(Cl)cc2-c2cc(=O)n([C@@H](CCOC(C)(C)C)C(=O)Nc3ccc(C(N)=O)cc3)cc2CO1]

Stereoisomer separation of 117 mg of 4-{[4-tert-butoxy-2-(11-chloro-7-methyl-2-oxo-7,8-dihydro-2H-[3]benzoxocino[5,6-c]pyridin-3(5H)-yl)butanoyl]amino}benzamide (mixture of stereoisomers), Example 23 gave
single stereoisomer 1 (chiral HPLC: Rₜ=5.94 min): 40 mg,
single stereoisomer 2 (the title compound Example 24) (chiral HPLC: Rₜ=7.46 min, 99% de): 26 mg,
single stereoisomer 3 (chiral HPLC: Rₜ=8.04 min): 23 mg,
single stereoisomer 4 (chiral HPLC: Rₜ=10.13 min): 32 mg.

First separation method: HPLC: column: Daicel Chiralpak ID 5 μm, 250 mm×20 mm; eluent: 50% n-heptane/50% 2-propanol with 0.2% trifluoroacetic acid; temperature: 40° C.; flow rate: 15 ml/min; UV detection: 220 nm.

Second separation method: Single stereoisomer 2 and single stereoisomer 3 eluted as a mixture in the first separation method. Their fractions were combined and the stereoisomers separated with the following conditions: HPLC: column: Daicel Chiralpak ID 5 μm, 250 mm×20 mm; eluent: 50% n-heptane/50% 2-propanol with 0.2% trifluoroacetic acid; temperature: 40° C.; flow rate: 15 ml/min; UV detection: 220 nm.

Analysis method: HPLC: column: Daicel Chiralpak ID 5 μm, 250 mm×4.6 mm; eluent: 50% 2-methylpentane/50% 2-propanol with 1% water and 0.2% trifluoroacetic acid; temperature: 40° C.; flow rate: 1 m/min; UV detection: 220 nm.

LC-MS (method 1): Rₜ=1.04 min; MS (ESIpos): m/z=552 [M+H]⁺

¹H-NMR (400 MHz, DMSO-d₆): δ [ppm]=10.63 (s, 1H), 7.99 (s, 1H), 7.88-7.80 (m, 3H), 7.72 (d, 2H), 7.50 (dd, 1H), 7.42-7.37 (m, 1H), 7.27-7.21 (br s, 1H), 7.23 (d, 1H), 6.40 (s, 1H), 5.77-5.66 (m, 1H), 4.57 (d, 1H), 3.64-3.50 (m, 2H), 3.42-3.34 (m, 1H), 3.28-3.19 (m, 1H), 2.82 (d, 1H), 2.44-2.22 (m, 3H), 1.26 (d, 3H), 1.03 (s, 9H). Additional signals of minor rotamers were also detected.

### Example 25

### 4-tert-Butoxy-2-(11-chloro-7-methyl-2-oxo-7,8-dihydro-2H-[3]benzoxocino[5,6-c]pyridin-3(5H)-yl)-N-([1,2,4]triazolo[1,5-a]pyridin-7-yl)butanamide (Mixture of Stereoisomers)

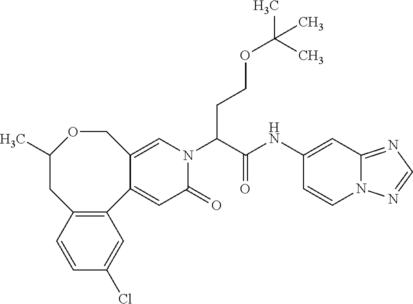 [SMILES: CC1Cc2ccc(Cl)cc2-c2cc(=O)n(C(CCOC(C)(C)C)C(=O)Nc3ccn4ncnc4c3)cc2CO1]

4-tert-Butoxy-2-(11-chloro-7-methyl-2-oxo-7,8-dihydro-2H-[3]benzoxocino[5,6-c]pyridin-3(5H)-yl)butanoic acid (mixture of stereoisomers) (35 mg, 0.08 mmol, 1.0 eq.) was dissolved in dichloromethane (2.5 ml) under argon atmosphere and 1-chloro-N,N,2-trimethylprop-1-en-1-amine (14 μl, 0.10 mmol, 1.3 eq.) was added. The reaction mixture was stirred at RT for 10 min, followed by addition of [1,2,4]triazolo[1,5-a]pyridin-7-amine (13.8 mg, 0.10 mmol, 1.3 eq.). Pyridine (32 μl, 0.4 mmol, 5.0 eq.) and T3P (141 μl, 50% solution in ethyl acetate, 0.24 mmol, 3.0 eq.) were added and the reaction mixture was stirred at 50° C. overnight. All volatiles were evaporated under reduced pressure and the residue was purified by preparative HPLC (reversed phase, eluent: water with 0.05% formic acid/acetonitrile 95:5 to 10:90). Yield: 28 mg (64% of theory).

LC-MS (method 3): Rₜ=1.33/1.36 min; MS (ESIneg): m/z=548 [M−H]⁻

¹H-NMR (400 MHz, DMSO-d₆): δ [ppm]=11.05/10.96 (2s, 1H), 8.90-8.84 (m, 1H), 8.41-8.35 (m, 1H), 8.27-8.16 (m, 1H), 8.07-7.99 (m, 1H), 7.60-7.44 (m, 1H), 7.43-7.22 (m, 3H), 6.48-6.35 (m, 1H), 5.84-5.70 (m, 1H), 4.58/4.53 (2d, 1H), 3.68-3.49 (m, 2H), 3.48-3.37 (m, 1H), 3.25-3.16 (m, 1H), 2.89-2.77 (m, 1H), 2.48-2.28 (m, 3H), 1.26/1.24 (2d, 3H), 1.03/1.02 (2s, 9H). Additional signals from minor rotamer were also detected.

### Example 26

### (2S)-4-tert-Butoxy-2-[(7R)-11-chloro-7-methyl-2-oxo-7,8-dihydro-2H-[3]benzoxocino[5,6-c]pyridin-3(5H)-yl]-N-([1,2,4]triazolo[1,5-a]pyridin-7-yl)butanamide (Single Stereoisomer)

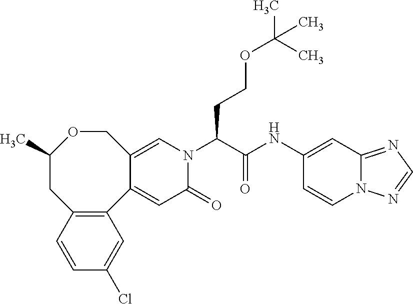 [SMILES: C[C@@H]1Cc2ccc(Cl)cc2-c2cc(=O)n([C@@H](CCOC(C)(C)C)C(=O)Nc3ccn4ncnc4c3)cc2CO1]

Stereoisomer separation of 28 mg of 4-tert-butoxy-2-(11-chloro-7-methyl-2-oxo-7,8-dihydro-2H-[3]benzoxocino[5,6-c]pyridin-3(5H)-yl)-N-([1,2,4]triazolo[1,5-a]pyridin-7-yl)-butanamide (mixture of stereoisomers), Example 25 gave
single stereoisomer 1 (chiral HPLC: Rₜ=6.31 min): 7 mg,
single stereoisomer 2 (chiral HPLC: Rₜ=8.14 min): 5 mg,
single stereoisomer 3 (the title compound Example 26): 8 mg (chiral HPLC: Rₜ=10.78 min, 99% de),
single stereoisomer 4 (chiral HPLC: Rₜ=14.38 min): 6 mg.

Separation method: HPLC: column: Daicel Chiralpak IF 5 μm, 250 mm×20 mm; eluent: 50% n-heptane/50% 2-propanol with 0.2% trifluoroacetic acid; temperature: 50° C.; flow rate: 15 ml/min; UV detection: 220 nm.

Analysis method: HPLC: column: Daicel Chiralpak IF 5 μm, 250 mm×4.6 mm; eluent: 50% n-heptane/50% 2-propanol with 1% water and 0.2% trifluoroacetic acid; temperature: 50° C.; flow rate: 1 ml/min; UV detection: 220 nm.

LC-MS (method 4): Rₜ=1.04 min; MS (ESIpos): m/z=550 [M+H]⁺

¹H-NMR (400 MHz, DMSO-d₆): δ [ppm]=11.00 (s, 1H), 8.90 (d, 1H), 8.47 (s, 1H), 8.25 (s, 1H), 8.01 (s, 1H), 7.51 (d, 1H), 7.35-7.44 (m, 2H), 7.24 (s, 1H), 6.42 (s, 1H), 5.79-5.67 (m, 1H), 4.58 (d, 1H), 3.57 (d, 2H), 3.48-3.35 (m, 1H), 3.28-3.19 (m, 1H), 2.83 (d, 1H), 2.43-2.25 (m, 3H), 1.27 (d, 3H), 1.03 (s, 9H). Additional signals of minor rotamers were also detected.

### Example 27

### 4-{[(2S)-2-[(7R)-11-Chloro-7-methyl-2-oxo-7,8-dihydro-2H-[3]benzoxocino[5,6-c]pyridin-3(5H)-yl]-4-(cyclopropyloxy)butanoyl]amino}benzamide (Single Stereoisomer)

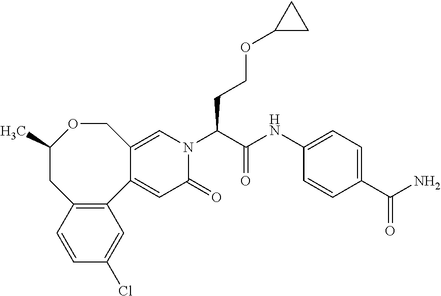 [SMILES: C[C@@H]1Cc2ccc(Cl)cc2-c2cc(=O)n([C@@H](CCOC3CC3)C(=O)Nc3ccc(C(N)=O)cc3)cc2CO1]

2-[(7R)-11-Chloro-7-methyl-2-oxo-7,8-dihydro-2H-[3]benzoxocino[5,6-c]pyridin-3(5H)-yl]-4-(cyclopropyloxy)butanoic acid (mixture of two diastereomers) (46 mg, 0.11 mmol), pyridine (10 μl, 0.12 mmol, 1.1 eq.) and T3P (96 μl, 50% solution in ethyl acetate, 0.17 mmol, 1.5 eq.) were added under argon atmosphere at RT to a solution of 4-aminobenzamide (16 mg, 0.12 mmol, 1.1 eq.) in tetrahydrofuran (3 ml). The reaction mixture was stirred at RT for 30 min before all volatiles were removed under reduced pressure. The residue was purified by preparative HPLC (reversed phase, eluent: acetonitrile/water gradient) to yield 40 mg of a mixture of 70% desired product 4-{2-[(7R)-11-chloro-7-methyl-2-oxo-7,8-dihydro-2H-[3]benzoxocino[5,6-c]pyridin-3(5H)-yl]-4-(cyclopropyloxy)butan-amido}benzamide (mixture of two diastereomers) and 30% starting material 2-[(7R)-11-chloro-7-methyl-2-oxo-7,8-dihydro-2H-[3]benzoxocino[5,6-c]pyridin-3(5H)-yl]-4-(cyclopropyloxy)butanoic acid (mixture of two diastereomers). This mixture, pyridine (3 μl, 0.03 mmol) and T3P (25 μl, 50% solution in ethyl acetate, 0.04 mmol) were added under argon atmosphere at RT to a solution of 4-aminobenzamide (4 mg, 0.03 mmol) in tetrahydrofuran (0.8 ml). The reaction mixture was stirred at RT for 30 min before all volatiles were removed under reduced pressure. The residue was purified by preparative HPLC (reversed phase, eluent: acetonitrile/water gradient) to yield
single stereoisomer 1 (Rₜ (method 3)=3.06 min): 4 mg (93% purity, 7% of theory),
single stereoisomer 2 (the title compound Example 27) (Rₜ (method 3)=3.15 min): 10 mg (>99% purity, 17% of theory).

LC-MS (method 3): Rₜ=3.15 min; MS (ESIpos): m/z=536 [M+H]⁺

### Example 28

### 4-({(2S)-2-[(7R)-11-Chloro-12-fluoro-7-methyl-2-oxo-7,8-dihydro-2H-[3]benzoxocino[5,6-c]pyridin-3(5H)-yl]butanoyl}amino)benzamide (Single Stereoisomer)

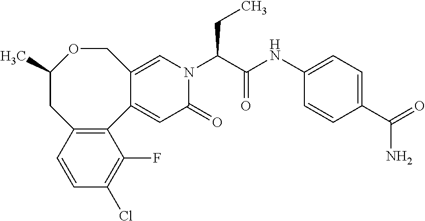 [SMILES: CC[C@@H](C(=O)Nc1ccc(C(N)=O)cc1)n1cc2c(cc1=O)-c1c(ccc(Cl)c1F)C[C@@H](C)OC2]

General Method 7 was carried out with 11-chloro-12-fluoro-7-methyl-3,5,7,8-tetrahydro-2H-[3]benzoxocino[5,6-c]pyridin-2-one (racemate) (10.7 mg, 36.4 μmol, 1.0 eq.), 4-{[(2R)-2-bromobutanoyl]amino}benzamide (single stereoisomer) (18.2 mg, 63.7 μmol, 1.8 eq.), 1,1,3,3-tetramethylguanidine (18 μl, 150 μmol, 4.0 eq.) in a mixture of 2-propanol/acetone (4:1, 0.34 ml) overnight including the following variations of the procedure: The crude mixture was purified by preparative HPLC (reversed phase, eluent: water with 0.05% formic acid/acetonitrile 80:20 to 5:95) and two diastereomers could be separated:
diastereomer 1 (LC-MS: Rₜ=0.88 min): 5.8 mg,
diastereomer 2 (the title compound 28) (LC-MS: Rₜ=0.93 min): 6.7 mg (81% purity, 30% of theory).

LC-MS (method 5): Rₜ=0.93 min; MS (ESIpos): m/z=498 [M+H]⁺

¹H-NMR (500 MHz, DMSO-d₆): δ [ppm]=10.74/10.73 (2s, 1H), 8.08 (s, 1H), 7.90-7.80 (m, 3H), 7.72-7.60 (m, 3H), 7.32-7.18 (m, 2H), 6.50/6.43 (2d, 1H), 5.67-5.54 (m, 1H), 4.58 (d, 1H), 4.11-3.96 (m, 1H), 3.64-3.48 (m, 2H), 2.87 (2d, 1H), 2.35-2.05 (m, 2H), 1.26 (d, 3H), 0.89 (t, 3H). Additional signals of minor rotamers were also detected.

### Example 29

### 4-({(2S,4S)-2-[(7R)-11-Chloro-12-fluoro-7-methyl-2-oxo-7,8-dihydro-2H-[3]benzoxocino[5,6-c]pyridin-3(5H)-yl]-4-methoxypentanoyl}amino)benzamide (Single Stereoisomer)

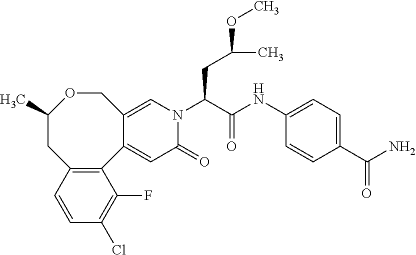 [SMILES: CO[C@@H](C)C[C@@H](C(=O)Nc1ccc(C(N)=O)cc1)n1cc2c(cc1=O)-c1c(ccc(Cl)c1F)C[C@@H](C)OC2]

1,1,3,3-Tetramethylguanidine (70 μl, 0.56 mmol, 3.0 eq.) was added under argon atmosphere at RT to a solution of 11-chloro-12-fluoro-7-methyl-3,5,7,8-tetrahydro-2H-[3]benzoxocino[5,6-c]pyridin-2-one (racemate) (55 mg, 0.19 mmol) in 2-propanol/acetone (4:1, 1.5 ml). The mixture was stirred at RT for 15 min, followed by addition of 4-{[(2R,4S)-2-bromo-4-methoxypentanoyl]amino}benzamide (single stereoisomer) (59 mg, 0.21 mmol, 1.1 eq.) and of further 2-propanol/acetone (4:1, 1.5 ml). The reaction mixture was stirred at RT for 5 days and concentrated under reduced pressure. The residue was purified by preparative HPLC (reversed phase, eluent: acetonitrile/water gradient) to give

4-{[(2S,4S)-2-(11-chloro-12-fluoro-7-methyl-2-oxo-7,8-dihydro-2H-[3]benzoxocino[5,6-c]pyridin-3(5H)-yl)-4-methoxypentanoyl]amino}-benzamide (mixture of two diastereomers): Yield. 64 mg (63% of theory) and

4-({(2S,4S)-2-[(7R)-11-chloro-12-fluoro-7-methyl-2-oxo-7,8-dihydro-2H-[3]benzoxocino[5,6-c]pyridin-3(5H)-yl]-4-methoxypentanoyl}amino)benzamide (single stereoisomer), the title compound Example 29: Yield. 12 mg (12% of theory)

LC-MS (method 1): Rₜ=0.92 min; MS (ESIpos): m/z=542 [M+H]⁺

¹H-NMR (600 MHz, DMSO-d₆): δ [ppm]=10.73-10.65 (m, 1H), 8.13/8.09 (2s, 1H), 7.89-7.80 (m, 3H), 7.74-7.67 (m, 2H), 7.66-7.57 (m, 1H), 7.29-7.16 (m, 2H), 6.50-6.40 (m, 1H), 5.87-5.80 (m, 1H), 4.59-4.52/4.28-4.23/4.04-3.99/3.62-3.56 (4m, 2H), 3.56-3.50 (m, 1H), 3.3-3.23 (m, 1H, partially concealed), 3.16/3.10 (2s, 3H), 2.89-2.80 (m, 1H), 2.36-2.16 (m, 3H), 1.28-1.22 (m, 3H), 1.18-1.10 (m, 3H). Additional signals of minor rotamers were also detected.

### Example 30

### 4-{2-[(7R)-11-Chloro-7-ethyl-2-oxo-7,8-dihydro-2H-[3]benzoxocino[5,6-c]pyridin-3(5H)-yl]-3-[(2S)-tetrahydro-2H-pyran-2-yl]propanamido}-2-fluorobenzamide (Mixture of Two Diastereomers)

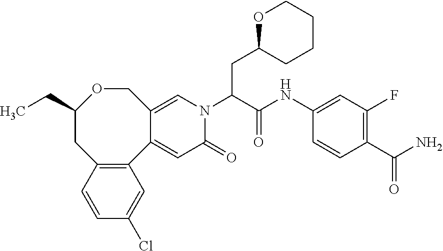 [SMILES: CC[C@@H]1Cc2ccc(Cl)cc2-c2cc(=O)n(C(C[C@@H]3CCCCO3)C(=O)Nc3ccc(C(N)=O)c(F)c3)cc2CO1]

2-[(7R)-11-Chloro-7-ethyl-2-oxo-7,8-dihydro-2H-[3]benzoxocino[5,6-c]pyridin-3(5H)-yl]-3-[(2S)-tetrahydro-2H-pyran-2-yl]propanoic acid (mixture of two diastereomers) (30 mg, 0.07 mmol) and 4-amino-2-fluorobenzamide (16 mg, 0.10 mmol, 1.5 eq.) were reacted according to General Method 11. Yield: 26 mg (66% of theory).

LC-MS (method 4): Rₜ=2.10/2.16 min; MS (ESIpos): m/z=582 [M+H]⁺

¹H-NMR (400 MHz, DMSO-d₆): δ [ppm]=10.81/10.79 (2s, 1H), 8.02/7.96 (2s, 1H), 7.73-7.60 (m, 2H), 7.57-7.44 (m, 4H), 7.42-7.35 (m, 2H), 6.39/6.38 (2s, 1H), 5.77-5.69 (m, 1H), 4.61/4.54 (2d, 1H), 3.84/3.78 (2d, 1H), 3.57/3.54 (2d, 1H), 3.28-3.09 (m, 2H, partially concealed), 2.85-2.77 (m, 1H), 2.34-2.25 (m, 2H), 2.12-2.05 (m, 1H), 1.80-1.70 (m, 1H), 1.65-1.54 (m, 3H), 1.47-1.32 (m, 4H), 1.31-1.20 (m, 1H), 0.95 (t, 3H). Additional signals of minor rotamers were also detected.

### Example 31

### 4-({(2S)-2-[(7R)-11-Chloro-7-ethyl-2-oxo-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (Single Stereoisomer)

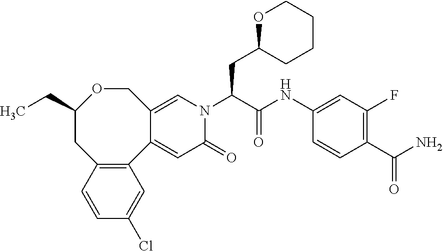 [SMILES: CC[C@@H]1Cc2ccc(Cl)cc2-c2cc(=O)n([C@@H](C[C@@H]3CCCCO3)C(=O)Nc3ccc(C(N)=O)c(F)c3)cc2CO1]

Diastereomer separation of 24 mg of 4-{2-(11-chloro-7-ethyl-2-oxo-7,8-dihydro-2H-[3]benzoxocino[5,6-c]pyridin-3(5H)-yl)-3-[(2S)-tetrahydro-2H-pyran-2-yl]propanamido}-2-fluorobenzamide (mixture of stereoisomers), Example 30 gave
single stereoisomer 1 (chiral HPLC: Rₜ=1.82 min): 6 mg,
single stereoisomer 2 (the title compound Example 31) (chiral HPLC: Rₜ=3.15 min, >99% de): 12 mg.

Separation method: SFC: column: Daicel Chiralpak AD SFC, 250 mm×20 mm; eluent: 75% carbon dioxide/25% 2-propanol; temperature: 40° C.; flow rate: 80 m/min; UV detection: 210 nm.

Analysis method: SFC: column: Daicel Chiralpak AD SFC, 250 mm×4.6 mm; eluent: 75% carbon dioxide/25% 2-propanol; temperature: 40° C.; flow rate: 3.0 ml/min; UV detection: 210 nm.

LC-MS (method 4): Rₜ=2.12 min; MS (ESIpos): m/z=582 [M+H]⁺

¹H-NMR (400 MHz, DMSO-d₆): δ [ppm]=10.78 (s, 1H), 8.02 (s, 1H), 7.72-7.65 (m, 2H), 7.57-7.44 (m, 4H), 7.38-7.33 (m, 2H), 6.39 (s, 1H), 5.74 (t, 1H), 4.61 (d, 1H), 3.84 (d, 1H), 3.54 (d, 1H), 3.28-3.18 (m, 2H, partially concealed), 2.81 (d, 1H), 2.34-2.25 (m, 2H), 2.12-2.04 (m, 1H), 1.77-1.70 (m, 1H), 1.65-1.53 (m, 3H), 1.47-1.36 (m, 4H), 1.30-1.20 (m, 1H), 0.95 (t, 3H). Additional signals of minor rotamers were also detected.

### Example 32

### 4-{2-[11-Chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanamido}-2-fluorobenzamide (Mixture of Stereoisomers)

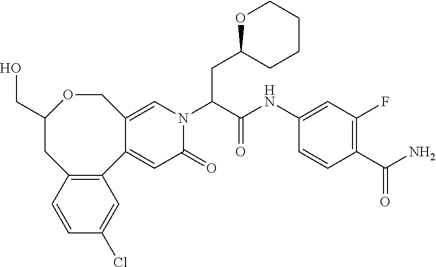 [SMILES: NC(=O)c1ccc(NC(=O)C(C[C@@H]2CCCCO2)n2cc3c(cc2=O)-c2cc(Cl)ccc2CC(CO)OC3)cc1F]

2-[11-Chloro-7-(hydroxymethyl)-2-oxo-7,8-dihydro-2H-[3]benz-oxocino[5,6-c]pyridin-3(5H)-yl]-3-[(2S)-tetrahydro-2H-pyran-2-yl]propanoic acid (mixture of stereoisomers) (125 mg, 0.28 mmol) and 4-amino-2-fluorobenzamide (64 mg, 0.42 mmol, 1.5 eq.) were reacted according to General Method 11. Yield (mixture of stereoisomers 1): 19 mg (12% of theory); Yield (mixture of stereoisomers 2): 21 mg (13% of theory).
mixture of stereoisomers 1:

LC-MS (method 10): Rₜ=1.35/1.38 min; MS (ESIpos): m/z=584 [M+H]⁺

¹H-NMR (600 MHz, DMSO-d₆): δ [ppm]=10.88/10.83 (s, 1H), 7.99/7.96 (2s, 1H), 7.74-7.60 (m, 2H), 7.60-7.50 (m, 3H), 7.47-7.31 (m, 3H), 6.41/6.40 (2s, 1H), 5.80/5.73 (2t, 1H), 4.97-4.88 (m, 1H), 4.57 (d, 1H), 3.86/3.77 (2d, 1H), 3.61-3.51 (m, 2H), 3.33-3.27 (m, 1H), 3.27-3.20 (m, 1H), 3.20-3.11 (m, 1H), 3.06-2.94 (m, 1H), 2.39-2.24 (m, 2H), 2.23-2.07 (m, 1H), 1.80-1.71 (m, 1H), 1.66-1.51 (m, 1H), 1.48-1.35 (m, 4H), 1.35-1.19 (m, 1H). Additional signals of minor rotamers were also detected.
mixture of stereoisomers 2:

LC-MS (method 10): Rₜ=1.45 min; MS (ESIpos): m/z=584 [M+H]⁺

¹H-NMR (600 MHz, DMSO-d₆): δ [ppm]=10.84/10.82 (2s, 1H), 8.01/8.00 (s, 1H), 7.74-7.61 (m, 2H), 7.60-7.50 (m, 3H), 7.49-7.42 (m, 1H), 7.38-7.35 (m, 1H), 7.34-7.31 (m, 1H), 6.41/6.40 (2s, 1H), 5.80-5.70 (m, 1H), 4.96-4.89 (m, 1H), 4.69-4.56 (m, 1H), 3.89-3.75 (m, 1H), 3.61-3.50 (m, 2H), 3.33-3.29 (m, 1H), 3.28-3.24 (m, 1H), 3.24-3.13 (m, 1H), 3.08-2.93 (m, 1H), 2.39-2.25 (m, 1H), 2.25-2.14 (m, 1H), 2.13-2.03 (m, 1H), 1.79-1.69 (m, 1H), 1.65-1.50 (m, 1H), 1.47-1.34 (m, 4H), 1.33-1.18 (m. 1H). Additional signals of minor rotamers were also detected.

### Example 33

### 4-({(2S)-2-[(7S)-11-Chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (Single Stereoisomer)

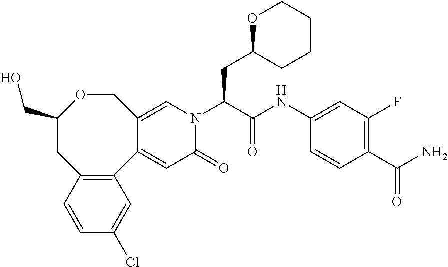 [SMILES: NC(=O)c1ccc(NC(=O)[C@H](C[C@@H]2CCCCO2)n2cc3c(cc2=O)-c2cc(Cl)ccc2C[C@@H](CO)OC3)cc1F]

Stereoisomer separation of 17 mg of 4-{2-[11-chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanamido}-2-fluorobenzamide (mixture of stereoisomers 2 of Example 32) gave
single stereoisomer 1 (the title compound Example 33) (chiral HPLC: Rₜ=1.67 min, 99% de): 4 mg,
single stereoisomer 2 (chiral HPLC: Rₜ=2.51 min): 5.3 mg.

Separation method: HPLC: column: Daicel Chiralpak IB 5 μm, 250 mm×20 mm; eluent: 80% n-heptane/20% ethanol; temperature: 40° C.; flow rate: 15 ml/min; UV detection: 220 nm.

Analysis method: HPLC: column: Daicel IB-3 3 μm, 50 mm×4.6 mm; eluent: 80% n-heptane/20% ethanol; temperature: 25° C.; flow rate: 1 ml/min; UV detection: 220 nm.

LC-MS (method 10): Rₜ=1.45 min; MS (ESIpos): m/z=584 [M+H]⁺

¹H-NMR (600 MHz, DMSO-d₆): δ [ppm]=10.84 (br s, 1H), 8.01 (s, 1H), 7.71-7.65 (m, 1H), 7.56 (br d, 2H), 7.52 (dd, 1H), 7.46 (d, 1H), 7.36 (d, 1H), 7.32 (d, 1H), 6.40 (s, 1H), 5.76 (t, 1H), 4.63 (d, 1H), 3.88-3.79 (m, 1H), 3.58-3.49 (m, 2H), 3.32-3.28 (m, 1H), 3.28-3.19 (m, 2H), 2.97 (d, 1H), 2.26 (br d, 1H), 2.19 (dd, 1H), 2.13-2.03 (m, 1H), 1.78-1.71 (m, 1H), 1.60 (d, 1H), 1.50-1.34 (m, 4H), 1.33-1.18 (m, 2H). Additional signals of minor rotamers were also detected.

### Example 34

### 4-{2-[11-Chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanamido}benzamide (Mixture of Stereoisomers)

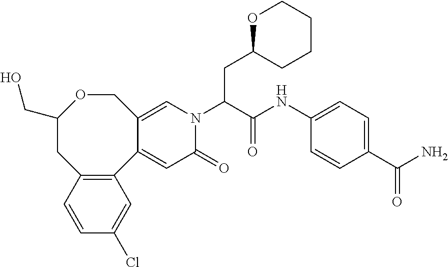 [SMILES: NC(=O)c1ccc(NC(=O)C(C[C@@H]2CCCCO2)n2cc3c(cc2=O)-c2cc(Cl)ccc2CC(CO)OC3)cc1]

2-[11-Chloro-7-(hydroxymethyl)-2-oxo-7,8-dihydro-2H-[3]benz-oxocino[5,6-c]pyridin-3(5H)-yl]-3-[(2S)-tetrahydro-2H-pyran-2-yl]propanoic acid (mixture of stereoisomers) (125 mg, 0.28 mmol, 1.0 eq.) and 4-aminobenzamide (57 mg, 0.42 mmol, 1.5 eq.) were reacted according to General Method 11. Yield (mixture of stereoisomers 1): 19 mg (12% of theory); Yield (mixture of stereoisomers 2): 20 mg (13% of theory).
mixture of stereoisomers 1:

LC-MS (method 10): Rₜ=1.30/1.33 min; MS (ESIpos): m/z=564 [M+H]⁺

¹H-NMR (600 MHz, DMSO-d₆): δ [ppm]=10.72/10.67 (2br s, 1H), 8.02-7.93 (m, 1H), 7.90-7.81 (m, 3H), 7.74-7.70 (m, 1H), 7.70-7.66 (m, 1H), 7.55-7.44 (m, 1H), 7.39-7.36 (m, 1H), 7.34-7.31 (m, 1H), 7.30-7.21 (m, 1H), 6.40/6.39 (2s, 1H), 5.83/5.77 (2t, 1H), 4.92 (t, 1H), 4.57 (d, 1H), 3.86/3.78 (2d, 1H), 3.62-3.46 (m, 2H), 3.45-3.38 (m, 1H), 3.34-3.20 (m, 2H), 3.19-3.10 (m, 1H), 3.06-2.94 (m, 1H), 2.35-2.24 (m, 2H), 2.24-2.07 (m, 1H), 1.77-1.74 (m, 1H), 1.61 (d, 1H), 1.47-1.34 (m, 3H), 1.34-1.20 (m, 1H). Additional signals of minor rotamers were also detected.
mixture of stereoisomers 2:

LC-MS (method 10): Rₜ=1.32/1.41 min; MS (ESIpos): m/z=564 [M+H]⁺

¹H-NMR (600 MHz, DMSO-d₆): δ [ppm]=10.70/10.62 (2s, 1H), 8.05-7.96 (m, 1H), 7.92-7.82 (m, 3H), 7.77-7.66 (m, 2H), 7.57-7.45 (m, 1H), 7.40-7.26 (m, 3H), 6.41/6.40 (2s, 1H), 5.85-5.73 (m, 1H), 4.93 (br s, 1H), 4.69-4.56 (m, 1H), 3.88-3.77 (m, 1H), 3.61-3.50 (m, 2H), 3.34-3.16 (m, 3H), 3.08-2.93 (m, 2H), 2.34-2.16 (m, 2H), 2.12-2.03 (m, 1H), 1.80-1.68 (m, 1H), 1.65-1.55 (m, 1H), 1.47-1.35 (m, 3H), 1.32-1.18 (m, 1H). Additional signals of minor rotamers were also detected.

### Example 35

### 4-({(2S)-2-[(7S)-11-Chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanoyl}amino)benzamide (Single Stereoisomer)

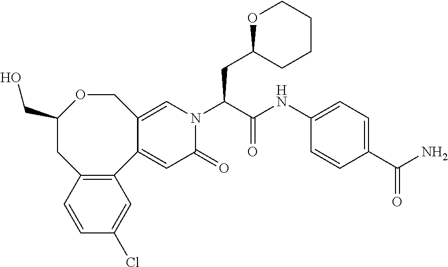 [SMILES: NC(=O)c1ccc(NC(=O)[C@H](C[C@@H]2CCCCO2)n2cc3c(cc2=O)-c2cc(Cl)ccc2C[C@@H](CO)OC3)cc1]

Stereoisomer separation of 16 mg of 4-{2-[11-chloro-7-(hydroxymethyl)-2-oxo-7,8-dihydro-2H-[3]benzoxocino[5,6-c]pyridin-3(5H)-yl]-3-[(2S)-tetrahydro-2H-pyran-2-yl]propanamido}-benzamide (mixture of stereoisomers 2 of Example 34) gave
single stereoisomer 1 (the title compound Example 35) (chiral HPLC: Rₜ=2.99 min, 99% de): 3.4 mg,
single stereoisomer 2 (chiral HPLC: Rₜ=3.76 min): 4.9 mg.

Separation method: HPLC: column: Daicel Chiralpak IB 5 μm, 250 mm×20 mm; eluent: 80% n-heptane/20% ethanol; temperature: 40° C.; flow rate: 15 m/min; UV detection: 220 nm.

Analysis method: HPLC: column: Daicel Chiraltek IB 3 μm, 250 mm×4.6 mm; eluent: 70% n-heptane/30% ethanol; temperature: 25° C.; flow rate: 1 ml/min; UV detection: 220 nm.

LC-MS (method 10): Rₜ=1.41 min; MS (ESIneg): m/z=566 [M−H]⁻

¹H-NMR (600 MHz, DMSO-d₆): δ [ppm]=10.70 (s, 1H), 8.02 (s, 1H), 7.89 (br s, 1H), 7.85 (d, 2H), 7.73 (d, 2H), 7.51 (dd, 1H), 7.36 (d, 1H), 7.32 (d, 1H), 7.30-7.24 (m, 1H), 6.40 (s, 1H), 5.80 (t, 1H), 4.98-4.88 (m, 1H), 4.63 (d, 1H), 3.89-3.81 (m, 1H), 3.56 (d, 1H), 3.54-3.49 (m, 1H), 3.32-3.17 (m, 3H), 2.97 (d, 1H), 2.31-2.23 (m, 1H), 2.19 (dd, 1H), 2.11-2.00 (m, 1H), 1.80-1.71 (m, 1H), 1.65-1.57 (m, 1H), 1.48-1.35 (m, 3H), 1.31-1.19 (m, 2H). Additional signals of minor rotamers were also detected.

### Example 36

### 4-{2-[11-Chloro-7-(hydroxymethyl)-2-oxo-7,8-dihydro-2H-[3]benzoxocino[5,6-c]pyridin-3(5H)-yl]-3-[(2R)-1,4-dioxan-2-yl]propanamido}benzamide (Mixture of Stereoisomers)

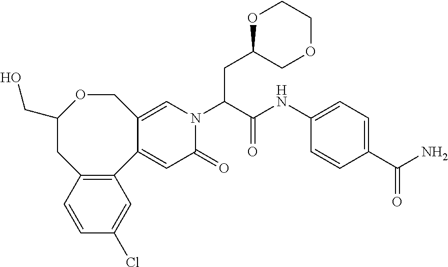 [SMILES: NC(=O)c1ccc(NC(=O)C(C[C@@H]2COCCO2)n2cc3c(cc2=O)-c2cc(Cl)ccc2CC(CO)OC3)cc1]

2-[11-Chloro-7-(hydroxymethyl)-2-oxo-7,8-dihydro-2H-[3]benzoxocino[5,6-c]pyridin-3(5H)-yl]-3-[(2R)-1,4-dioxan-2-yl]propanoic acid (mixture of stereoisomers) (105 mg, 92% purity, 0.22 mmol, 1.0 eq.) and 4-aminobenzamide (44 mg, 0.32 mmol, 1.5 eq.) were reacted according to General Method 11. Yield: 105 mg (94% purity, 81% of theory).

LC-MS (method 3): Rₜ=1.95/1.97/2.04/2.10 min; MS (ESIpos): m/z=568 [M+H]⁺

¹H-NMR (500 MHz, DMSO-d₆): δ [ppm]=10.73/10.66/10.62 (3s, 1H), 8.06-7.92 (m, 1H), 7.92-7.80 (m, 3H), 7.76-7.64 (m, 2H), 7.56-7.43 (m, 1H), 7.43-7.29 (m, 2H), 7.28-7.17 (m, 1H), 6.42-6.38 (m, 1H), 5.82-5.68 (m, 1H), 5.03-4.80 (br s, 1H), 4.69-4.51 (m, 1H), 3.84-3.64 (m, 3H), 3.62-3.53 (m, 3H), 3.48-3.25 (m, 12H), 3.19-3.13 (m, 3H), 3.06-2.87 (m, 1H), 2.30-2.10 (m, 3H), 1.87-1.70 (m, 1H), 1.60-1.49 (m, 4H), 1.48-1.38 (m, 1H), 1.38-1.23 (m, 4H), 0.94 (t, 6H), 0.91-0.84 (m, 1H). Additional signals of minor rotamers were also detected.

### Example 37

### 4-({(2S)-2-[(7S)-11-Chloro-7-(hydroxymethyl)-2-oxo-7,8-dihydro-2H-[3]benzoxocino[5,6-c]pyridin-3(5H)-yl]-3-[(2R)-1,4-dioxan-2-yl]propanoyl}amino)benzamide (Single Stereoisomer)

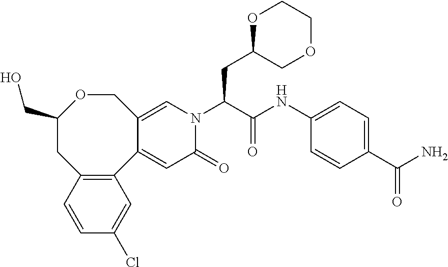 [SMILES: NC(=O)c1ccc(NC(=O)[C@H](C[C@@H]2COCCO2)n2cc3c(cc2=O)-c2cc(Cl)ccc2C[C@@H](CO)OC3)cc1]

Stereoisomer separation of 105 mg of 4-{2-[11-chloro-7-(hydroxymethyl)-2-oxo-7,8-dihydro-2H-[3]benzoxocino[5,6-c]pyridin-3(5H)-yl]-3-[(2R)-1,4-dioxan-2-yl]propanamido}benzamide (mixture of stereoisomers), Example 36 in analogy to Example 35 gave
single stereoisomer 1 (LC-MS (method 3): Rₜ=1.95 min): 6.7 mg,
single stereoisomer 2 (LC-MS (method 3): %=1.98): 4.1 mg, 56% purity,
single stereoisomer 3 (LC-MS (method 3): Rₜ=2.03): 19.2 mg, 67% purity,
single stereoisomer 4 (the title compound Example 37) (LC-MS (method 3): Rₜ=2.10 min, 99% de): 12.3 mg, 81% purity.

LC-MS (method 3): Rₜ=2.04/2.10 min; MS (ESIpos): m/z=568 [M+H]⁺

¹H-NMR (500 MHz, DMSO-d₆): δ [ppm]=10.85/10.77/10.66 (3s, 1H), 8.04-7.99 (m, 1H), 7.92-7.82 (m, 3H), 7.74-7.66 (m, 2H), 7.53-7.40 (m, 1H), 7.39-7.35 (m, 1H), 7.34-7.30 (m, 1H), 7.26-7.21 (m, 1H) 6.41/6.40 (2s, 1H), 5.81-5.70 (m, 1H), 5.11-4.80 (m, 1H), 4.62 (d, 1H), 3.76-3.66 (m, 2H), 3.64-3.55 (m, 2H), 3.54-3.41 (m, 4H), 3.30-3.22 (m, 2H), 3.19-3.12 (m, 4H), 2.98 (d, 1H), 2.33-2.03 (m, 3H), 1.88-1.68 (m, 1H), 1.62-1.51 (m, 5H), 1.48-1.37 (m, 1H), 1.35-1.27 (m, 4H), 0.94 (t, 7H), 0.91-0.86 (m, 1H). Additional signals of minor rotamers were also detected.

### Example 38

### 4-({2-(11-Chloro-7-isopropyl-2-oxo-7,8-dihydro-2H-[3]benzoxocino[5,6-c]pyridin-3(5H)-yl)-3-[(2S)-tetrahydro-2H-pyran-2-yl]propanoyl}amino)-2-fluorobenzamide (Mixture of Stereoisomers)

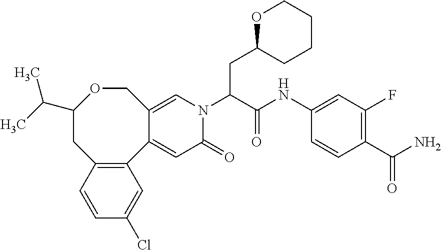 [SMILES: CC(C)C1Cc2ccc(Cl)cc2-c2cc(=O)n(C(C[C@@H]3CCCCO3)C(=O)Nc3ccc(C(N)=O)c(F)c3)cc2CO1]

2-(11-Chloro-7-isopropyl-2-oxo-7,8-dihydro-2H-[3]benzoxocino[5,6-c]pyridin-3(5H)-yl)-3-[(2S)-tetrahydro-2H-pyran-2-yl]propanoic acid (mixture of stereoisomers) (70 mg, 86% purity, 0.13 mmol) was dissolved in pyridine (1 ml) under argon atmosphere and T3P (58 μl, 50% solution in ethyl acetate, 0.24 mmol) was added. The reaction mixture was heated to 50° C. and 4-amino-2-fluorobenzamide (30 mg, 0.20 mmol, 1.63 eq.) was added. After stirring at 60° C. for 1 h, T3P (40 μl, 50% solution in ethyl acetate, 0.17 mmol) was added and the mixture was stirred for 1 h at 60° C. A mixture of acetonitrile (1 ml) and water (0.5 ml) was added. The forming precipitate was collected by filtration and the mother liquor was concentrated under reduced pressure. Both, precipitate and residue were each purified by preparative HPLC (reversed phase, eluent: water with 0.01% trifluoroacetic acid/acetonitrile gradient 90:10 to 10:90) and both product batches were combined. Yield: 71 mg (92% of theory).

LC-MS (method 5): Rₜ=1.51 min; MS (ESIpos): m/z=596 [M+H]⁺

### Example 39

### 4-({(2S)-2-[(7S)-11-Chloro-7-isopropyl-2-oxo-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (Single Stereoisomer)

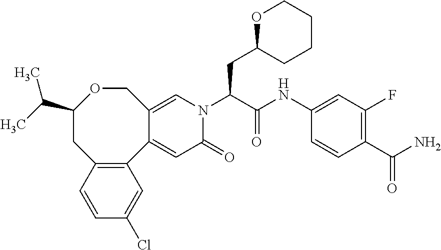 [SMILES: CC(C)[C@@H]1Cc2ccc(Cl)cc2-c2cc(=O)n([C@@H](C[C@@H]3CCCCO3)C(=O)Nc3ccc(C(N)=O)c(F)c3)cc2CO1]

Stereoisomer separation of 71 mg of 4-({2-(11-chloro-7-isopropyl-2-oxo-7,8-dihydro-2H-[3]benz-oxocino[5,6-c]pyridin-3(5H)-yl)-3-[(2S)-tetrahydro-2H-pyran-2-yl]propanoyl}amino)-2-fluoro-benzamide (mixture of stereoisomers), Example 38 gave
single stereoisomer 1 (the title compound Example 39) (chiral HPLC: Rₜ=3.22 min, >95% de): 18 mg,
single stereoisomer 2 (chiral HPLC: Rₜ=4.24 min): 7 mg,
single stereoisomer 3 (chiral HPLC: Rₜ=5.43 min): 12 mg, 60% purity,
single stereoisomer 4 (chiral HPLC: Rₜ=6.45 min): 20 mg.

Separation method: HPLC: column: Daicel Chiralpak IE 5 μm, 250 mm×20 mm; eluent: 40% n-heptane/60% ethanol; temperature: 30° C.; flow rate: 20 m/min; UV detection: 220 nm.

Analysis method: HPLC: column: Daicel Chiralpak IE 3 μm, 50 mm×4.6 mm; eluent: 50% n-heptane/50% ethanol; temperature: 30° C.; flow rate: 1 ml/min; UV detection: 220 nm.

LC-MS (method 7): Rₜ=6.56 min; MS (ESIpos): m/z=596 [M+H]⁺

¹H-NMR (400 MHz, DMSO-d₆): δ [ppm]=10.74 (s, 1H), 8.03 (s, 1H), 7.71-7.63 (m, 2H), 7.56-7.43 (m, 4H), 7.37-7.33 (m, 1H), 7.31 (d, 1H), 6.39 (s, 1H), 5.76-5.68 (m, 1H), 4.62 (d, 1H), 3.90-3.78 (m, 1H), 3.53 (d, 1H), 3.31-3.13 (m, 3H, partially concealed), 2.80 (d, 1H), 2.36-2.21 (m, 2H), 2.14-2.03 (m, 1H), 1.92-1.80 (m, 1H), 1.78-1.70 (m, 1H), 1.66-1.56 (m, 1H), 1.47-1.38 (m, 3H), 1.31-1.17 (m, 1H, partially concealed), 0.97-0.89 (m, 6H). Additional signals of minor rotamers were also detected.

### Example 40

### 4-({2-(11-Chloro-7-isopropyl-2-oxo-7,8-dihydro-2H-[3]benzoxocino[5,6-c]pyridin-3(5H)-yl)-3-[(2R)-1,4-dioxan-2-yl]propanoyl}amino)-2-fluorobenzamide (Mixture of Stereoisomers)

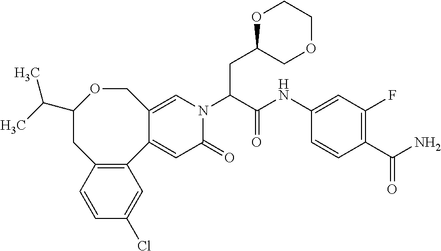 [SMILES: CC(C)C1Cc2ccc(Cl)cc2-c2cc(=O)n(C(C[C@@H]3COCCO3)C(=O)Nc3ccc(C(N)=O)c(F)c3)cc2CO1]

2-(11-Chloro-7-isopropyl-2-oxo-7,8-dihydro-2H-[3]benzoxocino[5,6-c]pyridin-3(5H)-yl)-3-[(2R)-1,4-dioxan-2-yl]propanoic acid (mixture of stereoisomers) (120 mg, 83% purity, 0.21 mmol) and 4-amino-2-fluorobenzamide (53 mg, 0.35 mmol, 1.63 eq.) were reacted according to General Method 11. Yield: 109 mg (82% of theory).

LC-MS (method 7): Rₜ=6.02/6.09 min; MS (ESIpos): m/z=598 [M+H]⁺

¹H NMR (400 MHz, DMSO-d₆): δ [ppm]=10.87/10.85/10.79/10.72 (4s, 1H), 8.03/8.00 (2s, 1H), 7.73-7.60 (m, 2H), 7.57-7.48 (m, 3H), 7.47-7.26 (m, 3H), 6.40 (s, 1H), 5.80-5.65 (m, 1H), 4.70-4.53 (m, 1H), 3.76-3.64 (m, 2H), 3.63-3.37 (m, 4H, partially concealed), 3.32-3.15 (m, 3H), 2.87-2.77 (m, 1H), 2.38-2.19 (m, 2H), 2.19-2.02 (m, 1H), 1.93-1.80 (m, 1H), 0.93 (m, 6H). Additional signals of minor rotamers were also detected.

### Example 41

### 4-({(2S)-2-[(7S)-11-Chloro-7-isopropyl-2-oxo-7,8-dihydro-2H-[3]benzoxocino[5,6-c]pyridin-3(5H)-yl]-3-[(2R)-1,4-dioxan-2-yl]propanoyl}amino)-2-fluorobenzamide (Single Stereoisomer)

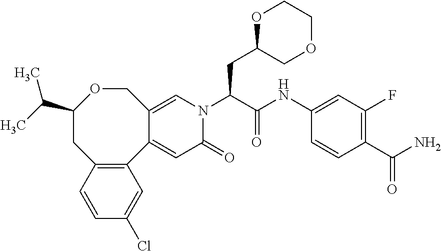 [SMILES: CC(C)[C@@H]1Cc2ccc(Cl)cc2-c2cc(=O)n([C@@H](C[C@@H]3COCCO3)C(=O)Nc3ccc(C(N)=O)c(F)c3)cc2CO1]

Stereoisomer separation of 105 mg of 4-({2-(11-chloro-7-isopropyl-2-oxo-7,8-dihydro-2H-[3]benz-oxocino[5,6-c]pyridin-3(5H)-yl)-3-[(2R)-1,4-dioxan-2-yl]propanoyl})amino)-2-fluorobenzamide (mixture of stereoisomers), Example 40 gave
single stereoisomer 1 (chiral HPLC: Rₜ=5.06 min): 14 mg,
single stereoisomer 2 (chiral HPLC: Rₜ=6.24 min): 37 mg.
single stereoisomer 3 (chiral HPLC: Rₜ=5.57 min): 11 mg,
single stereoisomer 4 (the title compound Example 41) (chiral HPLC: Rₜ=5.91 min, 99% de): 39 mg.

First separation method: HPLC: column: Daicel Chiralpak IF 5 μm, 250 mm×20 mm; eluent: 60% tert-butyl methyl ether/20% methanol/20% acetonitrile; temperature: 30° C.; flow rate: 15 ml/min; UV detection: 220 nm.

Second separation method: Single stereoisomer 1 and single stereoisomer 2 eluted as a mixture in the first separation. The mixture was purified according to the following conditions: HPLC: column: Daicel Chiralpak IE 5 μm 250 mm×20 mm; eluent: 50% tert-butyl methyl ether/50% methanol; temperature: 30° C.; flow rate: 15 ml/min; UV detection: 220 nm.

Analysis method: HPLC: column: Daicel Chiralpak IE 5 μm, 250 mm×4.6 mm; eluent: 50% ten-butyl methyl ether/50% methanol; temperature: 30° C.; flow rate: 1 ml/min; UV detection: 220 nm.

LC-MS (method 5): Rₜ=1.37 min; MS (ESIpos): m/z=598 [M+H]⁺

¹H-NMR (600 MHz, DMSO-d₆): δ [ppm]=10.71 (s, 1H), 8.02 (s, 1H), 7.73-7.61 (m, 2H), 7.55-7.47 (m, 3H), 7.47-7.41 (m, 1H), 7.39-7.34 (m, 1H), 7.33-7.27 (m, 1H), 6.40 (s, 1H), 5.74-5.66 (m, 1H), 4.65-4.47 (m, 1H), 3.76-3.71 (m, 1H), 3.70-3.65 (m, 1H), 3.62-3.58 (m, 1H), 3.57-3.41 (m, 4H), 3.28-3.22 (m, 1H, partially concealed), 3.20-3.15 (m, 1H), 2.85-2.75 (m, 1H), 2.30-2.21 (m, 2H), 2.12-2.04 (m, 1H), 1.89-1.80 (m, 1H), 0.96-0.88 (m, 6H). Additional signals of minor rotamers were also detected.

### Example 42

### 4-({2-(11-Chloro-7-isopropyl-2-oxo-7,8-dihydro-2H-[3]benzoxocino[5,6-c]pyridin-3(5H)-yl)-3-[(2R)-1,4-dioxan-2-yl]propanoyl}amino)benzamide (Mixture of Stereoisomers)

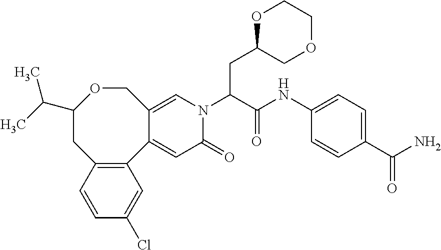 [SMILES: CC(C)C1Cc2ccc(Cl)cc2-c2cc(=O)n(C(C[C@@H]3COCCO3)C(=O)Nc3ccc(C(N)=O)cc3)cc2CO1]

2-(11-Chloro-7-isopropyl-2-oxo-7,8-dihydro-2H-[3]benzoxocino[5,6-c]pyridin-3(5H)-yl)-3-[(2R)-1,4-dioxan-2-yl]propanoic acid (mixture of stereoisomers) (100 mg, 83% purity, 0.18 mmol) and 4-aminobenzamide (40 mg, 0.29 mmol, 1.63 eq.) were reacted according to General Method 11. Yield: 92 mg (84% of theory).

LC-MS (method 7): Rₜ=5.88/5.94 min; MS (ESIpos): m/z=580 [M+H]⁺

¹H NMR (400 MHz, DMSO-d₆) δ [ppm]=10.70/10.69/10.64/10.60 (4s, 1H), 8.04/8.01/8.00 (3s, 1H), 7.91-7.81 (m, 3H), 7.72-7.64 (m, 2H), 7.54-7.47 (m, 1H), 7.40-7.34 (m, 1H), 7.34-7.28 (m, 1H), 7.27-7.20 (m, 1H), 6.40/6.40 (2s, 1H), 5.86-5.69 (m, 1H), 4.72-4.54 (m, 1H), 3.79-3.64 (m, 2H), 3.64-3.31 (m, 4H, partially concealed), 3.33-3.12 (m, 3H), 2.86-2.77 (m, 1H), 2.39-2.19 (m, 2H), 2.19-2.03 (m, 1H), 1.91-1.80 (m, 1H), 1.01-0.89 (m, 6H). Additional signals of minor rotamers were also detected.

### Example 43

### 4-({(2S)-2-[(7S)-11-Chloro-7-isopropyl-2-oxo-7,8-dihydro-2H-[3]benzoxocino[5,6-c]pyridin-3(5H)-yl]-3-[(2R)-1,4-dioxan-2-yl]propanoyl}amino)benzamide (Single Stereoisomer)

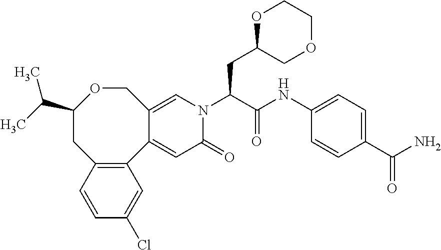 [SMILES: CC(C)[C@@H]1Cc2ccc(Cl)cc2-c2cc(=O)n([C@@H](C[C@@H]3COCCO3)C(=O)Nc3ccc(C(N)=O)cc3)cc2CO1]

Stereoisomer separation of 90 mg of 4-({2-(11-chloro-7-isopropyl-2-oxo-7,8-dihydro-2H-[3]benz-oxocino[5,6-c]pyridin-3(5H)-yl)-3-[(2R)-1,4-dioxan-2-yl]propanoyl}amino)benzamide (mixture of stereoisomers), Example 42 gave
single stereoisomer 1 (chiral HPLC: Rₜ=4.83 min): 7 mg,
single stereoisomer 2 (chiral HPLC: Rₜ=6.56 min): 5 mg, 86% purity,
single stereoisomer 3 (the title compound Example 43) (chiral HPLC: Rₜ=9.35 min, >95% de): 22 mg,
single stereoisomer 4 (chiral HPLC: Rₜ=5.91 min): 23 mg.

Separation method: HPLC: column: Daicel Chiralpak IE 5 μm, 250 mm×20 mm; eluent: 30% n-heptane/70% ethanol; temperature: 30° C.; flow rate: 20 m/min; UV detection: 265 nm.

Analysis method: HPLC: column: Daicel Chiralpak IE-3 μm, 50 mm×4.6 mm; eluent: 50% n-heptane/50% ethanol; temperature: 30° C.; flow rate: 1 ml/min; UV detection: 220 nm.

LC-MS (method 4): Rₜ=1.93 min; MS (ESIpos): m/z=580 [M+H]⁺

¹H-NMR (600 MHz, DMSO-d₆): δ [ppm]=10.57 (s, 1H), 8.03 (s, 1H), 7.89-7.79 (m, 3H), 7.73-7.67 (m, 2H), 7.52-7.48 (m, 1H), 7.49-7.35 (m, 1H), 7.33-7.28 (m, 1H), 7.29-7.16 (m, 1H), 6.40 (s, 1H), 5.76-5.71 (m, 1H), 4.61 (d, 1H), 3.76-3.72 (m, 1H), 3.70-3.66 (m, 1H), 3.63-3.58 (m, 1H), 3.56-3.41 (m, 4H), 3.28-3.23 (m, 1H, partially concealed), 3.19-3.15 (m, 1H), 2.82-2.76 (m, 1H), 2.29-2.23 (m, 2H), 2.11-2.03 (m, 1H), 1.90-1.81 (m, 1H), 0.95-0.89 (m, 6H). Additional signals of minor rotamers were also detected.

### Example 44

### 2-(11-Chloro-7-isopropyl-2-oxo-7,8-dihydro-2H-[3]benzoxocino[5,6-c]pyridin-3(5H)-yl)-3-[(2R)-1,4-dioxan-2-yl]-N-(2-methyl-2H-benzotriazol-5-yl)propanamide (Mixture of Stereoisomers)

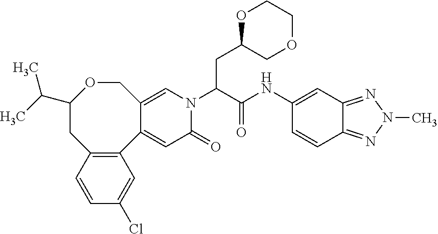 [SMILES: CC(C)C1Cc2ccc(Cl)cc2-c2cc(=O)n(C(C[C@@H]3COCCO3)C(=O)Nc3ccc4nn(C)nc4c3)cc2CO1]

2-(11-Chloro-7-isopropyl-2-oxo-7,8-dihydro-2H-[3]benzoxocino[5,6-c]pyridin-3(5H)-yl)-3-[(2R)-1,4-dioxan-2-yl]propanoic acid (mixture of stereoisomers) (55 mg, 83% purity, 0.18 mmol) and 2-methyl-2H-benzotriazol-5-amine (24 mg, 0.16 mmol, 1.63 eq.) were reacted according to General Method 11. Yield: 49 mg (79% of theory).

LC-MS (method 4): Rₜ=2.13/2.17 min; MS (ESIpos): m/z=592 [M+H]⁺

¹H-NMR (400 MHz, DMSO-d₆): δ [ppm]=10.74-10.62 (m, 1H), 8.36-8.30 (m, 1H), 8.10-8.02 (m, 1H), 7.90-7.84 (m, 1H), 7.56-7.47 (m, 2H), 7.43-7.35 (m, 1H), 7.35-7.29 (m, 1H), 6.41/6.41 (2s, 1H), 5.85-5.73 (m, 1H), 4.71-4.55 (m, 1H), 4.47-4.42 (m, 3H), 3.78-3.36 (m, 7H, partially concealed), 3.31-3.14 (m, 2H, partially concealed), 2.86-2.76 (m, 1H), 2.40-2.19 (m, 3H), 1.94-1.80 (m, 1H), 0.98-0.89 (m, 6H). Additional signals of minor rotamers were also detected.

### Example 45

### (2S)-2-[(7S)-11-Chloro-7-isopropyl-2-oxo-7,8-dihydro-2H-[3]benzoxocino[5,6-c]pyridin-3(5H)-yl]-3-[(2R)-1,4-dioxan-2-yl]-N-(2-methyl-2H-benzotriazol-5-yl)propanamide (Single Stereoisomer)

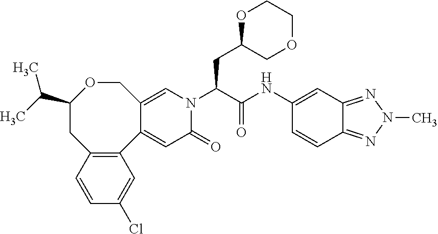 [SMILES: CC(C)[C@@H]1Cc2ccc(Cl)cc2-c2cc(=O)n([C@@H](C[C@@H]3COCCO3)C(=O)Nc3ccc4nn(C)nc4c3)cc2CO1]

Stereoisomer separation of 45 mg of 2-(11-chloro-7-isopropyl-2-oxo-7,8-dihydro-2H-[3]benzoxocino[5,6-c]pyridin-3(5H)-yl)-3-[(2R)-1,4-dioxan-2-yl]-N-(2-methyl-2H-benzotriazol-5-yl)propanamide (mixture of stereoisomers), Example 44 gave
mixture of single stereoisomers 1 and 2 (chiral HPLC: Rₜ=8.62/8.96 min): 7 mg,
single stereoisomer 3 (the title compound Example 45) (chiral HPLC: Rₜ=9.75 min, 99% de): 14 mg,
single stereoisomer 4 (chiral HPLC: Rₜ=11.35 min): 17 mg.

Separation method: HPLC: column: Daicel Chiralpak IC 5 μm, 250 mm×20 mm; eluent: 50% n-heptane/50% 2-propanol with 1% water and 0.2% trifluoroacetic acid; temperature: 40° C.; flow rate: 15 m/min; UV detection: 220 nm.

Analysis method: HPLC: column: Daicel Chiralpak IC 5 μm, 250 mm×4.6 mm; eluent: 50% n-heptane/50% 2-propanol with 1% water and 0.2% trifluoroacetic acid; temperature: 55° C.; flow rate: 1.0 ml/min; UV detection: 220 nm.

LC-MS (method 4): Rₜ=2.19 min; MS (ESIpos): m/z=592 [M+H]⁺

¹H-NMR (400 MHz, DMSO-d₆): δ [ppm]=10.63 (s, 1H), 8.34 (s, 1H), 8.08 (s, 1H), 7.87 (d, 1H), 7.55-7.48 (m, 2H), 7.40-7.36 (m, 1H), 7.31 (d, 1H), 6.41 (s, 1H), 5.82-5.74 (m, 1H), 4.63 (d, 1H), 4.45 (s, 3H), 3.79-3.66 (m, 2H), 3.64-3.41 (m, 5H), 3.30-3.23 (m, 1H, partially concealed), 3.20-3.15 (m, 1H), 2.80 (d, 1H), 2.32-2.20 (m, 2H), 2.15-2.04 (m, 1H), 1.92-1.79 (m, 1H), 0.97-0.88 (m, 6H). Additional signals of minor rotamers were also detected.

### Example 46

### 4-({(2S)-2-[11-Chloro-7-(difluoromethyl)-2-oxo-7,8-dihydro-2H-[3]benzoxocino[5,6-c]pyridin-3(5H)-yl]butanoyl}amino)-2-fluorobenzamide (Mixture of Two Diastereomers)

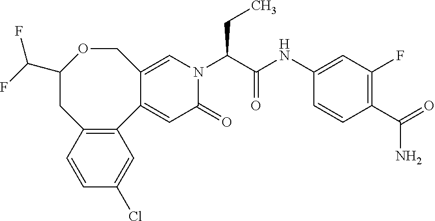 [SMILES: CC[C@@H](C(=O)Nc1ccc(C(N)=O)c(F)c1)n1cc2c(cc1=O)-c1cc(Cl)ccc1CC(C(F)F)OC2]

General Method 7 was carried out with 11-chloro-7-(difluoromethyl)-3,5,7,8-tetrahydro-2H-[3]benzoxocino[5,6-c]pyridin-2-one (racemate) (120 mg, 74% purity, 285 μmol, 1.0 eq.), 4-{[(2R)-2-bromobutanoyl]amino}-2-fluorobenzamide (single stereoisomer) (130 mg, 427 μmol, 1.5 eq.), 1,1,3,3-tetramethylguanidine (110 μl, 850 μmol, 3.0 eq.) in a mixture of 2-propanol/acetone (4:1, 3.2 ml) including the following variations of the procedure: The crude mixture was purified by preparative HPLC (reversed phase, eluent: water with 0.05% formic acid/acetonitrile 90:10 to 5:95). Yield: 121 mg (80% of theory).

LC-MS (method 3): Rₜ=3.06/3.16 min; MS (ESIpos): m/z=534 [M+H]⁺

### Example 47

### 4-({(2S)-2-[(7S)-11-Chloro-7-(difluoromethyl)-2-oxo-7,8-dihydro-2H-[3]benzoxocino[5,6-c]pyridin-3(5H)-yl]butanoyl}amino)-2-fluorobenzamide (Single Stereoisomer)

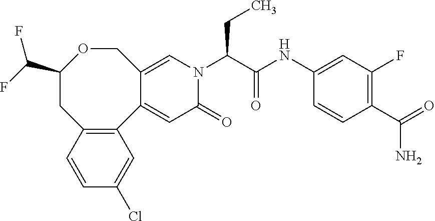 [SMILES: CC[C@@H](C(=O)Nc1ccc(C(N)=O)c(F)c1)n1cc2c(cc1=O)-c1cc(Cl)ccc1C[C@@H](C(F)F)OC2]

Diastereomer separation of 120 mg of 4-({(2S)-2-[11-chloro-7-(difluoromethyl)-2-oxo-7,8-dihydro-2H-[3]benzoxocino[5,6-c]pyridin-3(5H)-yl]butanoyl}amino)-2-fluorobenzamide (mixture of two diastereomers), Example 46 provides:
single stereoisomer 1 (the title compound 47) (chiral SFC: Rₜ=2.80 min, 99% de): 34.0 mg,
single stereoisomer 2 (chiral SFC: Rₜ=4.41 min): 28.5 mg.

Separation method: SFC: column: Daicel Chiralpak ID 5 μm, 250 mm×20 mm; eluent: 75% carbon dioxide/25% ethanol; temperature: 40° C.; flow rate: 80 ml/min; UV detection: 210 nm.

Analysis method: SFC: column: Daicel Chiralpak ID 5 μm, 250 mm×4.6 mm; eluent: 80% carbon dioxide/20% ethanol; temperature: 40° C.; flow rate: 3.0 ml/min; UV detection: 210 nm.

LC-MS (method 1): Rₜ=0.94 min; MS (ESIpos): m/z=534 [M+H]⁺

¹H-NMR (500 MHz, DMSO-d₆): δ [ppm]=10.90 (s, 1H), 8.05/8.00 (2s, 1H), 7.72-7.67 (m, 1H), 7.66-7.62 (m, 1H), 7.58-7.49 (m, 3H), 7.43-7.35 (m, 3H), 6.43/6.36 (2s, 1H), 6.27-5.96 (m, 1H), 5.54 (dd, 1H), 4.71 (d, 1H), 3.76-3.66 (m, 2H), 3.01 (d, 1H), 2.39-2.30 (m, 1H), 2.28-2.08 (m, 2H), 0.93 (t, 3H). Additional signals of minor rotamers were also detected.

### Example 48

### 4-{2-[11-Chloro-7-(difluoromethyl)-2-oxo-7,8-dihydro-2H-[3]benzoxocino[5,6-c]pyridin-3(5H)-yl]-3-(tetrahydro-2H-pyran-2-yl)propanamido}-2-fluorobenzamide (Mixture of Stereoisomers)

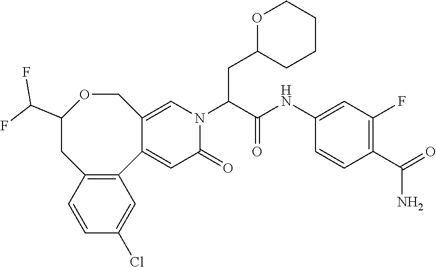 [SMILES: NC(=O)c1ccc(NC(=O)C(CC2CCCCO2)n2cc3c(cc2=O)-c2cc(Cl)ccc2CC(C(F)F)OC3)cc1F]

General Method 7 was carried out with 11-chloro-7-(difluoromethyl)-3,5,7,8-tetrahydro-2H-[3]benzoxocino[5,6-c]pyridin-2-one (racemate) (100 mg, 74% purity, 237 μmol, 1.0 eq.), 4-[2-bromo-3-(tetrahydro-2H-pyran-2-yl)propanamido]-2-fluorobenzamide (mixture of stereoisomers) (96.3 mg, 92% purity, 237 μmol, 1.0 eq.), 1,1,3,3-tetramethylguanidine (89 μl, 710 μmol, 3.0 eq.) in a mixture of 2-propanol/acetone (4:1, 2.6 ml) including the following variations of the procedure: The crude mixture was purified by preparative HPLC (reversed phase, eluent: water with 0.05% formic acid/acetonitrile 80:20 to 5:95). Yield: 101 mg (70% of theory).

LC-MS (method 1): Rₜ=1.04/1.06 min; MS (ESIpos): m/z=604 [M+H]⁺

### Example 49

### 4-({(2S)-2-[(7S)-11-Chloro-7-(difluoromethyl)-2-oxo-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (Single Stereoisomer)

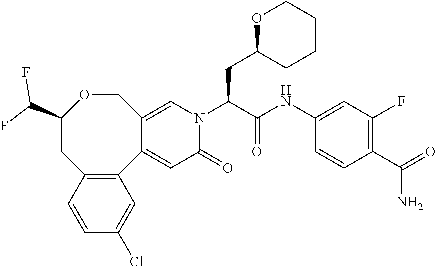 [SMILES: NC(=O)c1ccc(NC(=O)[C@H](C[C@@H]2CCCCO2)n2cc3c(cc2=O)-c2cc(Cl)ccc2C[C@@H](C(F)F)OC3)cc1F]

Stereoisomer separation of 101 mg of 4-{2-[11-chloro-7-(difluoromethyl)-2-oxo-7,8-dihydro-2H-[3]benzoxocino[5,6-c]pyridin-3(5H)-yl]-3-(tetrahydro-2H-pyran-2-yl)propanamido}-2-fluorobenzamide (mixture of stereoisomers), Example 48 provides:
mixture of stereoisomers 1 (chiral HPLC method 1: Rₜ=16.19/17.54 min): 30.0 mg,
mixture of stereoisomers 2 (chiral HPLC method 1: Rₜ=21.11 min): 22.5 mg,
single stereoisomer 1 (chiral HPLC method 1: Rₜ=28.78 min): 8.4 mg,
single stereoisomer 2 (the title compound 49) (chiral HPLC method 2: Rₜ=8.22 min): 12.2 mg (9% of theory, 99% de),
single stereoisomer 3 (chiral HPLC method 2: Rₜ=8.87 min): 5.4 mg.

Separation method 1: HPLC: column: YMC Chiralart Cellulose SC 5 μm, 250 mm×20 mm; eluent: 70% n-heptane/30% ethanol; temperature: 40° C.; flow rate: 15 ml/min; UV detection: 220 nm.

Separation method 2: single stereoisomer 2 and single stereoisomer 3 eluted as a mixture in the first separation (mixture of stereoisomers 1). The mixture was purified according to the following conditions: HPLC: column: Daicel Chiralpak IE 5 μm, 250 mm×20 mm; eluent: 50% n-heptane/50% ethanol; temperature: 50° C.; flow rate: 15 ml/min; UV detection: 220 nm.

Analysis method 1: HPLC: column: YMC Chiralart Cellulose SC 5 μm, 250 mm×4.6 mm; eluent: 80% iso-hexane/20% ethanol; temperature: 35° C.; flow rate: 1.0 ml/min; UV detection: 220 nm.

Analysis method 2: HPLC: column: Daicel Chiralpak IE 5 μm, 250 mm×4.6 mm; eluent: iso-hexane 50%/ethanol 50%; temperature: 50° C.; flow rate: 1.0 m/min; UV detection: 220 nm.

LC-MS (method 3): Rₜ=3.59 min; MS (ESIpos): m/z=604 [M+H]⁺

¹H-NMR (400 MHz, DMSO-d₆): δ [ppm]=10.84/10.79 (2s, 1H), 8.10/7.97 (2s, 1H), 7.73-7.64 (m, 2H), 7.59-7.43 (m, 4H), 7.42-7.32 (m, 2H), 6.41/6.31 (2s, 1H), 6.31-5.94 (m, 1H), 5.78-5.68 (m, 1H), 4.73 (d, 1H), 3.90-3.78 (m, 1H), 3.77-3.57 (m, 2H), 3.28-3.16 (m, 2H), 2.99 (d, 1H), 2.42-2.21 (m, 2H), 2.14-1.99 (m, 1H), 1.80-1.68 (m, 1H), 1.65-1.54 (m, 1H), 1.49-1.38 (m, 3H), 1.33-1.20 (m, 1H). Additional signals of minor rotamers were also detected.

### Example 50

### 4-({2-[11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (Mixture of Stereoisomers)

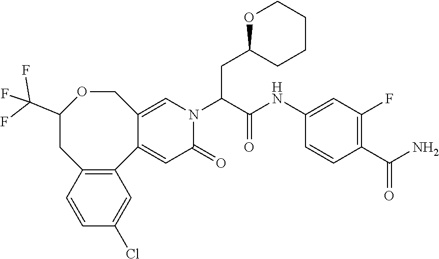 [SMILES: NC(=O)c1ccc(NC(=O)C(C[C@@H]2CCCCO2)n2cc3c(cc2=O)-c2cc(Cl)ccc2CC(C(F)(F)F)OC3)cc1F]

2-[11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoic acid (mixture of stereoisomers) (79 mg, 0.15 mmol) and 4-amino-2-fluorobenzamide (36 mg, 0.23 mmol, 1.5 eq.) were reacted according to General Method 11. Yield: 64 mg (63% of theory).

LC-MS (method 4): Rₜ=2.06 min; MS (ESIpos): m/z=622 [M+H]⁺

¹H-NMR (400 MHz, DMSO-d₆): δ [ppm]=10.85/10.84/10.77/10.75 (4s, 1H), 8.15-8.09 (m, 1H), 7.71-7.61 (m, 2H), 7.59-7.49 (m, 3H), 7.48-7.39 (m, 3H), 6.42 (s, 1H), 5.77-5.65 (m, 1H), 4.83-4.68 (m, 1H), 4.23-4.11 (m, 1H), 3.87-3.72 (m, 2H), 3.28-2.96 (m, 3H, partially concealed), 2.49-2.41 (m, 1H, partially concealed), 2.38-2.25 (m, 1H), 2.23-2.06 (m, 1H), 1.80-1.68 (m, 1H), 1.64-1.55 (m, 1H), 1.47-1.34 (m, 3H), 1.32-1.17 (m, 1H). Additional signals of minor rotamers were also detected.

### Example 51

### 4-({(2S)-2-[(7S)-11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (Single Stereoisomer)

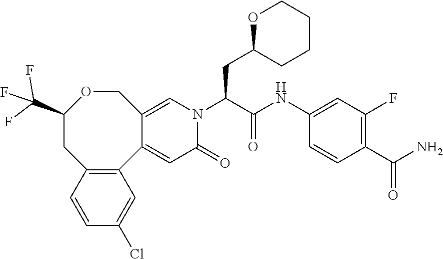 [SMILES: NC(=O)c1ccc(NC(=O)[C@H](C[C@@H]2CCCCO2)n2cc3c(cc2=O)-c2cc(Cl)ccc2C[C@@H](C(F)(F)F)OC3)cc1F]

Stereoisomer separation of 60 mg of 4-({2-[11-chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (mixture of stereoisomers), Example 50 gave
single stereoisomer 1 (the title compound Example 51) (chiral HPLC: Rₜ=8.4 min, 99% de): 13 mg,
single stereoisomer 2 (chiral HPLC: Rₜ=12.57 min): 18 mg,
single stereoisomer 3 (chiral HPLC: Rₜ=10.95 min): 6 mg,
single stereoisomer 4 (chiral HPLC: Rₜ=10.58 min): 4 mg.

First separation method: HPLC: column: Daicel Chiralpak IF 5 μm, 250 mm×20 mm; eluent: 50% n-heptane/50% ethanol with 0.2% acetic acid; temperature: 50° C.; flow rate: 15 mu/min; UV detection: 220 nm.

Second separation method: The fractions containing single stereoisomers 1 and 2 were combined and separated with the following conditions: HPLC: column: Daicel Chiralpak IE 5 μm, 250 mm×20 mm; eluent: 30% n-heptane/70% ethanol with 0.2% acetic acid; temperature: 60° C.; flow rate: 15 ml/min; UV detection: 220 nm. The fractions containing single stereoisomer 1 were combined and purified by preparative HPLC chromatography (reversed phase, eluent: 10-90% acetonitrile/water with 0.05% formic acid).

Analysis method: HPLC: column: Daicel Chiralpak IE 5 μm, 250 mm×4.6 mm; eluent: 50% n-heptane/50% ethanol with 1% water and 0.2% trifluoroacetic acid; temperature: 45° C.; flow rate: 1 ml/min; UV detection: 220 nm.

LC-MS (method 4): Rₜ=2.08 min; MS (ESIpos): m/z=622 [M+H]⁺

¹H-NMR (400 MHz, DMSO-d₆): δ [ppm]=10.77 (s, 1H), 8.13 (s, 1H), 7.71-7.64 (m, 2H), 7.58-7.50 (m, 3H), 7.48-7.44 (m, 1H), 7.43-7.39 (m, 2H), 6.42 (s, 1H), 5.76-5.68 (m, 1H), 4.79 (m, 1H), 4.21-4.12 (m, 1H), 3.86-3.80 (m, 1H), 3.75 (d, 1H), 3.30-3.20 (m, 2H, partially concealed), 3.11 (d, 1H), 2.49-2.45 (m, 1H, partially concealed), 2.32-2.25 (m, 1H), 2.14-2.05 (m, 1H), 1.77-1.71 (m, 1H), 1.63-1.57 (m, 1H), 1.46-1.36 (m, 3H), 1.30-1.21 (m, 1H). Additional signals of minor rotamers were also detected.

### Example 52

### 5-({2-[11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoyl}amino)pyridine-2-carboxamide (Mixture of Stereoisomers)

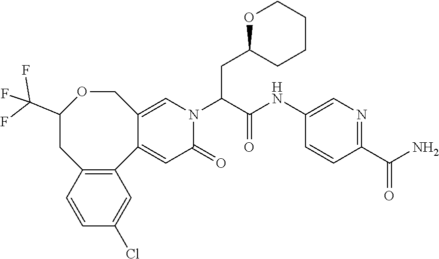 [SMILES: NC(=O)c1ccc(NC(=O)C(C[C@@H]2CCCCO2)n2cc3c(cc2=O)-c2cc(Cl)ccc2CC(C(F)(F)F)OC3)cn1]

2-[11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoic acid (mixture of stereoisomers)(70 mg, 0.14 mmol) and 5-aminopyridine-2-carboxamide (prepared according to WO 2017/005725, example 1.3B) (28 mg, 0.21 mmol, 1.5 eq.) were reacted according to General Method 11. Yield: 65 mg (78% of theory).

LC-MS (method 4): Rₜ=1.97 min; MS (ESIpos): m/z=605 [M+H]⁺

¹H-NMR (400 MHz, DMSO-d₆): δ [ppm]=10.94/10.93/10.85/10.84 (s, 1H), 8.89-8.82 (m, 1H), 8.27-8.20 (m, 1H), 8.17-8.10 (m, 1H), 8.04-7.97 (m, 2H), 7.60-7.55 (m, 1H), 7.55-7.49 (m, 1H), 7.47-7.39 (m, 2H), 6.43 (s, 1H), 5.81-5.69 (m, 1H), 4.82-4.71 (m, 1H), 4.24-4.12 (m, 1H), 3.88-3.71 (m, 2H), 3.29-2.98 (m, 3H, partially concealed), 2.48-2.06 (m, 3H), 1.79-1.69 (m, 1H), 1.66-1.55 (m, 1H), 1.46-1.34 (m, 3H), 1.30-1.18 (m, 1H). Additional signals of minor rotamers were also detected.

### Example 53

### 5-({(2S)-2-[(7S)-11-Chloro-2-oxo-7-(trifluoromethyl)-7,8-dihydro-2H-[3]benzoxocino[5.6-c]pyridin-3(5H)-yl]-3-[(2S)-tetrahydro-2H-pyran-2-yl]propanoyl}amino)pyridine-2-carboxamide (Single Stereoisomer)

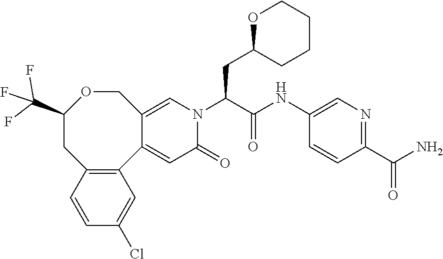 [SMILES: NC(=O)c1ccc(NC(=O)[C@H](C[C@@H]2CCCCO2)n2cc3c(cc2=O)-c2cc(Cl)ccc2C[C@@H](C(F)(F)F)OC3)cn1]

Stereoisomer separation of 62 mg of 5-({2-[11-chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoyl}amino)-pyridine-2-carboxamide (mixture of stereoisomers), Example 52 gave
single stereoisomer 1 (chiral HPLC: %=7.04 min): 26 mg,
single stereoisomer 2 (the title compound Example 53) (chiral HPLC: Rₜ=5.79 min, 99% de): 21 mg,
single stereoisomer 3 (chiral HPLC: Rₜ=7.74 min): 9 mg,
single stereoisomer 4 (chiral HPLC: Rₜ=10.62 min): 5 mg.

First separation method: HPLC: column: Daicel Chiralpak IG 5 μm, 250 mm×20 mm; eluent: 100% ethanol; temperature: 70° C.; flow rate: 15 ml/min; UV detection: 220 nm.

Second separation method: Single stereoisomer 2 and single stereoisomer 3 eluted as a mixture with single stereoisomer 1 in the first separation method. The respective fractions were combined and again purified according to the conditions of the first separation method. Single stereoisomer 2 and single stereoisomer 3 eluted as a mixture. Their fractions were combined and the stereoisomers separated with the following conditions: HPLC: column: Daicel Chiralpak ID 5 μm 250 mm×20 mm; eluent: 50% n-heptane/50% ethanol; temperature: 70° C.; flow rate: 15 ml/min; UV detection: 220 nm.

Analysis method: HPLC: column: Daicel Chiralpak ID 5 μm, 250 mm×4.6 mm; eluent: 50% iso-hexane/50% ethanol; temperature: 60° C.; flow rate: 1 ml/min; UV detection: 220 nm.

LC-MS (method 4): Rₜ=1.98 min; MS (ESIpos): m/z=605 [M+H]⁺

¹H-NMR (400 MHz, DMSO-d₆): δ [ppm]=10.85 (s, 1H), 8.89-8.85 (m, 1H), 8.27-8.22 (m, 1H), 8.17-8.13 (m, 1H), 8.05-7.98 (m, 2H), 7.60-7.50 (m, 2H), 7.44-7.38 (m, 2H), 6.43 (s, 1H), 5.81-5.71 (m, 1H), 4.79 (d, 1H), 4.22-4.12 (m, 1H), 3.87-3.79 (m, 1H), 3.75 (d, 1H), 3.30-3.22 (m, 2H, partially concealed), 3.14-3.06 (m, 1H), 2.49-2.43 (m, 1H, partially concealed), 2.38-2.27 (m, 1H), 2.17-2.06 (m, 1H), 1.79-1.70 (m, 1H), 1.65-1.58 (m, 1H), 1.47-1.36 (m, 3H), 1.31-1.20 (m, 1H). Additional signals of minor rotamers were also detected.

### Example 54

### 4-({2-[11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoyl}amino)benzamide (Mixture of Stereoisomers)

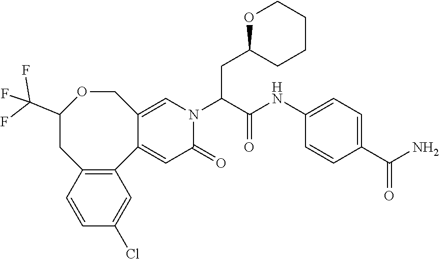 [SMILES: NC(=O)c1ccc(NC(=O)C(C[C@@H]2CCCCO2)n2cc3c(cc2=O)-c2cc(Cl)ccc2CC(C(F)(F)F)OC3)cc1]

2-[11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoic acid (mixture of stereoisomers) (50 mg, 0.098 mmol) and 4-aminobenzamide (20 mg, 0.15 mmol, 1.5 eq.) were reacted according to General Method 11. Yield: 47 mg (78% of theory).

LC-MS (method 4): Rₜ=1.95/1.96 min; MS (ESIpos): m/z=604 [M+H]⁺

¹H-NMR (400 MHz, DMSO-d₆): δ [ppm]=10.68/10.67/10.62/10.60 (4s, 1H), 8.16-8.09 (m, 1H), 7.89-7.80 (m, 3H), 7.74-7.65 (m, 2H), 7.60-7.54 (m, 1H), 7.46-7.39 (m, 2H), 7.27-7.21 (m, 1H), 6.42/6.42 (2s, 1H), 5.81-5.69 (m, 1H), 4.83-4.70 (m, 1H), 4.23-4.11 (m, 1H), 3.88-3.71 (m, 2H), 3.28-2.98 (m, 3H, partially concealed), 2.49-2.43 (m, 1H, partially concealed), 2.36-2.05 (m, 2H), 1.81-1.68 (m, 1H), 1.65-1.55 (m, 1H), 1.48-1.33 (m, 3H), 1.33-1.17 (m, 1H). Additional signals of minor rotamers were also detected.

### Example 55

### 4-({(2S)-2-[(7S)-11-Chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoyl}amino)benzamide (Single Stereoisomer)

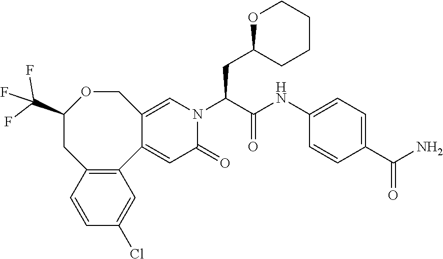 [SMILES: NC(=O)c1ccc(NC(=O)[C@H](C[C@@H]2CCCCO2)n2cc3c(cc2=O)-c2cc(Cl)ccc2C[C@@H](C(F)(F)F)OC3)cc1]

Stereoisomer separation of 105 mg of 4-({2-[11-chloro-2-oxo-7-(trifluoromethyl)-7,8-dihydro-2H-[3]benzoxocino[5,6-c]pyridin-3(5H)-yl]-3-[(2S)-tetrahydro-2H-pyran-2-yl]propanoyl}amino)-benzamide (mixture of stereoisomers), Example 54 gave
single stereoisomer 1 (the title compound Example 55) (chiral HPLC: Rₜ=6.10 min, 99% de): 36 mg,
single stereoisomer 2 (chiral HPLC: Rₜ=8.15 min): 46 mg,
single stereoisomer 3 (chiral HPLC: Rₜ=9.01 min): 9 mg,
single stereoisomer 4 (chiral HPLC: Rₜ=7.18 min): 4 mg.

First separation method: HPLC: column: Daicel Chiralpak IF 5 μm, 250 mm×20 mm; eluent: 50% n-heptane/50% 2-propanol; temperature: 45° C.; flow rate: 15 ml/min; UV detection: 220 nm.

Second separation method: Single stereoisomer 1 and single stereoisomer 2 eluted as a mixture in the first separation method. Their fractions were combined and the stereoisomers separated with the following conditions: HPLC: column: Daicel Chiralpak IE 5 μm 250 mm×20 mm; eluent: 40% n-heptane/60% 2-propanol; temperature: 55° C.; flow rate: 15 ml/min; UV detection: 220 nm.

Analysis method: HPLC: column: Daicel Chiralpak IE 5 μm, 250 mm×4.6 mm; eluent: 50% iso-hexane/50% 2-propanol; temperature: 60° C.; flow rate: 1 ml/min; UV detection: 220 nm.

LC-MS (method 4): Rₜ=1.97 min; MS (ESIpos): m/z=604 [M+H]⁺

¹H-NMR (400 MHz, DMSO-d₆): δ [ppm]=10.62 (s, 1H), 8.14 (s, 1H), 7.89-7.81 (m, 3H), 7.74-7.68 (m, 2H), 7.57 (dd, 1H), 7.43-7.39 (m, 2H), 7.28-7.20 (m, 1H), 6.42 (s, 1H), 5.81-5.73 (m, 1H), 4.79 (d, 1H), 4.21-4.11 (m, 1H), 3.87-3.81 (m, 1H), 3.75 (d, 1H), 3.28-3.19 (m, 2H, partially concealed), 3.13-3.05 (m, 1H), 2.49-2.45 (m, 1H, partially concealed), 2.31-2.24 (m, 1H), 2.14-2.06 (m, 1H), 1.78-1.71 (m, 1H), 1.65-1.57 (m, 1H), 1.47-1.34 (m, 3H), 1.32-1.21 (m, 1H). Additional signals of minor rotamers were also detected.

### Example 56

### 4-({2-[11-Chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoyl}amino)-2-fluorobenzamide (mixture of stereoisomers)

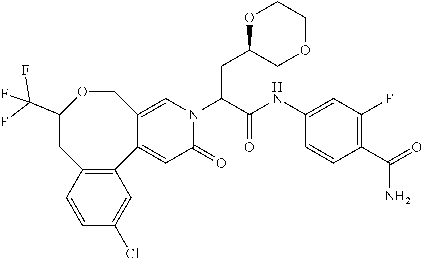 [SMILES: NC(=O)c1ccc(NC(=O)C(C[C@@H]2COCCO2)n2cc3c(cc2=O)-c2cc(Cl)ccc2CC(C(F)(F)F)OC3)cc1F]

2-[11-Chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoic acid (mixture of stereoisomers) (100 mg, 0.21 mmol) and 4-amino-2-fluorobenzamide (47 mg, 0.31 mmol, 1.5 eq.) were reacted according to General Method 11. Yield: 86 mg (66% of theory).

LC-MS (method 4): Rₜ=1.78/1.80 min; MS (ESIpos): m/z=624 [M+H]⁺

¹H-NMR (400 MHz, DMSO-d₆): δ [ppm]=10.88/10.84/10.77 (3s, 1H), 8.15-8.08 (m, 1H), 7.73-7.59 (m, 2H), 7.59-7.45 (m, 3H), 7.48-7.35 (m, 3H), 6.44/6.43 (2s, 1H), 5.75-5.65 (m, 1H), 4.84-4.69 (m, 1H), 4.23-4.12 (m, 1H), 3.81-3.63 (m, 3H), 3.63-3.57 (m, 1H), 3.53-3.40 (m, 3H), 3.28-3.19 (m, 1H, partially concealed), 3.15-3.07 (m, 1H), 2.49-2.43 (m, 1H, partially concealed), 2.33-2.20 (m, 1H), 2.20-2.04 (m, 1H). Additional signals of minor rotamers were also detected.

### Example 57

### 4-({(2S)-2-[(7S)-11-Chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoyl}amino)-2-fluorobenzamide (Single Stereoisomer)

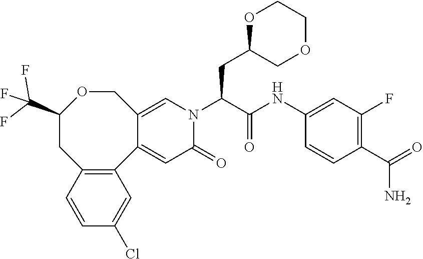 [SMILES: NC(=O)c1ccc(NC(=O)[C@H](C[C@@H]2COCCO2)n2cc3c(cc2=O)-c2cc(Cl)ccc2C[C@@H](C(F)(F)F)OC3)cc1F]

Stereoisomer separation of 80 mg of 4-({2-[11-chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoyl})amino)-2-fluoro-benzamide (mixture of stereoisomers), Example 56 gave
single stereoisomer 1 (chiral HPLC: Rₜ=11.47 min): 7 mg,
single stereoisomer 2 (chiral HPLC: Rₜ=12.99 min): 9 mg,
single stereoisomer 3 (the title compound Example 57) (chiral HPLC: Rₜ=15.60 min, 99% de): 24 mg,
single stereoisomer 4 (chiral HPLC: Rₜ=16.03 min): 25 mg.

First separation method: HPLC: column: Daicel Chiralpak IE 5 μm, 250 mm×20 mm; eluent: 50% n-heptane/50% ethanol with 0.2% acetic acid; temperature: 60° C.; flow rate: 15 ml/min; UV detection: 220 nm.

Second separation method: The fractions containing single stereoisomer 3 were combined and again purified with the following conditions: HPLC: column: Daicel Chiralpak IE 5 μm, 250 mm×20 mm; eluent: 50% n-heptane/50% ethanol with 0.2% acetic acid; temperature: 60° C.; flow rate: 15 ml/min; UV detection: 220 nm. The fractions containing single stereoisomer 3 were combined and purified by preparative HPLC chromatography (reversed phase, eluent: 10-90% acetonitrile/water with 0.05% formic acid).

Analysis method: HPLC: column: Daicel Chiralpak IE 5 μm, 250 mm×4.6 mm; eluent: 50% n-heptane/50% ethanol with 1% water and 0.2% trifluoroacetic acid; temperature: 60° C.; flow rate: 1 ml/min; UV detection: 220 nm.

LC-MS (method 4): Rₜ=1.80 min; MS (ESIpos): m/z=624 [M+H]⁺

¹H-NMR (400 MHz, DMSO-d₆): δ [ppm]=10.76 (s, 1H), 8.14 (s, 1H), 7.71-7.63 (m, 2H), 7.59-7.50 (m, 3H), 7.46-7.39 (m, 3H), 6.43 (s, 1H), 5.72-5.65 (m, 1H), 4.77 (d, 1H), 4.20-4.12 (m, 1H), 3.78-3.70 (m, 2H), 3.69-3.58 (m, 2H), 3.53-3.41 (m, 3H), 3.28-3.22 (m, 1H), 3.14-3.08 (m, 1H), 2.49-2.44 (m, 1H), 2.30-2.22 (m, 1H), 2.13-2.05 (m, 1H). Additional signals of minor rotamers were also detected.

### Example 58

### 4-({2-[11-Chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoyl}amino)benzamide (Mixture of Stereoisomers)

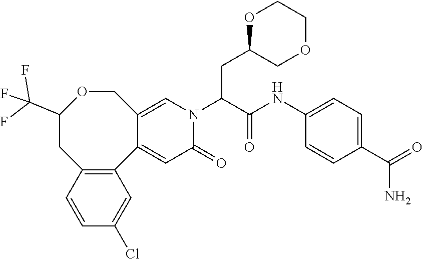 [SMILES: NC(=O)c1ccc(NC(=O)C(C[C@@H]2COCCO2)n2cc3c(cc2=O)-c2cc(Cl)ccc2CC(C(F)(F)F)OC3)cc1]

2-[11-Chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoic acid (mixture of stereoisomers) (120 mg, 0.246 mmol) and 4-aminobenzamide (50 mg, 0.37 mmol, 1.5 eq.) were reacted according to General Method 11. Yield: 101 mg (67% of theory).

LC-MS (method 4): Rₜ=1.72/1.74 min; MS (ESIpos): m/z=606 [M+H]⁺

¹H-NMR (400 MHz, DMSO-d₆): δ [ppm]=10.71/10.69/10.62 (3s, 1H), 8.16-8.10 (m, 1H), 7.89-7.81 (m, 3H), 7.72-7.65 (m, 2H), 7.60-7.55 (m, 1H), 7.46-7.39 (m, 2H), 7.27-7.20 (m, 1H), 6.43 (s, 1H), 5.78-5.69 (m, 1H), 4.85-4.68 (m, 1H), 4.22-4.12 (m, 1H), 3.82-3.57 (m, 4H), 3.52-3.38 (m, 2H), 3.29-3.19 (m, 2H, partially concealed), 3.16-3.07 (m, 1H), 2.48-2.41 (m, 1H, partially concealed), 2.37-2.05 (m, 2H). Additional signals of minor rotamers were also detected.

### Example 59

### 4-({(2S)-2-[(7S)-11-Chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoyl}amino)benzamide (Single Stereoisomer)

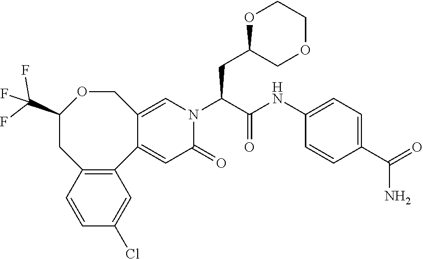 [SMILES: NC(=O)c1ccc(NC(=O)[C@H](C[C@@H]2COCCO2)n2cc3c(cc2=O)-c2cc(Cl)ccc2C[C@@H](C(F)(F)F)OC3)cc1]

Stereoisomer separation of 98 mg of 4-({2-[11-chloro-2-oxo-7-(trifluoromethyl)-7,8-dihydro-2H-[3]benzoxocino[5,6-c]pyridin-3(5H)-yl]-3-[(2R)-1,4-dioxan-2-yl]propanoyl}amino)benzamide (mixture of stereoisomers), Example 58 gave
single stereoisomer 1 (the title compound Example 59) (chiral HPLC: Rₜ=7.21 min, 99% de): 24 mg,
single stereoisomer 2 (chiral HPLC: Rₜ=13.07 min): 10 mg,
single stereoisomer 3 (chiral HPLC: Rₜ=9.29 min): 20 mg,
single stereoisomer 4 (chiral HPLC: Rₜ=7.85 min): 7 mg.

First separation method: HPLC: column: Daicel Chiralpak IG 5 μm, 250 mm×20 mm; eluent: 50% n-heptane/50% ethanol with 0.2% acetic acid; temperature: 50° C.; flow rate: 15 ml/min; UV detection: 220 nm.

Second separation method: Single stereoisomers 1 and 2 eluted as a mixture in the first separation method. Their fractions were combined and the stereoisomers were separated under the following conditions: HPLC: column: Daicel Chiralpak OZ 5 μm, 250 mm×20 mm; eluent: 30% n-heptane/70% ethanol with 0.2% acetic acid; temperature: 70° C.; flow rate: 15 ml/min; UV detection: 220 nm.

The fractions containing single stereoisomer 1 were combined and purified by preparative HPLC chromatography (reversed phase, eluent: 10-90% acetonitrile/water with 0.05% formic acid).

Analysis method: HPLC: column: Daicel Chiralpak OZ-H 5 μm, 250 mm×4.6 mm; eluent: 50% iso-hexane/50% ethanol with 1% water and 0.2% trifluoroacetic acid; temperature: 70° C.; flow rate: 1 ml/min; UV detection: 220 nm.

LC-MS (method 4): Rₜ=1.74 min; MS (ESIpos): m/z=606 [M+H]⁺

¹H-NMR (400 MHz, DMSO-d₆): δ [ppm]=10.62 (s, 1H), 8.15 (s, 1H), 7.91-7.82 (m, 3H), 7.72-7.67 (m, 2H), 7.59-7.55 (m, 1H), 7.45-7.39 (m, 2H), 7.28-7.22 (m, 1H), 6.43 (s, 1H), 5.78-5.69 (m, 1H), 4.78 (m, 1H), 4.21-4.12 (m, 1H), 3.78-3.58 (m, 4H), 3.54-3.41 (m, 3H), 3.29-3.21 (m, 1H), 3.10 (d, 1H), 2.49-2.44 (m, 1H, partially concealed), 2.29-2.21 (m, 1H), 2.14-2.05 (m, 1H). Additional signals of minor rotamers were also detected.

### Example 60

### 4-({2-[11-Chloro-2-oxo-7-(trifluoromethyl)-7,8-dihydro-2H-[3]benzoxocino[5,6-c]pyridin-3(5H)-yl]-4-methoxybutanoyl}amino)-2-fluorobenzamide (Mixture of Stereoisomers)

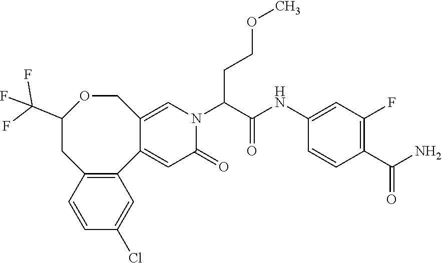 [SMILES: COCCC(C(=O)Nc1ccc(C(N)=O)c(F)c1)n1cc2c(cc1=O)-c1cc(Cl)ccc1CC(C(F)(F)F)OC2]

2-[11-Chloro-2-oxo-7-(trifluoromethyl)-7,8-dihydro-2H-[3]benzoxocino[5,6-c]pyridin-3(5H)-yl]-4-methoxybutanoic acid (mixture of stereoisomers) (80 mg, 0.18 mmol) and 4-amino-2-fluorobenzamide (41 mg, 0.27 mmol, 1.5 eq.) were reacted according to General Method 11. In a second experiment, 2-[11-chloro-2-oxo-7-(trifluoromethyl)-7,8-dihydro-2H-[3]benzoxocino[5,6-c]pyridin-3(5H)-yl]-4-methoxybutanoic acid (mixture of stereoisomers) (10 mg, 0.022 mmol) and 4-amino-2-fluorobenzamide (5 mg, 0.034 mmol, 1.5 eq.) were reacted according to General Method 11. In a third experiment, 1-chloro-N,N,2-trimethylprop-1-en-1-amine (4 μl, 0.029 mmol, 1.3 eq.) was added to a mixture of 2-[11-chloro-2-oxo-7-(trifluoromethyl)-7,8-dihydro-2H-[3]benzoxocino[5,6-c]pyridin-3(5H)-yl]-4-methoxybutanoic acid (mixture of stereoisomers) (10 mg, 0.022 mmol) in dichloromethane (0.5 ml). The reaction mixture was stirred at RT for 10 min, followed by the addition of 4-amino-2-fluorobenzamide (5.0 mg, 0.029 mmol, 1.3 eq.). The reaction mixture was stirred at RT overnight and concentrated under reduced pressure. All three experiments were combined and purified together according to General Method 11. Yield: 81 mg (76% of theory).

LC-MS (method 4): Rₜ=1.87 min; MS (ESIneg): m/z=580 [M−H]⁻

¹H-NMR (400 MHz, DMSO-d₆): δ [ppm]=10.88/10.82 (2s, 1H), 8.13 (s, 1H), 7.71-7.61 (m, 2H), 7.60-7.50 (m, 3H), 7.46-7.38 (m, 3H), 6.43 (s, 1H), 5.75-5.65 (m, 1H), 4.79/4.74 (2d, 1H), 4.23-4.13 (m, 1H), 3.80-3.71 (m, 1H), 3.47-3.33 (m, 1H), 3.27-3.21 (m, 1H), 3.20/3.18 (2s, 3H), 3.16-3.07 (m, 1H), 2.50-2.29 (m, 3H, partially concealed). Additional signals of minor rotamers were also detected.

### Example 61

### 4-({(2S)-2-[(7S)-11-Chloro-2-oxo-7-(trifluoromethyl)-7,8-dihydro-2H-[3]benzoxocino[5,6-c]pyridin-3(5H)-yl]-4-methoxybutanoyl}amino)-2-fluorobenzamide (Single Stereoisomer)

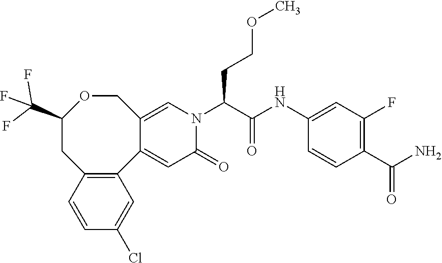 [SMILES: COCC[C@@H](C(=O)Nc1ccc(C(N)=O)c(F)c1)n1cc2c(cc1=O)-c1cc(Cl)ccc1C[C@@H](C(F)(F)F)OC2]

Stereoisomer separation of 93 mg of 4-({2-[11-chloro-2-oxo-7-(trifluoromethyl)-7,8-dihydro-2H-[3]benzoxocino[5,6-c]pyridin-3(5H)-yl]-4-methoxybutanoyl}amino)-2-fluoro-benzamide (mixture of stereoisomers), Example 60 gave
single stereoisomer 1 (chiral HPLC: Rₜ=4.93 min): 37 mg,
single stereoisomer 2 (the title compound Example 61) (chiral HPLC: Rₜ=5.30 min, 94% de): 33 mg,
single stereoisomer 3 (chiral HPLC: Rₜ=6.50 min): 9 mg,
single stereoisomer 4 (chiral HPLC: Rₜ=8.02 min): 9 mg.

First separation method: HPLC: column: Daicel Chiralpak IF 5 μm, 250 mm×20 mm; eluent: 50% n-heptane/50% 2-propanol with 1% water and 0.2% trifluoroacetic acid; temperature: 50° C.; flow rate: 15 m/min; UV detection: 220 nm.

Second separation method: Single stereoisomer 1 and single stereoisomer 2 eluted as a mixture in the first separation method. Their fractions were combined and the stereoisomers separated with the following conditions: HPLC: column: Daicel Chiralpak IF 5 μm 250 mm×20 mm; eluent: 55% n-heptane/45% 2-propanol with 1% water and 0.2% trifluoroacetic acid; temperature: 50° C.; flow rate: 15 m/min; UV detection: 220 nm.

Analysis method: HPLC: column: Daicel Chiralpak IF 5 μm, 250 mm×4.6 mm; eluent: 50% n-heptane/50% 2-propanol with 1% water and 0.2% trifluoroacetic acid; temperature: 50° C.; flow rate: 1 ml/min; UV detection: 220 nm.

LC-MS (method 4): Rₜ=1.84 min; MS (ESIpos): m/z=582 [M+H]⁺

¹H-NMR (400 MHz, DMSO-d₆): δ [ppm]=10.82 (s, 1H), 8.13 (s, 1H), 7.72-7.63 (m, 2H), 7.60-7.50 (m, 3H), 7.46-7.38 (m, 3H), 6.43 (s, 1H), 5.72-5.66 (m, 1H), 4.79 (d, 1H), 4.22-4.13 (m, 1H), 3.76 (d, 1H), 3.41-3.35 (m, 1H, partially concealed), 3.28-3.21 (m, 1H), 3.19 (s, 3H), 3.17-3.07 (m, 1H), 2.47-2.28 (m, 3H). Additional signals of minor rotamers were also detected.

### Example 62

### 4-({2-[11-Chloro-2-oxo-7-(trifluoromethyl)-7,8-dihydro-2H-[3]benzoxocino[5,6-c]pyridin-3(5H)-yl]-4-methoxybutanoyl}amino)benzamide (Mixture of Stereoisomers)

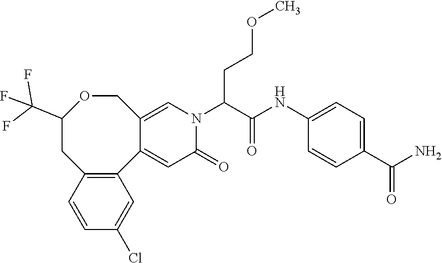 [SMILES: COCCC(C(=O)Nc1ccc(C(N)=O)cc1)n1cc2c(cc1=O)-c1cc(Cl)ccc1CC(C(F)(F)F)OC2]

2-[11-Chloro-2-oxo-7-(trifluoromethyl)-7,8-dihydro-2H-[3]benzoxocino[5,6-c]pyridin-3(5H)-yl]-4-methoxybutanoic acid (mixture of stereoisomers) (50 mg, 0.11 mmol) and 4-aminobenzamide (23 mg, 0.17 mmol, 1.5 eq.) were reacted according to General Method 11. Yield: 50 mg (78% of theory).

LC-MS (method 4): Rₜ=1.78 min; MS (ESIpos): m/z=564 [M+H]⁺

¹H-NMR (400 MHz, DMSO-d₆): δ [ppm]=10.71/10.67 (2s, 1H), 8.14/8.13 (2s, 1H), 7.89-7.82 (m, 3H), 7.73-7.65 (m, 2H), 7.60-7.55 (m, 1H), 7.45-7.40 (m, 2H), 7.27-7.22 (m, 1H), 6.43/6.31 (2s, 1H), 5.77-5.70 (m, 1H), 4.80/4.74 (2d, 1H), 4.23-4.12 (m, 1H), 3.81-3.72 (m, 1H), 3.46-3.33 (m, 1H), 3.28-3.22 (m, 1H), 3.20/3.19 (2s, 3H), 3.12 (d, 1H), 2.47-2.27 (m, 3H, partially concealed). Additional signals of minor rotamers were also detected.

### Example 63

### 4-({(25)-2-[(7S)-11-Chloro-2-oxo-7-(trifluoromethyl)-7,8-dihydro-2H-[3]benzoxocino[5,6-c]pyridin-3(5H)-yl]-4-methoxybutanoyl}amino)benzamide (Single Stereoisomer)

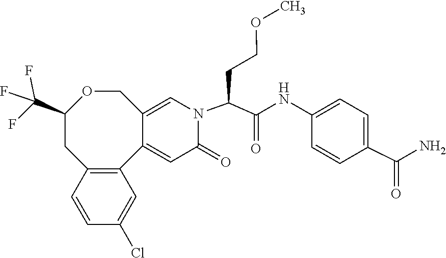 [SMILES: COCC[C@@H](C(=O)Nc1ccc(C(N)=O)cc1)n1cc2c(cc1=O)-c1cc(Cl)ccc1C[C@@H](C(F)(F)F)OC2]

Stereoisomer separation of 49 mg of 4-({2-[11-chloro-2-oxo-7-(trifluoromethyl)-7,8-dihydro-2H-[3]benzoxocino[5,6-c]pyridin-3(5H)-yl]-4-methoxybutanoyl}amino)benzamide (mixture of stereoisomers), Example 62 gave
single stereoisomer 1 (chiral HPLC: %=5.21 min): 17 mg,
single stereoisomer 2 (chiral HPLC: Rₜ=7.21 min): 3 mg,
single stereoisomer 3 (the title compound Example 63) (chiral HPLC: Rₜ=5.72 min, 99% de): 15 mg,
single stereoisomer 4 (chiral HPLC: Rₜ=8.77 min): 4 mg.

First separation method: HPLC: column: Daicel Chiralpak IG 5 μm, 250 mm×20 mm; eluent: 50% n-heptane/50% ethanol; temperature: 55° C.; flow rate: 15 ml/min; UV detection: 220 nm. The fractions containing the respective stereoisomers were each loaded on Agilent Bond Elut C-18 (500 mg, volume 6 ml), washed with water and eluted with a mixture of acetonitrile and dichloromethane (1:1).

Second separation method: Single stereoisomer 2 and single stereoisomer 3 eluted as a mixture in the first separation. The mixture was purified according to the following conditions: HPLC: column: Daicel Chiralpak IG 5 μm, 250 mm×20 mm; eluent: 50% n-heptane/50% ethanol; temperature: 55° C.; flow rate: 15 ml/min; UV detection: 220 nm.

Analysis method: HPLC: column: Daicel Chiralpak IF 5 μm, 250 mm×4.6 mm; eluent: 50% n-heptane/50% 2-propanol with 1% water and 0.2% trifluoroacetic acid; temperature: 40° C.; flow rate: 1 ml/min; UV detection: 220 nm.

LC-MS (method 4): Rₜ=1.77 min; MS (ESIpos): m/z=564 [M+H]⁺

¹H-NMR (400 MHz, DMSO-d₆): δ [ppm]=10.67 (s, 1H), 8.14 (s, 1H), 7.91-7.81 (m, 3H), 7.73-7.66 (m, 2H), 7.60-7.54 (m, 1H), 7.45-7.39 (m, 2H), 7.28-7.19 (m, 1H), 6.43 (s, 1H), 5.77-5.70 (m, 1H), 4.80 (d, 1H), 4.23-4.12 (m, 1H), 3.75 (d, 1H), 3.41-3.34 (m, 1H), 3.29-3.21 (m, 1H), 3.20 (s, 3H), 3.14-3.09 (m, 1H), 2.45-2.29 (m, 2H). Additional signals of a minor rotamer were also detected.

### Example 64

### 2-[11-Chloro-2-oxo-7-(trifluoromethyl)-7,8-dihydro-2H-[3]benzoxocino[5,6-c]pyridin-3(5H)-yl]-4-methoxy-N-([1,2,4]triazolo[1,5-a]pyridin-7-yl)butanamide (Mixture of Stereoisomers)

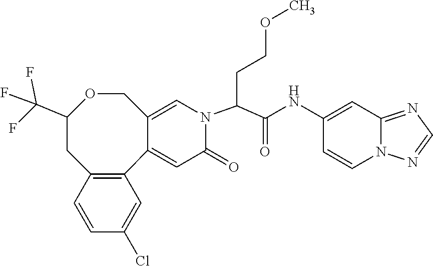 [SMILES: COCCC(C(=O)Nc1ccn2ncnc2c1)n1cc2c(cc1=O)-c1cc(Cl)ccc1CC(C(F)(F)F)OC2]

2-[11-Chloro-2-oxo-7-(trifluoromethyl)-7,8-dihydro-2H-[3]benzoxocino[5,6-c]pyridin-3(5H)-yl]-4-methoxybutanoic acid (mixture of stereoisomers) (75 mg, 0.17 mmol) was dissolved in pyridine (1.8 ml) and T3P (200 μl, 50% solution in ethyl acetate, 0.34 mmol, 2.0 eq.) was added. The reaction mixture was stirred at RT for 10 min before [1,2,4]triazolo[1,5-a]pyridin-7-amine hydrochloride (43 mg, 0.25 mmol, 1.5 eq.) was added. The reaction mixture was stirred at RT for 20 h. T3P (200 μl, 50% solution in ethyl acetate, 0.34 mmol, 2.0 eq.) and [1,2,4]triazolo[1,5-a]pyridin-7-amine hydrochloride (29 mg, 0.17 mmol, 1.0 eq.) were added and the reaction mixture stirred at RT for 3 days. Additional T3P (200 μl, 50% solution in ethyl acetate, 0.34 mmol, 2.0 eq.) and [1,2,4]triazolo[1,5-a]pyridin-7-amine hydrochloride (29 mg, 0.17 mmol, 1.0 eq.) were added. The reaction mixture stirred for 5 h and concentrated in vacuo. The crude product was purified by preparative HPLC (reversed phase, water with 0.05% formic acid/acetonitrile 90:10 to 10:90). Yield: 35 mg (37% of theory).

LC-MS (method 1): Rₜ=0.95 min; MS (ESIpos): m/z=562 [M+H]⁺

¹H-NMR (400 MHz, DMSO-d₆): δ [ppm]=11.04/10.99 (2s, 1H), 8.89-8.84 (m, 1H), 8.40-8.37 (m, 1H), 8.20-8.14 (m, 2H), 7.59-7.55 (m, 1H), 7.46-7.40 (m, 2H), 7.35-7.27 (m, 1H), 6.44 (s, 1H), 5.76-5.68 (m, 1H), 4.84-4.71 (m, 1H), 4.23-4.14 (m, 1H), 3.82-3.73 (m, 1H), 3.49-3.36 (m, 1H), 3.29-3.18 (m, 4H, partially concealed), 3.16-3.09 (m, 1H), 2.48-2.31 (m, 3H, partially concealed). Additional signals of minor rotamers were also detected.

### Example 65

### (2S)-2-[(7S)-11-Chloro-2-oxo-7-(trifluoromethyl)-7,8-dihydro-2H-[3]benzoxocino[5,6-c]pyridin-3(5H)-yl]-4-methoxy-N-([1,2,4]triazolo[1,5-a]pyridin-7-yl)butanamide (Single Stereoisomer)

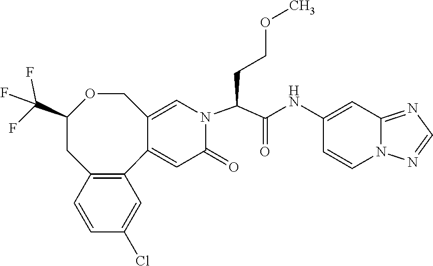 [SMILES: COCC[C@@H](C(=O)Nc1ccn2ncnc2c1)n1cc2c(cc1=O)-c1cc(Cl)ccc1C[C@@H](C(F)(F)F)OC2]

Stereoisomer separation of 33 mg of 2-[11-chloro-2-oxo-7-(trifluoromethyl)-7,8-dihydro-2H-[3]benzoxocino[5,6-c]pyridin-3(5H)-yl]-4-methoxy-N-([1,2,4]triazolo[1,5-a]pyridin-7-yl)butanamide (mixture of stereoisomers), Example 64 gave
single stereoisomer 1 (chiral HPLC: %=6.76 min): 7 mg,
single stereoisomer 2 (chiral HPLC: Rₜ=7.18 min): 4 mg,
single stereoisomer 3 (the title compound Example 65) (chiral HPLC: Rₜ=7.51 min, 99% de): 8 mg,
single stereoisomer 4 (chiral HPLC: Rₜ=8.28 min): 3 mg.

Separation method: HPLC: column: Daicel Chiralpak IE 5 μm, 250 mm×20 mm; eluent: 40% n-heptane/60% ethanol; temperature: 45° C.; flow rate: 15 m/min; UV detection: 220 nm.

Analysis method: HPLC: column: Daicel Chiralpak IE 5 μm, 250 mm×4.6 mm; eluent: 25% iso-hexane/75% ethanol; temperature: 45° C.; flow rate: 1 ml/min; UV detection: 220 nm.

LC-MS (method 1): Rₜ=0.97 min; MS (ESIpos): m/z=562 [M+H]⁺

¹H-NMR (400 MHz, DMSO-d₆): δ [ppm]=10.99 (s, 1H), 8.86 (d, 1H), 8.39 (s, 1H), 8.20-8.15 (m, 2H), 7.60-7.55 (m, 1H), 7.46-7.41 (m, 2H), 7.35-7.30 (m, 1H), 6.45 (s, 1H), 5.76-5.68 (m, 1H), 4.81 (d, 1H), 4.23-4.14 (m, 1H), 3.76 (d, 1H), 3.44-3.37 (m, 1H), 3.29-3.22 (m, 1H, partially concealed), 3.20 (s, 3H), 3.12 (d, 1H), 2.49-2.31 (m, 3H, partially concealed). Additional signals of a minor rotamer were also detected.

### Example 66

### 4-({(4S)-2-[(7S)-11-Chloro-2-oxo-7-(trifluoromethyl)-7,8-dihydro-2H-[3]benzoxocino[5,6-c]pyridin-3(5H)-yl]-4-methoxypentanoyl}amino)benzamide (Mixture of Two Stereoisomers)

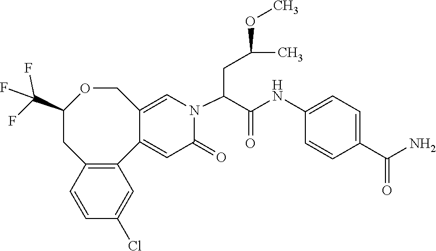 [SMILES: CO[C@@H](C)CC(C(=O)Nc1ccc(C(N)=O)cc1)n1cc2c(cc1=O)-c1cc(Cl)ccc1C[C@@H](C(F)(F)F)OC2]

(2F)-2-[(7S)-11-Chloro-2-oxo-7-(trifluoromethyl)-7,8-dihydro-2H-[3]benzoxocino[5,6-c]pyridin-3(5H)-yl]-2,3,5-trideoxy-4-O-methyl-L-glycero-pentonic acid (mixture of two diastereomers) (94 mg, 0.20 mmol), pyridine (18 μl, 0.23 mmol, 1.1 eq.) and T3P (179 μl, 50% solution in ethyl acetate, 0.31 mmol, 1.5 eq.) were added under argon atmosphere at RT to a solution of 4-aminobenzamide (28 mg, 0.20 mmol, 1.0 eq.) in tetrahydrofuran (4 ml). The reaction mixture was stirred at RT for 30 min before water was added and all volatiles were removed under reduced pressure. The residue was purified by preparative HPLC (reversed phase, eluent: acetonitrile/water gradient). Yield: 101 mg (85% of theory).

LC-MS (method 4): Rₜ=1.88/1.90 min; MS (ESIpos): m/z=578 [M+H]⁺

¹H-NMR (600 MHz, DMSO-d₆): δ [ppm]=10.71-10.62 (m, 1H), 8.19/8.12 (2s, 1H), 7.88-7.81 (m, 3H), 7.74-7.66 (m, 2H), 7.56 (dd, 1H), 7.45-7.39 (m, 2H), 7.25-7.19 (m, 1H), 6.43/6.43 (2s, 1H), 5.85-5.78 (m, 1H), 4.80/4.73 (2d, 1H), 4.22-4.13 (m, 1H), 3.78/3.75 (2d, 1H), 3.27-3.07 (m, 3H), 3.17/3.09 (2s, 3H), 2.46-2.20 (m, 2H), 1.16/1.14 (2d, 3H). Additional signals of minor rotamers were also detected.

### Example 67

### 4-({(2S,4S)-2-[(7S)-11-Chloro-2-oxo-7-(trifluoromethyl)-7,8-dihydro-2H-[3]benzoxocino[5,6-c]pyridin-3(5H)-yl]-4-methoxypentanoyl}amino)benzamide (Single Stereoisomer)

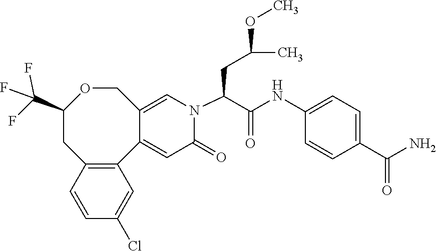 [SMILES: CO[C@@H](C)C[C@@H](C(=O)Nc1ccc(C(N)=O)cc1)n1cc2c(cc1=O)-c1cc(Cl)ccc1C[C@@H](C(F)(F)F)OC2]

Diastereomer separation of 97 mg of 4-({(4S)-2-[(7S)-11-chloro-2-oxo-7-(trifluoromethyl)-7.8-dihydro-2H-[3]benzoxocino[5,6-c]pyridin-3(5H)-yl]-4-methoxypentanoyl}amino)benzamide (mixture of two stereoisomers), Example 66 gave
single stereoisomer 1 (the title compound Example 67) (chiral HPLC: Rₜ=1.44 min, 95% de): 16 mg,
single stereoisomer 2 (chiral HPLC: Rₜ=2.00 min, 99% de): 44 mg.

Separation method: HPLC: column: Daicel Chiralcel OD-H 5 μm, 250 mm×20 mm; eluent: 80% n-heptane/20% 2-propanol; temperature: 40° C.; flow rate: 20 ml/min; UV detection: 210 nm.

Analysis method: HPLC: column: Phen. Cellulose-1 3 μm, 50 mm×4.6 mm; eluent: 70% n-heptane/30% 2-propanol; temperature: 30° C.; flow rate: 1 m/min; UV detection: 220 nm.

¹H-NMR (400 MHz, DMSO-d₆): δ [ppm]=10.74-10.65 (m, 1H), 8.19 (s, 1H), 7.92-7.81 (m, 3H), 7.75-7.68 (m, 2H), 7.57 (dd, 1H), 7.45-7.38 (m, 2H), 7.30-7.20 (m, 1H), 6.43 (s, 1H), 5.81 (t, 1H), 4.80 (d, 1H), 4.23-4.12 (m, 1H), 3.75 (d, 1H), 3.28-3.05 (m, 3H), 3.17 (s, 3H), 2.28-2.18 (m, 2H), 1.14 (d, 3H). Additional signals of minor rotamers were also detected.

### Example 68

### 4-({(2S)-2-[(7S)-11-Chloro-12-fluoro-2-oxo-7-(trifluoromethyl)-7,8-dihydro-2H-[3]benzoxocino[5,6-c]pyridin-3(5H)-yl]butanoyl}amino)benzamide (Single Stereoisomer)

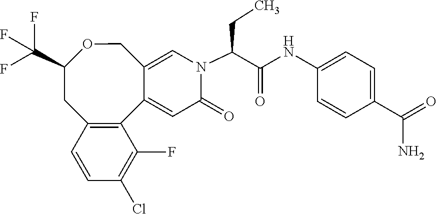 [SMILES: CC[C@@H](C(=O)Nc1ccc(C(N)=O)cc1)n1cc2c(cc1=O)-c1c(ccc(Cl)c1F)C[C@@H](C(F)(F)F)OC2]

General Method 7 was carried out with (7S)-11-chloro-12-fluoro-7-(trifluoromethyl)-3,5,7,8-tetrahydro-2H-[3]benzoxocino[5,6-c]pyridin-2-one (single stereoisomer) (175 mg, 503 μmol, 1.0 eq.), 4-{[(2R)-2-bromobutanoyl]amino}benzamide (single stereoisomer) (215 mg, 755 μmol, 1.5 eq.), 1,1,3,3-tetramethylguanidine (190 μl, 1.5 mmol, 3.0 eq.) in a mixture of 2-propanol/acetone (4:1, 5.0 ml) overnight including the following variations of the procedure: The crude mixture was purified by preparative HPLC (reversed phase, eluent: water with 0.05% formic acid/acetonitrile 80:20 to 5:95). Yield: 95.4 mg (31% of theory).

LC-MS (method 5): Rₜ=1.26 min; MS (ESIneg): m/z=550 [M−H]⁻

¹H-NMR (400 MHz, DMSO-d₆): δ [ppm]=10.74 (s, 1H), 8.21 (s, 1H), 7.91-7.81 (m, 3H), 7.75-7.64 (m, 3H), 7.29 (d, 1H), 7.25 (br s, 1H), 6.54 (d, 1H), 5.60 (dd, 1H), 4.81 (d, 1H), 4.24-4.11 (m, 1H), 3.82 (d, 1H), 3.17 (d, 1H), 2.47-2.42 (m, 1H), 2.27-2.06 (m, 2H), 0.89 (t, 3H). Additional signals of minor rotamers were also detected.

### Example 69

### 4-({(2S)-2-[(7S)-11-Chloro-12-fluoro-2-oxo-7-(trifluoromethyl)-7,8-dihydro-2H-[3]benzoxocino[5,6-c]pyridin-3(5H)-yl]-3-[(2S)-oxan-2-yl]propanoyl}amino)benzamide (Single Stereoisomer)

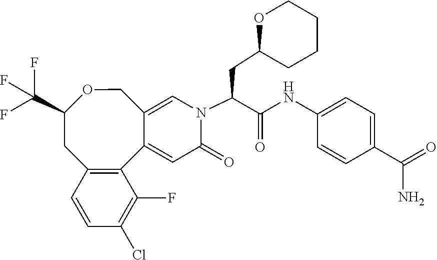 [SMILES: NC(=O)c1ccc(NC(=O)[C@H](C[C@@H]2CCCCO2)n2cc3c(cc2=O)-c2c(ccc(Cl)c2F)C[C@@H](C(F)(F)F)OC3)cc1]

General Method 7 was carried out with (7S)-11-chloro-12-fluoro-7-(trifluoromethyl)-3,5,7,8-tetrahydro-2H-[3]benzoxocino[5,6-c]pyridin-2-one (single stereoisomer) (30.0 mg, 86.3 μmol, 1.0 eq.), 4-({(2R)-2-bromo-3-[(2S)-oxan-2-yl]propanoyl}amino)benzamide (single stereoisomer) (46.0 mg, 129 μmol, 1.5 eq.) and 1,1,3,3-tetramethylguanidine (32 μl, 260 μmol, 3.0 eq.) in a mixture of 2-propanol/acetone (4:1, 1.0 ml) including the following variations of the procedure: The crude mixture was purified by preparative HPLC (reversed phase, eluent: water with 0.05% formic acid/acetonitrile 90:10 to 5:95). Yield: 15.0 mg (28% of theory).

LC-MS (method 5): Rₜ=1.34 min; MS (ESIneg): m/z=620 [M−H]⁻

¹H-NMR (400 MHz, DMSO-d₆): δ [ppm]=10.64 (s, 1H), 8.22 (s, 1H), 7.91-7.80 (m, 3H), 7.75-7.68 (m, 3H), 7.32-7.22 (m, 2H), 6.51 (d, 1H), 5.80 (t, 1H), 4.77 (d, 1H), 4.18-4.09 (m, 1H), 3.85-3.77 (m, 2H), 3.29-3.21 (m, 2H), 3.15 (d, 1H), 2.39-2.25 (m, 2H), 2.17-2.07 (m, 1H), 1.80-1.71 (m, 1H), 1.60 (d, 1H), 1.46-1.35 (m, 3H), 1.34-1.18 (m, 1H). Additional signals of minor rotamers were also detected.

### Example 70

### 4-({(2S)-2-[(7S)-11,12-Dichloro-2-oxo-7-(trifluoromethyl)-7,8-dihydro-2H-[3]benzoxocino[5,6-c]pyridin-3(5H)-yl]butanoyl}amino)benzamide (Single Stereoisomer)

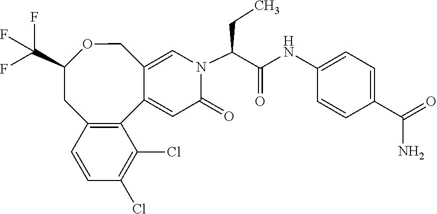 [SMILES: CC[C@@H](C(=O)Nc1ccc(C(N)=O)cc1)n1cc2c(cc1=O)-c1c(ccc(Cl)c1Cl)C[C@@H](C(F)(F)F)OC2]

General Method 7 was carried out with (7S)-11,12-dichloro-7-(trifluoromethyl)-3,5,7,8-tetrahydro-2H-[3]benzoxocino[5,6-c]pyridin-2-one (single stereoisomer) (30.0 mg, 82.4 μmol, 1.0 eq.), 4-{[(2R)-2-bromobutanoyl]amino}benzamide (single stereoisomer)(35.2 mg, 124 μmol, 1.5 eq.) and 1,1,3,3-tetramethylguanidine (31 μl, 250 μmol, 3.0 eq.) in a mixture of 2-propanol/acetone (4:1, 0.75 ml) including the following variations of the procedure: The crude mixture was purified by preparative HPLC (reversed phase, eluent: water with 0.05% formic acid/acetonitrile 90:10 to 5:95). Yield: 10.0 mg (21% of theory).

LC-MS (method 3): Rₜ=3.31 min; MS (ESIpos): m/z=568 [M+H]⁺

¹H-NMR (400 MHz, DMSO-d₆): δ [ppm]=10.73 (s, 1H), 8.22 (s, 1H), 7.90-7.82 (m, 3H), 7.77 (d, 1H), 7.69 (d, 2H), 7.41 (d, 1H), 7.25 (br s, 1H), 6.48 (s, 1H), 5.61 (dd, 1H), 4.77 (d, 1H), 4.16-4.08 (m, 1H), 3.79 (d, 1H), 3.17 (d, 1H), 2.48-2.42 (m, 1H), 2.27-2.16 (m, 1H), 2.16-2.06 (m, 1H), 0.89 (t, 3H). Additional signals of minor rotamers were also detected.

### Example 71

### 4-({(2S)-2-[(5S,7R)-11-Chloro-5,7-dimethyl-2-oxo-7,8-dihydro-2H-[3]benzoxocino[5,6-c]pyridin-3(5H)-yl]butanoyl}amino)-2-fluorobenzamide (Single Stereoisomer)

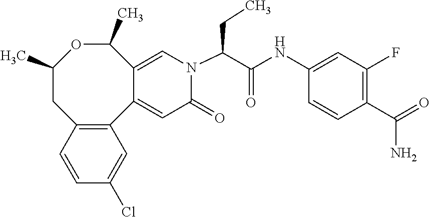 [SMILES: CC[C@@H](C(=O)Nc1ccc(C(N)=O)c(F)c1)n1cc2c(cc1=O)-c1cc(Cl)ccc1C[C@@H](C)O[C@H]2C]

General Method 7 was carried out with (5S,7R)-11-chloro-5,7-dimethyl-3,5,7,8-tetrahydro-2H-[3]benzoxocino[5,6-c]pyridin-2-one (single stereoisomer) (10.0 mg, 34.5 μmol, 1.0 eq.), 4-{[(2R)-2-bromobutanoyl]amino}-2-fluorobenzamide (single stereoisomer) (15.7 mg, 51.8 μmol, 1.5 eq.) and 1,1,3,3-tetramethylguanidine (13 μl, 100 μmol, 3.0 eq.) in a mixture of 2-propanol/acetone (4:1, 0.4 ml) including the following variations of the procedure: The crude mixture was purified by preparative HPLC (reversed phase, eluent: water with 0.05% formic acid/acetonitrile 80:20 to 1:99). Yield: 11.0 mg (62% of theory).

¹H-NMR (400 MHz, DMSO-d₆): δ [ppm]=10.88/10.86 (2 br s, 1H), 7.86 (s, 1H), 7.73-7.60 (m, 2H), 7.58-7.16 (m, 6H), 6.38/6.12 (2s, 1H), 5.62-5.44 (m, 1H), 4.80-4.66/3.72-3.58 (2m, 1H), 3.97-3.83 (m, 1H), 2.91-2.66 (m, 1H), 2.36-2.09 (m, 3H), 1.36/1.08 (2d, 3H), 1.24/1.06 (2d, 3H), 0.89 (t, 3H). Additional signals of minor rotamers were also detected.

### Example 72

### 4-({(2S)-2-[(5S,7R)-11-Chloro-5,7-dimethyl-2-oxo-7,8-dihydro-2H-[3]benzoxocino[5,6-c]pyridin-3(5H)-yl]-3-[(2S)-tetrahydro-2H-pyran-2-yl]propanoyl}amino)-2-fluorobenzamide (Single Stereoisomer)

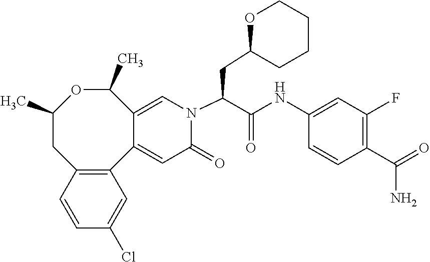 [SMILES: C[C@@H]1Cc2ccc(Cl)cc2-c2cc(=O)n([C@@H](C[C@@H]3CCCCO3)C(=O)Nc3ccc(C(N)=O)c(F)c3)cc2[C@H](C)O1]

General Method 7 was carried out with (5S,7R)-11-chloro-2-methoxy-5,7-dimethyl-7,8-dihydro-5H-[3]benzoxocino[5,6-c]pyridine (single stereoisomer) (40.0 mg, 138 μmol, 1.0 eq.), 4-({(2R)-2-bromo-3-[(2S)-oxan-2-yl]propanoyl}amino)-2-fluorobenzamide (single stereoisomer) (56.7 mg, 152 μmol, 1.1 eq.) and 1,1,3,3-tetramethylguanidine (52 μl, 410 μmol, 3.0 eq.) in a mixture of 2-propanol/acetone (4:1, 1.0 ml) including the following variations of the procedure: The crude mixture was purified by preparative HPLC (reversed phase, eluent: water with 0.05% formic acid/acetonitrile 90:10 to 1:99). Yield: 17.7 mg (22% of theory).

LC-MS (method 1): Rₜ=1.12 min; MS (ESIpos): m/z=582 [M+H]⁺

¹H-NMR (400 MHz, DMSO-d₆): δ [ppm]=10.79/10.76 (2br s, 1H), 7.91 (s, 1H), 7.77-7.61 (m, 2H), 7.56-7.14 (m, 6H), 6.36/6.10 (2s, 1H), 5.77-5.68 (m, 1H), 4.74-4.65/3.68-3.60 (2m, 1H), 3.93-3.80 (m, 2H), 3.29-3.18 (m, 2H), 2.95-2.65 (m, 1H), 2.37-2.23 (m, 2H), 2.18-2.07 (m, 1H), 1.80-1.70 (m, 1H), 1.65-1.55 (m, 1H), 1.47-1.36 (m, 3H), 1.35/1.07 (2d, 3H), 1.31-1.25 (m, 1H), 1.22/1.05 (2d, 3H). Additional signals of minor rotamers were also detected.

### Example 73

### 4-({(2S)-2-[(5S,7R)-11-Chloro-5,7-dimethyl-2-oxo-7,8-dihydro-2H-[3]benzoxocino[5,6-c]pyridin-3(5H)-yl]-3-[(2S)-tetrahydro-2H-pyran-2-yl]propanoyl}amino)benzamide (Single Stereoisomer)

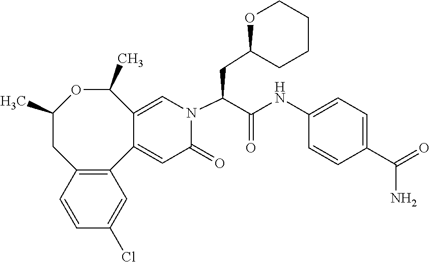 [SMILES: C[C@@H]1Cc2ccc(Cl)cc2-c2cc(=O)n([C@@H](C[C@@H]3CCCCO3)C(=O)Nc3ccc(C(N)=O)cc3)cc2[C@H](C)O1]

General Method 7 was carried out with (5S,7R)-11-chloro-5,7-dimethyl-3,5,7,8-tetrahydro-2H-[3]benzoxocino[5,6-c]pyridin-2-one (single stereoisomer) (79.0 mg, 273 μmol, 1.0 eq.), 4-({(2R)-2-bromo-3-[(2S)-oxan-2-yl]propanoyl}amino)benzamide (single stereoisomer) (145 mg, 409 μmol, 1.5 eq.) and 1,1,3,3-tetramethylguanidine (100 μl, 820 μmol, 3.0 eq.) in a mixture of 2-propanol/acetone (4:1, 4.0 ml) including the following variations of the procedure: The crude mixture was purified by preparative HPLC (reversed phase, eluent: water with 0.05% formic acid/acetonitrile 80:20 to 5:95). Yield: 61.5 mg (40% of theory).

LC-MS (method 1): Rₜ=1.07 min; MS (ESIpos): m/z=564 [M+H]⁺

¹H-NMR (500 MHz, DMSO-d₆): δ [ppm]=10.62 (br s, 1H), 7.93 (s, 1H), 7.90-7.82 (m, 3H), 7.77-7.67 (m, 2H), 7.52-7.11 (m, 4H), 6.36/6.10 (2s, 1H), 5.82-5.72 (m, 1H), 4.75-4.65/3.70-3.56 (m, 1H), 3.97-3.86 (m, 1H), 3.83 (d, 1H), 3.28-3.18 (m, 2H), 2.96-2.73 (m, 1H), 2.35-2.21 (m, 2H), 2.19-2.05 (m, 1H), 1.78-1.70 (m, 1H), 1.66-1.54 (m, 1H), 1.47-1.38 (m, 3H), 1.35/1.07 (2d, 3H), 1.32-1.24 (m, 1H), 1.22/1.05 (2d, 3H). Additional signals of minor rotamers were also detected.

### Example 74

### 4-({(2S,4S)-2-[(5S,7R)-11-Chloro-5,7-dimethyl-2-oxo-7,8-dihydro-2H-[3]benzoxocino[5,6-c]pyridin-3(5H)-yl]-4-methoxypentanoyl}amino)benzamide (Single Stereoisomer)

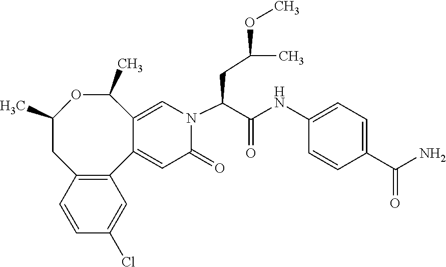 [SMILES: CO[C@@H](C)C[C@@H](C(=O)Nc1ccc(C(N)=O)cc1)n1cc2c(cc1=O)-c1cc(Cl)ccc1C[C@@H](C)O[C@H]2C]

General Method 7 was carried out with (5S,7R)-11-chloro-5,7-dimethyl-3,5,7,8-tetrahydro-2H-[3]benzoxocino[5,6-c]pyridin-2-one (single stereoisomer) (82.4 mg, 284 μmol, 1.3 eq.), 4-{[(2R,4S)-2-bromo-4-methoxypentanoyl]amino}benzamide (single stereoisomer)(72.0 mg, 219 μmol, 1.0 eq.), 1,1,3,3-tetramethylguanidine (82 μl, 660 μmol, 3.0 eq.) in a mixture of 2-propanol/acetone (4:1, 3.2 ml) for 2 days including the following variations of the procedure: The crude mixture was purified by preparative HPLC (reversed phase, eluent: water with 0.05% formic acid/acetonitrile 80:20 to 5:95). Yield: 76.9 mg (65% of theory).

LC-MS (method 1): Rₜ=1.02 min; MS (ESIpos): m/z=538 [M+H]⁺

¹H-NMR (500 MHz, CDCl₃): δ [ppm]=9.98/9.86 (2s, 1H), 7.89-7.76 (m, 2H), 7.75-7.61 (m, 3H), 7.42-7.21 (m, 2H), 7.18-7.01 (m, 1H), 6.58/6.39 (2s, 1H), 6.22-5.87 (m, 2H), 5.87-5.79 (m, 1H), 4.62-4.52/4.05-3.96 (2m, 1H), 3.95-3.86/3.72-3.63 (2m, 1H), 3.54-3.41 (m, 1H), 3.33 (s, 3H), 2.72-2.56 (m, 2H), 2.54-2.37 (m, 1H), 2.00-1.88 (m, 1H), 1.42 (d, 3H), 1.28/1.19 (2d, 3H), 1.26/1.16 (2d, 3H). Additional signals of minor rotamers were also detected.

### Example 75

### 4-({(2S)-2-[(5S,7R)-11-Chloro-12-fluoro-5,7-dimethyl-2-oxo-7,8-dihydro-2H-[3]benzoxocino[5,6-c]pyridin-3(5H)-yl]butanoyl}amino)benzamide (Single Stereoisomer)

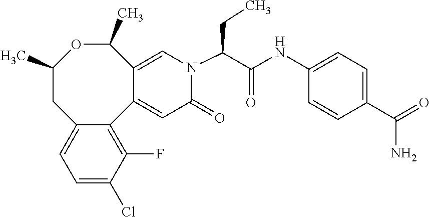 [SMILES: CC[C@@H](C(=O)Nc1ccc(C(N)=O)cc1)n1cc2c(cc1=O)-c1c(ccc(Cl)c1F)C[C@@H](C)O[C@H]2C]

General Method 7 was carried out with (7R)-11-chloro-12-fluoro-5,7-dimethyl-3,5,7,8-tetrahydro-2H-[3]benzoxocino[5,6-c]pyridin-2-one (mixture of two diastereomers) (15.0 mg, 48.7 μmol, 1.0 eq.), 4-{[(2R)-2-bromobutanoyl]amino}benzamide (single stereoisomer) (15.3 mg, 53.6 μmol, 1.1 eq.), 1,1,3,3-tetramethylguanidine (18 μl, 150 μmol, 3.0 eq.) in a mixture of 2-propanol/acetone (4:1, 0.75 ml) overnight including the following variations of the procedure: The crude mixture was purified by preparative HPLC (reversed phase, eluent: water with 0.05% formic acid/acetonitrile 80:20 to 5:95) and two diastereomers could be separated:
diastereomer 1 (LC-MS (method 1): Rₜ=0.93 min): 2.5 mg,
diastereomer 2 (the title compound Example 75) (LC-MS (method 1): Rₜ=0.99 min): 8.5 mg (34% of theory).

LC-MS (method 1): Rₜ=0.99 min; MS (ESIpos): m/z=512 [M+H]⁺

¹H-NMR (500 MHz, CDCl₃): δ [ppm]=9.80 (br s, 1H), 7.81-7.72 (m, 3H), 7.67 (br d, 2H), 7.47-7.35 (m, 1H), 6.97 (d, 1H), 6.68 (s, 1H), 6.20-5.56 (m, 2H), 5.49 (t, 1H), 3.99 (br d, 1H), 3.69-3.61 (m, 1H), 2.64 (d, 1H), 2.54-2.35 (m, 2H), 2.06-1.96 (m, 1H), 1.43 (d, 3H), 1.27 (d, 3H), 1.06 (t, 3H). Additional signals of minor rotamers were also detected.

### Example 76

### 4-({(2S)-2-[(7R)-11-Chloro-7-methyl-2-oxo-7,8-dihydro-2H-[3]benzoxocino[5,6-c]pyridin-3(5H)-yl]propanoyl}amino)benzamide (Single Stereoisomer)

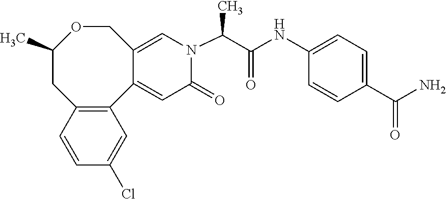 [SMILES: C[C@@H]1Cc2ccc(Cl)cc2-c2cc(=O)n([C@@H](C)C(=O)Nc3ccc(C(N)=O)cc3)cc2CO1]

1,1,3,3-Tetramethylguanidine (23 μl, 0.18 mmol, 3.0 eq.) was added under argon atmosphere at RT to a solution of (7R)-11-chloro-7-methyl-3,5,7,8-tetrahydro-2H-[3]benzoxocino[5,6-c]pyridin-2-one (single stereoisomer) (17 mg, 0.06 mmol) in 2-propanol/acetone (4:1, 1.0 ml). The mixture was stirred at RT for 15 min, followed by addition of 4-{[(2R)-2-bromopropanoyl]amino}benzamide (single stereoisomer) (18 mg, 0.07 mmol, 1.1 eq.) and of further 2-propanol/acetone (4:1, 1.0 ml). The reaction mixture was stirred at RT overnight and concentrated under reduced pressure. The residue was purified by preparative HPLC (reversed phase, eluent: acetonitrile/water gradient). Yield: 19 mg (68% of theory).

LC-MS (method 4): Rₜ=1.69 min; MS (ESIpos): m/z=466 [M+H]⁺

¹H-NMR (600 MHz, DMSO-d₆): δ [ppm]=10.61-10.54 (m, 1H), 7.89-7.81 (m, 3H), 7.79-7.74 (m, 1H), 7.70-7.64 (m, 2H), 7.48-7.28 (m, 3H), 7.22 (br s, 1H), 6.38-6.30 (m, 1H), 5.62-5.54 (m, 1H), 4.52-4.44/4.29-4.24/3.94-3.87/3.37-3.3 (4m, 2H, partially concealed), 2.90-2.81/2.76-2.70 (2m, 1H), 2.35-2.27 (m, 1H), 2.10-1.97 (m, 1H), 1.72-1.63 (m, 3H), 0.96-0.85 (m, 3H).

### Example 77

### 4-({(25)-2-[(7S)-11-Chloro-12-fluoro-2-oxo-7-(trifluoromethyl)-7,8-dihydro-2H-[3]benz-oxocino[5,6-c]pyridin-3(5H)-yl]propanoyl}amino)benzamide (Single Stereoisomer)

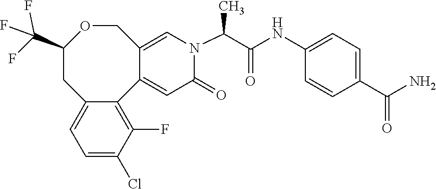 [SMILES: C[C@@H](C(=O)Nc1ccc(C(N)=O)cc1)n1cc2c(cc1=O)-c1c(ccc(Cl)c1F)C[C@@H](C(F)(F)F)OC2]

General Method 7 was carried out with (7S)-11-chloro-12-fluoro-7-(trifluoromethyl)-3,5,7,8-tetrahydro-2H-[3]benzoxocino[5,6-c]pyridin-2-one (single stereoisomer) (35.0 mg, 92% purity, 92.6 μmol, 1.0 eq.), 4-{[(2R)-2-bromopropanoyl]amino}benzamide (single stereoisomer) (30.1 mg, 111 μmol, 1.2 eq.), 1,1,3,3-tetramethylguanidine (35 μl, 280 μmol, 3.0 eq.) in a mixture of 2-propanol/acetone (4:1, 1.75 ml) overnight including the following variations of the procedure: The crude mixture was purified by preparative HPLC (reversed phase, eluent: water with 0.05% formic acid/acetonitrile 90:10 to 5:95). Yield: 40.0 mg (80% of theory).

LC-MS (method 3): Rₜ=2.75/2.80 min; MS (ESIneg): m/z=536 [M−H]⁻

¹H-NMR (400 MHz, DMSO-d₆): δ [ppm]=10.66 (s, 1H), 8.20/8.02 (2s, 1H), 7.85 (br d, 3H), 7.76-7.61 (m, 3H), 7.30 (d, 1H), 7.24 (br s, 1H), 6.53/6.41 (2d, 1H), 5.62-5.53 (m, 1H), 4.80 (d, 1H), 4.22-4.10 (m, 1H), 3.83 (d, 1H), 3.19 (d, 1H), 2.48-2.41 (m, 1H), 1.72 (d, 3H). Additional signals of minor rotamers were also detected.

### Example 78

### 4-({(2S,4S)-2-[(7S)-11-Chloro-12-fluoro-2-oxo-7-(trifluoromethyl)-7,8-dihydro-2H-[3]benz-oxocino[5,6-c]pyridin-3(5H)-yl]-4-methoxypentanoyl}amino)benzamide (Single Stereoisomer)

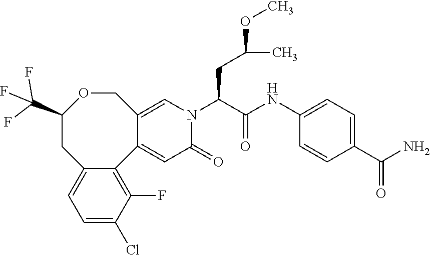 [SMILES: CO[C@@H](C)C[C@@H](C(=O)Nc1ccc(C(N)=O)cc1)n1cc2c(cc1=O)-c1c(ccc(Cl)c1F)C[C@@H](C(F)(F)F)OC2]

General Method 7 was carried out with (7S)-11-chloro-12-fluoro-7-(trifluoromethyl)-3,5,7,8-tetrahydro-2H-[3]benzoxocino[5,6-c]pyridin-2-one (single stereoisomer) (35.0 mg, 92% purity, 92.6 μmol, 1.0 eq.), 4-{[(2R,4S)-2-bromo-4-methoxypentanoyl]amino}benzamide (single stereoisomer) (36.6 mg, 111 μmol, 1.2 eq.), 1,1,3,3-tetramethylguanidine (35 μl, 280 μmol, 3.0 eq.) in a mixture of 2-propanol/acetone (4:1, 1.75 ml) overnight including the following variations of the procedure: The crude mixture was purified by preparative HPLC (reversed phase, eluent: water with 0.05% formic acid/acetonitrile 90:10 to 5:95). Yield: 35.0 mg (56% of theory).

LC-MS (method 3): Rₜ=3.08/3.13 min; MS (ESIpos): m/z=596 [M+H]⁺

¹H-NMR (400 MHz, DMSO-d₆): δ [ppm]=10.74/10.69 (2s, 1H), 8.27 (s, 1H), 7.91-7.81 (m, 3H), 7.75-7.67 (m, 3H), 7.29 (d, 1H), 7.25 (br s, 1H), 6.53/6.43 (2d, 1H), 5.83 (t, 1H), 4.78 (d, 1H), 4.14 (quin, 1H), 3.81 (d, 1H), 3.29-3.25 (m, 1H), 3.21-3.12 (m, 1H), 3.16-3.11 (m, 3H), 2.48-2.43 (m, 1H), 2.31-2.18 (m, 2H), 1.14 (d, 3H). Additional signals of minor rotamers were also detected.

### Example 79

### 4-({(2S,4S)-2-[(5S,7R)-11-Chloro-12-fluoro-5,7-dimethyl-2-oxo-7,8-dihydro-2H-[3]benzoxocino[5,6-c]pyridin-3(5H)-yl]-4-methoxypentanoyl}amino)benzamide (Single Stereoisomer)

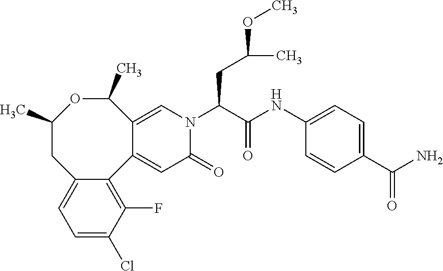 [SMILES: CO[C@@H](C)C[C@@H](C(=O)Nc1ccc(C(N)=O)cc1)n1cc2c(cc1=O)-c1c(ccc(Cl)c1F)C[C@@H](C)O[C@H]2C]

General Method 7 was carried out with (7R)-11-chloro-12-fluoro-5,7-dimethyl-3,5,7,8-tetrahydro-2H-[3]benzoxocino[5,6-c]pyridin-2-one (mixture of two diastereomers) (31.0 mg, 101 μmol, 1.0 eq.), 4-{[(2R,4S)-2-bromo-4-methoxypentanoyl]amino}benzamide (single stereoisomer) (33.2 mg, 101 μmol, 1.0 eq.), 1,1,3,3-tetramethylguanidine (38 μl, 300 μmol, 3.0 eq.) in a mixture of 2-propanol/acetone (4:1, 1.5 ml) overnight including the following variations of the procedure: The crude mixture was purified by preparative HPLC (reversed phase, eluent: water with 0.05% formic acid/acetonitrile 80:20 to 5:95) and two diastereomers could be separated:
diastereomer 1 (LC-MS (method 1): Rₜ=0.92 min): 10.1 mg,
diastereomer 2 (the title compound Example 79) (LC-MS (method 1): Rₜ=0.98 min): 16.3 mg (29% of theory).

LC-MS (method 1): Rₜ=0.98 min; MS (ESIpos): m/z=556 [M+H]⁺

¹H-NMR (500 MHz, DMSO-d₆): δ [ppm]=10.69 (s, 1H), 8.04 (s, 1H), 7.91-7.81 (m, 3H), 7.72 (d, 2H), 7.64 (dd, 1H), 7.30-7.20 (m, 2H), 6.47 (d, 1H), 5.83 (dd, 1H), 3.92 (q, 1H), 3.66-3.58 (m, 1H), 3.25-3.18 (m, 1H), 3.15 (s, 3H), 2.82 (br d, 1H), 2.36-2.18 (m, 3H), 1.38 (d, 3H), 1.22 (d, 3H), 1.14 (d, 3H).

### Example 80

### 4-({(2S,4S)-2-[(7S)-11,12-Dichloro-2-oxo-7-(trifluoromethyl)-7,8-dihydro-2H-[3]benzoxocino[5,6-c]pyridin-3(5H)-yl]-4-methoxypentanoyl}amino)benzamide (Single Stereoisomer)

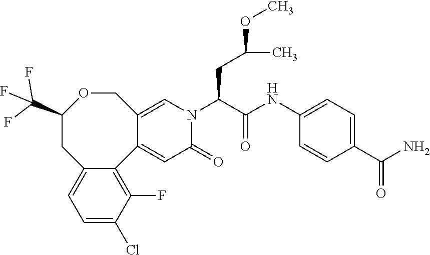 [SMILES: CO[C@@H](C)C[C@@H](C(=O)Nc1ccc(C(N)=O)cc1)n1cc2c(cc1=O)-c1c(ccc(Cl)c1F)C[C@@H](C(F)(F)F)OC2]

General Method 7 was carried out with (7S)-11,12-dichloro-7-(trifluoromethyl)-3,5,7,8-tetrahydro-2H-[3]benzoxocino[5,6-c]pyridin-2-one (single stereoisomer) (36.0 mg, 98.9 μmol, 1.0 eq.), 4-{[(2R,4S)-2-bromo-4-methoxypentanoyl]amino}benzamide (single stereoisomer) (32.5 mg, 98.9 μmol, 1.0 eq.), 1,1,3,3-tetramethylguanidine (37 μl, 300 μmol, 3.0 eq.) in a mixture of 2-propanol/acetone (4:1, 1.4 ml) overnight including the following variations of the procedure: The crude mixture was purified by preparative HPLC (reversed phase, eluent: water with 0.05% formic acid/acetonitrile 80:20 to 5:95). Yield: 32.5 mg (91% purity, 53% of theory).

LC-MS (method 3): Rₜ=3.19/3.25 min; MS (ESIpos): m/z=612 [M+H]⁺

¹H-NMR (500 MHz, DMSO-d₆): δ [ppm]=10.77/10.70 (2br s, 1H), 8.28/8.10 (2br s, 1H), 7.90-7.67 (m, 6H), 7.54-7.33 (m, 1H), 7.26 (br s, 1H), 6.47/6.33 (2br s, 1H), 5.92-5.76 (2m, 1H), 4.76/4.63 (2d, 1H), 4.27-4.05 (m, 1H), 3.79 (d, 1H), 3.30-3.20 (m, 1H), 3.21/3.16 (2s, 3H), 3.16-3.01 (m, 1H), 2.49-2.42 (m, 1H), 2.39-2.18 (m, 2H), 1.30-1.10 (m, 3H). Additional signals of minor rotamers were also detected.

### B) ASSESSMENT OF PHYSIOLOGICAL EFFICACY

The suitability of the compounds according to the invention for treating thromboembolic disorders can be demonstrated in the following assay systems:

### a) Test Descriptions (In Vitro)

### a.1) Measurement of FXIa Inhibition

The factor XIa inhibition of the substances according to the invention is determined using a biochemical test system which utilizes the reaction of a peptidic factor XIa substrate to determine the enzymatic activity of human factor XIa. Here, factor XIa cleaves from the peptidic factor XIa substrate the C-terminal aminomethylcoumarin (AMC), the fluorescence of which is measured. The determinations are carried out in microtitre plates.

Test substances are dissolved in dimethyl sulfoxide and serially diluted in dimethyl sulfoxide (3000 μM to 0.0078 μM; resulting final concentrations in the test: 50 μM to 0.00013 μM). In each case 1 μl of the diluted substance solutions is placed into the wells of white microtitre plates from Greiner (384 wells). 20 μl of assay buffer (50 mM of Tris/HCl pH 7.4; 100 mM of sodium chloride; 5 mM of calcium chloride; 0.1% of bovine serum albumin) and 20 μl of factor XIa from Kordia (0.45 nM in assay buffer) are then added successively. After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulfoxide instead of test substance in dimethyl sulfoxide), and IC₅₀ values are calculated from the concentration/activity relationships. Activity data from this test are listed in Table A below (some as mean values from multiple independent individual determinations):

TABLE A

| Example No. | IC₅₀ [nM] | Example No. | IC₅₀ [nM] |
|---|---|---|---|
| 1 | 11 | 2 | 5.5 |
| 3 | 6.0 | 4 | 2.4 |
| 5 | 4.3 | 6 | 1.4 |
| 7 | 14 | 8 | 6.6 |
| 9 | 4.5 | 10 | 1.2 |
| 11 | 19 | 12 | 3.3 |
| 13 | 4.6 | 14 | 3.9 |
| 15 | 3.4 | 16 | 3.5 |
| 17 | 2.0 | 18 | 3.7 |
| 19 | 3.6 | 20 | 6.6 |
| 21 | 63 | 22 | 19 |
| 23 | 22 | 24 | 5.4 |
| 25 | nd | 26 | 28 |
| 27 | 6.6 | 28 | 12 |
| 29 | 1.6 | 30 | 25 |
| 31 | 13 | 32 | 30 |
| 33 | 9.1 | 34 | 6.9 |
| 35 | 2.1 | 36 | 12 |
| 37 | 6.0 | 38 | nd |
| 39 | 21 | 40 | 41 |
| 41 | 10 | 42 | 3.6 |
| 43 | 1.7 | 44 | 49 |
| 45 | 17 | 46 | 110 |
| 47 | 56 | 48 | nd |
| 49 | 4.7 | 50 | 14 |
| 51 | 4.2 | 52 | 6.5 |
| 53 | 2.3 | 54 | 2.8 |
| 55 | 0.5 | 56 | 7.6 |
| 57 | 3.8 | 58 | 2.1 |
| 59 | 0.9 | 60 | 26 |
| 61 | 10 | 62 | 8.1 |
| 63 | 2.5 | 64 | 42 |
| 65 | 6.8 | 66 | 5.1 |
| 67 | 3.5 | 68 | 6.9 |
| 69 | 1.1 | 70 | 64 |
| 71 | 3.5 | 72 | 2.5 |
| 73 | 0.8 | 74 | 1.4 |
| 75 | 5.9 | 76 | 9.8 |
| 77 | 9.6 | 78 | 1.5 |
| 79 | 26 | 80 | 9.5 |

nd: not determined

### a.2) Determination of the Selectivity

To demonstrate the selectivity of the substances with respect to FXIa inhibition, the test substances are examined for their potential to inhibit other human serine proteases, such as factor Xa, trypsin and plasmin. To determine the enzymatic activity of factor Xa (1.3 nmol/I from Kordia), trypsin (83 mU/ml from Sigma) and plasmin (0.1 μg/ml from Kordia), these enzymes are dissolved (50 mmol/l of Tris buffer [C,C,C-tris(hydroxymethyl)aminomethane], 100 mmol/l of NaCl, 0.1% BSA [bovine serum albumin], 5 mmol/l of calcium chloride, pH 7.4) and incubated for 15 min with test substance in various concentrations in dimethyl sulfoxide and also with dimethyl sulfoxide without test substance. The enzymatic reaction is then started by addition of the appropriate substrates (5 μmol/I of Boc-Ile-Glu-Gly-Arg-AMC from Bachem for factor Xa and trypsin, 50 μmol/I of MeOSuc-Ala-Phe-Lys-AMC from Bachem for plasmin). After an incubation time of 30 min at 22° C., fluorescence is measured (excitation: 360 nm, emission: 460 nm). The measured emissions of the test mixtures with test substance are compared to the control mixtures without test substance (only dimethyl sulfoxide instead of test substance in dimethyl sulfoxide) and IC₅₀ values are calculated from the concentration/activity relationships.

### a.3) Thrombin Generation Assay (Thrombogram)

The effect of the test substances in the thrombin generation assay according to Hemker is determined in vitro in human plasma (Octaplas® from Octapharma).

In the thrombin generation assay according to Hemker, the activity of thrombin plasma is determined by measuring the fluorescent cleavage products of the substrate I-1140 (Z-Gly-Gly-Arg-AMC, Bachem). The reactions are carried out in the presence of varying concentrations of test substance or the corresponding solvent. To start the reaction, reagents from Thrombinoscope (30 μM to 0.1 μM recombinant tissue factor, 24 μM phospholipids in HEPES) are used. In addition, a thrombin calibrator from Thrombinoscope is used, of which the amidolytic activity is required for calculating the thrombin activity in a sample containing an unknown amount of thrombin. The test is carried out according to the manufacturer's instructions (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM Hepes, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nm filter pair and a dispenser.

Using the Thrombinoscope software, the thrombogram is calculated and represented graphically. The following parameters are calculated: lag time, time to peak, peak, ETP (endogenous thrombin potential) and start tail.

### a.4) Determination of Anticoagulatory Activity

The anticoagulatory activity of the test substances is determined in vitro in human plasma and rat plasma. Fresh whole blood is drawn directly into a mixing ratio of sodium citrate/blood of 1:9 using a 0.11 molar sodium citrate solution as receiver. Immediately after the blood has been drawn, it is mixed thoroughly and centrifuged at about 4000 g for 15 minutes. The supernatant is collected as (platelet-poor) plasma.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with plasma at 37° C. for 3 minutes. Coagulation is then started by addition of thromboplastin, and the timepoint, at which clotting of the sample occurs is determined. The concentration of test substance which effects a doubling of the prothrombin time is determined.

The activated partial thromboplastin time (APTT) is determined in the presence of varying concentrations of test substance or the corresponding solvent using a commercial test kit (PIT reagent from Roche). The test compounds are incubated with the plasma and the PITT reagent (cephalin, kaolin) at 37° C. for 3 minutes. Coagulation is then started by addition of 25 mM calcium chloride, and the time when coagulation occurs is determined. The concentration of test substance which leads to an extension by 50% or a doubling of the APTT is determined.

### a.5) Determination of the Plasma Kallikrein Activity

To determine the plasma kallikrein inhibition of the substances according to the invention, a biochemical test system is used which utilizes the reaction of a peptidic plasma kallikrein substrate to determine the enzymatic activity of human plasma kallikrein. Here, plasma kallikrein cleaves from the peptidic plasma kallikrein substrate the C-terminal aminomethylcoumarin (AMC), the fluorescence of which is measured. The determinations are carried out in microtitre plates.

Test substances are dissolved in dimethyl sulfoxide and serially diluted in dimethyl sulfoxide (3000 μM to 0.0078 μM; resulting final concentrations in the test: 50 μM to 0.00013 μM). In each case 1 μl of the diluted substance solutions is placed into the wells of white microtitre plates from Greiner (384 wells). 20 μl of assay buffer (50 mM Tris/HCl pH 7.4; 100 mM sodium chloride solution; 5 mM of calcium chloride solution; 0.1% of bovine serum albumin) and 20 μl of plasma kallikrein from Kordia (0.6 nM in assay buffer) are then added successively. After 15 min of incubation, the enzyme reaction is started by addition of 20 μl of the substrate H-Pro-Phe-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulfoxide instead of test substance in dimethyl sulfoxide), and IC₅₀ values are calculated from the concentration/activity relationships. Activity data from this test are listed in Table B below (some as mean values from multiple independent individual determinations):

TABLE B

| Example No. | IC₅₀ [nM] | Example No. | IC₅₀ [nM] |
|---|---|---|---|
| 1 | 630 | 2 | 320 |
| 3 | 510 | 4 | 420 |
| 5 | 400 | 6 | 140 |
| 7 | 780 | 8 | 320 |
| 9 | 260 | 10 | 87 |
| 11 | 190 | 12 | 42 |
| 13 | 205 | 14 | 220 |
| 15 | 140 | 16 | 110 |
| 17 | 72 | 18 | 140 |
| 19 | 110 | 20 | 93 |
| 21 | 830 | 22 | 280 |
| 23 | 410 | 24 | 110 |
| 25 | nd | 26 | 170 |
| 27 | 71 | 28 | 190 |
| 29 | 47 | 30 | 1300 |
| 31 | 980 | 32 | 1300 |
| 33 | 480 | 34 | 490 |
| 35 | 170 | 36 | 1000 |
| 37 | 320 | 38 | nd |
| 39 | 2000 | 40 | 4800 |
| 41 | 1600 | 42 | 810 |
| 43 | 380 | 44 | 1600 |
| 45 | 660 | 46 | 2300 |
| 47 | 1500 | 48 | nd |
| 49 | 370 | 50 | 1100 |
| 51 | 360 | 52 | 790 |
| 53 | 300 | 54 | 400 |
| 55 | 91 | 56 | 740 |
| 57 | 310 | 58 | 230 |
| 59 | 89 | 60 | 780 |
| 61 | 340 | 62 | 420 |
| 63 | 110 | 64 | 580 |
| 65 | 120 | 66 | 320 |
| 67 | 220 | 68 | 170 |
| 69 | 55 | 70 | 580 |
| 71 | 225 | 72 | 65 |
| 73 | 27 | 74 | 30 |
| 75 | 40 | 76 | 190 |
| 77 | 220 | 78 | 74 |
| 79 | 250 | 80 | 280 |

nd: not determined

### b) Determination of Antithrombotic Activity (In Vivo)

b.1) Arterial Thrombosis Model (Iron(II) Chloride-Induced Thrombosis) in Combination with Ear Bleeding Time in Rabbits

The antithrombotic activity of the FXIa inhibitors is tested in an arterial thrombosis model. Thrombus formation is triggered here by causing chemical injury to a region in the carotid artery in rabbits. Simultaneously, the ear bleeding time is determined.

Male rabbits (Crl:KBL (NZW)BR, Charles River) receiving a normal diet and having a body weight of 2.2-2.5 kg are anaesthetized by intramuscular administration of xylazine and ketamine (Rompun, Bayer, 5 mg/kg and Ketavet, Pharmacia & Upjohn GmbH, 40 mg/kg body weight). Anaesthesia is maintained by intravenous administration of the same preparations (continuous infusion) via the right auricular vein.

The right carotid artery is exposed and the vessel injury is caused by wrapping a piece of filter paper (10 mm×10 mm) on a Parafilm® strip (25 mm×12 mm) around the carotid artery without disturbing the blood flow. The filter paper contains 100 μL of a 13% strength solution of iron(II) chloride (Sigma) in water. After 5 min, the filter paper is removed and the vessel is rinsed twice with aqueous 0.9% strength sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h, respectively, before the injury.

Ear bleeding time is determined 2 min after injury to the carotid artery. To this end, the left ear is shaved and a defined 3-mm-long incision (blade Art. Number 10-150-10, Martin, Tuttlingen, Germany) is made parallel to the longitudinal axis of the ear. Care is taken not to damage any visible vessels. Any blood that extravasates is taken up in 15 second intervals using accurately weighed filter paper pieces, without touching the wound directly. Bleeding time is calculated as the time from making the incision to the point in time when no more blood can be detected on the filter paper. The volume of the extravasated blood is calculated after weighing of the filter paper pieces.

### c) Determination of Permeability (Caco Assay)

The Caco cells (obtained from the Deutsche Sammlung für Mikroorganismen and Zellkulturen, DSMZ) are cultivated in 24-well Transwell plates for 15 or 16 days. The test is carried out using a Hamilton robot. The density of the cell monolayers is ensured by measuring the Lucifer yellow permeability. The test compounds are dissolved in DMSO and then diluted with assay buffer to a concentration of 2 μM (final DMSO concentration 1%). The permeability is examined in both directions by addition of the substance solutions to the apical or basolateral compartment. The covered plates are incubated at 37° C. for 2 hours. The concentrations in the two compartments are determined by LC-MS/MS and the Papp values are calculated according to Artursson and Karlsson (PMID: 1673839).

### d) Determination of Pharmacokinetic Parameters Following Intravenous Administration

To examine the pharmacokinetic properties of a test substance, the respective test substances are administered to animals as a bolus injection, infusion or via oral administration. In the case of rats, the preferred formulation for intravenous administration of the test substances is plasma/dimethyl sulfoxide in a ratio of 99:1. The infusion solution of the test substance in the case of dogs and monkeys consists of polyethylene glycol/ethanol/water in a ratio of 50/10/40. Formulations for oral administration can be polyethylene glycol/ethanol/water or solutol/ethanol/water in a ratio of 50/10/40, or other formulations as appropriate (e.g. water, tylose, self-emulsifying drug dispering systems, etc.). The administration volume for rats is 2-10 ml/kg, for dogs and monkeys 0.5-5 ml/kg.

Blood samples are removed from the test animals into sodium EDTA (or other anticoagulant)-containing tubes: in the case of bolus administration, blood samples are usually taken at 0.033, 0.083, 0.167, 0.25, 0.283, 0.333, 0.5, 0.75, 1, 2, 3, 5, 7, 24 hours after administration of the test substance. In the case of infusions, blood samples are usually taken at 0.083, 0.167, 0.25, 0.283, 0.333, 0.5, 0.75, 1, 2, 3, 5, 7, 24 hours after administration of the test substance. In the case of oral administration, blood samples are usually taken at 0.083, 0.25, 0.5, 0.75, 1, 2, 3, 5, 7, 24 hours after administration of the test substance. Other time points might be chosen as appropriate.

After removal, the blood samples are centrifuged at 1280 g for 10 minutes. The supernatant (plasma) is taken off and either directly processed further or frozen for later sample preparation. For sample preparation, 50 μl of plasma are mixed with 250 μl of acetonitrile (the precipitating agent acetonitrile also contains the internal standard ISTD for later analytical determination) and then allowed to stand at room temperature for 5 minutes. The mixture is then centrifuged at 16 000 g for 3 minutes. The supernatant is taken off, and 500 μl of a buffer suitable for the mobile phase are added. The samples are then examined by LC-MS/MS analysis (e.g. liquid chromatography using a Gemini 5 μM C18 110A 50 mm×3 mm (or 150 mm×3 mm) column from Phenomenex; by mass spectrometry using an API 5500 or API 6500; SCIEX, Canada) to determine the concentration of the test substance in the individual samples.

In addition to the the plasma concentrations, the concentration ratio whole blood to plasma for the test substance in question is determined. To this end, the test substance is incubated at a certain concentration in whole blood for 20 minutes. The samples are then processed as described above to determine the concentration of the test substance in the plasma. The concentration set divided by the concentration measured in the plasma gives the parameter Cb/Cp.

The pharmacokinetic parameters are calculated by non-compartmental analysis (NCA). The algorithms for calculating the parameters are defined in an internal process description and are based on rules published in general textbooks of pharmacokinetics.

The primary pharmacokinetic parameters clearance (CL) and distribution volume (Vss) are calculated as follows:

| Parameter | Formula |
|---|---|
| CLplasma | CLplasma = dose/AUC (AUC = area |
| (plasma clearance) | under the curve) |
| CLblood | CLblood = CLplasma/(Cb/Cp) |
| (blood clearance) | |
| Vss | Vss = CLplasma * MRTiv |
| MRTiv | MRTiv = AUMC/AUC |
| AUMC | AUMC = AUMC(0-tₗₐₛₜ)*C_{last,calculated}/λ_{z} + |
| | C_{last,calculated}/λ_{z}² |
| λ_{z} | Rate constant for the terminal phase; |
| | calculated from the logarithmic-linear |
| | regression of unweighted data from the |
| | terminal phase with data |
| | points above the detection limit |
| AUC | AUC = AUC(0-tlast) + C_{last,calculatec}/λ_{z} |
| AUCnorm | AUC divided by dose (mg) per kg |
| | body weight |

### C) WORKING EXAMPLES OF PHARMACEUTICAL COMPOSITIONS

The substances according to the invention can be converted to pharmaceutical preparations as follows:

### Tablet:

### Composition:

100 mg of the compound of Example 1, 50 mg of lactose (monohydrate), 50 mg of maize starch, 10 mg of polyvinylpyrrolidone (PVP 25) (from BASF, Germany) and 2 mg of magnesium stearate.

Tablet weight 212 mg. Diameter 8 mm, radius of curvature 12 mm.

### Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tabletting press (see above for format of the tablet).

### Oral Suspension:

### Composition:

1000 mg of the compound of Example 1, 1000 mg of ethanol (96%), 400 mg of Rhodigel (xanthan gum) (from FMC, USA) and 99 g of water. 10 ml of oral suspension correspond to a single dose of 100 mg of the compound of the invention.

### Production:

The Rhodigel is suspended in ethanol, and the compound of Example 1 is added to the suspension. The water is added while stirring. The mixture is stirred for about 6 h until swelling of the Rhodigel is complete.

1. A compound of formula 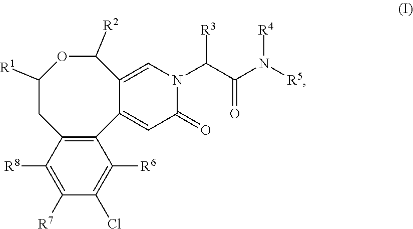
in which
R¹ represents methyl, ethyl, iso-propyl, hydroxymethyl, difluoromethyl or trifluoromethyl,
R² represents hydrogen or methyl,
R³ represents methyl, ethyl or n-propyl,
where methyl may be substituted with one substituent selected from the group consisting of cyclopropyl, cyclobutyl, oxetan-2-yl, oxetan-3-yl, tetrahydrofuran-2-yl, tetrahydro-2H-pyran-2-yl, tetrahydro-2H-pyran-4-yl and 1,4-dioxan-2-yl,
where oxetan-2-yl, tetrahydrofuran-2-yl, tetrahydro-2H-pyran-2-yl and 1,4-dioxan-2-yl may be substituted by 1 to 2 substituents independently of one another selected from the group consisting of fluorine and methyl,
or
where methyl may be substituted with one substituent of the group of formula 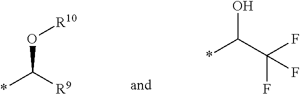
where
* is the attachment site to the methyl group,
R⁹ represents methyl, ethyl, iso-propyl, cyclopropyl, difluoromethyl or trifluoromethyl,
R¹⁰ represents methyl or difluoromethyl,
and
where ethyl may be substituted with one substituent selected from the group consisting of methoxy, ethoxy, iso-propoxy, tert-butoxy, difluoromethoxy, trifluoromethoxy, 2,2-difluoroethoxy, 2,2,2-trifluoroethoxy, cyclopropyloxy and cyclobutyloxy,
where cyclopropyloxy and cyclobutyloxy may be substituted with one substituent selected from the group consisting of fluorine and methyl,
R⁴ represents hydrogen,
R⁵ represents a group of formula 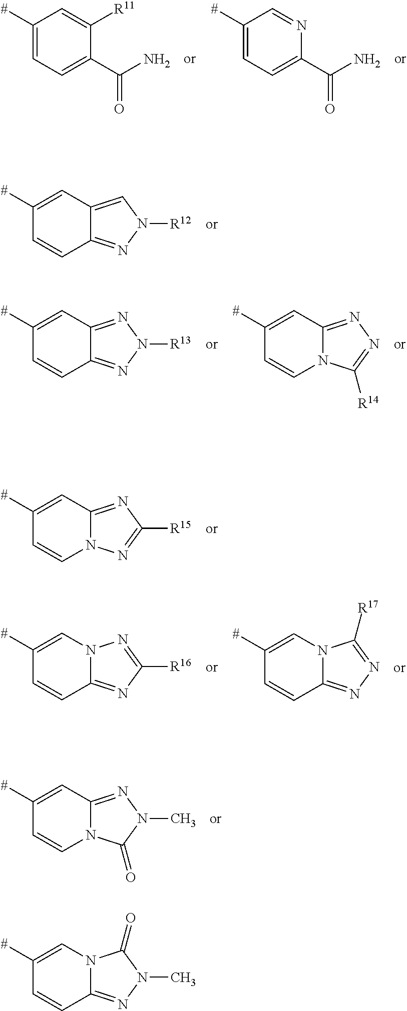
where
# is the attachment site to the nitrogen atom,
R¹¹ represents hydrogen or fluorine,
R¹² represents methyl, difluoromethyl or trifluoromethyl,
R¹³ represents methyl, difluoromethyl or trifluoromethyl,
R¹⁴ represents hydrogen or methyl,
R¹⁵ represents hydrogen or methyl,
R¹⁶ represents hydrogen or methyl,
R¹⁷ represents hydrogen or methyl,
R⁶, R⁷ and R⁸ represent the following:
R⁶ represents hydrogen, fluorine or chlorine,
R⁷ represents hydrogen,
R⁸ represents hydrogen,
or
R⁶ represents hydrogen,
R⁷ represents fluorine or chlorine,
R⁸ represents hydrogen,
or
R⁶ represents hydrogen,
R⁷ represents hydrogen,
R⁸ represents fluorine,
And/or a salt thereof, solvate thereof and/or solvate of a salt thereof.

2. The compound according to claim 1, wherein
R¹ represents methyl, ethyl, iso-propyl, hydroxymethyl, difluoromethyl or trifluoromethyl,
R² represents hydrogen or methyl,
R³ represents methyl or ethyl,
where methyl may be substituted with one substituent selected from the group consisting of tetrahydro-2H-pyran-2-yl and 1,4-dioxan-2-yl,
or
where methyl may be substituted with one substituent of the group of formula 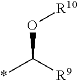
where
* is the attachment site to the methyl group,
R⁹ represents methyl, cyclopropyl or difluoromethyl,
R¹⁰ represents methyl or difluoromethyl,
and
where ethyl may be substituted with one substituent selected from the group consisting of methoxy, iso-propoxy, tert-butoxy, difluoromethoxy and cyclopropyloxy,
R⁴ represents hydrogen,
R⁵ represents a group of formula 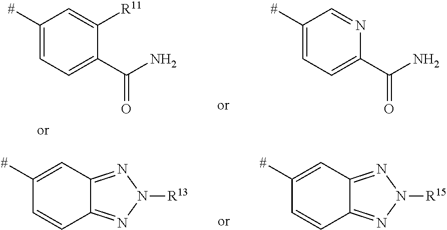
where
# is the attachment site to the nitrogen atom,
R¹¹ represents hydrogen or fluorine,
R¹³ represents methyl,
R¹⁵ represents hydrogen,
R⁶, R⁷ and R⁸ represent the following:
R⁶ represents hydrogen, fluorine or chlorine,
R⁷ represents hydrogen,
R⁸ represents hydrogen,
And/or a salt thereof, solvate thereof and/or solvate of a salt thereof.

3. The compound according to claim 1, wherein
R¹ represents methyl, difluoromethyl or trifluoromethyl,
R² represents hydrogen or methyl,
R³ represents methyl or ethyl,
where methyl is substituted with one substituent selected from the group consisting of tetrahydro-2H-pyran-2-yl and 1,4-dioxan-2-yl,
or
where methyl is substituted with one substituent of the group of formula 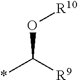
where
* is the attachment site to the methyl group,
R⁹ represents methyl,
R¹⁰ represents methyl or difluoromethyl,
and
where ethyl is substituted with one substituent selected from the group consisting of methoxy, iso-propoxy, tert-butoxy, difluoromethoxy and cyclopropyloxy,
R⁴ represents hydrogen,
R⁵ represents a group of formula 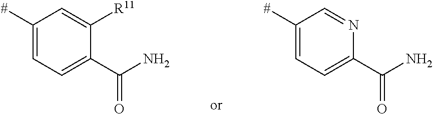
where
# is the attachment site to the nitrogen atom,
R¹¹ represents hydrogen or fluorine,
R⁶, R⁷ and R⁸ represent the following:
R⁶ represents hydrogen or fluorine,
R⁷ represents hydrogen,
R⁸ represents hydrogen,
And/or a salt thereof, solvate thereof and/or solvate of a salt thereof.

4. The compound according to claim 1, comprising formula (Ia)

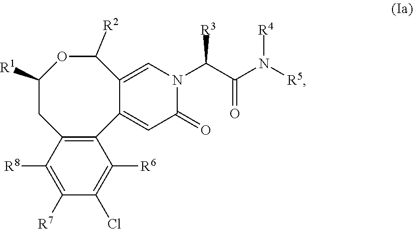

5. 4-({(2S,4S)-2-[(5S,7R)-11-Chloro-5,7-dimethyl-2-oxo-7,8-dihydro-2H-[3]benzoxo-cino[5,6-c]pyridin-3(5H)-yl]-4-methoxypentanoyl}-amino)benzamide (single stereoisomer) according to claim 1 of formula below 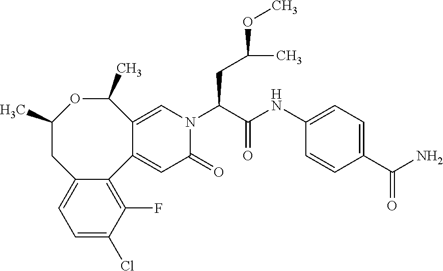 [SMILES: CO[C@@H](C)C[C@@H](C(=O)Nc1ccc(C(N)=O)cc1)n1cc2c(cc1=O)-c1c(ccc(Cl)c1F)C[C@@H](C)O[C@H]2C]
And/or a salt thereof, solvate thereof and/or solvate of a salt thereof.

6. A process for preparing a compound of formula (I) and/or a salt thereof, solvate thereof and/or solvate of a salt thereof, according to claim 1 comprising
[A] reacting a compound of formula 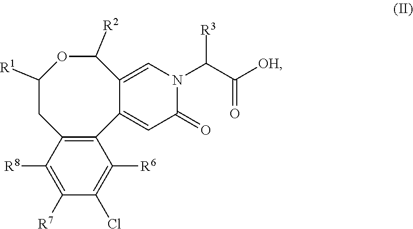
with a compound of formula 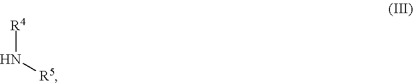
in the presence of a dehydrating agent to give a compound of formula (I)
or
[B] converting a compound of formula (II) in a one-pot reaction to an acid chloride of the compound of formula (II) and then the acid chloride is reacted with a compound of formula (III) to give a compound of formula (I)
or
[C] reacting a compound of formula 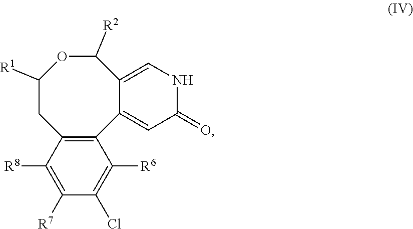
with a compound of formula 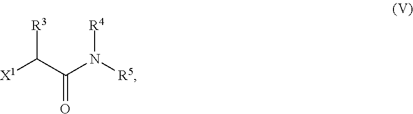
in which
X¹ represents bromine, iodine or trifluoromethane-sulfonyloxy,
in the presence of a base to give a compound of formula (I).

7. The compound according to claim 1 for treatment and/or prophylaxis of a disease.

8. A product comprising a compound according to claim 1 for treatment and/or prophylaxis of a disease.

9. A product comprising a compound according to claim 1 for treatment and/or prophylaxis of a thrombotic or thromboembolic disorder.

10. A product comprising a compound according to claim 1 for treatment and/or prophylaxis of a disorder in a coronary artery of the heart, optionally acute coronary syndrome (ACS), myocardial infarction with ST segment elevation (STEMI) and without ST segment elevation (non-STEMI), stable angina pectoris, unstable angina pectoris, stent thrombosis, reocclusions and restenoses after coronary interventions optionally angioplasty, stent implantation or aortocoronary bypass, disorders in the cerebrovascular arteries, optionally transitory ischaemic attacks (TIA), ischemic strokes including cardioembolic strokes, optionally strokes due to atrial fibrillation, non-cardioembolic strokes, such as lacunar stroke, strokes due to large or small artery diseases, or strokes due to undetermined cause, cryptogenic strokes, embolic strokes, embolic strokes of undetermined source, or events of thrombotic and/or thromboembolic origin leading to stroke or TIA, and disorders of peripheral arteries, leading to peripheral artery disease, including peripheral artery occlusion, acute limb ischemia, amputation, reocclusions and restenoses after interventions optionally angioplasty, stent implantation or surgery and bypass.

11. A medicament comprising a compound according to claim 1 in combination with an inert, nontoxic, pharmaceutically suitable excipient.

12. The medicament according to claim 11 for the treatment and/or prophylaxis of a thrombotic or thromboembolic disorder.

13. The compound according to claim 1 for treatment and/or prophylaxis of a thrombotic or thromboembolic disorder in a therapeutically effective amount thereof.

14. A method for treatment and/or prophylaxis of a thrombotic or thromboembolic disorder in a human and/or animal comprising administering a therapeutically effective amount of at least one compound according to claim 1, of a medicament thereof.
